(19) United States
(12) Patent Application Publication
(10) Pub. No.: US 2026/0275379 A1
(43) Pub. Date: Sep. 17, 2026

(54) ADENO-ASSOCIATED VIRUS COMPOSITIONS FOR THE TREATMENT OF FACIOSCAPULOHUMERAL MUSCULAR DYSTROPHY

(71) Applicant: KATE THERAPEUTICS, INC., San Diego, CA (US)

(72) Inventors: Saurav SESHADRI, San Diego, CA (US); Sharif TABEBORDBAR, San Diego, CA (US); Shayan TABEBORDBAR, San Diego, CA (US); Simon YE, San Diego, CA (US)

(21) Appl. No.: 19/643,597

(22) Filed: Apr. 9, 2026

Related U.S. Application Data
(63) Continuation of application No. PCT/US2025/039526, filed on Jul. 28, 2025.
(60) Provisional application No. 63/676,790, filed on Jul. 29, 2024.
(60) Provisional application No. 63/687,992, filed on Aug. 28, 2024.
(60) Provisional application No. 63/788,362, filed on Apr. 14, 2025.

(51) Int. Cl. C12N 15/86; A61P 21/00; C07K 14/005; C12N 15/113

(52) U.S. Cl. CPC C12N 15/86; A61P 21/00; C07K 14/005; C12N 15/113; C12N 2310/141; C12N 2310/531; C12N 2750/14122; C12N 2750/14143; C12N 2830/008; C12N 2830/50; C12N 2830/60

(57) ABSTRACT

The present invention provides novel gene therapy compositions for use in treatment of facioscapulohumeral muscular dystrophy (FSHD).

### CLAIM OF PRIORITY

This application claims the benefit of U.S. Provisional Patent Application No. 63/676,790, filed Jul. 29, 2024; U.S. Provisional Patent Application No. 63/687,992, filed Aug. 28, 2024; and U.S. Provisional Patent Application No. 63/788,362, filed Apr. 14, 2025; each of which is incorporated by reference herein in its entirety.

### SEQUENCE LISTING

This application contains a Sequence Listing that has been submitted electronically as an XML file named PAT059899-WO-PCT_SL.xml. The XML file, created on Jul. 16, 2025, is 3,505,945 bytes in size. The material in the XML file is hereby incorporated by reference in its entirety.

### FIELD OF DISCLOSURE

This disclosure relates to compositions for the treatment of Facioscapulohumeral Muscular Dystrophy (FSHD).

### BACKGROUND

Genetic disorders are a major source of disease burden, and many of them have few or no medical or curative treatments. Along with other genetically linked muscle wasting disorders, facioscapulohumeral muscular dystrophy (FSHD) is a genetic disorder that causes progressively increasing weakness and atrophy of the muscles. FSHD is so named because the muscles in the face, shoulder, and upper arm are the most affected, but it also affects other muscles in the body, including the legs, eyes, heart, hip, or abdominal muscles. FSHD causes progressively worsening symptoms that may result in asymmetric weakness

FSHD is a genetic disorder caused by a genetic mutation that leads to inappropriate expression of the DUX4 gene on chromosome 4. DUX4 is the double homeobox protein 4 gene. FSHD can be inherited by just one parent because it is an autosomal dominant genetic disorder. FSHD often affects patients before the age of 20 and has an estimated prevalence in the United States of 4 cases per 100,000 individuals. There is currently no medical treatment to arrest or reverse the muscular effects of FSHD.

### SUMMARY

The present disclosure is directed to compositions (e.g., adeno-associated viral (AAV) compositions) and methods of use of such compositions for treating facioscapulohumeral muscular dystrophy (FSHD).

Accordingly, aspects of the invention provide a composition comprising an AAV particle comprising (e.g., encapsidating) a recombinant nucleic acid comprising a promoter and an engineered primary microRNA (pri-miRNA), which comprises a microRNA (miRNA) scaffold comprising the miR-138 or miR-139 scaffold and an artificial miRNA that targets a human double homeobox protein 4 (DUX4) transcript.

Aspects of the invention provide a composition comprising an adeno-associated virus (AAV) particle comprising an engineered capsid protein comprising the amino acid sequence RGDR. The capsid protein encapsidated a recombinant nucleic acid molecule (e.g., a cargo) comprising a promoter and a primary microRNA (pri-miRNA) scaffold having the scaffold sequence of miR-138 or miR-139 and an artificial miRNA guide sequence within the scaffold is that targets a DUX4 transcript. The recombinant nucleic acid may further comprise a dorsal root ganglia (DRG) de-targeting miRNA binding site(s) and/or a heart de-targeting miRNA binding site(s).

For example, the guide sequence (e.g. the DUX4 targeting sequence) comprises or consists of a nucleic acid sequence of any one of SEQ ID NOs: 2399-3159 and 3952-3953.

The DRG de-targeting miRNA binding site(s) may comprise or consist of a nucleic acid sequence of any one of SEQ ID NOs: 3921-3923.

The heart de-targeting miRNA binding site may comprise or consist of a nucleic acid sequence of any one of SEQ ID NO: 3924 and SEQ ID NO: 3925.

The promoter may comprise or consist of a sequence having at least 95% sequence identity with a nucleic acid sequence of any one of SEQ ID NOs: 3926-3928. For example, the nucleic acid molecule may comprise in order:
a first inverted terminal repeat (ITR) region;
the promoter;
the primary microRNA (pri-miRNA) comprising in order:
   an upstream scaffold;
   the guide sequence;
   a short hairpin loop;
   a passenger sequence; and
   a downstream scaffold,
a first DRG de-targeting miRNA binding site;
a first heart de-targeting miRNA binding site;
a second DRG de-targeting miRNA binding site;
a second heart de-targeting miRNA binding site;
a poly adenylation signal;
a second ITR region. The ITR regions may be AAV2 ITR regions derived from an AAV2.

For example, the full nucleic acid molecule may comprise a sequence selected from among SEQ ID NOs: 3940-3947.

Advantageously, when administered to a subject with a FSHD, that results in toxic DUX4 expression, the composition is effective at restoring a healthy phenotype.

The engineered capsid protein may advantageously exhibit decreased liver tropism and/or increased muscle tropism.

For example, the capsid protein may comprise an amino acid sequence selected from is Table 1a in hypervariable region IV (HVR IV) relative to wild-type AAV9. The capsid protein may comprise substitutions at amino acids 451-455 relative to a wild-type AAV9 vector capsid selected from column 1 of Table 1b and a 7-mer insert selected from column 2 of Table 1b inserted after amino acid 455 relative to a wild-type AAV9 vector. The capsid protein may further comprise a deletion of G267 relative to wild-type AAV9 capsid or equivalent position in another AAV capsid.

Accordingly, aspects of the invention further provide methods and uses of the compositions of the invention (for example in the preparation of a medicament) for treating a subject suffering from FSHD. The methods and uses may comprise providing to a subject a composition of the invention as described above.

Aspects of the present disclosure provide a recombinant nucleic acid comprising a primary microRNA (pri-miRNA) comprising:
a scaffold sequence;
a guide strand that is at least partially complementary to a double homeobox 4 (DUX4) sequence; and
a passenger strand that is at least partially complementary to the guide strand;
wherein the guide strand and the passenger strand are positioned within the scaffold sequence.

In some embodiments, the scaffold sequence is derived from an endogenous primary microRNA (pri-miRNA). In some embodiments, the endogenous pri-miRNA is pri-miR-138 or pri-miR-139. In some embodiments, the scaffold sequence comprises an upstream scaffold sequence, a loop scaffold sequence, and a downstream scaffold sequence. In some embodiments, the guide strand is positioned between the upstream scaffold sequence and the loop scaffold sequence, and the passenger strand is positioned between the loop scaffold sequence and the downstream scaffold sequence.

In some embodiments, the upstream scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933; the loop scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and the downstream scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935.

In some embodiments, the guide strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953. In some embodiments, the guide strand comprises or consists of the nucleic acid sequence of any one of SEQ ID NOs: 2399-3159 and 3952-3953.

In some embodiments, the passenger strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920. In some embodiments, the passenger strand comprises or consists of the nucleic acid sequence of any one of SEQ ID NOs: 3160-3920.

In some embodiments, the pri-miRNA comprises:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920;
wherein the guide strand is positioned between the upstream scaffold sequence and the loop scaffold sequence, and the passenger strand is positioned between the loop scaffold sequence and the downstream scaffold sequence.

In some embodiments, the recombinant nucleic acid is selected from:
(a) a recombinant nucleic acid comprising:
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
(b) a recombinant nucleic acid comprising:
   a upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920;
(c) a recombinant nucleic acid comprising:
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
(d) a recombinant nucleic acid comprising:
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
(e) a recombinant nucleic acid comprising:
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630; and
(f) a recombinant nucleic acid comprising:
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429; and
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190.

In some embodiments, the recombinant nucleic acid is selected from:
(a) a recombinant nucleic acid comprising:
   an upstream scaffold sequence comprising or consisting of the nucleic acid sequence of SEQ ID NO: 3933;
   a loop scaffold sequence comprising or consisting of the nucleic acid sequence of SEQ ID NO: 3934;
   a downstream scaffold sequence comprising or consisting of the nucleic acid sequence of SEQ ID NO: 3935;
   a guide strand comprising or consisting of the nucleic acid sequence of SEQ ID NO: 2475; and
   a passenger strand comprising or consisting of the nucleic acid sequence of SEQ ID NO: 3236;
(b) a recombinant nucleic acid comprising:
   an upstream scaffold sequence comprising or consisting of the nucleic acid sequence of SEQ ID NO: 3933;
   a loop scaffold sequence comprising or consisting of the nucleic acid sequence of SEQ ID NO: 3934;
   a downstream scaffold sequence comprising or consisting of the nucleic acid sequence of SEQ ID NO: 3935;
   a guide strand comprising or consisting of the nucleic acid sequence of SEQ ID NO: 3159; and
   a passenger strand comprising or consisting of the nucleic acid sequence of SEQ ID NO: 3920;
(c) a recombinant nucleic acid comprising:
   an upstream scaffold sequence comprising or consisting of the nucleic acid sequence of SEQ ID NO: 3929;
   a loop scaffold sequence comprising or consisting of the nucleic acid sequence of SEQ ID NO: 3930;
   a downstream scaffold sequence comprising or consisting of the nucleic acid sequence of SEQ ID NO: 3931;
   a guide strand comprising or consisting of the nucleic acid sequence of SEQ ID NO: 3047; and
   a passenger strand comprising or consisting of the nucleic acid sequence of SEQ ID NO: 3808;
(d) a recombinant nucleic acid comprising:
   an upstream scaffold sequence comprising or consisting of the nucleic acid sequence of SEQ ID NO: 3929;
   a loop scaffold sequence comprising or consisting of the nucleic acid sequence of SEQ ID NO: 3930;
   a downstream scaffold sequence comprising or consisting of the nucleic acid sequence of SEQ ID NO: 3931;
   a guide strand comprising or consisting of the nucleic acid sequence of SEQ ID NO: 2475; and
   a passenger strand comprising or consisting of the nucleic acid sequence of SEQ ID NO: 3236;
(e) a recombinant nucleic acid comprising:
   an upstream scaffold sequence comprising or consisting of the nucleic acid sequence of SEQ ID NO: 3933;
   a loop scaffold sequence comprising or consisting of the nucleic acid sequence of SEQ ID NO: 3934;
   a downstream scaffold sequence comprising or consisting of the nucleic acid sequence of SEQ ID NO: 3935;
   a guide strand comprising or consisting of the nucleic acid sequence of SEQ ID NO: 2869; and
   a passenger strand comprising or consisting of the nucleic acid sequence of SEQ ID NO: 3630; and
(f) a recombinant nucleic acid comprising:
   an upstream scaffold sequence comprising or consisting of the nucleic acid sequence of SEQ ID NO: 3933;
   a loop scaffold sequence comprising or consisting of the nucleic acid sequence of SEQ ID NO: 3934;
   a downstream scaffold sequence comprising or consisting of the nucleic acid sequence of SEQ ID NO: 3935;
   a guide strand comprising or consisting of the nucleic acid sequence of SEQ ID NO: 2429; and
   a passenger strand comprising or consisting of the nucleic acid sequence of SEQ ID NO: 3190.

In some embodiments, the recombinant nucleic acid further comprises at least one dorsal root ganglia (DRG) de-targeting miRNA binding site. In some embodiments, the at least one DRG de-targeting miRNA binding site comprises a binding site for miR-338, miR-138, or miR-9. In some embodiments, the at least one DRG de-targeting miRNA binding site comprises a binding site for miR-338-3p, miR-138-5p, or miR-9-5p. In some embodiments, the at least one DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923. In some embodiments, the at least one DRG de-targeting miRNA binding site comprises or consists of the nucleic acid sequence of any one of SEQ ID NOs: 3921-3923. In some embodiments, the at least one DRG de-targeting miRNA binding site comprises a first and a second DRG de-targeting miRNA binding site. In some embodiments, each of the first and the second DRG de-targeting miRNA binding sites comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923. In some embodiments, each of the first and the second DRG de-targeting miRNA binding sites comprises or consists of the nucleic acid sequence of any one of SEQ ID NOs: 3921-3923. In some embodiments, each of the first and the second DRG de-targeting miRNA binding sites comprises or consists of the nucleic acid sequence of SEQ ID NO: 3921.

In some embodiments, the recombinant nucleic acid further comprises at least one heart de-targeting miRNA binding site. In some embodiments, the at least one heart de-targeting miRNA binding site comprises a binding site for miR-221 or miR-499. In some embodiments, the at least one heart de-targeting miRNA binding site comprises a binding site for miR-221-3p or miR-499a. In some embodiments, the at least one heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925. In some embodiments, the at least one heart de-targeting miRNA binding site comprises or consists of the nucleic acid sequence of SEQ ID NO: 3924 or SEQ ID NO: 3925. In some embodiments, the at least one heart de-targeting miRNA binding site comprises a first and a second heart de-targeting miRNA binding site. In some embodiments, each of the first and the second heart de-targeting miRNA binding sites comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925. In some embodiments, each of the first and the second heart de-targeting miRNA binding sites comprises or consists of the nucleic acid sequence of SEQ ID NO: 3924 or SEQ ID NO: 3925. In some embodiments, each of the first and the second heart de-targeting miRNA binding sites comprises or consists of the nucleic acid sequence of SEQ ID NO: 3924. In some embodiments, the recombinant nucleic acid comprises at least two DRG de-targeting miRNA binding sites and at least two heart de-targeting miRNA binding sites, which are in alternating positions.

In some embodiments, the recombinant nucleic acid comprises, from 5′ to 3′:
a first DRG de-targeting miRNA binding site,
a first heart de-targeting miRNA binding site,
a second DRG de-targeting miRNA binding site, and
a second heart de-targeting miRNA binding site.

In some embodiments, the recombinant nucleic acid further comprises a muscle specific promoter. In some embodiments, the muscle specific promoter comprises a CK8 promoter, a desmin promoter, a Mb promoter, a MCK promoter, a MHCK7 promoter, a skeletal muscle alpha actin promoter, or a TTNI2 promoter. In some embodiments, the muscle specific promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928. In some embodiments, the muscle specific promoter comprises or consists of the nucleic acid sequence of any one of SEQ ID NOs: 3926-3928. In some embodiments, the muscle specific promoter comprises or consists of the nucleic acid sequence of SEQ ID NO: 3926.

In some embodiments, the recombinant nucleic acid further comprises a polyadenylation (PolyA) signal. In some embodiments, the PolyA signal comprises a bovine growth hormone polyadenylation (bgh-PolyA) signal, a synthetic PolyA signal, or an SV40 PolyA signal. In some embodiments, the PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938. In some embodiments, the PolyA signal comprises or consists of the nucleic acid sequence of SEQ ID NO: 3938.

In some embodiments, the recombinant nucleic acid further comprises a 5′ inverted terminal repeat (ITR) and a 3′ ITR. In some embodiments, the 5′ ITR and/or the 3′ ITR is an adeno-associated virus 2 (AAV2) ITR, or a variant or fragment thereof. In some embodiments, the 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937. In some embodiments, the 5′ ITR comprises or consists of the nucleic acid sequence of SEQ ID NO: 3937. In some embodiments, the 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939. In some embodiments, the 3′ ITR comprises or consists of the nucleic acid sequence of SEQ ID NO: 3939.

In some embodiments, the recombinant nucleic acid comprises, optionally from 5′ to 3′:
a 5′ ITR;
a muscle specific promoter;
an upstream scaffold sequence;
a guide strand targeting a DUX4 sequence;
a loop scaffold sequence;
a passenger strand;
a downstream scaffold sequence;
a first DRG de-targeting miRNA binding site;
a first heart de-targeting miRNA binding site;
a second DRG de-targeting miRNA binding site;
a second heart de-targeting miRNA binding site;
a PolyA signal; and
a 3′ ITR.

In some embodiments, the recombinant nucleic acid comprises, optionally from 5′ to 3′:
the 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
the muscle specific promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928;
the upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933;
the guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and
the passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920;
the downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935;
the first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
the first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
the second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
the second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
the PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
the 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In some embodiments, the recombinant nucleic acid is selected from:
(a) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(b) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(c) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3928;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(d) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(e) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(f) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(g) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939; and
(h) a recombinant nucleic acid comprising:
   a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
   a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
   an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
   a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
   a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
   a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
   a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
   a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
   a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
   a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
   a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
   a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
   a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In some embodiments, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3940-3947.

In some embodiments, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3941.

In some embodiments, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3945. In some embodiments, the recombinant nucleic acid comprises or consists of the nucleic acid sequence of any one of SEQ ID NOs: 3940-3947.

In some embodiments, the recombinant nucleic acid comprises or consists of the nucleic acid sequence of SEQ ID NO: 3941.

In some embodiments, the recombinant nucleic acid comprises or consists of the nucleic acid sequence of SEQ ID NO: 3945.

Aspects of the present disclosure provide an adeno-associated virus (AAV) particle comprising any one of the recombinant nucleic acids described herein and a capsid protein, e.g., a capsid protein known in the art or described herein.

In some embodiments, the capsid protein is derived from a serotype selected from AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, and AAV7m8, or a derivative, hybrid or chimeric serotype thereof.

In some embodiments, the AAV particle has increased muscle tropism compared to a wild type AAV9 particle. In some embodiments, the AAV particle has reduced liver tropism compared to a wild type AAV9 particle.

In some embodiments, the capsid protein comprises an amino acid sequence of formula (I):

X₁NX₂X₃X₄RGDRX₅X₆L, (I)
wherein each of X₁-X₆ is any amino acid.

In some embodiments, the amino acid sequence of formula (I) is in a hypervariable region IV (HVR IV) relative to a wild type AAV9 capsid. In some embodiments, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L is inserted after amino acid 455.

In some embodiments,
X₁ is A, I, F, G, H, L, M, Q, S, T, or V;
X₂ is A, G, S, T, or Y;
X₃ is S, N, G, or P;
X₄ is A, G, H, I, M, S, T, or V;
X₅ is A, G, or Q; and
X₆ is A, I, L, M, N, S, or Y.

In some embodiments, the amino acid sequence of formula (I) comprises of any one of SEQ ID NOs: 801-1599. In some embodiments, the amino acid sequence of formula (I) comprises of any one of SEQ ID NOs: 1600-2398. In some embodiments, the amino acid sequence of formula (I) comprises or consists of any one of SEQ ID NOs: 2-800. In some embodiments, the amino acid sequence of formula (I) comprises or consists of SEQ ID NOs: 191, 198, 199, 215, 256, 379, 544, 550, or 748.

In some embodiments, the capsid protein further comprises a deletion of amino acid G267 relative to a wild type AAV9 vector. In some embodiments, the amino acid sequence of formula (I) comprises or consists of SEQ ID NO: 215, the capsid protein further comprises a deletion of amino acid G267 relative to a wild type AAV9 vector, and the remainder of the capsid is otherwise identical to the wild type AAV9.

Aspects of the present disclosure provide a pharmaceutical composition comprising any one of the recombinant nucleic acids described herein or any one of the adeno-associated virus (AAV) particles described herein, and a pharmaceutically acceptable carrier.

Aspects of the present disclosure provide a method of treating facioscapulohumeral muscular dystrophy (FSHD) in a subject in need thereof, the method comprising administering to the subject an effective amount of any one of the recombinant nucleic acids described herein, any one of the AAV particles described herein, or any of the pharmaceutical compositions described herein.

In some embodiments, the subject is a human subject. In some embodiments, the subject has one or more deletions of a D4Z4 repeat in chromosome 4. In some embodiments, the subject has one or more deletions in a SMCHD1 gene. In some embodiments, the subject has one or more deletions in a DNMT3B gene. In some embodiments, the subject has FSHD type 1 (FSHD1), FSHD type 2 (FSHD2), or a combination thereof (FSHD1+2). In some embodiments, the administering comprises intramuscular administration, subcutaneous injection, intravenous injection, or intravenous (IV) administration.

Aspects of the present disclosure provide use of any one of the recombinant nucleic acids described herein, any one of the AAV particles described herein, or any of the pharmaceutical compositions described herein for the manufacture of a medicament for treating FSHD.

Aspects of the present disclosure provide any one of the recombinant nucleic acids described herein, any one of the AAV particles described herein, or any of the pharmaceutical compositions described herein for use in treating FSHD.

Aspects of the present disclosure provide a kit comprising any one of the recombinant nucleic acids described herein, any one of the AAV particles described herein, or any of the pharmaceutical compositions described herein. In some embodiments, the kit further comprises instructions for use in treating FSHD in a subject in need thereof.

Aspects of the recombinant nucleic acid molecules (e.g., cargo) and capsid proteins are described in further detail below.

For sequences disclosed throughout this application, it is understood that nucleic acid molecules and peptides may comprise one or more substitutions, for example conservative substitutions, that allow sequences to continue to function. Accordingly, sequences may have at least 80%, 85%, 90%, 95%, 96%, 97%, 98%, or 99% sequence identity to the sequence disclosed.

A conservative substitution refers to amino acid substitutions that do not significantly affect or alter binding characteristics of a particular protein. Generally, conservative substitutions are ones in which a substituted amino acid residue is replaced with an amino acid residue having a similar side chain. For example, conservative substitutions may include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp.

### miR Scaffolds and Guides

The present invention provides recombinant nucleic acids comprising a primary microRNA (pri-mRNA) and uses of such recombinant nucleic acids for treating FSHD.

The primary microRNAs (pri-mRNAs) for use in compositions and methods described herein comprise a microRNA (miRNA) scaffold (e.g., a scaffold sequence derived from miR-138 or miR-139), a passenger strand, and a guide strand that targets a DUX4 gene transcript that is capable of reducing DUX4 mRNA levels and interfering with translation of the DUX4 gene transcript.

By the present invention, it was discovered that miR-138 and miR-139 scaffolds can be highly expressed and efficiently processed from primary miRs (pri-miRNAs) into mature miR transcripts in muscle tissue, for example, skeletal muscle tissue and cardiac tissue. This allows the present invention to provide for the expression of a mature microRNAs (miRs) comprising a nucleic acid guide targeting a DUX4 transcript within skeletal and cardiac muscle, resulting in interference/silencing of DUX4 gene expression. Advantageously, without being bound by a mechanism of action, the miR-138 and miR-139 scaffolds provide a construct that protects the DUX4 guide sequence (for example within the cytoplasm of cells) and allows for efficient processing of the DUX4 guide sequence into a mature miR.

It should be understood that a scaffold sequence derived from any endogenous miRNA can be used in a pri-miRNA for use in compositions and methods described herein. Non-limiting examples of endogenous pri-miRNA from which a scaffold sequence can be derived include pri-miR-21, pri-miR-22, pri-miR-26a, pri-miR-30a, pri-miR-33, pri-miR-122, pri-miR-138, pri-miR-139, pri-miR-375, pri-miR-199, pri-miR-99, pri-miR-194, pri-miR-155, and pri-miR-451.

Accordingly, the present invention provides a nucleic acid molecule comprising a pri-miRNA scaffold having the scaffold sequence of miR-138 or miR-139 and an artificial miRNA guide sequence within the scaffold that targets a gene transcript.

The pri-miRNA scaffolds described herein together with their guide and passenger sequence may form a hairpin loop structure. The hairpin loop structure may be greater than 250 nucleotides in length (for example between 250-270 nucleotides in length). The pri-miRNA may have the structure, in order, of a first (“upstream”) scaffold sequence, the guide sequence, a hairpin loop, a sequence complimentary or partially complementary to the guide sequence (called a “passenger” sequence), and a second (“downstream”) scaffold sequence. The guide sequence and the passenger sequence form a double stranded RNA (dsRNA), with the first and second scaffold sequence being single stranded RNA (sRNA) on either end of the double stranded molecule (referred to as “arms”). The passenger strand may be fully complementary to the guide sequence or may have one or more mismatched nucleotides (e.g., 1 mismatched nucleotide, 2 mismatched nucleotides, 3 mismatched nucleotides, 4 mismatched nucleotides, 5 or more mismatched nucleotides) to the guide strand. For example, mismatches or additional nucleotides may result in “bulges” in the pri-miRNA or the pre-miRNA, while maintaining overall hybridization between the guide strand and passenger strand. The hairpin loop is found at the opposite end of the dsRNA from the first and second scaffold sequences, connecting the 5′ and 3′ end of the guide and passenger. An example of a pri-miRNA including a scaffold derived from miR-139 and guide and passenger stands is shown below:

pri-miRNA #1
(SEQ ID NO: 3948)
GGGGACCAGCTATGGTAAGGGATGAGAGAAGCCAACAGTTGTCCCAAGG
GTCCCCCTCAGGGAGTGGAGGTTCCCCTGGGAGGCGCCAGGACAGGCGC
AGGTGTATGAAACCAGATCTGAATCCTGGTGTGGCTCGGAGGCCCAGGA
TTACGCTCTGGTTTCTAACAACTGAAGCCAGAGTCTTCGAAGGGTGGTG
GGCTAAGGGGGAAGAGGGCATGGTGGGCAAAAGAAAGGGGGTGGGGGAG
AGAGAAAAGGAGGACAAGGGGG
pri-miRNA #2
(SEQ ID NO: 3949)
GGGGACCAGCTATGGTAAGGGATGAGAGAAGCCAACAGTTGTCCCAAGG
GTCCCCCTCAGGGAGTGGAGGTTCCCCTGGGAGGCGCCAGGACAGGCGC
AGGTGTATTTCGATTCTGAAACCAGATCTTGTGGCTCGGAGGCAGATCT
GGAGTAAGAATCGAATAACAACTGAAGCCAGAGTCTTCGAAGGGTGGTG
GGCTAAGGGGGAAGAGGGCATGGTGGGCAAAAGAAAGGGGGTGGGGGAG
AGAGAAAAGGAGGACAAGGGGG

The hairpin structure may be processed by cleaving near the junction between dsRNA and ssRNA arms. After processing, which may result in substantially cleaving of the ssRNA arms, the pri-miRNAs loop structure is generally 60-100 nucleotides long, and is referred to as a precursor miRNA (pre-miRNA).

The pre-miRNA may be exported to the cytoplasm and further processed by an enzyme, for example (Dicer). The enzyme may process the pre-miRNA at the 5′ and 3′ ends of the hairpin by cutting away the loop joining the 3′ and 5′ arms, resulting in an miRNA(guide):miRNA (passenger) duplex about 21 nucleotides in length.

The duplexed miRNA may then by processed to form a precursor to an RNA-induced silencing complex (RISC). The complex may unwind the duplex and the passenger RNA strand may then be discarded, leaving behind a mature RISC carrying the mature, single stranded guide miRNA.

In aspects of the invention, the pri-miR scaffold comprises the scaffold sequence of miR-138.

For example, the nucleic acid molecule (e.g. pri-miR) may comprising in order:
a first scaffold sequence of SEQ ID NO: 3929;
a guide sequence (e.g., any one of SEQ ID NOs: 2399-3159 and 3952-3953);
a loop scaffold sequence of SEQ ID NO: 3930;
a passenger sequence to the guide sequence (e.g., any one of SEQ ID NOs: 3160-3920); and
a second scaffold sequence of SEQ ID NO: 3931.

Together, the nucleic acid molecule may comprise a sequence having at least 95% sequence identity with the defined nucleic acids in SEQ ID NO: 3932, where N is any nucleic acid and indicates the position of the guide and the passenger strands within the scaffold. In such instances, the first series of Ns can be a sequence of a guide strand (e.g., a sequence of any one of SEQ ID NOs: 2399-3159 and 3952-3953) and the second series of Ns can be a sequence of a passenger strand (e.g., a sequence of any one of SEQ ID NOs: 3160-3920), or vice versa.

| SEQ ID | pri-miR 138 | |
|---|---|---|
| NO: | Description | Sequence |
| 3932 | pri-miR 138 | GGACCACCCAGCAGTGATGCACGTAGAGCAGAGGTGCAGTGATGCACGTACAGCAG |
| | sequence | AGGCGACCCCTTCTCCATACTTCAGAGACCTCTAGCATGGTGTTGTGGGACNNNNN |
| | | NNNNNNNNNNNNNNNNTTGCCAATCAGAGAANNNNNNNNNNNNNNNNNNNNNCCAC |
| | | ACTGCACTGCAAGCAGCGAGCAGCAAGCAACAAGCATCCAGACACCTGAGCACCAG |
| | | GATGCCAGCAAGAGCACCTGCCTGTGAGGAAACCTCCCAGCC |
| 3929 | upstream | GGACCACCCAGCAGTGATGCACGTAGAGCAGAGGTGCAGTGATGCACGTACAGCAG |
| | scaffold | AGGCGACCCCTTCTCCATACTTCAGAGACCTCTAGCATGGTGTTGTGGGAC |
| 3930 | short hairpin | TTGCCAATCAGAGAA |
| | loop | |
| 3931 | downstream | CCACACTGCACTGCAAGCAGCGAGCAGCAAGCAACAAGCATCCAGACACCTGAGCA |
| | scaffold | CCAGGATGCCAGCAAGAGCACCTGCCTGTGAGGAAACCTCCCAGCC |

In aspects of the invention, the pri-miR scaffold comprises the scaffold sequence of miR-139.

For example, the nucleic acid molecule (e.g. pri-miR) may comprising in order:
a first scaffold sequence of SEQ ID NO: 3933;
a guide sequence (e.g., any one of SEQ ID NOs: 2399-3159 and 3952-3953);
a loop scaffold sequence of SEQ ID NO: 3934;
a passenger sequence to the guide sequence (e.g., any one of SEQ ID NOs: 3160-3920); and
   a second scaffold sequence of SEQ ID NO: 3935.

Together, the nucleic acid molecule may comprise a sequence having at least 95% sequence identity with the sequence of SEQ ID NO: 3936, where N is any nucleic acid and indicates the position of the guide and the passenger strands within the scaffold. In such instances, the first series of Ns can be a sequence of a guide strand (e.g., a sequence of any one of SEQ ID NOs: 2399-3159 and 3952-3953) and the second series of Ns can be a sequence of a passenger strand (e.g., a sequence of any one of SEQ ID NOs: 3160-3920), or vice versa.

| SEQ ID | pri-miR 139 | |
|---|---|---|
| NO: | Description | Sequence |
| 3936 | pri-miR 139 | GGGGACCAGCTATGGTAAGGGATGAGAGAAGCCAACAGTTGTCCCAAGGGTCCCCC |
| | sequence | TCAGGGAGTGGAGGTTCCCCTGGGAGGCGCCAGGACAGGCGCAGGTGTATNNNNNN |
| | | NNNNNNNNNNNNNNNTGTGGCTCGGAGGCNNNNNNNNNNNNNNNNNNNNNTAACAA |
| | | CTGAAGCCAGAGTCTTCGAAGGGTGGTGGGCTAAGGGGGAAGAGGGCATGGTGGGC |
| | | AAAAGAAAGGGGGTGGGGGAGAGAGAAAAGGAGGACAAGGGGG |
| 3933 | upstream | GGGGACCAGCTATGGTAAGGGATGAGAGAAGCCAACAGTTGTCCCAAGGGTCCCCC |
| | scaffold | TCAGGGAGTGGAGGTTCCCCTGGGAGGCGCCAGGACAGGCGCAGGTGTAT |
| 3934 | short hairpin | TGTGGCTCGGAGGC |
| | loop | |
| 3935 | downstream | TAACAACTGAAGCCAGAGTCTTCGAAGGGTGGTGGGCTAAGGGGGAAGAGGGCATG |
| | scaffold | GTGGGCAAAAGAAAGGGGGTGGGGGAGAGAGAAAAGGAGGACAAGGGGG |

Typically, guide sequences are approximately 21-22 nucleotides in length. In aspects of the invention, the guide sequence may be 21 nucleotides in length.

Advantageously, the guide sequence targets a gene transcript for a disease-causing muscle protein, for example, double homeobox 4 (DUX4).

Exemplary miR guide sequences for targeting a DUX4 are provided in Table 1 below. Lower case nucleic acids indicate where a mismatched nucleic acid can be positioned, e.g., a mismatched nucleic acid when a passenger strand (e.g., SEQ ID NO: 3160) base pairs with a guide strand (e.g., SEQ ID NO: 2399).

TABLE 1
Exemplary miRNA sequences targeting a DUX4 transcript

| SEQ ID NO: | Target (Guide Strand) | SEQ ID NO: | Antisense (Passenger Strand) |
|---|---|---|---|
| 2399 | GCCGTCCTCGTCCCCGGGCTT | 3160 | AAGCCCGGacAaGAGGACGGC |
| 2400 | GCCGTCCTCGTCCCCGGGCTT | 3161 | AAGCCCGGttAaGAGGACGGC |
| 2401 | GCCGTCCTCGTCCCCGGGCTT | 3162 | AAGCCCGGtaAgGAGGACGGC |
| 2402 | GCCGTCCTCGTCCCCGGGCTT | 3163 | AAGCCCGGcaAgGAGGACGGC |
| 2403 | GCCGTCCTCGTCCCCGGGCTT | 3164 | AAGCCCGGatAgGAGGACGGC |
| 2404 | GCCGTCCTCGTCCCCGGGCTT | 3165 | AAGCCCGGctAgGAGGACGGC |
| 2405 | GCCGTCCTCGTCCCCGGGCTT | 3166 | AAGCCCGGacAgGAGGACGGC |
| 2406 | GCCGTCCTCGTCCCCGGGCTT | 3167 | AAGCCCGGtcAgGAGGACGGC |
| 2407 | GGCCTCGCTTTGGCTCGGGGT | 3168 | ACCCCGAGaaAtAGCGAGGCC |
| 2408 | GGCCTCGCTTTGGCTCGGGGT | 3169 | ACCCCGAGgaAtAGCGAGGCC |
| 2409 | GGCCTCGCTTTGGCTCGGGGT | 3170 | ACCCCGAGagAtAGCGAGGCC |
| 2410 | GGCCTCGCTTTGGCTCGGGGT | 3171 | ACCCCGAGggAtAGCGAGGCC |
| 2411 | GGCCTCGCTTTGGCTCGGGGT | 3172 | ACCCCGAGaaAcAGCGAGGCC |
| 2412 | GGCCTCGCTTTGGCTCGGGGT | 3173 | ACCCCGAGgaAcAGCGAGGCC |
| 2413 | GGCCTCGCTTTGGCTCGGGGT | 3174 | ACCCCGAGagAcAGCGAGGCC |
| 2414 | GGCCTCGCTTTGGCTCGGGGT | 3175 | ACCCCGAGggAcAGCGAGGCC |
| 2415 | CAGGGCCTCGCTTTGGCTCGG | 3176 | CCGAGCCAttGaGAGGCCCTG |
| 2416 | CAGGGCCTCGCTTTGGCTCGG | 3177 | CCGAGCCActGaGAGGCCCTG |
| 2417 | CAGGGCCTCGCTTTGGCTCGG | 3178 | CCGAGCCAtcGaGAGGCCCTG |
| 2418 | CAGGGCCTCGCTTTGGCTCGG | 3179 | CCGAGCCAccGaGAGGCCCTG |
| 2419 | CAGGGCCTCGCTTTGGCTCGG | 3180 | CCGAGCCAttGgGAGGCCCTG |
| 2420 | CAGGGCCTCGCTTTGGCTCGG | 3181 | CCGAGCCActGgGAGGCCCTG |
| 2421 | CAGGGCCTCGCTTTGGCTCGG | 3182 | CCGAGCCAtcGgGAGGCCCTG |
| 2422 | CAGGGCCTCGCTTTGGCTCGG | 3183 | CCGAGCCAccGgGAGGCCCTG |
| 2423 | GGCCTTCTGGCGGGCCGCGTC | 3184 | GACGCGGCggGaCAGAAGGCC |
| 2424 | GGCCTTCTGGCGGGCCGCGTC | 3185 | GACGCGGCaaGaCAGAAGGCC |
| 2425 | GGCCTTCTGGCGGGCCGCGTC | 3186 | GACGCGGCgaGaCAGAAGGCC |
| 2426 | GGCCTTCTGGCGGGCCGCGTC | 3187 | GACGCGGCagGaCAGAAGGCC |
| 2427 | GGCCTTCTGGCGGGCCGCGTC | 3188 | GACGCGGCaaGgCAGAAGGCC |
| 2428 | GGCCTTCTGGCGGGCCGCGTC | 3189 | GACGCGGCgaGgCAGAAGGCC |
| 2429 | GGCCTTCTGGCGGGCCGCGTC | 3190 | GACGCGGCagGgCAGAAGGCC |
| 2430 | GGCCTTCTGGCGGGCCGCGTC | 3191 | GACGCGGCggGgCAGAAGGCC |
| 2431 | AGCTCCTCCCGGGGGGCGATG | 3192 | CATCGCCGagCcGGAGGAGCT |
| 2432 | AGCTCCTCCCGGGCGGCGATG | 3193 | CATCGCCGgaCaGGAGGAGCT |
| 2433 | AGCTCCTCCCGGGCGGCGATG | 3194 | CATCGCCGggCaGGAGGAGCT |
| 2434 | AGCTCCTCCCGGGCGGCGATG | 3195 | CATCGCCGaaCtGGAGGAGCT |
| 2435 | AGCTCCTCCCGGGCGGCGATG | 3196 | CATCGCCGgaCtGGAGGAGCT |
| 2436 | AGCTCCTCCCGGGCGGCGATG | 3197 | CATCGCCGggCtGGAGGAGCT |
| 2437 | AGCTCCTCCCGGGGGGCGATG | 3198 | CATCGCCGaaCcGGAGGAGCT |
| 2438 | AGCTCCTCCCGGGCGGCGATG | 3199 | CATCGCCGgaCcGGAGGAGCT |
| 2439 | AGGCCGCCTGCCTGCGCGGGC | 3200 | GCCCGCGCctGaAGGCGGCCT |
| 2440 | AGGCCGCCTGCCTGCGCGGGC | 3201 | GCCCGCGCtaGaAGGCGGCCT |
| 2441 | AGGCCGCCTGCCTGCGCGGGC | 3202 | GCCCGCGCcaGaAGGCGGCCT |
| 2442 | AGGCCGCCTGCCTGCGCGGGC | 3203 | GCCCGCGCccGaAGGCGGCCT |
| 2443 | AGGCCGCCTGCCTGCGCGGGC | 3204 | GCCCGCGCttGgAGGCGGCCT |
| 2444 | AGGCCGCCTGCCTGCGCGGGC | 3205 | GCCCGCGCctGgAGGCGGCCT |
| 2445 | AGGCCGCCTGCCTGCGCGGGC | 3206 | GCCCGCGCtcGgAGGCGGCCT |
| 2446 | AGGCCGCCTGCCTGCGCGGGC | 3207 | GCCCGCGCccGgAGGCGGCCT |
| 2447 | CCGGGGGCCGCGCTGCACAGG | 3208 | CCTGTGCAagGaGGCCCCCGG |
| 2448 | CCGGGGGCCGCGCTGCACAGG | 3209 | CCTGTGCAtaGaGGCCCCCGG |
| 2449 | CCGGGGGCCGCGCTGCACAGG | 3210 | CCTGTGCAcaGaGGCCCCCGG |
| 2450 | CCGGGGGCCGCGCTGCACAGG | 3211 | CCTGTGCAtgGaGGCCCCCGG |
| 2451 | CCGGGGGCCGCGCTGCACAGG | 3212 | CCTGTGCAcgGaGGCCCCCGG |
| 2452 | CCGGGGGCCGCGCTGCACAGG | 3213 | CCTGTGCAaaGgGGCCCCCGG |
| 2453 | CCGGGGGCCGCGCTGCACAGG | 3214 | CCTGTGCAtaGgGGCCCCCGG |
| 2454 | CCGGGGGCCGCGCTGCACAGG | 3215 | CCTGTGCAagGgGGCCCCCGG |
| 2455 | CGAGGGAGCAGGGTGACCCCC | 3216 | GGGGGTCAgaCgGCTCCCTCG |
| 2456 | CGAGGGAGCAGGGTGACCCCC | 3217 | GGGGGTCAaaCaGCTCCCTCG |
| 2457 | CGAGGGAGCAGGGTGACCCCC | 3218 | GGGGGTCAggCaGCTCCCTCG |
| 2458 | CGAGGGAGCAGGGTGACCCCC | 3219 | GGGGGTCAaaCcGCTCCCTCG |
| 2459 | CGAGGGAGCAGGGTGACCCCC | 3220 | GGGGGTCAgaCcGCTCCCTCG |
| 2460 | CGAGGGAGCAGGGTGACCCCC | 3221 | GGGGGTCAggCcGCTCCCTCG |
| 2461 | CGAGGGAGCAGGGTGACCCCC | 3222 | GGGGGTCAagCgGCTCCCTCG |
| 2462 | CGAGGGAGCAGGGTGACCCCC | 3223 | GGGGGTCAggCgGCTCCCTCG |
| 2463 | TGGGCGAAGGCGACCCACGAG | 3224 | CTCGTGGGggGgCTTCGCCCA |
| 2464 | TGGGCGAAGGCGACCCACGAG | 3225 | CTCGTGGGaaGaCTTCGCCCA |
| 2465 | TGGGCGAAGGCGACCCACGAG | 3226 | CTCGTGGGcaGaCTTCGCCCA |
| 2466 | TGGGCGAAGGCGACCCACGAG | 3227 | CTCGTGGGagGaCTTCGCCCA |
| 2467 | TGGGCGAAGGCGACCCACGAG | 3228 | CTCGTGGGcgGaCTTCGCCCA |
| 2468 | TGGGCGAAGGCGACCCACGAG | 3229 | CTCGTGGGggGaCTTCGCCCA |
| 2469 | TGGGCGAAGGCGACCCACGAG | 3230 | CTCGTGGGgaGgCTTCGCCCA |
| 2470 | TGGGCGAAGGCGACCCACGAG | 3231 | CTCGTGGGagGgCTTCGCCCA |
| 2471 | CCGGTGTGGGCGAAGGCGACC | 3232 | GGTCGCCTagGaCCACACCGG |
| 2472 | CCGGTGTGGGCGAAGGCGACC | 3233 | GGTCGCCTcaGaCCACACCGG |
| 2473 | CCGGTGTGGGCGAAGGCGACC | 3234 | GGTCGCCTgaGaCCACACCGG |
| 2474 | CCGGTGTGGGCGAAGGCGACC | 3235 | GGTCGCCTcgGaCCACACCGG |
| 2475 | CCGGTGTGGGCGAAGGCGACC | 3236 | GGTCGCCTaaGgCCACACCGG |
| 2476 | CCGGTGTGGGCGAAGGCGACC | 3237 | GGTCGCCTcaGgCCACACCGG |
| 2477 | CCGGTGTGGGCGAAGGCGACC | 3238 | GGTCGCCTgaGgCCACACCGG |
| 2478 | CCGGTGTGGGCGAAGGCGACC | 3239 | GGTCGCCTggGgCCACACCGG |
| 2479 | CGCCGGTGTGGGCGAAGGCGA | 3240 | TCGCCTTCaaCgACACCGGCG |
| 2480 | CGCCGGTGTGGGCGAAGGCGA | 3241 | TCGCCTTCtgCgACACCGGCG |
| 2481 | CGCCGGTGTGGGCGAAGGCGA | 3242 | TCGCCTTCaaCaACACCGGCG |
| 2482 | CGCCGGTGTGGGCGAAGGCGA | 3243 | TCGCCTTCcaCaACACCGGCG |
| 2483 | CGCCGGTGTGGGCGAAGGCGA | 3244 | TCGCCTTCagCaACACCGGCG |
| 2484 | CGCCGGTGTGGGCGAAGGCGA | 3245 | TCGCCTTCtgCaACACCGGCG |
| 2485 | CGCCGGTGTGGGCGAAGGCGA | 3246 | TCGCCTTCcgCaACACCGGCG |
| 2486 | CGCCGGTGTGGGCGAAGGCGA | 3247 | TCGCCTTCtaCgACACCGGCG |
| 2487 | AGCCCCGTTCCCCACGCGCCG | 3248 | CGGCGCGTatGtAACGGGGCT |
| 2488 | AGCCCCGTTCCCCACGCGCCG | 3249 | CGGCGCGTaaGaAACGGGGCT |
| 2489 | AGCCCCGTTCCCCACGCGCCG | 3250 | CGGCGCGTctGaAACGGGGCT |
| 2490 | AGCCCCGTTCCCCACGCGCCG | 3251 | CGGCGCGTaaGtAACGGGGCT |
| 2491 | AGCCCCGTTCCCCACGCGCCG | 3252 | CGGCGCGTttGtAACGGGGCT |
| 2492 | AGCCCCGTTCCCCACGCGCCG | 3253 | CGGCGCGTaaGcAACGGGGCT |
| 2493 | AGCCCCGTTCCCCACGCGCCG | 3254 | CGGCGCGTatGcAACGGGGCT |
| 2494 | AGCCCCGTTCCCCACGCGCCG | 3255 | CGGCGCGTctGcAACGGGGCT |
| 2495 | CCGTCCGGAGGAGCCGGGGCG | 3256 | CGCCCCGGggCaTCCGGACGG |
| 2496 | CCGTCCGGAGGAGCCGGGGCG | 3257 | CGCCCCGGaaCaTCCGGACGG |
| 2497 | CCGTCCGGAGGAGCCGGGGCG | 3258 | CGCCCCGGgaCaTCCGGACGG |
| 2498 | CCGTCCGGAGGAGCCGGGGCG | 3259 | CGCCCCGGacCaTCCGGACGG |
| 2499 | CCGTCCGGAGGAGCCGGGGCG | 3260 | CGCCCCGGaaCgTCCGGACGG |
| 2500 | CCGTCCGGAGGAGCCGGGGCG | 3261 | CGCCCCGGgaCgTCCGGACGG |
| 2501 | CCGTCCGGAGGAGCCGGGGCG | 3262 | CGCCCCGGgcCgTCCGGACGG |
| 2502 | CCGTCCGGAGGAGCCGGGGCG | 3263 | CGCCCCGGggCgTCCGGACGG |
| 2503 | AGAGCGCCCCGTCCGGAGGAG | 3264 | CTCCTCCGtcCtGGGCGCTCT |
| 2504 | AGAGCGCCCCGTCCGGAGGAG | 3265 | CTCCTCCGatCaGGGCGCTCT |
| 2505 | AGAGCGCCCCGTCCGGAGGAG | 3266 | CTCCTCCGttCtGGGCGCTCT |
| 2506 | AGAGCGCCCCGTCCGGAGGAG | 3267 | CTCCTCCGctCtGGGCGCTCT |
| 2507 | AGAGCGCCCCGTCCGGAGGAG | 3268 | CTCCTCCGacCtGGGCGCTCT |
| 2508 | AGAGCGCCCCGTCCGGAGGAG | 3269 | CTCCTCCGatCcGGGCGCTCT |
| 2509 | AGAGCGCCCCGTCCGGAGGAG | 3270 | CTCCTCCGttCcGGGCGCTCT |
| 2510 | AGAGCGCCCCGTCCGGAGGAG | 3271 | CTCCTCCGtcCcGGGCGCTCT |
| 2511 | CCGGGGGCGCGGGCTGGGGAG | 3272 | CTCCCCAGgaCaCGCCCCCGG |
| 2512 | CCGGGGGCGCGGGCTGGGGAG | 3273 | CTCCCCAGagCaCGCCCCCGG |
| 2513 | CCGGGGGCGCGGGCTGGGGAG | 3274 | CTCCCCAGggCaCGCCCCCGG |
| 2514 | CCGGGGGCGCGGGCTGGGGAG | 3275 | CTCCCCAGaaCtCGCCCCCGG |
| 2515 | CCGGGGGCGCGGGCTGGGGAG | 3276 | CTCCCCAGggCtCGCCCCCGG |
| 2516 | CCGGGGGCGCGGGCTGGGGAG | 3277 | CTCCCCAGaaCcCGCCCCCGG |
| 2517 | CCGGGGGCGCGGGCTGGGGAG | 3278 | CTCCCCAGgaCcCGCCCCCGG |
| 2518 | CCGGGGGCGCGGGCTGGGGAG | 3279 | CTCCCCAGggCcCGCCCCCGG |
| 2519 | GGCGGAGGCGTCCGGGGGCGC | 3280 | GCGCCCCCccAgGCCTCCGCC |
| 2520 | GGCGGAGGCGTCCGGGGGCGC | 3281 | GCGCCCCCatAaGCCTCCGCC |
| 2521 | GGCGGAGGCGTCCGGGGGCGC | 3282 | GCGCCCCCctAaGCCTCCGCC |
| 2522 | GGCGGAGGCGTCCGGGGGCGC | 3283 | GCGCCCCCacAaGCCTCCGCC |
| 2523 | GGCGGAGGCGTCCGGGGGCGC | 3284 | GCGCCCCCtcAaGCCTCCGCC |
| 2524 | GGCGGAGGCGTCCGGGGGCGC | 3285 | GCGCCCCCcaAgGCCTCCGCC |
| 2525 | GGCGGAGGCGTCCGGGGGCGC | 3286 | GCGCCCCCatAgGCCTCCGCC |
| 2526 | GGCGGAGGCGTCCGGGGGCGC | 3287 | GCGCCCCCacAgGCCTCCGCC |
| 2527 | AGACCCCGCGTCCTAAAGCTC | 3288 | GAGCTTTAtaAgGCGGGGTCT |
| 2528 | AGACCCCGCGTCCTAAAGCTC | 3289 | GAGCTTTAtaAaGCGGGGTCT |
| 2529 | AGACCCCGCGTCCTAAAGCTC | 3290 | GAGCTTTAatAaGCGGGGTCT |
| 2530 | AGACCCCGCGTCCTAAAGCTC | 3291 | GAGCTTTActAaGCGGGGTCT |
| 2531 | AGACCCCGCGTCCTAAAGCTC | 3292 | GAGCTTTAtcAaGCGGGGTCT |
| 2532 | AGACCCCGCGTCCTAAAGCTC | 3293 | GAGCTTTAatAgGCGGGGTCT |
| 2533 | AGACCCCGCGTCCTAAAGCTC | 3294 | GAGCTTTAttAgGCGGGGTCT |
| 2534 | AGACCCCGCGTCCTAAAGCTC | 3295 | GAGCTTTAtcAgGCGGGGTCT |
| 2535 | GGGCCTAGACCCCGCGTCCTA | 3296 | TAGGACGCcaGaTCTAGGCCC |
| 2536 | GGGCCTAGACCCCGCGTCCTA | 3297 | TAGGACGCatGtTCTAGGCCC |
| 2537 | GGGCCTAGACCCCGCGTCCTA | 3298 | TAGGACGCttGtTCTAGGCCC |
| 2538 | GGGCCTAGACCCCGCGTCCTA | 3299 | TAGGACGCctGtTCTAGGCCC |
| 2539 | GGGCCTAGACCCCGCGTCCTA | 3300 | TAGGACGCtcGtTCTAGGCCC |
| 2540 | GGGCCTAGACCCCGCGTCCTA | 3301 | TAGGACGCaaGcTCTAGGCCC |
| 2541 | GGGCCTAGACCCCGCGTCCTA | 3302 | TAGGACGCctGcTCTAGGCCC |
| 2542 | GGGCCTAGACCCCGCGTCCTA | 3303 | TAGGACGCccGcTCTAGGCCC |
| 2543 | TGGCTCACGAAAGCCCCCTGT | 3304 | ACAGGGGGggTcCGTGAGCCA |
| 2544 | TGGCTCACGAAAGCCCCCTGT | 3305 | ACAGGGGGacTaCGTGAGCCA |
| 2545 | TGGCTCACGAAAGCCCCCTGT | 3306 | ACAGGGGGagTaCGTGAGCCA |
| 2546 | TGGCTCACGAAAGCCCCCTGT | 3307 | ACAGGGGGaaTcCGTGAGCCA |
| 2547 | TGGCTCACGAAAGCCCCCTGT | 3308 | ACAGGGGGacTcCGTGAGCCA |
| 2548 | TGGCTCACGAAAGCCCCCTGT | 3309 | ACAGGGGGgcTcCGTGAGCCA |
| 2549 | TGGCTCACGAAAGCCCCCTGT | 3310 | ACAGGGGGgaTgCGTGAGCCA |
| 2550 | TGGCTCACGAAAGCCCCCTGT | 3311 | ACAGGGGGacTgCGTGAGCCA |
| 2551 | TGCCTGGCTCACGAAAGCCCC | 3312 | GGGGCTTTaaTtAGCCAGGCA |
| 2552 | TGCCTGGCTCACGAAAGCCCC | 3313 | GGGGCTTTgaTaAGCCAGGCA |
| 2553 | TGCCTGGCTCACGAAAGCCCC | 3314 | GGGGCTTTgtTaAGCCAGGCA |
| 2554 | TGCCTGGCTCACGAAAGCCCC | 3315 | GGGGCTTTacTaAGCCAGGCA |
| 2555 | TGCCTGGCTCACGAAAGCCCC | 3316 | GGGGCTTTatTtAGCCAGGCA |
| 2556 | TGCCTGGCTCACGAAAGCCCC | 3317 | GGGGCTTTgcTtAGCCAGGCA |
| 2557 | TGCCTGGCTCACGAAAGCCCC | 3318 | GGGGCTTTgaTcAGCCAGGCA |
| 2558 | TGCCTGGCTCACGAAAGCCCC | 3319 | GGGGCTTTgcTcAGCCAGGCA |
| 2559 | TCGCTGCCTGGCTCACGAAAG | 3320 | CTTTCGTGttCaAGGCAGCGA |
| 2560 | TCGCTGCCTGGCTCACGAAAG | 3321 | CTTTCGTGttCgAGGCAGCGA |
| 2561 | TCGCTGCCTGGCTCACGAAAG | 3322 | CTTTCGTGtcCaAGGCAGCGA |
| 2562 | TCGCTGCCTGGCTCACGAAAG | 3323 | CTTTCGTGtaCgAGGCAGCGA |
| 2563 | TCGCTGCCTGGCTCACGAAAG | 3324 | CTTTCGTGcaCgAGGCAGCGA |
| 2564 | TCGCTGCCTGGCTCACGAAAG | 3325 | CTTTCGTGctCgAGGCAGCGA |
| 2565 | TCGCTGCCTGGCTCACGAAAG | 3326 | CTTTCGTGtcCgAGGCAGCGA |
| 2566 | TCGCTGCCTGGCTCACGAAAG | 3327 | CTTTCGTGccCgAGGCAGCGA |
| 2567 | TGGGCTGCAGCGCGGGGGGGG | 3328 | CCGCCCCCagGaTGCAGCCCA |
| 2568 | TGGGCTGCAGCGCGGGGGGGG | 3329 | CCGCCCCCaaGaTGCAGCCCA |
| 2569 | TGGGCTGCAGCGCGGGGGCGG | 3330 | CCGCCCCCtgGaTGCAGCCCA |
| 2570 | TGGGCTGCAGCGCGGGGGGGG | 3331 | CCGCCCCCcgGaTGCAGCCCA |
| 2571 | TGGGCTGCAGCGCGGGGGCGG | 3332 | CCGCCCCCtaGgTGCAGCCCA |
| 2572 | TGGGCTGCAGCGCGGGGGGGG | 3333 | CCGCCCCCcaGgTGCAGCCCA |
| 2573 | TGGGCTGCAGCGCGGGGGGGG | 3334 | CCGCCCCCtgGgTGCAGCCCA |
| 2574 | TGGGCTGCAGCGCGGGGGCGG | 3335 | CCGCCCCCcgGgTGCAGCCCA |
| 2575 | CGCCGGGGCAGGTTGGGAGAT | 3336 | ATCTCCCAtgCcGCCCCGGCG |
| 2576 | CGCCGGGGCAGGTTGGGAGAT | 3337 | ATCTCCCAtaCaGCCCCGGCG |
| 2577 | CGCCGGGGCAGGTTGGGAGAT | 3338 | ATCTCCCAcaCaGCCCCGGCG |
| 2578 | CGCCGGGGCAGGTTGGGAGAT | 3339 | ATCTCCCAtgCaGCCCCGGCG |
| 2579 | CGCCGGGGCAGGTTGGGAGAT | 3340 | ATCTCCCAcgCaGCCCCGGCG |
| 2580 | CGCCGGGGCAGGTTGGGAGAT | 3341 | ATCTCCCAtaCcGCCCCGGCG |
| 2581 | CGCCGGGGCAGGTTGGGAGAT | 3342 | ATCTCCCAtgCgGCCCCGGCG |
| 2582 | CGCCGGGGCAGGTTGGGAGAT | 3343 | ATCTCCCAcgCgGCCCCGGCG |
| 2583 | AATCCCCGCGCGCCGGGGCAG | 3344 | CTGCCCCGcgGaGCGGGGATT |
| 2584 | AATCCCCGCGCGCCGGGGCAG | 3345 | CTGCCCCGtaGaGCGGGGATT |
| 2585 | AATCCCCGCGCGCCGGGGCAG | 3346 | CTGCCCCGagGaGCGGGGATT |
| 2586 | AATCCCCGCGCGCCGGGGCAG | 3347 | CTGCCCCGtgGaGCGGGGATT |
| 2587 | AATCCCCGCGCGCCGGGGCAG | 3348 | CTGCCCCGaaGgGCGGGGATT |
| 2588 | AATCCCCGCGCGCCGGGGCAG | 3349 | CTGCCCCGcaGgGCGGGGATT |
| 2589 | AATCCCCGCGCGCCGGGGCAG | 3350 | CTGCCCCGagGgGCGGGGATT |
| 2590 | AATCCCCGCGCGCCGGGGCAG | 3351 | CTGCCCCGcgGgGCGGGGATT |
| 2591 | GGCGGCGTAGGCGAAATCCCC | 3352 | GGGGATTTacCaTACGCCGCC |
| 2592 | GGCGGCGTAGGCGAAATCCCC | 3353 | GGGGATTTaaCaTACGCCGCC |
| 2593 | GGCGGCGTAGGCGAAATCCCC | 3354 | GGGGATTTgaCaTACGCCGCC |
| 2594 | GGCGGCGTAGGCGAAATCCCC | 3355 | GGGGATTTgtCaTACGCCGCC |
| 2595 | GGCGGCGTAGGCGAAATCCCC | 3356 | GGGGATTTgaCgTACGCCGCC |
| 2596 | GGCGGCGTAGGCGAAATCCCC | 3357 | GGGGATTTgtCgTACGCCGCC |
| 2597 | GGCGGCGTAGGCGAAATCCCC | 3358 | GGGGATTTacCgTACGCCGCC |
| 2598 | GGCGGCGTAGGCGAAATCCCC | 3359 | GGGGATTTgcCgTACGCCGCC |
| 2599 | GTCCGGAGGAGCCGGGGCGGC | 3360 | GCCGCCCCccCgCCTCCGGAC |
| 2600 | GTCCGGAGGAGCCGGGGCGGC | 3361 | GCCGCCCCatCaCCTCCGGAC |
| 2601 | GTCCGGAGGAGCCGGGGCGGC | 3362 | GCCGCCCCtaCcCCTCCGGAC |
| 2602 | GTCCGGAGGAGCCGGGGCGGC | 3363 | GCCGCCCCcaCcCCTCCGGAC |
| 2603 | GTCCGGAGGAGCCGGGGCGGC | 3364 | GCCGCCCCatCcCCTCCGGAC |
| 2604 | GTCCGGAGGAGCCGGGGCGGC | 3365 | GCCGCCCCctCcCCTCCGGAC |
| 2605 | GTCCGGAGGAGCCGGGGCGGC | 3366 | GCCGCCCCacCcCCTCCGGAC |
| 2606 | GTCCGGAGGAGCCGGGGCGGC | 3367 | GCCGCCCCtcCcCCTCCGGAC |
| 2607 | CCTGAGGGTGGGAGAGCGCCC | 3368 | GGGCGCTCaaCgACCCTCAGG |
| 2608 | CCTGAGGGTGGGAGAGCGCCC | 3369 | GGGCGCTCcaCaACCCTCAGG |
| 2609 | CCTGAGGGTGGGAGAGCGCCC | 3370 | GGGCGCTCgaCaACCCTCAGG |
| 2610 | CCTGAGGGTGGGAGAGCGCCC | 3371 | GGGCGCTCagCaACCCTCAGG |
| 2611 | CCTGAGGGTGGGAGAGCGCCC | 3372 | GGGCGCTCcgCaACCCTCAGG |
| 2612 | CCTGAGGGTGGGAGAGCGCCC | 3373 | GGGCGCTCggCaACCCTCAGG |
| 2613 | CCTGAGGGTGGGAGAGCGCCC | 3374 | GGGCGCTCcgCgACCCTCAGG |
| 2614 | CCTGAGGGTGGGAGAGCGCCC | 3375 | GGGCGCTCggCgACCCTCAGG |
| 2615 | GCCTGAGGGTGGGAGAGCGCC | 3376 | GGCGCTCTaaCtCCCTCAGGC |
| 2616 | GCCTGAGGGTGGGAGAGCGCC | 3377 | GGCGCTCTgaCtCCCTCAGGC |
| 2617 | GCCTGAGGGTGGGAGAGCGCC | 3378 | GGCGCTCTagCtCCCTCAGGC |
| 2618 | GCCTGAGGGTGGGAGAGCGCC | 3379 | GGCGCTCTggCtCCCTCAGGC |
| 2619 | GCCTGAGGGTGGGAGAGCGCC | 3380 | GGCGCTCTaaCcCCCTCAGGC |
| 2620 | GCCTGAGGGTGGGAGAGCGCC | 3381 | GGCGCTCTgaCcCCCTCAGGC |
| 2621 | GCCTGAGGGTGGGAGAGCGCC | 3382 | GGCGCTCTagCcCCCTCAGGC |
| 2622 | GCCTGAGGGTGGGAGAGCGCC | 3383 | GGCGCTCTggCcCCCTCAGGC |
| 2623 | AGGCCACCGAGGAGCCTGAGG | 3384 | CCTCAGGCaaCaCGGTGGCCT |
| 2624 | AGGCCACCGAGGAGCCTGAGG | 3385 | CCTCAGGCagCaCGGTGGCCT |
| 2625 | AGGCCACCGAGGAGCCTGAGG | 3386 | CCTCAGGCcgCaCGGTGGCCT |
| 2626 | AGGCCACCGAGGAGCCTGAGG | 3387 | CCTCAGGCaaCcCGGTGGCCT |
| 2627 | AGGCCACCGAGGAGCCTGAGG | 3388 | CCTCAGGCcaCcCGGTGGCCT |
| 2628 | AGGCCACCGAGGAGCCTGAGG | 3389 | CCTCAGGCagCcCGGTGGCCT |
| 2629 | AGGCCACCGAGGAGCCTGAGG | 3390 | CCTCAGGCggCcCGGTGGCCT |
| 2630 | AGGCCACCGAGGAGCCTGAGG | 3391 | CCTCAGGCaaCgCGGTGGCCT |
| 2631 | CCTGCGGCCCCGCTTGAGCGG | 3392 | CCGCTCAAaaGaGGCCGCAGG |
| 2632 | CCTGCGGCCCCGCTTGAGCGG | 3393 | CCGCTCAAcaGaGGCCGCAGG |
| 2633 | CCTGCGGCCCCGCTTGAGCGG | 3394 | CCGCTCAAcgGaGGCCGCAGG |
| 2634 | CCTGCGGCCCCGCTTGAGCGG | 3395 | CCGCTCAAaaGtGGCCGCAGG |
| 2635 | CCTGCGGCCCCGCTTGAGCGG | 3396 | CCGCTCAAcaGtGGCCGCAGG |
| 2636 | CCTGCGGCCCCGCTTGAGCGG | 3397 | CCGCTCAAagGtGGCCGCAGG |
| 2637 | CCTGCGGCCCCGCTTGAGCGG | 3398 | CCGCTCAAaaGcGGCCGCAGG |
| 2638 | CCTGCGGCCCCGCTTGAGCGG | 3399 | CCGCTCAAtaGcGGCCGCAGG |
| 2639 | GAGCTGCCCCGGCTTGGGGTT | 3400 | AACCCCAAaaCaGGGCAGCTC |
| 2640 | GAGCTGCCCCGGCTTGGGGTT | 3401 | AACCCCAAtgCaGGGCAGCTC |
| 2641 | GAGCTGCCCCGGCTTGGGGTT | 3402 | AACCCCAAagCtGGGCAGCTC |
| 2642 | GAGCTGCCCCGGCTTGGGGTT | 3403 | AACCCCAAtgCtGGGCAGCTC |
| 2643 | GAGCTGCCCCGGCTTGGGGTT | 3404 | AACCCCAAaaCcGGGCAGCTC |
| 2644 | GAGCTGCCCCGGCTTGGGGTT | 3405 | AACCCCAAagCcGGGCAGCTC |
| 2645 | GAGCTGCCCCGGCTTGGGGTT | 3406 | AACCCCAAtgCcGGGCAGCTC |
| 2646 | GAGCTGCCCCGGCTTGGGGTT | 3407 | AACCCCAAcgCcGGGCAGCTC |
| 2647 | CCTGCCGCGCGGAGGCGGAGG | 3408 | CCTCCGCCggCcCGCGGCAGG |
| 2648 | CCTGCCGCGCGGAGGCGGAGG | 3409 | CCTCCGCCagCaCGCGGCAGG |
| 2649 | CCTGCCGCGCGGAGGCGGAGG | 3410 | CCTCCGCCaaCtCGCGGCAGG |
| 2650 | CCTGCCGCGCGGAGGCGGAGG | 3411 | CCTCCGCCcaCtCGCGGCAGG |
| 2651 | CCTGCCGCGCGGAGGCGGAGG | 3412 | CCTCCGCCggCtCGCGGCAGG |
| 2652 | CCTGCCGCGCGGAGGCGGAGG | 3413 | CCTCCGCCcaCcCGCGGCAGG |
| 2653 | CCTGCCGCGCGGAGGCGGAGG | 3414 | CCTCCGCCgaCcCGCGGCAGG |
| 2654 | CCTGCCGCGCGGAGGCGGAGG | 3415 | CCTCCGCCagCcCGCGGCAGG |
| 2655 | CCCTGCCGCGCGGAGGCGGAG | 3416 | CTCCGCCTaaGaGCGGCAGGG |
| 2656 | CCCTGCCGCGCGGAGGCGGAG | 3417 | CTCCGCCTgaGaGCGGCAGGG |
| 2657 | CCCTGCCGCGCGGAGGCGGAG | 3418 | CTCCGCCTagGaGCGGCAGGG |
| 2658 | CCCTGCCGCGCGGAGGCGGAG | 3419 | CTCCGCCTggGaGCGGCAGGG |
| 2659 | CCCTGCCGCGCGGAGGCGGAG | 3420 | CTCCGCCTaaGgGCGGCAGGG |
| 2660 | CCCTGCCGCGCGGAGGCGGAG | 3421 | CTCCGCCTgaGgGCGGCAGGG |
| 2661 | CCCTGCCGCGCGGAGGCGGAG | 3422 | CTCCGCCTagGgGCGGCAGGG |
| 2662 | CCCTGCCGCGCGGAGGCGGAG | 3423 | CTCCGCCTggGgGGGCAGGG |
| 2663 | ATCTGCCCCTGCCGCGCGGAG | 3424 | CTCCGCGCtcCcGGGGCAGAT |
| 2664 | ATCTGCCCCTGCCGCGCGGAG | 3425 | CTCCGCGCtaCtGGGGCAGAT |
| 2665 | ATCTGCCCCTGCCGCGCGGAG | 3426 | CTCCGCGCttCtGGGGCAGAT |
| 2666 | ATCTGCCCCTGCCGCGCGGAG | 3427 | CTCCGCGCacCtGGGGCAGAT |
| 2667 | ATCTGCCCCTGCCGCGCGGAG | 3428 | CTCCGCGCtcCtGGGGCAGAT |
| 2668 | ATCTGCCCCTGCCGCGCGGAG | 3429 | CTCCGCGCccCtGGGGCAGAT |
| 2669 | ATCTGCCCCTGCCGCGCGGAG | 3430 | CTCCGCGCaaCcGGGGCAGAT |
| 2670 | ATCTGCCCCTGCCGCGCGGAG | 3431 | CTCCGCGCcaCcGGGGCAGAT |
| 2671 | CTTGCATCTGCCCCTGCCGCG | 3432 | CGCGGCAGaaGgAGATGCAAG |
| 2672 | CTTGCATCTGCCCCTGCCGCG | 3433 | CGCGGCAGaaGaAGATGCAAG |
| 2673 | CTTGCATCTGCCCCTGCCGCG | 3434 | CGCGGCAGtaGaAGATGCAAG |
| 2674 | CTTGCATCTGCCCCTGCCGCG | 3435 | CGCGGCAGctGaAGATGCAAG |
| 2675 | CTTGCATCTGCCCCTGCCGCG | 3436 | CGCGGCAGacGaAGATGCAAG |
| 2676 | CTTGCATCTGCCCCTGCCGCG | 3437 | CGCGGCAGccGaAGATGCAAG |
| 2677 | CTTGCATCTGCCCCTGCCGCG | 3438 | CGCGGCAGctGgAGATGCAAG |
| 2678 | CTTGCATCTGCCCCTGCCGCG | 3439 | CGCGGCAGacGgAGATGCAAG |
| 2679 | AGCGCCTGGGAGGGCGCCGGG | 3440 | CCCGGCGCggTaCCAGGCGCT |
| 2680 | AGCGCCTGGGAGGGCGCCGGG | 3441 | CCCGGCGCagTgCCAGGCGCT |
| 2681 | AGCGCCTGGGAGGGCGCCGGG | 3442 | CCCGGCGCaaTaCCAGGCGCT |
| 2682 | AGCGCCTGGGAGGGCGCCGGG | 3443 | CCCGGCGCgaTaCCAGGCGCT |
| 2683 | AGCGCCTGGGAGGGCGCCGGG | 3444 | CCCGGCGCagTaCCAGGCGCT |
| 2684 | AGCGCCTGGGAGGGCGCCGGG | 3445 | CCCGGCGCaaTgCCAGGCGCT |
| 2685 | AGCGCCTGGGAGGGCGCCGGG | 3446 | CCCGGCGCgaTgCCAGGCGCT |
| 2686 | AGCGCCTGGGAGGGCGCCGGG | 3447 | CCCGGCGCggTgCCAGGCGCT |
| 2687 | CAGACCAGGGCGCCGGCTCCT | 3448 | AGGAGCCGaaGaCCTGGTCTG |
| 2688 | CAGACCAGGGCGCCGGCTCCT | 3449 | AGGAGCCGtaGaCCTGGTCTG |
| 2689 | CAGACCAGGGCGCCGGCTCCT | 3450 | AGGAGCCGagGaCCTGGTCTG |
| 2690 | CAGACCAGGGCGCCGGCTCCT | 3451 | AGGAGCCGcgGaCCTGGTCTG |
| 2691 | CAGACCAGGGCGCCGGCTCCT | 3452 | AGGAGCCGtaGgCCTGGTCTG |
| 2692 | CAGACCAGGGCGCCGGCTCCT | 3453 | AGGAGCCGagGgCCTGGTCTG |
| 2693 | CAGACCAGGGCGCCGGCTCCT | 3454 | AGGAGCCGtgGgCCTGGTCTG |
| 2694 | CAGACCAGGGCGCCGGCTCCT | 3455 | AGGAGCCGcgGgCCTGGTCTG |
| 2695 | TCCGGGCTCGCCAGGAGCTCA | 3456 | TGAGCTCCgaGaGAGCCCGGA |
| 2696 | TCCGGGCTCGCCAGGAGCTCA | 3457 | TGAGCTCCaaGaGAGCCCGGA |
| 2697 | TCCGGGCTCGCCAGGAGCTCA | 3458 | TGAGCTCCatGaGAGCCCGGA |
| 2698 | TCCGGGCTCGCCAGGAGCTCA | 3459 | TGAGCTCCacGaGAGCCCGGA |
| 2699 | TCCGGGCTCGCCAGGAGCTCA | 3460 | TGAGCTCCaaGgGAGCCCGGA |
| 2700 | TCCGGGCTCGCCAGGAGCTCA | 3461 | TGAGCTCCcaGgGAGCCCGGA |
| 2701 | TCCGGGCTCGCCAGGAGCTCA | 3462 | TGAGCTCCgaGgGAGCCCGGA |
| 2702 | TCCGGGCTCGCCAGGAGCTCA | 3463 | TGAGCTCCccGgGAGCCCGGA |
| 2703 | CAGAAACTCCGGGCTCGCCAG | 3464 | CTGGCGAGaaCaGAGTTTCTG |
| 2704 | CAGAAACTCCGGGCTCGCCAG | 3465 | CTGGCGAGgaCaGAGTTTCTG |
| 2705 | CAGAAACTCCGGGCTCGCCAG | 3466 | CTGGCGAGggCaGAGTTTCTG |
| 2706 | CAGAAACTCCGGGCTCGCCAG | 3467 | CTGGCGAGgaCtGAGTTTCTG |
| 2707 | CAGAAACTCCGGGCTCGCCAG | 3468 | CTGGCGAGagCtGAGTTTCTG |
| 2708 | CAGAAACTCCGGGCTCGCCAG | 3469 | CTGGCGAGaaCcGAGTTTCTG |
| 2709 | CAGAAACTCCGGGCTCGCCAG | 3470 | CTGGCGAGagCcGAGTTTCTG |
| 2710 | CAGAAACTCCGGGCTCGCCAG | 3471 | CTGGCGAGggCcGAGTTTCTG |
| 2711 | GTTGCGCCTGCTGCAGAAACT | 3472 | AGTTTCTGgcGaAGGCGCAAC |
| 2712 | GTTGCGCCTGCTGCAGAAACT | 3473 | AGTTTCTGatGaAGGCGCAAC |
| 2713 | GTTGCGCCTGCTGCAGAAACT | 3474 | AGTTTCTGgtGaAGGCGCAAC |
| 2714 | GTTGCGCCTGCTGCAGAAACT | 3475 | AGTTTCTGacGaAGGCGCAAC |
| 2715 | GTTGCGCCTGCTGCAGAAACT | 3476 | AGTTTCTGatGgAGGCGCAAC |
| 2716 | GTTGCGCCTGCTGCAGAAACT | 3477 | AGTTTCTGgtGgAGGCGCAAC |
| 2717 | GTTGCGCCTGCTGCAGAAACT | 3478 | AGTTTCTGacGgAGGCGCAAC |
| 2718 | GTTGCGCCTGCTGCAGAAACT | 3479 | AGTTTCTGgcGgAGGCGCAAC |
| 2719 | TTTCTAGGAGAGGTTGCGCCT | 3480 | AGGCGCAAgaTaTCCTAGAAA |
| 2720 | TTTCTAGGAGAGGTTGCGCCT | 3481 | AGGCGCAAaaTaTCCTAGAAA |
| 2721 | TTTCTAGGAGAGGTTGCGCCT | 3482 | AGGCGCAAagTaTCCTAGAAA |
| 2722 | TTTCTAGGAGAGGTTGCGCCT | 3483 | AGGCGCAAggTaTCCTAGAAA |
| 2723 | TTTCTAGGAGAGGTTGCGCCT | 3484 | AGGCGCAAaaTgTCCTAGAAA |
| 2724 | TTTCTAGGAGAGGTTGCGCCT | 3485 | AGGCGCAAgaTgTCCTAGAAA |
| 2725 | TTTCTAGGAGAGGTTGCGCCT | 3486 | AGGCGCAAagTgTCCTAGAAA |
| 2726 | TTTCTAGGAGAGGTTGCGCCT | 3487 | AGGCGCAAggTgTCCTAGAAA |
| 2727 | GCCTCCGTTTCTAGGAGAGGT | 3488 | ACCTCTCCatGtAACGGAGGC |
| 2728 | GCCTCCGTTTCTAGGAGAGGT | 3489 | ACCTCTCCccGtAACGGAGGC |
| 2729 | GCCTCCGTTTCTAGGAGAGGT | 3490 | ACCTCTCCgcGtAACGGAGGC |
| 2730 | GCCTCCGTTTCTAGGAGAGGT | 3491 | ACCTCTCCatGcAACGGAGGC |
| 2731 | GCCTCCGTTTCTAGGAGAGGT | 3492 | ACCTCTCCctGcAACGGAGGC |
| 2732 | GCCTCCGTTTCTAGGAGAGGT | 3493 | ACCTCTCCgtGcAACGGAGGC |
| 2733 | GCCTCCGTTTCTAGGAGAGGT | 3494 | ACCTCTCCacGcAACGGAGGC |
| 2734 | GCCTCCGTTTCTAGGAGAGGT | 3495 | ACCTCTCCccGcAACGGAGGC |
| 2735 | CCCCGGGGCCTCCGTTTCTAG | 3496 | CTAGAAACccAtGCCCCGGGG |
| 2736 | CCCCGGGGCCTCCGTTTCTAG | 3497 | CTAGAAACtaAaGCCCCGGGG |
| 2737 | CCCCGGGGCCTCCGTTTCTAG | 3498 | CTAGAAACcaAaGCCCCGGGG |
| 2738 | CCCCGGGGCCTCCGTTTCTAG | 3499 | CTAGAAACatAaGCCCCGGGG |
| 2739 | CCCCGGGGCCTCCGTTTCTAG | 3500 | CTAGAAACacAaGCCCCGGGG |
| 2740 | CCCCGGGGCCTCCGTTTCTAG | 3501 | CTAGAAACatAtGCCCCGGGG |
| 2741 | CCCCGGGGCCTCCGTTTCTAG | 3502 | CTAGAAACttAcGCCCCGGGG |
| 2742 | CCCCGGGGCCTCCGTTTCTAG | 3503 | CTAGAAACacAcGCCCCGGGG |
| 2743 | TCCGTCGCCGTCCTCGTCCCC | 3504 | GGGGACGAttAaGGCGACGGA |
| 2744 | TCCGTCGCCGTCCTCGTCCCC | 3505 | GGGGACGAaaAgGGCGACGGA |
| 2745 | TCCGTCGCCGTCCTCGTCCCC | 3506 | GGGGACGAatAgGGCGACGGA |
| 2746 | TCCGTCGCCGTCCTCGTCCCC | 3507 | GGGGACGAttAgGGCGACGGA |
| 2747 | TCCGTCGCCGTCCTCGTCCCC | 3508 | GGGGACGActAgGGCGACGGA |
| 2748 | TCCGTCGCCGTCCTCGTCCCC | 3509 | GGGGACGAtcAgGGCGACGGA |
| 2749 | TCCGTCGCCGTCCTCGTCCCC | 3510 | GGGGACGAccAgGGCGACGGA |
| 2750 | TCCGTCGCCGTCCTCGTCCCC | 3511 | GGGGACGACCAtGGCGACGGA |
| 2751 | AACGAGTCTCCGTCGCCGTCC | 3512 | GGACGGCGtaGaAGACTCGTT |
| 2752 | AACGAGTCTCCGTCGCCGTCC | 3513 | GGACGGCGcaGaAGACTCGTT |
| 2753 | AACGAGTCTCCGTCGCCGTCC | 3514 | GGACGGCGtgGaAGACTCGTT |
| 2754 | AACGAGTCTCCGTCGCCGTCC | 3515 | GGACGGCGtaGtAGACTCGTT |
| 2755 | AACGAGTCTCCGTCGCCGTCC | 3516 | GGACGGCGtgGtAGACTCGTT |
| 2756 | AACGAGTCTCCGTCGCCGTCC | 3517 | GGACGGCGcgGtAGACTCGTT |
| 2757 | AACGAGTCTCCGTCGCCGTCC | 3518 | GGACGGCGtgGcAGACTCGTT |
| 2758 | AACGAGTCTCCGTCGCCGTCC | 3519 | GGACGGCGcgGcAGACTCGTT |
| 2759 | AAACGAGTCTCCGTCGCCGTC | 3520 | GACGGCGAgaGtGACTCGTTT |
| 2760 | AAACGAGTCTCCGTCGCCGTC | 3521 | GACGGCGAgtGtGACTCGTTT |
| 2761 | AAACGAGTCTCCGTCGCCGTC | 3522 | GACGGCGAacGtGACTCGTTT |
| 2762 | AAACGAGTCTCCGTCGCCGTC | 3523 | GACGGCGAatGcGACTCGTTT |
| 2763 | AAACGAGTCTCCGTCGCCGTC | 3524 | GACGGCGAgtGcGACTCGTTT |
| 2764 | AAACGAGTCTCCGTCGCCGTC | 3525 | GACGGCGAacGcGACTCGTTT |
| 2765 | AAACGAGTCTCCGTCGCCGTC | 3526 | GACGGCGAgcGcGACTCGTTT |
| 2766 | AAACGAGTCTCCGTCGCCGTC | 3527 | GACGGCGAGCGgGACTCGTTT |
| 2767 | CAAACGAGTCTCCGTCGCCGT | 3528 | ACGGCGACaaAaACTCGTTTG |
| 2768 | CAAACGAGTCTCCGTCGCCGT | 3529 | ACGGCGACtaAaACTCGTTTG |
| 2769 | CAAACGAGTCTCCGTCGCCGT | 3530 | ACGGCGACcaAaACTCGTTTG |
| 2770 | CAAACGAGTCTCCGTCGCCGT | 3531 | ACGGCGACatAaACTCGTTTG |
| 2771 | CAAACGAGTCTCCGTCGCCGT | 3532 | ACGGCGACttAaACTCGTTTG |
| 2772 | CAAACGAGTCTCCGTCGCCGT | 3533 | ACGGCGACctAaACTCGTTTG |
| 2773 | CAAACGAGTCTCCGTCGCCGT | 3534 | ACGGCGACacAaACTCGTTTG |
| 2774 | CAAACGAGTCTCCGTCGCCGT | 3535 | ACGGCGACccAaACTCGTTTG |
| 2775 | CCAAACGAGTCTCCGTCGCCG | 3536 | CGGCGACGatGtCTCGTTTGG |
| 2776 | CCAAACGAGTCTCCGTCGCCG | 3537 | CGGCGACGttGtCTCGTTTGG |
| 2777 | CCAAACGAGTCTCCGTCGCCG | 3538 | CGGCGACGctGtCTCGTTTGG |
| 2778 | CCAAACGAGTCTCCGTCGCCG | 3539 | CGGCGACGacGtCTCGTTTGG |
| 2779 | CCAAACGAGTCTCCGTCGCCG | 3540 | CGGCGACGtcGtCTCGTTTGG |
| 2780 | CCAAACGAGTCTCCGTCGCCG | 3541 | CGGCGACGccGtCTCGTTTGG |
| 2781 | CCAAACGAGTCTCCGTCGCCG | 3542 | CGGCGACGatGcCTCGTTTGG |
| 2782 | CCAAACGAGTCTCCGTCGCCG | 3543 | CGGCGACGCTGgCTCGTTTGG |
| 2783 | TCCAAACGAGTCTCCGTCGCC | 3544 | GGCGACGGttAaTCGTTTGGA |
| 2784 | TCCAAACGAGTCTCCGTCGCC | 3545 | GGCGACGGctAaTCGTTTGGA |
| 2785 | TCCAAACGAGTCTCCGTCGCC | 3546 | GGCGACGGccAaTCGTTTGGA |
| 2786 | TCCAAACGAGTCTCCGTCGCC | 3547 | GGCGACGGtaAgTCGTTTGGA |
| 2787 | TCCAAACGAGTCTCCGTCGCC | 3548 | GGCGACGGttAgTCGTTTGGA |
| 2788 | TCCAAACGAGTCTCCGTCGCC | 3549 | GGCGACGGctAgTCGTTTGGA |
| 2789 | TCCAAACGAGTCTCCGTCGCC | 3550 | GGCGACGGtcAgTCGTTTGGA |
| 2790 | TCCAAACGAGTCTCCGTCGCC | 3551 | GGCGACGGTCAtTCGTTTGGA |
| 2791 | GTCCAAACGAGTCTCCGTCGC | 3552 | GCGACGGAatCaCGTTTGGAC |
| 2792 | GTCCAAACGAGTCTCCGTCGC | 3553 | GCGACGGActCaCGTTTGGAC |
| 2793 | GTCCAAACGAGTCTCCGTCGC | 3554 | GCGACGGAatCcCGTTTGGAC |
| 2794 | GTCCAAACGAGTCTCCGTCGC | 3555 | GCGACGGActCcCGTTTGGAC |
| 2795 | GTCCAAACGAGTCTCCGTCGC | 3556 | GCGACGGAatCgCGTTTGGAC |
| 2796 | GTCCAAACGAGTCTCCGTCGC | 3557 | GCGACGGAttCgCGTTTGGAC |
| 2797 | GTCCAAACGAGTCTCCGTCGC | 3558 | GCGACGGActCgCGTTTGGAC |
| 2798 | GTCCAAACGAGTCTCCGTCGC | 3559 | GCGACGGAacCgCGTTTGGAC |
| 2799 | AAAGCAGGCTCGCAGGGCCTC | 3560 | GAGGCCCTcaGtGCCTGCTTT |
| 2800 | AAAGCAGGCTCGCAGGGCCTC | 3561 | GAGGCCCTagGtGCCTGCTTT |
| 2801 | AAAGCAGGCTCGCAGGGCCTC | 3562 | GAGGCCCTcgGtGCCTGCTTT |
| 2802 | AAAGCAGGCTCGCAGGGCCTC | 3563 | GAGGCCCTaaGcGCCTGCTTT |
| 2803 | AAAGCAGGCTCGCAGGGCCTC | 3564 | GAGGCCCTtaGcGCCTGCTTT |
| 2804 | AAAGCAGGCTCGCAGGGCCTC | 3565 | GAGGCCCTcaGcGCCTGCTTT |
| 2805 | AAAGCAGGCTCGCAGGGCCTC | 3566 | GAGGCCCTcgGcGCCTGCTTT |
| 2806 | AAAGCAGGCTCGCAGGGCCTC | 3567 | GAGGCCCTCGGgGCCTGCTTT |
| 2807 | CTCAAAGCAGGCTCGCAGGGC | 3568 | GCCCTGCGtaCaTGCTTTGAG |
| 2808 | CTCAAAGCAGGCTCGCAGGGC | 3569 | GCCCTGCGcaCaTGCTTTGAG |
| 2809 | CTCAAAGCAGGCTCGCAGGGC | 3570 | GCCCTGCGttCaTGCTTTGAG |
| 2810 | CTCAAAGCAGGCTCGCAGGGC | 3571 | GCCCTGCGctCaTGCTTTGAG |
| 2811 | CTCAAAGCAGGCTCGCAGGGC | 3572 | GCCCTGCGtcCaTGCTTTGAG |
| 2812 | CTCAAAGCAGGCTCGCAGGGC | 3573 | GCCCTGCGccCaTGCTTTGAG |
| 2813 | CTCAAAGCAGGCTCGCAGGGC | 3574 | GCCCTGCGtaCgTGCTTTGAG |
| 2814 | CTCAAAGCAGGCTCGCAGGGC | 3575 | GCCCTGCGTCCtTGCTTTGAG |
| 2815 | TCTCTGGCCAGCTCCTCCCGG | 3576 | CCGGGAGGtaCaGGCCAGAGA |
| 2816 | TCTCTGGCCAGCTCCTCCCGG | 3577 | CCGGGAGGcaCaGGCCAGAGA |
| 2817 | TCTCTGGCCAGCTCCTCCCGG | 3578 | CCGGGAGGttCaGGCCAGAGA |
| 2818 | TCTCTGGCCAGCTCCTCCCGG | 3579 | CCGGGAGGctCaGGCCAGAGA |
| 2819 | TCTCTGGCCAGCTCCTCCCGG | 3580 | CCGGGAGGtcCaGGCCAGAGA |
| 2820 | TCTCTGGCCAGCTCCTCCCGG | 3581 | CCGGGAGGccCaGGCCAGAGA |
| 2821 | TCTCTGGCCAGCTCCTCCCGG | 3582 | CCGGGAGGtaCcGGCCAGAGA |
| 2822 | TCTCTGGCCAGCTCCTCCCGG | 3583 | CCGGGAGGtcCcGGCCAGAGA |
| 2823 | CGTCTCTCTGGCCAGCTCCTC | 3584 | GAGGAGCTaaCaAGAGAGACG |
| 2824 | CGTCTCTCTGGCCAGCTCCTC | 3585 | GAGGAGCTtaCaAGAGAGACG |
| 2825 | CGTCTCTCTGGCCAGCTCCTC | 3586 | GAGGAGCTcaCaAGAGAGACG |
| 2826 | CGTCTCTCTGGCCAGCTCCTC | 3587 | GAGGAGCTatCaAGAGAGACG |
| 2827 | CGTCTCTCTGGCCAGCTCCTC | 3588 | GAGGAGCTttCaAGAGAGACG |
| 2828 | CGTCTCTCTGGCCAGCTCCTC | 3589 | GAGGAGCTctCaAGAGAGACG |
| 2829 | CGTCTCTCTGGCCAGCTCCTC | 3590 | GAGGAGCTtcCgAGAGAGACG |
| 2830 | CGTCTCTCTGGCCAGCTCCTC | 3591 | GAGGAGCTCCCtAGAGAGACG |
| 2831 | AGATCTGAATCCTGGACTCCG | 3592 | CGGAGTCCtaGtTTCAGATCT |
| 2832 | AGATCTGAATCCTGGACTCCG | 3593 | CGGAGTCCcaGtTTCAGATCT |
| 2833 | AGATCTGAATCCTGGACTCCG | 3594 | CGGAGTCCttGtTTCAGATCT |
| 2834 | AGATCTGAATCCTGGACTCCG | 3595 | CGGAGTCCctGtTTCAGATCT |
| 2835 | AGATCTGAATCCTGGACTCCG | 3596 | CGGAGTCCtcGtTTCAGATCT |
| 2836 | AGATCTGAATCCTGGACTCCG | 3597 | CGGAGTCCccGtTTCAGATCT |
| 2837 | AGATCTGAATCCTGGACTCCG | 3598 | CGGAGTCCtaGcTTCAGATCT |
| 2838 | AGATCTGAATCCTGGACTCCG | 3599 | CGGAGTCCTCGgTTCAGATCT |
| 2839 | CAGATCTGAATCCTGGACTCC | 3600 | GGAGTCCAaaAaTCAGATCTG |
| 2840 | CAGATCTGAATCCTGGACTCC | 3601 | GGAGTCCAtaAaTCAGATCTG |
| 2841 | CAGATCTGAATCCTGGACTCC | 3602 | GGAGTCCAcaAaTCAGATCTG |
| 2842 | CAGATCTGAATCCTGGACTCC | 3603 | GGAGTCCAatAaTCAGATCTG |
| 2843 | CAGATCTGAATCCTGGACTCC | 3604 | GGAGTCCAttAaTCAGATCTG |
| 2844 | CAGATCTGAATCCTGGACTCC | 3605 | GGAGTCCActAaTCAGATCTG |
| 2845 | CAGATCTGAATCCTGGACTCC | 3606 | GGAGTCCAacAaTCAGATCTG |
| 2846 | CAGATCTGAATCCTGGACTCC | 3607 | GGAGTCCAccAcTCAGATCTG |
| 2847 | CCAGATCTGAATCCTGGACTC | 3608 | GAGTCCAGatTaCAGATCTGG |
| 2848 | CCAGATCTGAATCCTGGACTC | 3609 | GAGTCCAGttTaCAGATCTGG |
| 2849 | CCAGATCTGAATCCTGGACTC | 3610 | GAGTCCAGctTaCAGATCTGG |
| 2850 | CCAGATCTGAATCCTGGACTC | 3611 | GAGTCCAGacTaCAGATCTGG |
| 2851 | CCAGATCTGAATCCTGGACTC | 3612 | GAGTCCAGtcTaCAGATCTGG |
| 2852 | CCAGATCTGAATCCTGGACTC | 3613 | GAGTCCAGccTaCAGATCTGG |
| 2853 | CCAGATCTGAATCCTGGACTC | 3614 | GAGTCCAGatTcCAGATCTGG |
| 2854 | CCAGATCTGAATCCTGGACTC | 3615 | GAGTCCAGctTcCAGATCTGG |
| 2855 | AAACCAGATCTGAATCCTGGA | 3616 | TCCAGGATaaAaATCTGGTTT |
| 2856 | AAACCAGATCTGAATCCTGGA | 3617 | TCCAGGATcaAaATCTGGTTT |
| 2857 | AAACCAGATCTGAATCCTGGA | 3618 | TCCAGGATgaAaATCTGGTTT |
| 2858 | AAACCAGATCTGAATCCTGGA | 3619 | TCCAGGATagAaATCTGGTTT |
| 2859 | AAACCAGATCTGAATCCTGGA | 3620 | TCCAGGATcgAaATCTGGTTT |
| 2860 | AAACCAGATCTGAATCCTGGA | 3621 | TCCAGGATggAaATCTGGTTT |
| 2861 | AAACCAGATCTGAATCCTGGA | 3622 | TCCAGGATaaAtATCTGGTTT |
| 2862 | AAACCAGATCTGAATCCTGGA | 3623 | TCCAGGATcaAtATCTGGTTT |
| 2863 | GAAACCAGATCTGAATCCTGG | 3624 | CCAGGATTatGtTCTGGTTTC |
| 2864 | GAAACCAGATCTGAATCCTGG | 3625 | CCAGGATTgtGtTCTGGTTTC |
| 2865 | GAAACCAGATCTGAATCCTGG | 3626 | CCAGGATTacGtTCTGGTTTC |
| 2866 | GAAACCAGATCTGAATCCTGG | 3627 | CCAGGATTgcGtTCTGGTTTC |
| 2867 | GAAACCAGATCTGAATCCTGG | 3628 | CCAGGATTatGcTCTGGTTTC |
| 2868 | GAAACCAGATCTGAATCCTGG | 3629 | CCAGGATTgtGcTCTGGTTTC |
| 2869 | GAAACCAGATCTGAATCCTGG | 3630 | CCAGGATTacGcTCTGGTTTC |
| 2870 | GAAACCAGATCTGAATCCTGG | 3631 | CCAGGATTGTGgTCTGGTTTC |
| 2871 | TGAAACCAGATCTGAATCCTG | 3632 | CAGGATTCtaAaCTGGTTTCA |
| 2872 | TGAAACCAGATCTGAATCCTG | 3633 | CAGGATTCcaAaCTGGTTTCA |
| 2873 | TGAAACCAGATCTGAATCCTG | 3634 | CAGGATTCttAaCTGGTTTCA |
| 2874 | TGAAACCAGATCTGAATCCTG | 3635 | CAGGATTCctAaCTGGTTTCA |
| 2875 | TGAAACCAGATCTGAATCCTG | 3636 | CAGGATTCtcAaCTGGTTTCA |
| 2876 | TGAAACCAGATCTGAATCCTG | 3637 | CAGGATTCccAaCTGGTTTCA |
| 2877 | TGAAACCAGATCTGAATCCTG | 3638 | CAGGATTCtaAcCTGGTTTCA |
| 2878 | TGAAACCAGATCTGAATCCTG | 3639 | CAGGATTCtcAcCTGGTTTCA |
| 2879 | TCTGAAACCAGATCTGAATCC | 3640 | GGATTCAGtaCaGGTTTCAGA |
| 2880 | TCTGAAACCAGATCTGAATCC | 3641 | GGATTCAGcaCaGGTTTCAGA |
| 2881 | TCTGAAACCAGATCTGAATCC | 3642 | GGATTCAGtcCaGGTTTCAGA |
| 2882 | TCTGAAACCAGATCTGAATCC | 3643 | GGATTCAGccCaGGTTTCAGA |
| 2883 | TCTGAAACCAGATCTGAATCC | 3644 | GGATTCAGtgCaGGTTTCAGA |
| 2884 | TCTGAAACCAGATCTGAATCC | 3645 | GGATTCAGcgCaGGTTTCAGA |
| 2885 | TCTGAAACCAGATCTGAATCC | 3646 | GGATTCAGtaCcGGTTTCAGA |
| 2886 | TCTGAAACCAGATCTGAATCC | 3647 | GGATTCAGcaCcGGTTTCAGA |
| 2887 | CCCCCGCCGGGGGCCGCGCTG | 3648 | CAGCGCGGaaCaCGGCGGGGG |
| 2888 | CCCCCGCCGGGGGCCGCGCTG | 3649 | CAGCGCGGgaCaCGGCGGGGG |
| 2889 | CCCCCGCCGGGGGCCGCGCTG | 3650 | CAGCGCGGagCaCGGCGGGGG |
| 2890 | CCCCCGCCGGGGGCCGCGCTG | 3651 | CAGCGCGGggCaCGGCGGGGG |
| 2891 | CCCCCGCCGGGGGCCGCGCTG | 3652 | CAGCGCGGaaCgCGGCGGGGG |
| 2892 | CCCCCGCCGGGGGCCGCGCTG | 3653 | CAGCGCGGgaCgCGGCGGGGG |
| 2893 | CCCCCGCCGGGGGCCGCGCTG | 3654 | CAGCGCGGagCgCGGCGGGGG |
| 2894 | CCCCCGCCGGGGGCCGCGCTG | 3655 | CAGCGCGGGGCtCGGCGGGGG |
| 2895 | TTCCCCACGCGCCGGTGTGGG | 3656 | CCCACACCaaCaCGTGGGGAA |
| 2896 | TTCCCCACGCGCCGGTGTGGG | 3657 | CCCACACCtaCaCGTGGGGAA |
| 2897 | TTCCCCACGCGCCGGTGTGGG | 3658 | CCCACACCcaCaCGTGGGGAA |
| 2898 | TTCCCCACGCGCCGGTGTGGG | 3659 | CCCACACCatCaCGTGGGGAA |
| 2899 | TTCCCCACGCGCCGGTGTGGG | 3660 | CCCACACCttCaCGTGGGGAA |
| 2900 | TTCCCCACGCGCCGGTGTGGG | 3661 | CCCACACCctCaCGTGGGGAA |
| 2901 | TTCCCCACGCGCCGGTGTGGG | 3662 | CCCACACCacCaCGTGGGGAA |
| 2902 | TTCCCCACGCGCCGGTGTGGG | 3663 | CCCACACCccCaCGTGGGGAA |
| 2903 | CACGAAAGCCCCCTGTGGGAG | 3664 | CTCCCACAaaGaGCTTTCGTG |
| 2904 | CACGAAAGCCCCCTGTGGGAG | 3665 | CTCCCACAtaGaGCTTTCGTG |
| 2905 | CACGAAAGCCCCCTGTGGGAG | 3666 | CTCCCACAcaGaGCTTTCGTG |
| 2906 | CACGAAAGCCCCCTGTGGGAG | 3667 | CTCCCACAatGaGCTTTCGTG |
| 2907 | CACGAAAGCCCCCTGTGGGAG | 3668 | CTCCCACAttGaGCTTTCGTG |
| 2908 | CACGAAAGCCCCCTGTGGGAG | 3669 | CTCCCACActGaGCTTTCGTG |
| 2909 | CACGAAAGCCCCCTGTGGGAG | 3670 | CTCCCACAacGaGCTTTCGTG |
| 2910 | CACGAAAGCCCCCTGTGGGAG | 3671 | CTCCCACAccGaGCTTTCGTG |
| 2911 | AGGCGAAATCCCCGCGCGCCG | 3672 | CGGCGCGCaaGaATTTCGCCT |
| 2912 | AGGCGAAATCCCCGCGCGCCG | 3673 | CGGCGCGCtaGaATTTCGCCT |
| 2913 | AGGCGAAATCCCCGCGCGCCG | 3674 | CGGCGCGCcaGaATTTCGCCT |
| 2914 | AGGCGAAATCCCCGCGCGCCG | 3675 | CGGCGCGCatGaATTTCGCCT |
| 2915 | AGGCGAAATCCCCGCGCGCCG | 3676 | CGGCGCGCttGaATTTCGCCT |
| 2916 | AGGCGAAATCCCCGCGCGCCG | 3677 | CGGCGCGCctGaATTTCGCCT |
| 2917 | AGGCGAAATCCCCGCGCGCCG | 3678 | CGGCGCGCacGaATTTCGCCT |
| 2918 | AGGCGAAATCCCCGCGCGCCG | 3679 | CGGCGCGCccGaATTTCGCCT |
| 2919 | TAGGCGAAATCCCCGCGCGCC | 3680 | GGCGCGCGtaGtTTTCGCCTA |
| 2920 | TAGGCGAAATCCCCGCGCGCC | 3681 | GGCGCGCGatGtTTTCGCCTA |
| 2921 | TAGGCGAAATCCCCGCGCGCC | 3682 | GGCGCGCGacGtTTTCGCCTA |
| 2922 | TAGGCGAAATCCCCGCGCGCC | 3683 | GGCGCGCGaaGcTTTCGCCTA |
| 2923 | TAGGCGAAATCCCCGCGCGCC | 3684 | GGCGCGCGtaGcTTTCGCCTA |
| 2924 | TAGGCGAAATCCCCGCGCGCC | 3685 | GGCGCGCGttGcTTTCGCCTA |
| 2925 | TAGGCGAAATCCCCGCGCGCC | 3686 | GGCGCGCGacGcTTTCGCCTA |
| 2926 | TAGGCGAAATCCCCGCGCGCC | 3687 | GGCGCGCGCCGgTTTCGCCTA |
| 2927 | GTAGGCGAAATCCCCGCGCGC | 3688 | GCGCGCGGacAaTTCGCCTAC |
| 2928 | GTAGGCGAAATCCCCGCGCGC | 3689 | GCGCGCGGcaAcTTCGCCTAC |
| 2929 | GTAGGCGAAATCCCCGCGCGC | 3690 | GCGCGCGGatAcTTCGCCTAC |
| 2930 | GTAGGCGAAATCCCCGCGCGC | 3691 | GCGCGCGGctAcTTCGCCTAC |
| 2931 | GTAGGCGAAATCCCCGCGCGC | 3692 | GCGCGCGGacAcTTCGCCTAC |
| 2932 | GTAGGCGAAATCCCCGCGCGC | 3693 | GCGCGCGGtcAcTTCGCCTAC |
| 2933 | GTAGGCGAAATCCCCGCGCGC | 3694 | GCGCGCGGccAcTTCGCCTAC |
| 2934 | GTAGGCGAAATCCCCGCGCGC | 3695 | GCGCGCGGttAgTTCGCCTAC |
| 2935 | CCACCGAGGAGCCTGAGGGTG | 3696 | CACCCTCAaaCaCCTCGGTGG |
| 2936 | CCACCGAGGAGCCTGAGGGTG | 3697 | CACCCTCAtaCaCCTCGGTGG |
| 2937 | CCACCGAGGAGCCTGAGGGTG | 3698 | CACCCTCAatCaCCTCGGTGG |
| 2938 | CCACCGAGGAGCCTGAGGGTG | 3699 | CACCCTCAccCcCCTCGGTGG |
| 2939 | CCACCGAGGAGCCTGAGGGTG | 3700 | CACCCTCAaaCgCCTCGGTGG |
| 2940 | CCACCGAGGAGCCTGAGGGTG | 3701 | CACCCTCAttCgCCTCGGTGG |
| 2941 | CCACCGAGGAGCCTGAGGGTG | 3702 | CACCCTCActCgCCTCGGTGG |
| 2942 | CCACCGAGGAGCCTGAGGGTG | 3703 | CACCCTCAccCgCCTCGGTGG |
| 2943 | TGCGGAGGCCACCGAGGAGCC | 3704 | GGCTCCTCaaTaGCCTCCGCA |
| 2944 | TGCGGAGGCCACCGAGGAGCC | 3705 | GGCTCCTCtaTaGCCTCCGCA |
| 2945 | TGCGGAGGCCACCGAGGAGCC | 3706 | GGCTCCTCcaTaGCCTCCGCA |
| 2946 | TGCGGAGGCCACCGAGGAGCC | 3707 | GGCTCCTCatTaGCCTCCGCA |
| 2947 | TGCGGAGGCCACCGAGGAGCC | 3708 | GGCTCCTCttTaGCCTCCGCA |
| 2948 | TGCGGAGGCCACCGAGGAGCC | 3709 | GGCTCCTCctTaGCCTCCGCA |
| 2949 | TGCGGAGGCCACCGAGGAGCC | 3710 | GGCTCCTCccTaGCCTCCGCA |
| 2950 | TGCGGAGGCCACCGAGGAGCC | 3711 | GGCTCCTCttTtGCCTCCGCA |
| 2951 | TCCCGGTCCTCCCGGCTTTTG | 3712 | CAAAAGCCaaGtGGACCGGGA |
| 2952 | TCCCGGTCCTCCCGGCTTTTG | 3713 | CAAAAGCCtaGtGGACCGGGA |
| 2953 | TCCCGGTCCTCCCGGCTTTTG | 3714 | CAAAAGCCcaGtGGACCGGGA |
| 2954 | TCCCGGTCCTCCCGGCTTTTG | 3715 | CAAAAGCCatGtGGACCGGGA |
| 2955 | TCCCGGTCCTCCCGGCTTTTG | 3716 | CAAAAGCCttGtGGACCGGGA |
| 2956 | TCCCGGTCCTCCCGGCTTTTG | 3717 | CAAAAGCCctGtGGACCGGGA |
| 2957 | TCCCGGTCCTCCCGGCTTTTG | 3718 | CAAAAGCCccGtGGACCGGGA |
| 2958 | TCCCGGTCCTCCCGGCTTTTG | 3719 | CAAAAGCCCCGgGGACCGGGA |
| 2959 | CTTGAGCGGGCCCAGGCTGTG | 3720 | CACAGCCTaaGaCCGCTCAAG |
| 2960 | CTTGAGCGGGCCCAGGCTGTG | 3721 | CACAGCCTtaGaCCGCTCAAG |
| 2961 | CTTGAGCGGGCCCAGGCTGTG | 3722 | CACAGCCTcaGaCCGCTCAAG |
| 2962 | CTTGAGCGGGCCCAGGCTGTG | 3723 | CACAGCCTatGaCCGCTCAAG |
| 2963 | CTTGAGCGGGCCCAGGCTGTG | 3724 | CACAGCCTttGaCCGCTCAAG |
| 2964 | CTTGAGCGGGCCCAGGCTGTG | 3725 | CACAGCCTctGaCCGCTCAAG |
| 2965 | CTTGAGCGGGCCCAGGCTGTG | 3726 | CACAGCCTacGaCCGCTCAAG |
| 2966 | CTTGAGCGGGCCCAGGCTGTG | 3727 | CACAGCCTCCGtCCGCTCAAG |
| 2967 | TGGCCCTGCGGCCCCGCTTGA | 3728 | TCAAGCGGaaCaGCAGGGCCA |
| 2968 | TGGCCCTGCGGCCCCGCTTGA | 3729 | TCAAGCGGtaCaGCAGGGCCA |
| 2969 | TGGCCCTGCGGCCCCGCTTGA | 3730 | TCAAGCGGcaCaGCAGGGCCA |
| 2970 | TGGCCCTGCGGCCCCGCTTGA | 3731 | TCAAGCGGatCaGCAGGGCCA |
| 2971 | TGGCCCTGCGGCCCCGCTTGA | 3732 | TCAAGCGGttCaGCAGGGCCA |
| 2972 | TGGCCCTGCGGCCCCGCTTGA | 3733 | TCAAGCGGctCaGCAGGGCCA |
| 2973 | TGGCCCTGCGGCCCCGCTTGA | 3734 | TCAAGCGGacCaGCAGGGCCA |
| 2974 | TGGCCCTGCGGCCCCGCTTGA | 3735 | TCAAGCGGCCCtGCAGGGCCA |
| 2975 | TGCCTGGCCCTTCGATTCTGA | 3736 | TCAGAATCatAaGGCCAGGCA |
| 2976 | TGCCTGGCCCTTCGATTCTGA | 3737 | TCAGAATCttAaGGCCAGGCA |
| 2977 | TGCCTGGCCCTTCGATTCTGA | 3738 | TCAGAATCctAaGGCCAGGCA |
| 2978 | TGCCTGGCCCTTCGATTCTGA | 3739 | TCAGAATCacAaGGCCAGGCA |
| 2979 | TGCCTGGCCCTTCGATTCTGA | 3740 | TCAGAATCtcAaGGCCAGGCA |
| 2980 | TGCCTGGCCCTTCGATTCTGA | 3741 | TCAGAATCccAaGGCCAGGCA |
| 2981 | TGCCTGGCCCTTCGATTCTGA | 3742 | TCAGAATCatAtGGCCAGGCA |
| 2982 | TGCCTGGCCCTTCGATTCTGA | 3743 | TCAGAATCttAtGGCCAGGCA |
| 2983 | GTGCCTGGCCCTTCGATTCTG | 3744 | CAGAATCGttGaGCCAGGCAC |
| 2984 | GTGCCTGGCCCTTCGATTCTG | 3745 | CAGAATCGctGaGCCAGGCAC |
| 2985 | GTGCCTGGCCCTTCGATTCTG | 3746 | CAGAATCGtcGaGCCAGGCAC |
| 2986 | GTGCCTGGCCCTTCGATTCTG | 3747 | CAGAATCGccGaGCCAGGCAC |
| 2987 | GTGCCTGGCCCTTCGATTCTG | 3748 | CAGAATCGttGtGCCAGGCAC |
| 2988 | GTGCCTGGCCCTTCGATTCTG | 3749 | CAGAATCGctGtGCCAGGCAC |
| 2989 | GTGCCTGGCCCTTCGATTCTG | 3750 | CAGAATCGtcGtGCCAGGCAC |
| 2990 | GTGCCTGGCCCTTCGATTCTG | 3751 | CAGAATCGccGtGCCAGGCAC |
| 2991 | GGTGCCTGGCCCTTCGATTCT | 3752 | AGAATCGAtaGaCCAGGCACC |
| 2992 | GGTGCCTGGCCCTTCGATTCT | 3753 | AGAATCGAcaGaCCAGGCACC |
| 2993 | GGTGCCTGGCCCTTCGATTCT | 3754 | AGAATCGAttGaCCAGGCACC |
| 2994 | GGTGCCTGGCCCTTCGATTCT | 3755 | AGAATCGActGaCCAGGCACC |
| 2995 | GGTGCCTGGCCCTTCGATTCT | 3756 | AGAATCGAtcGaCCAGGCACC |
| 2996 | GGTGCCTGGCCCTTCGATTCT | 3757 | AGAATCGAccGaCCAGGCACC |
| 2997 | GGTGCCTGGCCCTTCGATTCT | 3758 | AGAATCGAtaGtCCAGGCACC |
| 2998 | GGTGCCTGGCCCTTCGATTCT | 3759 | AGAATCGAcaGtCCAGGCACC |
| 2999 | GGGTGCCTGGCCCTTCGATTC | 3760 | GAATCGAAaaGaCAGGCACCC |
| 3000 | GGGTGCCTGGCCCTTCGATTC | 3761 | GAATCGAAtaGaCAGGCACCC |
| 3001 | GGGTGCCTGGCCCTTCGATTC | 3762 | GAATCGAAcaGaCAGGCACCC |
| 3002 | GGGTGCCTGGCCCTTCGATTC | 3763 | GAATCGAAatGaCAGGCACCC |
| 3003 | GGGTGCCTGGCCCTTCGATTC | 3764 | GAATCGAAttGaCAGGCACCC |
| 3004 | GGGTGCCTGGCCCTTCGATTC | 3765 | GAATCGAActGaCAGGCACCC |
| 3005 | GGGTGCCTGGCCCTTCGATTC | 3766 | GAATCGAAacGaCAGGCACCC |
| 3006 | GGGTGCCTGGCCCTTCGATTC | 3767 | GAATCGAAtcGaCAGGCACCC |
| 3007 | CGGGTGCCTGGCCCTTCGATT | 3768 | AATCGAAGaaCaAGGCACCCG |
| 3008 | CGGGTGCCTGGCCCTTCGATT | 3769 | AATCGAAGtaCaAGGCACCCG |
| 3009 | CGGGTGCCTGGCCCTTCGATT | 3770 | AATCGAAGcaCaAGGCACCCG |
| 3010 | CGGGTGCCTGGCCCTTCGATT | 3771 | AATCGAAGatCaAGGCACCCG |
| 3011 | CGGGTGCCTGGCCCTTCGATT | 3772 | AATCGAAGttCaAGGCACCCG |
| 3012 | CGGGTGCCTGGCCCTTCGATT | 3773 | AATCGAAGctCaAGGCACCCG |
| 3013 | CGGGTGCCTGGCCCTTCGATT | 3774 | AATCGAAGacCaAGGCACCCG |
| 3014 | CGGGTGCCTGGCCCTTCGATT | 3775 | AATCGAAGtcCaAGGCACCCG |
| 3015 | CCGGGTGCCTGGCCCTTCGAT | 3776 | ATCGAAGGaaCtGGCACCCGG |
| 3016 | CCGGGTGCCTGGCCCTTCGAT | 3777 | ATCGAAGGtaCtGGCACCCGG |
| 3017 | CCGGGTGCCTGGCCCTTCGAT | 3778 | ATCGAAGGcaCtGGCACCCGG |
| 3018 | CCGGGTGCCTGGCCCTTCGAT | 3779 | ATCGAAGGagCtGGCACCCGG |
| 3019 | CCGGGTGCCTGGCCCTTCGAT | 3780 | ATCGAAGGtgCtGGCACCCGG |
| 3020 | CCGGGTGCCTGGCCCTTCGAT | 3781 | ATCGAAGGcgCtGGCACCCGG |
| 3021 | CCGGGTGCCTGGCCCTTCGAT | 3782 | ATCGAAGGaaCcGGCACCCGG |
| 3022 | CCGGGTGCCTGGCCCTTCGAT | 3783 | ATCGAAGGtaCcGGCACCCGG |
| 3023 | CCCGGGTGCCTGGCCCTTCGA | 3784 | TCGAAGGGaaAaGCACCCGGG |
| 3024 | CCCGGGTGCCTGGCCCTTCGA | 3785 | TCGAAGGGgaAaGCACCCGGG |
| 3025 | CCCGGGTGCCTGGCCCTTCGA | 3786 | TCGAAGGGagAaGCACCCGGG |
| 3026 | CCCGGGTGCCTGGCCCTTCGA | 3787 | TCGAAGGGggAaGCACCCGGG |
| 3027 | CCCGGGTGCCTGGCCCTTCGA | 3788 | TCGAAGGGaaAtGCACCCGGG |
| 3028 | CCCGGGTGCCTGGCCCTTCGA | 3789 | TCGAAGGGgaAtGCACCCGGG |
| 3029 | CCCGGGTGCCTGGCCCTTCGA | 3790 | TCGAAGGGagAtGCACCCGGG |
| 3030 | CCCGGGTGCCTGGCCCTTCGA | 3791 | TCGAAGGGggAtGCACCCGGG |
| 3031 | TCCCGGGTGCCTGGCCCTTCG | 3792 | CGAAGGGCatGaCACCCGGGA |
| 3032 | TCCCGGGTGCCTGGCCCTTCG | 3793 | CGAAGGGCgtGaCACCCGGGA |
| 3033 | TCCCGGGTGCCTGGCCCTTCG | 3794 | CGAAGGGCacGaCACCCGGGA |
| 3034 | TCCCGGGTGCCTGGCCCTTCG | 3795 | CGAAGGGCgcGaCACCCGGGA |
| 3035 | TCCCGGGTGCCTGGCCCTTCG | 3796 | CGAAGGGCatGtCACCCGGGA |
| 3036 | TCCCGGGTGCCTGGCCCTTCG | 3797 | CGAAGGGCgtGtCACCCGGGA |
| 3037 | TCCCGGGTGCCTGGCCCTTCG | 3798 | CGAAGGGCacGtCACCCGGGA |
| 3038 | TCCCGGGTGCCTGGCCCTTCG | 3799 | CGAAGGGCgcGtCACCCGGGA |
| 3039 | GTCCCGGGTGCCTGGCCCTTC | 3800 | GAAGGGCCtaGaACCCGGGAC |
| 3040 | GTCCCGGGTGCCTGGCCCTTC | 3801 | GAAGGGCCcaGaACCCGGGAC |
| 3041 | GTCCCGGGTGCCTGGCCCTTC | 3802 | GAAGGGCCttGaACCCGGGAC |
| 3042 | GTCCCGGGTGCCTGGCCCTTC | 3803 | GAAGGGCCctGaACCCGGGAC |
| 3043 | GTCCCGGGTGCCTGGCCCTTC | 3804 | GAAGGGCCtcGaACCCGGGAC |
| 3044 | GTCCCGGGTGCCTGGCCCTTC | 3805 | GAAGGGCCccGaACCCGGGAC |
| 3045 | GTCCCGGGTGCCTGGCCCTTC | 3806 | GAAGGGCCtaGgACCCGGGAC |
| 3046 | GTCCCGGGTGCCTGGCCCTTC | 3807 | GAAGGGCCcaGgACCCGGGAC |
| 3047 | TGTCCCGGGTGCCTGGCCCTT | 3808 | AAGGGCCAaaCtCCCGGGACA |
| 3048 | TGTCCCGGGTGCCTGGCCCTT | 3809 | AAGGGCCAtaCtCCCGGGACA |
| 3049 | TGTCCCGGGTGCCTGGCCCTT | 3810 | AAGGGCCAcaCtCCCGGGACA |
| 3050 | TGTCCCGGGTGCCTGGCCCTT | 3811 | AAGGGCCAatCtCCCGGGACA |
| 3051 | TGTCCCGGGTGCCTGGCCCTT | 3812 | AAGGGCCAttCtCCCGGGACA |
| 3052 | TGTCCCGGGTGCCTGGCCCTT | 3813 | AAGGGCCActCtCCCGGGACA |
| 3053 | TGTCCCGGGTGCCTGGCCCTT | 3814 | AAGGGCCAacCtCCCGGGACA |
| 3054 | TGTCCCGGGTGCCTGGCCCTT | 3815 | AAGGGCCAtcCtCCCGGGACA |
| 3055 | CCTGTCCCGGGTGCCTGGCCC | 3816 | GGGCCAGGatCaCGGGACAGG |
| 3056 | CCTGTCCCGGGTGCCTGGCCC | 3817 | GGGCCAGGgtCaCGGGACAGG |
| 3057 | CCTGTCCCGGGTGCCTGGCCC | 3818 | GGGCCAGGacCaCGGGACAGG |
| 3058 | CCTGTCCCGGGTGCCTGGCCC | 3819 | GGGCCAGGgcCaCGGGACAGG |
| 3059 | CCTGTCCCGGGTGCCTGGCCC | 3820 | GGGCCAGGatCgCGGGACAGG |
| 3060 | CCTGTCCCGGGTGCCTGGCCC | 3821 | GGGCCAGGgtCgCGGGACAGG |
| 3061 | CCTGTCCCGGGTGCCTGGCCC | 3822 | GGGCCAGGacCgCGGGACAGG |
| 3062 | CCTGTCCCGGGTGCCTGGCCC | 3823 | GGGCCAGGgcCgCGGGACAGG |
| 3063 | CCCTGTCCCGGGTGCCTGGCC | 3824 | GGCCAGGCtaCaGGGACAGGG |
| 3064 | CCCTGTCCCGGGTGCCTGGCC | 3825 | GGCCAGGCcaCaGGGACAGGG |
| 3065 | CCCTGTCCCGGGTGCCTGGCC | 3826 | GGCCAGGCtgCaGGGACAGGG |
| 3066 | CCCTGTCCCGGGTGCCTGGCC | 3827 | GGCCAGGCcgCaGGGACAGGG |
| 3067 | CCCTGTCCCGGGTGCCTGGCC | 3828 | GGCCAGGCtaCgGGGACAGGG |
| 3068 | CCCTGTCCCGGGTGCCTGGCC | 3829 | GGCCAGGCcaCgGGGACAGGG |
| 3069 | CCCTGTCCCGGGTGCCTGGCC | 3830 | GGCCAGGCtgCgGGGACAGGG |
| 3070 | CCCTGTCCCGGGTGCCTGGCC | 3831 | GGCCAGGCcgCgGGGACAGGG |
| 3071 | ACCCTGTCCCGGGTGCCTGGC | 3832 | GCCAGGCAaaCaGGACAGGGT |
| 3072 | ACCCTGTCCCGGGTGCCTGGC | 3833 | GCCAGGCAgaCaGGACAGGGT |
| 3073 | ACCCTGTCCCGGGTGCCTGGC | 3834 | GCCAGGCAagCaGGACAGGGT |
| 3074 | ACCCTGTCCCGGGTGCCTGGC | 3835 | GCCAGGCAggCaGGACAGGGT |
| 3075 | ACCCTGTCCCGGGTGCCTGGC | 3836 | GCCAGGCAaaCtGGACAGGGT |
| 3076 | ACCCTGTCCCGGGTGCCTGGC | 3837 | GCCAGGCAgaCtGGACAGGGT |
| 3077 | ACCCTGTCCCGGGTGCCTGGC | 3838 | GCCAGGCAagCtGGACAGGGT |
| 3078 | ACCCTGTCCCGGGTGCCTGGC | 3839 | GCCAGGCAggCtGGACAGGGT |
| 3079 | CACCCTGTCCCGGGTGCCTGG | 3840 | CCAGGCACaaGaGACAGGGTG |
| 3080 | CACCCTGTCCCGGGTGCCTGG | 3841 | CCAGGCACgaGaGACAGGGTG |
| 3081 | CACCCTGTCCCGGGTGCCTGG | 3842 | CCAGGCACagGaGACAGGGTG |
| 3082 | CACCCTGTCCCGGGTGCCTGG | 3843 | CCAGGCACggGaGACAGGGTG |
| 3083 | CACCCTGTCCCGGGTGCCTGG | 3844 | CCAGGCACaaGtGACAGGGTG |
| 3084 | CACCCTGTCCCGGGTGCCTGG | 3845 | CCAGGCACgaGtGACAGGGTG |
| 3085 | CACCCTGTCCCGGGTGCCTGG | 3846 | CCAGGCACagGtGACAGGGTG |
| 3086 | CACCCTGTCCCGGGTGCCTGG | 3847 | CCAGGCACggGtGACAGGGTG |
| 3087 | CCACCCTGTCCCGGGTGCCTG | 3848 | CAGGCACCaaGaACAGGGTGG |
| 3088 | CCACCCTGTCCCGGGTGCCTG | 3849 | CAGGCACCgaGaACAGGGTGG |
| 3089 | CCACCCTGTCCCGGGTGCCTG | 3850 | CAGGCACCatGaACAGGGTGG |
| 3090 | CCACCCTGTCCCGGGTGCCTG | 3851 | CAGGCACCgtGaACAGGGTGG |
| 3091 | CCACCCTGTCCCGGGTGCCTG | 3852 | CAGGCACCacGaACAGGGTGG |
| 3092 | CCACCCTGTCCCGGGTGCCTG | 3853 | CAGGCACCgcGaACAGGGTGG |
| 3093 | CCACCCTGTCCCGGGTGCCTG | 3854 | CAGGCACCaaGtACAGGGTGG |
| 3094 | CCACCCTGTCCCGGGTGCCTG | 3855 | CAGGCACCgaGtACAGGGTGG |
| 3095 | GCCACCCTGTCCCGGGTGCCT | 3856 | AGGCACCCaaGtCAGGGTGGC |
| 3096 | GCCACCCTGTCCCGGGTGCCT | 3857 | AGGCACCCtaGtCAGGGTGGC |
| 3097 | GCCACCCTGTCCCGGGTGCCT | 3858 | AGGCACCCcaGtCAGGGTGGC |
| 3098 | GCCACCCTGTCCCGGGTGCCT | 3859 | AGGCACCCatGtCAGGGTGGC |
| 3099 | GCCACCCTGTCCCGGGTGCCT | 3860 | AGGCACCCttGtCAGGGTGGC |
| 3100 | GCCACCCTGTCCCGGGTGCCT | 3861 | AGGCACCCctGtCAGGGTGGC |
| 3101 | GCCACCCTGTCCCGGGTGCCT | 3862 | AGGCACCCacGtCAGGGTGGC |
| 3102 | GCCACCCTGTCCCGGGTGCCT | 3863 | AGGCACCCtcGtCAGGGTGGC |
| 3103 | TGCCACCCTGTCCCGGGTGCC | 3864 | GGCACCCGaaAaAGGGTGGCA |
| 3104 | TGCCACCCTGTCCCGGGTGCC | 3865 | GGCACCCGtaAaAGGGTGGCA |
| 3105 | TGCCACCCTGTCCCGGGTGCC | 3866 | GGCACCCGcaAaAGGGTGGCA |
| 3106 | TGCCACCCTGTCCCGGGTGCC | 3867 | GGCACCCGatAaAGGGTGGCA |
| 3107 | TGCCACCCTGTCCCGGGTGCC | 3868 | GGCACCCGttAaAGGGTGGCA |
| 3108 | TGCCACCCTGTCCCGGGTGCC | 3869 | GGCACCCGctAaAGGGTGGCA |
| 3109 | TGCCACCCTGTCCCGGGTGCC | 3870 | GGCACCCGacAaAGGGTGGCA |
| 3110 | TGCCACCCTGTCCCGGGTGCC | 3871 | GGCACCCGtcAaAGGGTGGCA |
| 3111 | CTGCCACCCTGTCCCGGGTGC | 3872 | GCACCCGGatCtGGGTGGCAG |
| 3112 | CTGCCACCCTGTCCCGGGTGC | 3873 | GCACCCGGttCtGGGTGGCAG |
| 3113 | CTGCCACCCTGTCCCGGGTGC | 3874 | GCACCCGGctCtGGGTGGCAG |
| 3114 | CTGCCACCCTGTCCCGGGTGC | 3875 | GCACCCGGacCtGGGTGGCAG |
| 3115 | CTGCCACCCTGTCCCGGGTGC | 3876 | GCACCCGGtcCtGGGTGGCAG |
| 3116 | CTGCCACCCTGTCCCGGGTGC | 3877 | GCACCCGGccCtGGGTGGCAG |
| 3117 | CTGCCACCCTGTCCCGGGTGC | 3878 | GCACCCGGatCcGGGTGGCAG |
| 3118 | CTGCCACCCTGTCCCGGGTGC | 3879 | GCACCCGGttCcGGGTGGCAG |
| 3119 | CCTGCCACCCTGTCCCGGGTG | 3880 | CACCCGGGtaAaGGTGGCAGG |
| 3120 | CCTGCCACCCTGTCCCGGGTG | 3881 | CACCCGGGcaAaGGTGGCAGG |
| 3121 | CCTGCCACCCTGTCCCGGGTG | 3882 | CACCCGGGtgAaGGTGGCAGG |
| 3122 | CCTGCCACCCTGTCCCGGGTG | 3883 | CACCCGGGcgAaGGTGGCAGG |
| 3123 | CCTGCCACCCTGTCCCGGGTG | 3884 | CACCCGGGtaAtGGTGGCAGG |
| 3124 | CCTGCCACCCTGTCCCGGGTG | 3885 | CACCCGGGcaAtGGTGGCAGG |
| 3125 | CCTGCCACCCTGTCCCGGGTG | 3886 | CACCCGGGtgAtGGTGGCAGG |
| 3126 | CCTGCCACCCTGTCCCGGGTG | 3887 | CACCCGGGcgAtGGTGGCAGG |
| 3127 | GCCCTGCCACCCTGTCCCGGG | 3888 | CCCGGGACtaGaTGGCAGGGC |
| 3128 | GCCCTGCCACCCTGTCCCGGG | 3889 | CCCGGGACcaGaTGGCAGGGC |
| 3129 | GCCCTGCCACCCTGTCCCGGG | 3890 | CCCGGGACttGaTGGCAGGGC |
| 3130 | GCCCTGCCACCCTGTCCCGGG | 3891 | CCCGGGACctGaTGGCAGGGC |
| 3131 | GCCCTGCCACCCTGTCCCGGG | 3892 | CCCGGGACtcGaTGGCAGGGC |
| 3132 | GCCCTGCCACCCTGTCCCGGG | 3893 | CCCGGGACccGaTGGCAGGGC |
| 3133 | GCCCTGCCACCCTGTCCCGGG | 3894 | CCCGGGACtaGtTGGCAGGGC |
| 3134 | GCCCTGCCACCCTGTCCCGGG | 3895 | CCCGGGACcaGtTGGCAGGGC |
| 3135 | CGCCCTGCCACCCTGTCCCGG | 3896 | CCGGGACAaaGaGGCAGGGCG |
| 3136 | CGCCCTGCCACCCTGTCCCGG | 3897 | CCGGGACAtaGaGGCAGGGCG |
| 3137 | CGCCCTGCCACCCTGTCCCGG | 3898 | CCGGGACAcaGaGGCAGGGCG |
| 3138 | CGCCCTGCCACCCTGTCCCGG | 3899 | CCGGGACAatGaGGCAGGGCG |
| 3139 | CGCCCTGCCACCCTGTCCCGG | 3900 | CCGGGACAttGaGGCAGGGCG |
| 3140 | CGCCCTGCCACCCTGTCCCGG | 3901 | CCGGGACActGaGGCAGGGCG |
| 3141 | CGCCCTGCCACCCTGTCCCGG | 3902 | CCGGGACAacGaGGCAGGGCG |
| 3142 | CGCCCTGCCACCCTGTCCCGG | 3903 | CCGGGACAtcGaGGCAGGGCG |
| 3143 | GCGCCCTGCCACCCTGTCCCG | 3904 | CGGGACAGaaTaGCAGGGCGC |
| 3144 | GCGCCCTGCCACCCTGTCCCG | 3905 | CGGGACAGtaTaGCAGGGCGC |
| 3145 | GCGCCCTGCCACCCTGTCCCG | 3906 | CGGGACAGcaTaGCAGGGCGC |
| 3146 | GCGCCCTGCCACCCTGTCCCG | 3907 | CGGGACAGatTaGCAGGGCGC |
| 3147 | GCGCCCTGCCACCCTGTCCCG | 3908 | CGGGACAGttTaGCAGGGCGC |
| 3148 | GCGCCCTGCCACCCTGTCCCG | 3909 | CGGGACAGctTaGCAGGGCGC |
| 3149 | GCGCCCTGCCACCCTGTCCCG | 3910 | CGGGACAGacTaGCAGGGCGC |
| 3150 | GCGCCCTGCCACCCTGTCCCG | 3911 | CGGGACAGtcTaGCAGGGCGC |
| 3151 | GGCGCCCTGCCACCCTGTCCC | 3912 | GGGACAGGaaGaCAGGGCGCC |
| 3152 | GGCGCCCTGCCACCCTGTCCC | 3913 | GGGACAGGtaGaCAGGGCGCC |
| 3153 | GGCGCCCTGCCACCCTGTCCC | 3914 | GGGACAGGcaGaCAGGGCGCC |
| 3154 | GGCGCCCTGCCACCCTGTCCC | 3915 | GGGACAGGacGaCAGGGCGCC |
| 3155 | GGCGCCCTGCCACCCTGTCCC | 3916 | GGGACAGGtcGaCAGGGCGCC |
| 3156 | GGCGCCCTGCCACCCTGTCCC | 3917 | GGGACAGGccGaCAGGGCGCC |
| 3157 | GGCGCCCTGCCACCCTGTCCC | 3918 | GGGACAGGagGaCAGGGCGCC |
| 3158 | GGCGCCCTGCCACCCTGTCCC | 3919 | GGGACAGGtgGaCAGGGCGCC |
| 3159 | TTCGATTCTGAAACCAGATCT | 3920 | AGATCTGGAGTAAGAATCGAA |

DRG De-Targeting miRNAs

The present invention provides recombinant nucleic acids and recombinant expression vectors (e.g., AAV vectors) that encode a transgene (e.g., an engineered pri-miRNA) and the binding site(s) for specific micro RNAs (miRs) that direct the miRs to selectively inhibit expression of the same transgene (e.g., the same engineered pri-miRNA). By the present invention, it was discovered that miR-338-3p, miR-138-5p, and miR-9-5p are highly expressed in dorsal root ganglial (DRG) cells and not expressed, or expressed at substantially low levels, in muscle cells (including skeletal and heart muscle cells).

Accordingly, aspects of the invention provide a recombinant nucleic acid and an engineered vector comprising a first nucleic acid sequence encoding a transgene (e.g., an engineered pri-miRNA) and a second nucleic acid sequence encoding for the binding site(s) of one or more miRs selected from among miR-338-3p, miR-138-5p, or miR-9-5p. In such instances, the second nucleic acid sequence can be referred to as DRG de-targeting miRNA binding site. Thus, as used herein, the term “DRG de-targeting binding site” refers to a nucleic acid sequence encoding for a binding site of a miR (e.g., miR-338-3p, miR-138-5p, and miR-9-5p) that is highly expressed in DRG cells but not expressed, or expressed at significantly lower levels, in muscle cells. The nucleic acid sequence encoding a binding site of a miR is sufficiently complementary with the nucleic acid sequence encoding the miR to effect base pairing (binding) between the DRG de-targeting binding site and the miR. Advantageously, the second nucleic acid sequence directs the miR, when present, to repress expression of the transgene (e.g., an engineered pri-miRNA). As a result, the selected miRs (miR-338-3p, miR-138-5p, miR-9-5p) in cells transduced by the vectors of the invention will be directed to inhibit expression of the transgene (e.g., the pri-miRNA).

For example, the transgene (e.g., a pri-miRNA) may provide a therapeutic effect when expressed in skeletal and/or heart muscle but provide a toxic effect when expressed in DRG cells. Without being bound to a mechanism of action, transduction of both muscle cells and DRG cells by vectors of the invention results in transcription of both transgene mRNA (e.g., transcription of the pri-miRNA) and the binding site(s) of the select miR. In DRG cells with substantial endogenous expression of miR-338-3p, miR-138-5p, and miR-9-5p, transgene expression (e.g., pri-miRNA expression) will be substantially repressed. In muscle cells with limited to no expression of miR-338-3p, miR-138-5p, miR-9-5p, transgene expression (e.g., pri-miRNA expression) will not be substantially repressed. As a result, the therapeutic effect of the transgene (e.g., the pri-miRNA) will continue in muscle cells and the toxic effect in DRG cells will be abated.

In aspects of the invention, the second nucleic acid sequence may be operably linked to the first nucleic acid sequence, for example, the second nucleic acid sequence may be operably linked to the 3′ or 5′ end of the first nucleic acid sequence. Advantageously, this may result in increased transcription of both nucleic acid sequences.

The engineered vector may be a viral vector. For example, the vector may be an adeno-associated viral (AAV) vector. Further advantageously, the engineered vector may comprise a capsid protein with modified tropism for skeletal and/or heart muscle in comparison with the wild-type AAV vector. For example, transduction and expression of the transgene (e.g., the pri-miRNA) may be increased in muscle tissue while limited expression of the transgene (e.g., the pri-miRNA) in off-target tissue types, for example, DRG may be observed.

The first nucleic acid sequence and second nucleic acid sequence may also be operably linked to tissue specific promoters. Advantageously, the first nucleic acid sequence may be operably linked to a muscle specific promoter. In aspects of the invention, the second nucleic acid is 3′ or 5′ of the first nucleic acid. For example, the second nucleic acid may be incorporated into a 3′ or 5′ untranslated region (UTR) of the transgene mRNA (e.g., the pri-miRNA) when transcribed.

In muscle tissue, where miR-338-3p, miR-138-5p, miR-9-5p expression is limited, the muscle specific promoter promotes transcription of the transgene (e.g., the pri-miRNA) and the miR binding site has limited to no effect due to the absence of the requisite miR. In cells expressing miR-338-3p, miR-138-5p, miR-9-5p, for example DRG, the muscle specific promoter limits transcription of the transgene (e.g., the pri-miRNA) while the miR binding site is still present in the transcript, and translation of any transgene mRNA transcribed (e.g., any pri-miRNA transcribed) can be inhibited by miR binding to the miR binding site in the transcript. Aspects of the invention may provide any number of miR binding site(s) to further inhibit expression of the transgene (e.g., the pri-miRNA).

Aspects of the invention also provide methods and uses of the recombinant nucleic acids and the recombinant expression vectors of the invention, for example, in therapeutic compositions. Accordingly, aspects of the invention provide a method or use of a recombinant nucleic acid or a recombinant expression vector, the method or use comprising providing to a subject a recombinant nucleic acid or a recombinant expression vector (e.g., an engineered vector (e.g., an AAV vector)) comprising a first nucleic acid sequence encoding a transgene and a second nucleic acid sequence expressing the binding site of a miR selected from among miR-338-3p, miR-138-5p, or miR-9-5p. The second nucleic acid sequence thereby directs the miR, when present (for example endogenously), to repress expression of the transgene.

The transgene (e.g., the pri-miRNA) may be expressed in heart and/or skeletal muscle, thereby providing a therapeutic effect. The second nucleic acid sequence may be expressed in dorsal root ganglia cells and direct endogenous miR-338-3p, miR-138-5p, and/or miR-9-5p to repress expression of the transgene (e.g., the pri-miRNA) in dorsal root ganglia cells. For example, the transgene (e.g., the pri-miRNA) may provide a therapeutic effect when expressed in skeletal and/or heart muscle but provide a toxic effect when expressed in DRG cells.

Accordingly, in aspects of the invention, the DRG de-targeting miRNA may comprise a sequence have at least 90% or at least 95% of the sequence identity of one or more of

(SEQ ID NO: 3921; miR-338-3p)
CAACAAAATCACTGATGCTGGA;
(SEQ ID NO: 3922; mir-138-5p)
CGGCCTGATTCACAACACCAGCT,
or
(SEQ ID NO: 3923; miR-9-5p)
TCATACAGCTAGATAACCAAAGA.

Heart De-Targeting miRNAs

The present invention provides recombinant nucleic acids and recombinant expression vectors (e.g., AAV vectors) that encode a transgene (e.g., an engineered pri-miRNA) and the binding site(s) for specific micro RNAs (miRs) that direct the endogenous miRs to selectively inhibit expression of the same transgene (e.g., the same engineered pri-miRNA). For example, miR-221-3p and miR-499a, may be highly expressed in cardiac/heart cells and expressed at substantially lower levels, in skeletal muscle cells.

Accordingly, aspects of the invention provide a recombinant nucleic acid and an engineered vector comprising a first nucleic acid sequence encoding a transgene (e.g., an pri-miRNA) and a second nucleic acid sequence encoding for the binding site(s) of miR-221 or miR-499. In such instances, the second nucleic acid sequence can be referred to as heart de-targeting miRNA binding site. Thus, as used herein, the term “heart de-targeting binding site” refers to a nucleic acid sequence encoding for a binding site of a miR (e.g., miR-221-3p, miR-499a) that is highly expressed in heart cells but not expressed, or expressed at significantly lower levels, in skeletal muscle cells. The nucleic acid sequence encoding a binding site of a miR is sufficiently complementary with the nucleic acid sequence encoding the miR to effect base pairing (binding) between the heart de-targeting binding site and the miR (e.g., miR-221-3p, miR-499a).

Advantageously, the second nucleic acid sequence directs the miR, when present, to repress expression of the transgene (e.g., the pri-miRNA). As a result, miR-221 (e.g., miR-221-3p) or miR-499 (e.g., miR-499a) in cells transduced by the recombinant nucleic acids or vectors of the invention will be directed to inhibit expression of the transgene (e.g., the pri-miRNA). Aspects of the invention may provide any number of miR binding site(s) to further inhibit expression of the transgene (e.g., the pri-miRNA).

For example, the (e.g., the pri-miRNA) may provide a therapeutic effect when expressed in skeletal muscle but provide a toxic effect when expressed in heart cells. Without being bound to a mechanism of action, transduction of both skeletal muscle cells and heart cells by recombinant nucleic acids or vectors of the invention results in transcription of both transgene mRNA (e.g., transcription of the pri-miRNA) and the binding site(s) of the select miR. In heart cells with substantial endogenous expression of miR-221-3p and miR-499a, transgene expression (e.g., pri-miRNA expression) will be substantially repressed. In skeletal muscle cells with limited expression of miR-221-3p and miR-499a, transgene expression is (e.g., pri-miRNA expression) will not be substantially repressed. As a result, the therapeutic effect of the transgene (e.g., the pri-miRNA) will continue in skeletal muscle cells and the potential toxic effect in heart cells will be abated.

In aspects of the invention, the second nucleic acid sequence may be operably linked to the first nucleic acid sequence, for example, the second nucleic acid sequence may be operably linked to the 3′ or 5′ end of the first nucleic acid sequence. Advantageously, this may result in increased transcription of both nucleic acid sequences.

Accordingly, in aspects of the invention, the heart de-targeting miRNA may comprise the sequence: GAAACCCAGCAGACAATGTAGCT (SEQ ID NO: 3924; miR-221-3p) or AAACATCACTGCAAGTCTTAA (SEQ ID NO: 3925; miR-499a).

### Novel Promoters

The present invention provides novel promoters, that may be linked to a nucleic acid sequence encoding a pri-miRNA so that the transcription preferably occurs within myocytes. Promoter regions enable the host cells to replicate the AAV delivered nucleic acid only in those cell types and tissues or organs in which the desired pri-miRNA should be produced. Here, the muscle specific promoter is included because it is principally desired that the pri-miRNA only be translated in myocytes. Specificity of the cell type into which the recombinant nucleic acid is delivered and thus the pri-miRNA translated is desired because of the adverse effects that may ensue from delivering the recombinant nucleic acid and having it translated in cells in which that recombinant nucleic acid and thus the pri-miRNA is not needed.

Accordingly, aspects of the present invention provide a promoter having a sequence of any one of SEQ ID NOs: 3926-3928.

Promoter 1
(SEQ ID NO: 3926)
GGAAGAGAAGGTGACCCTTACCCAGTTGTTCAACTCACCCTTCAG
ATTAAAAATAACTAAGGTAAGGGCCTGGGTAGGGGAGGTGGTGTG
AGAGGGTCCTGTCTCTCCTCTATCTGCCCATCGGCCCTTTGGGGA
GGAGGAATGTGCCCAAGGACTAAAAAAAGGCCCTGGAGCCAGAGG
GGCGAGGGCAGCAGACCTTTCATGGGCAAACCTCAGGGCTGCTGG
CCACTACGGGTCTAGGCTGCCCATGTAAGGAGGCAAGGCCTGGGG
ACACCCGAGATGCCTGGTTATAATTAACCCAGACATGTGGCTGCC
CCCCCCCCCCCAACACCTGCTGCCTGCTAAAAATAACCCTGTCCC
TGGTGGATATCAAGGCTGTGGGGGACTGAGGGCAGGCTGTAACAG
GCTTGGGGGCCAGGGCTTATACGTGCCTGGGACTCCCAAAGTATT
ACTGTTCCATGTTCCCGGCGAAGGGCCAGCTGTCCCCCGCCAGCT
AGACTCAGCACTTAGTTTAGGAACCAGTGAGCAAGTCAGCCCTTG
GGGCAGCCCATACAAGGCCATGGGGCTGGGCAAGCTGCACGCCTG
GGTCCGGGGTGGGCACGGTGCCCGGGCAACGAGCTGAAAGCTCAT
CTACTCTCAGGGGCCCCTCCCTGGGGACAGCCCCTCCTGGCTAGT
CACACCCTGTAGGCTCCTCTATATAACCCAGGGGCACAGGGGCTG
CCCTCATTCTACCACCACCTCCACAGCACAGACAGACACTCACGA
GCCAGC
Promoter 2
(SEQ ID NO: 3927)
GTAGCTTTAGCCGGCTTGCTTGCTTTTGGAGTCTGCCCCTCGTAT
GTACAGATCGGGGTCAGCCTGAGACGTGGGCGGGTTCCTTCCCAG
GCGTTGCAGCTCTCTTTCCTCCCTCACTCTCCAGTTCCTCTGGCT
CAGTGAGGGATTTGGGATTTCTGTGTGTGGGGCATGGACAGATAC
TACTCTTAGTGAAAAGCTGGACAAGGTGGAGTAGAGCATCAAGGG
TGAGTGCCACTACGGGTCTAGGCTGCCCATGTAAGGAGGCAAGGC
CTGGGGACACCCGAGATGCCTGGTTATAATTAACCCAGACATGTG
GCTGCCCCCCCCCCCCCAACACCTGCTGCCTGCTAAAAATAACCC
TGTCCCTGGTGGATATCAAGGCTGTGGGGGACTGAGGGCAGGCTG
TAACAGGCTTGGGGGCCAGGGCTTATACGTGCCTGGGACTCCCAA
AGTATTACTGTTCCATGTTCCCGGCGAAGGGCCAGCTGTCCCCCG
CCAGCTAGACTCAGCACTTAGTTTAGGAACCAGTGAGCAAGTCAG
CCCTTGGGGCAGCCCATACAAGGCCATGGGGCTGGGCAAGCTGCA
CGCCTGGGTCCGGGGTGGGCACGGTGCCCGGGCAACGAGCTGAAA
GCTCATCTACTCTCAGGGGCCCCTCCCTGGGGACAGCCCCTCCTG
GCTAGTCACACCCTGTAGGCTCCTCTATATAACCCAGGGGCACAG
GGGCTGCCCTCATTCTACCACCACCTCCACAGCACAGACAGACAC
TCAGGAGCCAGCCAGC
Promoter 3
(SEQ ID NO: 3928)
CCCCTGACCTTTGCGGTGGAAATCCTGAGCCCAGGCTGAGATGAG
CTCAGCTGATCCTTCTCTCAGAGACAGCTGGAGTACAGTGCTGCT
CAGCCCCCAGCTCCTGAGTGTCTGGAGCAAACAGCTGGGAGAGAG
GTAGAATTCACTCCTGGATTAGAGCCAAAAACTGCCTGGCAGAAA
CCCCCAGGATCCTGTTTCTCCTGAGGGAGGAGCGGGGGAGCCCGG
GGGCGAGAAACCCACAGCACAGGGAAGGGAAGGGCAGCCTTGAAA
CTGCAAGTTGGGAGATCACGGTTTCCTCACGTGGCCACCATGAGG
GCTGAGGCTGTAGGCTGGCCGCTCCTGCCCCTCCCGACCACCCCC
TCAGGCTTCCAGCCTCCTTGCTCAGCCACCTAGGCTGGGCTTTCA
GGTTCACAGGCAGCCGGCCCTACAACATGGAGGCCCAGGCTGGGC
TTTCTTCAGGAGTGGCATCTAAACTTAGGAGAGGGGAACACGTTT
CACCCGAATCTGGGCTAGCAGTGTGTTGGTTAAATGGCCCCTGAG
TAGGTGTGGGGCATGGCAGAGGTGCAGGAGTGGGTGTCCGAGGAT
GCAGCCAACTCATCTCCAAACAGCTCCTTTTCTTTTCCCCAGTGG
GCTGCATTCCCAGACTGGTATTTTAGTTGGTGTGACAGAGTCCCA
GAGTCATCTGGCCCAGGGGCTTGCACCTGCGCCAGTCCTTCCCTG
TCCACCCCTGCCCGTCTCCAGGCGCTCCTATAAAGGGCTGAGCAG
TTCAGCTCTTCTCACTGGGGGGTGTGAGGAACAGG

In some embodiments, the recombinant nucleic acid comprises a muscle specific promoter. Non-limiting examples of muscle specific promoters include a CK8 promoter, a desmin promotor, a Mb promoter, a MCK promoter, a MHCK7 promoter, a skeletal muscle alpha actin promoter, and a TTNI2 promoter.

In some embodiments, the recombinant nucleic acid comprises or consists of the nucleic acid sequence of that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928. In some embodiments, the recombinant nucleic acid comprises or consists of the nucleic acid sequence of any one of SEQ ID NOs: 3926-3928.

In some embodiments, the recombinant nucleic acid comprises or consists of the nucleic acid sequence of SEQ ID NO: 3926. In some embodiments, the recombinant nucleic acid comprises or consists of the nucleic acid sequence of SEQ ID NO: 3927. In some embodiments, the recombinant nucleic acid comprises or consists of the nucleic acid sequence of SEQ ID NO: 3928.

### Engineered Capsid Proteins

The present invention also provides novel capsid protein variants for viral particles that de-target liver tissue and target skeletal muscle and heart tissue at the same time. Aspects of the present invention provide adeno-associated virus (AAV) vectors comprising a capsid protein comprising an insert comprising the amino acid sequence RGDR (SEQ ID NO: 3950). In the capsid protein, RGDR (SEQ ID NO: 3950) may be inserted after amino acid 455 in reference to an AAV9 capsid or an equivalent position in another AAV capsid. AAV vectors may comprise the amino acid sequence X₁NX₂X₃X₄RGDRX₅X₆L (SEQ ID NO: 1), wherein X₁, X₂, X₃, X₄, X₅, and X₆ is any amino acid.

In aspects of the invention, X₁ is an amino acid selected from the group consisting of A, I, F, G, H, L, M, Q, S, T, and V. In some aspects of the invention, X₁ is an amino acid selected from the group consisting of: A, I, L, M, S, and V.

In aspects of the invention, X₂ is an amino acid selected from the group consisting of A, G, S, T, and Y.

In aspects of the invention, X₃ is an amino acid selected from the group consisting of S, N, G, and P. In some aspects of the invention, X₃ is S.

In aspects of the invention, X₄ is an amino acid selected from the group consisting of A, G, H, I, M, S, T, and V.

In aspects of the invention, X₅ is an amino acid selected from the group consisting of A, G, and Q.

In aspects of the invention, X₆ is an amino acid selected from the group consisting of A, I, L, M, N, and Y. In some aspects of the invention, X₆ is selected from the group consisting of A, S, and Y.

In some aspects of the invention, X₁ is located at amino acid 451, X₂ is located at amino acid 453, X₃ is located at amino acid 454, X₄ is located amino acid 455, and RGDRX₅X₆L (SEQ ID NO: 3951) is inserted after amino acid 455 in reference to an AAV9 capsid or an equivalent position in another AAV capsid.

In aspects of the invention, two amino acids from X₁, X₂, X₃, and X₄ are wild type amino acids in reference to an AAV9 capsid or an equivalent position in another AAV capsid and two amino acids from X₁, X₂, X₃, and X₄ are not wild type amino acids.

For example, capsid proteins variants of the invention may comprise a sequence as set forth in Table 2a, e.g., a capsid protein can comprise any one of SEQ ID NOs: 2-800. Notably, capsid protein variants on the invention comprise deletions, substitutions, and/or insertions relative to wild-type viral vector capsids.

In aspects of the invention, the capsid protein comprises an amino acid sequence selected from Table 2a and the amino acid sequence is in hypervariable region IV (HVR IV) relative to wild-type AAV9. The capsid protein variant can comprise substitutions at amino acids 451-455 relative to a wild-type AAV9 vector capsid. For example, the substitutions at amino acids 451-455 relative to a wild-type AAV9 vector capsid can be an amino acid sequence selected from column 1 of Table 2b, e.g., the substitutions relative to a wild type AAV9 capsid can be an amino acid sequence selected from any one of SEQ ID NOs: 801-1599. In aspects of the invention, the capsid protein variant can further comprise an insert. For example, the capsid protein can comprise a 7-mer insert selected from column 2 of Table 2b, e.g., the substitutions relative to a wild type AAV9 capsid can be an amino acid sequence selected from any one of SEQ ID NOs: 801-1599. The insert can be in the location after amino acid 455 relative to a wild-type AAV9 vector.

Advantageously, viral vectors comprising an amino acid sequence of the invention exhibit muscle tropism as compared to a wild-type AAV vector (e.g., a wild type AAV9 vector).

In aspects of the invention, the capsid protein further comprises a deletion of G267 in reference to an AAV9 capsid or an equivalent position in another AAV capsid. Advantageously, the capsid protein comprising a deletion of G267 may exhibit reduced liver tropism as compared to a wild-type AAV capsid protein.

As described, for the HVR IV variants, the 5 amino acids upstream are at positions 451-455, shown in column 1 of Table 2b. The 7-mer insert for HVR IV variants starts with “RGDR” (SEQ ID NO: 3950) and is inserted after amino acid 455, shown in column 2 of Table 2b.

In some embodiments, the capsid protein comprises, relative to the wild type AAV9 capsid protein, the amino acid sequence of LNASI (SEQ ID NO: 990) at positions 451-455, the amino acid sequence of RGDRQLL (SEQ ID NO: 1789) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNASM (SEQ ID NO: 997) at positions 451-455, the amino acid sequence of RGDRQAL (SEQ ID NO: 1796) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNASM (SEQ ID NO: 998) at positions 451-455, the amino acid sequence of RGDRQSL (SEQ ID NO: 1797) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNAST (SEQ ID NO: 1014) at positions 451-455, the amino acid sequence of RGDRQYL (SEQ ID NO: 1813) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNGST (SEQ ID NO: 1055) at positions 451-455, the amino acid sequence of RGDRQYL (SEQ ID NO: 1854) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of LNYST (SEQ ID NO: 1178) at positions 451-455, the amino acid sequence of RGDRQAL (SEQ ID NO: 1977) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of SNAST (SEQ ID NO: 1343) at positions 451-455, the amino acid sequence of RGDRQYL (SEQ ID NO: 2142) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of SNASV (SEQ ID NO: 1349) at positions 451-455, the amino acid sequence of RGDRQAL (SEQ ID NO: 2148) inserted after amino acid 455, a deletion of the amino acid G267, and the remainder of the capsid is otherwise identical to wild type AAV9.

In some embodiments, the capsid protein comprises, relative to the wild type AAV9 capsid protein, the amino acid sequence of VNGST (SEQ ID NO: 1547) at positions 451-455, the amino acid sequence of RGDRQYL (SEQ ID NO: 2346) inserted after amino acid 455, a deletion of the amino acid G267, and the remainder of the capsid is otherwise identical to wild type AAV9.

TABLE 2a
HVR IV Capsid Variants

| | SEQ |
|---|---|
| Amino Acid | ID |
| Sequence | NO: |
| ANASARGDRAAL | 2 |
| ANASARGDRGSL | 3 |
| ANASARGDRGYL | 4 |
| ANASARGDRQAL | 5 |
| ANASARGDRQSL | 6 |
| ANASARGDRQYL | 7 |
| ANASGRGDRAAL | 8 |
| ANASGRGDRASL | 9 |
| ANASGRGDRAYL | 10 |
| ANASGRGDRGAL | 11 |
| ANASGRGDRGSL | 12 |
| ANASGRGDRGYL | 13 |
| ANASGRGDRQAL | 14 |
| ANASGRGDRQSL | 15 |
| ANASGRGDRQYL | 16 |
| ANASMRGDRAAL | 17 |
| ANASMRGDRASL | 18 |
| ANASMRGDRAYL | 19 |
| ANASMRGDRQAL | 20 |
| ANASMRGDRQSL | 21 |
| ANASSRGDRGSL | 22 |
| ANASSRGDRGYL | 23 |
| ANASSRGDRQAL | 24 |
| ANASSRGDRQSL | 25 |
| ANASSRGDRQYL | 26 |
| ANASTRGDRASL | 27 |
| ANASTRGDRAYL | 28 |
| ANASTRGDRGAL | 29 |
| ANASTRGDRGSL | 30 |
| ANASTRGDRGYL | 31 |
| ANASTRGDRQAL | 32 |
| ANASTRGDRQSL | 33 |
| ANASTRGDRQYL | 34 |
| ANASVRGDRASL | 35 |
| ANASVRGDRGYL | 36 |
| ANASVRGDRQAL | 37 |
| ANASVRGDRQSL | 38 |
| ANASVRGDRQYL | 39 |
| ANGNFRGDRQNL | 40 |
| ANGNVRGDRGML | 41 |
| ANGSARGDRAYL | 42 |
| ANGSARGDRGSL | 43 |
| ANGSARGDRGYL | 44 |
| ANGSARGDROAL | 45 |
| ANGSARGDRQYL | 46 |
| ANGSGRGDRAAL | 47 |
| ANGSGRGDRAYL | 48 |
| ANGSGRGDRGAL | 49 |
| ANGSGRGDRGSL | 50 |
| ANGSGRGDRGYL | 51 |
| ANGSGRGDRQAL | 52 |
| ANGSGRGDRQSL | 53 |
| ANGSGRGDRQYL | 54 |
| ANGSMRGDRGYL | 55 |
| ANGSMRGDRQAL | 56 |
| ANGSMRGDRQSL | 57 |
| ANGSMRGDRQYL | 58 |
| ANGSSRGDRAYL | 59 |
| ANGSSRGDRGSL | 60 |
| ANGSSRGDRGYL | 61 |
| ANGSSRGDRQAL | 62 |
| ANGSSRGDRQSL | 63 |
| ANGSSRGDRQYL | 64 |
| ANGSTRGDRAAL | 65 |
| ANGSTRGDRAYL | 66 |
| ANGSTRGDRGAL | 67 |
| ANGSTRGDRGYL | 68 |
| ANGSTRGDRQAL | 69 |
| ANGSTRGDRQSL | 70 |
| ANGSTRGDRQYL | 71 |
| ANGSVRGDRAYL | 72 |
| ANGSVRGDRGYL | 73 |
| ANGSVRGDRQAL | 74 |
| ANGSVRGDRQYL | 75 |
| ANSGMRGDRGAL | 76 |
| ANSSARGDRGAL | 77 |
| ANSSARGDRGSL | 78 |
| ANSSARGDRGYL | 79 |
| ANSSARGDRQAL | 80 |
| ANSSARGDRQSL | 81 |
| ANSSARGDRQYL | 82 |
| ANSSGRGDRAAL | 83 |
| ANSSGRGDRASL | 84 |
| ANSSGRGDRAYL | 85 |
| ANSSGRGDRGAL | 86 |
| ANSSGRGDRGSL | 87 |
| ANSSGRGDRGYL | 88 |
| ANSSGRGDRQAL | 89 |
| ANSSGRGDRQSL | 90 |
| ANSSGRGDRQYL | 91 |
| ANSSMRGDRAYL | 92 |
| ANSSMRGDRQAL | 93 |
| ANSSMRGDRQSL | 94 |
| ANSSMRGDRQYL | 95 |
| ANSSSRGDRGSL | 96 |
| ANSSSRGDRGYL | 97 |
| ANSSSRGDRQAL | 98 |
| ANSSSRGDRQSL | 99 |
| ANSSSRGDRQYL | 100 |
| ANSSTRGDRAAL | 101 |
| ANSSTRGDRGSL | 102 |
| ANSSTRGDRQAL | 103 |
| ANSSTRGDRQSL | 104 |
| ANSSTRGDRQYL | 105 |
| ANSSVRGDRAYL | 106 |
| ANSSVRGDRGYL | 107 |
| ANSSVRGDRQAL | 108 |
| ANSSVRGDRQSL | 109 |
| ANSSVRGDRQYL | 110 |
| ANTSARGDRAYL | 111 |
| ANTSARGDRGAL | 112 |
| ANTSARGDRGSL | 113 |
| ANTSARGDRGYL | 114 |
| ANTSARGDRQAL | 115 |
| ANTSARGDRQYL | 116 |
| ANTSGRGDRASL | 117 |
| ANTSGRGDRGAL | 118 |
| ANTSGRGDRGSL | 119 |
| ANTSGRGDRGYL | 120 |
| ANTSGRGDRQAL | 121 |
| ANTSGRGDRQSL | 122 |
| ANTSGRGDRQYL | 123 |
| ANTSMRGDRAYL | 124 |
| ANTSMRGDRQAL | 125 |
| ANTSMRGDRQSL | 126 |
| ANTSMRGDRQYL | 127 |
| ANTSSRGDRGSL | 128 |
| ANTSSRGDRQAL | 129 |
| ANTSSRGDRQYL | 130 |
| ANTSTRGDRAAL | 131 |
| ANTSTRGDRQAL | 132 |
| ANTSTRGDRQSL | 133 |
| ANTSTRGDRQYL | 134 |
| ANTSVRGDRAYL | 135 |
| ANTSVRGDRGSL | 136 |
| ANTSVRGDRQAL | 137 |
| ANTSVRGDRQSL | 138 |
| ANTSVRGDRQYL | 139 |
| ANYPHRGDRGAL | 140 |
| ANYSARGDRAAL | 141 |
| ANYSARGDRASL | 142 |
| ANYSARGDRGAL | 143 |
| ANYSARGDRGSL | 144 |
| ANYSARGDRQAL | 145 |
| ANYSARGDRQSL | 146 |
| ANYSGRGDRAAL | 147 |
| ANYSGRGDRASL | 148 |
| ANYSGRGDRGAL | 149 |
| ANYSGRGDRGSL | 150 |
| ANYSGRGDRQAL | 151 |
| ANYSGRGDRQSL | 152 |
| ANYSGRGDRQYL | 153 |
| ANYSMRGDRAAL | 154 |
| ANYSMRGDRASL | 155 |
| ANYSMRGDRAYL | 156 |
| ANYSMRGDRQAL | 157 |
| ANYSMRGDRQSL | 158 |
| ANYSSRGDRGAL | 159 |
| ANYSSRGDRGSL | 160 |
| ANYSSRGDRQAL | 161 |
| ANYSSRGDRQSL | 162 |
| ANYSSRGDRQYL | 163 |
| ANYSTRGDRQAL | 164 |
| ANYSTRGDRQSL | 165 |
| ANYSTRGDRQYL | 166 |
| ANYSVRGDRQAL | 167 |
| ANYSVRGDRQSL | 168 |
| ANYSVRGDRQYL | 169 |
| FNSGMRGDRGAL | 170 |
| GNSGMRGDRGAL | 171 |
| HNSGMRGDRGAL | 172 |
| LNASARGDRAAL | 173 |
| LNASARGDRASL | 174 |
| LNASARGDRAYL | 175 |
| LNASARGDRGAL | 176 |
| LNASARGDRGSL | 177 |
| LNASARGDRGYL | 178 |
| LNASARGDRQAL | 179 |
| LNASARGDRQSL | 180 |
| LNASARGDRQYL | 181 |
| LNASGRGDRAAL | 182 |
| LNASGRGDRASL | 183 |
| LNASGRGDRAYL | 184 |
| LNASGRGDRGAL | 185 |
| LNASGRGDRGSL | 186 |
| LNASGRGDRGYL | 187 |
| LNASGRGDRQAL | 188 |
| LNASGRGDRQSL | 189 |
| LNASGRGDRQYL | 190 |
| LNASIRGDRQLL | 191 |
| LNASMRGDRAAL | 192 |
| LNASMRGDRASL | 193 |
| LNASMRGDRAYL | 194 |
| LNASMRGDRGAL | 195 |
| LNASMRGDRGSL | 196 |
| LNASMRGDRGYL | 197 |
| LNASMRGDRQAL | 198 |
| LNASMRGDRQSL | 199 |
| LNASMRGDRQYL | 200 |
| LNASSRGDRAAL | 201 |
| LNASSRGDRASL | 202 |
| LNASSRGDRGAL | 203 |
| LNASSRGDRGSL | 204 |
| LNASSRGDRQAL | 205 |
| LNASSRGDRQSL | 206 |
| LNASSRGDRQYL | 207 |
| LNASTRGDRAAL | 208 |
| LNASTRGDRASL | 209 |
| LNASTRGDRGAL | 210 |
| LNASTRGDRGSL | 211 |
| LNASTRGDRGYL | 212 |
| LNASTRGDRQAL | 213 |
| LNASTRGDRQSL | 214 |
| LNASTRGDRQYL | 215 |
| LNASVRGDRAAL | 216 |
| LNASVRGDRASL | 217 |
| LNASVRGDRGAL | 218 |
| LNASVRGDRGSL | 219 |
| LNASVRGDRQAL | 220 |
| LNASVRGDRQSL | 221 |
| LNASVRGDRQYL | 222 |
| LNGNFRGDRQNL | 223 |
| LNGNVRGDRGML | 224 |
| LNGSARGDRAAL | 225 |
| LNGSARGDRAYL | 226 |
| LNGSARGDRGSL | 227 |
| LNGSARGDRGYL | 228 |
| LNGSARGDRQAL | 229 |
| LNGSARGDRQSL | 230 |
| LNGSARGDRQYL | 231 |
| LNGSGRGDRAAL | 232 |
| LNGSGRGDRAYL | 233 |
| LNGSGRGDRGAL | 234 |
| LNGSGRGDRGSL | 235 |
| LNGSGRGDRGYL | 236 |
| LNGSGRGDRQAL | 237 |
| LNGSGRGDRQYL | 238 |
| LNGSMRGDRASL | 239 |
| LNGSMRGDRGYL | 240 |
| LNGSMRGDRQAL | 241 |
| LNGSMRGDRQSL | 242 |
| LNGSMRGDRQYL | 243 |
| LNGSSRGDRAAL | 244 |
| LNGSSRGDRAYL | 245 |
| LNGSSRGDRGAL | 246 |
| LNGSSRGDRQYL | 247 |
| LNGSTRGDRAAL | 248 |
| LNGSTRGDRASL | 249 |
| LNGSTRGDRAYL | 250 |
| LNGSTRGDRGAL | 251 |
| LNGSTRGDRGSL | 252 |
| LNGSTRGDRGYL | 253 |
| LNGSTRGDRQAL | 254 |
| LNGSTRGDRQSL | 255 |
| LNGSTRGDRQYL | 256 |
| LNGSVRGDRASL | 257 |
| LNGSVRGDRAYL | 258 |
| LNGSVRGDRGYL | 259 |
| LNGSVRGDRQAL | 260 |
| LNGSVRGDRQSL | 261 |
| LNGSVRGDRQYL | 262 |
| LNSGMRGDRGAL | 263 |
| LNSSARGDRASL | 264 |
| LNSSARGDRAYL | 265 |
| LNSSARGDRGAL | 266 |
| LNSSARGDRGIL | 267 |
| LNSSARGDRGSL | 268 |
| LNSSARGDRGYL | 269 |
| LNSSARGDRQAL | 270 |
| LNSSARGDRQSL | 271 |
| LNSSARGDRQYL | 272 |
| LNSSGRGDRAAL | 273 |
| LNSSGRGDRASL | 274 |
| LNSSGRGDRAYL | 275 |
| LNSSGRGDRGAL | 276 |
| LNSSGRGDRGSL | 277 |
| LNSSGRGDRGYL | 278 |
| LNSSGRGDRQAL | 279 |
| LNSSGRGDRQSL | 280 |
| LNSSGRGDRQYL | 281 |
| LNSSMRGDRAAL | 282 |
| LNSSMRGDRASL | 283 |
| LNSSMRGDRAYL | 284 |
| LNSSMRGDRGAL | 285 |
| LNSSMRGDRGSL | 286 |
| LNSSMRGDRQAL | 287 |
| LNSSMRGDRQSL | 288 |
| LNSSMRGDRQYL | 289 |
| LNSSSRGDRAAL | 290 |
| LNSSSRGDRGAL | 291 |
| LNSSSRGDRGSL | 292 |
| LNSSSRGDRQAL | 293 |
| LNSSSRGDRQSL | 294 |
| LNSSSRGDRQYL | 295 |
| LNSSTRGDRAAL | 296 |
| LNSSTRGDRASL | 297 |
| LNSSTRGDRGAL | 298 |
| LNSSTRGDRGSL | 299 |
| LNSSTRGDRQAL | 300 |
| LNSSTRGDRQSL | 301 |
| LNSSTRGDRQYL | 302 |
| LNSSVRGDRAAL | 303 |
| LNSSVRGDRASL | 304 |
| LNSSVRGDRGAL | 305 |
| LNSSVRGDRGSL | 306 |
| LNSSVRGDRGYL | 307 |
| LNSSVRGDRQAL | 308 |
| LNSSVRGDRQSL | 309 |
| LNSSVRGDRQYL | 310 |
| LNTSARGDRAAL | 311 |
| LNTSARGDRASL | 312 |
| LNTSARGDRAYL | 313 |
| LNTSARGDRGAL | 314 |
| LNTSARGDRGSL | 315 |
| LNTSARGDRGYL | 316 |
| LNTSARGDRQAL | 317 |
| LNTSARGDRQSL | 318 |
| LNTSARGDRQYL | 319 |
| LNTSGRGDRAAL | 320 |
| LNTSGRGDRASL | 321 |
| LNTSGRGDRAYL | 322 |
| LNTSGRGDRGAL | 323 |
| LNTSGRGDRGSL | 324 |
| LNTSGRGDRGYL | 325 |
| LNTSGRGDRQAL | 326 |
| LNTSGRGDRQSL | 327 |
| LNTSGRGDRQYL | 328 |
| LNTSMRGDRAAL | 329 |
| LNTSMRGDRASL | 330 |
| LNTSMRGDRAYL | 331 |
| LNTSMRGDRGAL | 332 |
| LNTSMRGDRGSL | 333 |
| LNTSMRGDRQAL | 334 |
| LNTSMRGDRQSL | 335 |
| LNTSMRGDRQYL | 336 |
| LNTSSRGDRAAL | 337 |
| LNTSSRGDRGSL | 338 |
| LNTSSRGDRQAL | 339 |
| LNTSSRGDRQSL | 340 |
| LNTSSRGDRQYL | 341 |
| LNTSTRGDRAAL | 342 |
| LNTSTRGDRASL | 343 |
| LNTSTRGDRGAL | 344 |
| LNTSTRGDRGSL | 345 |
| LNTSTRGDRQAL | 346 |
| LNTSTRGDRQSL | 347 |
| LNTSTRGDRQYL | 348 |
| LNTSVRGDRAAL | 349 |
| LNTSVRGDRASL | 350 |
| LNTSVRGDRAYL | 351 |
| LNTSVRGDRGAL | 352 |
| LNTSVRGDRGSL | 353 |
| LNTSVRGDRQAL | 354 |
| LNTSVRGDRQSL | 355 |
| LNYPHRGDRGAL | 356 |
| LNYSARGDRGAL | 357 |
| LNYSARGDRGSL | 358 |
| LNYSARGDRQAL | 359 |
| LNYSARGDRQSL | 360 |
| LNYSARGDRQYL | 361 |
| LNYSGRGDRAAL | 362 |
| LNYSGRGDRASL | 363 |
| LNYSGRGDRGAL | 364 |
| LNYSGRGDRGSL | 365 |
| LNYSGRGDRQAL | 366 |
| LNYSGRGDRQSL | 367 |
| LNYSGRGDRQYL | 368 |
| LNYSMRGDRQAL | 369 |
| LNYSMRGDRQSL | 370 |
| LNYSSRGDRGAL | 371 |
| LNYSSRGDRGSL | 372 |
| LNYSSRGDRQAL | 373 |
| LNYSSRGDRQSL | 374 |
| LNYSSRGDRQYL | 375 |
| LNYSTRGDRASL | 376 |
| LNYSTRGDRGAL | 377 |
| LNYSTRGDRGSL | 378 |
| LNYSTRGDRQAL | 379 |
| LNYSTRGDRQSL | 380 |
| LNYSTRGDRQYL | 381 |
| LNYSVRGDRQAL | 382 |
| LNYSVRGDRQSL | 383 |
| MNASARGDRAAL | 384 |
| MNASARGDRAYL | 385 |
| MNASARGDRGSL | 386 |
| MNASARGDRGYL | 387 |
| MNASARGDRQAL | 388 |
| MNASARGDRQSL | 389 |
| MNASARGDRQYL | 390 |
| MNASGRGDRAAL | 391 |
| MNASGRGDRASL | 392 |
| MNASGRGDRAYL | 393 |
| MNASGRGDRGSL | 394 |
| MNASGRGDRQAL | 395 |
| MNASGRGDRQSL | 396 |
| MNASGRGDRQYL | 397 |
| MNASIRGDROLL | 398 |
| MNASMRGDRAAL | 399 |
| MNASMRGDRAYL | 400 |
| MNASMRGDRGAL | 401 |
| MNASMRGDRQAL | 402 |
| MNASMRGDRQSL | 403 |
| MNASSRGDRAAL | 404 |
| MNASTRGDRAAL | 405 |
| MNASTRGDRASL | 406 |
| MNASTRGDRAYL | 407 |
| MNASTRGDRGAL | 408 |
| MNASTRGDRGSL | 409 |
| MNASTRGDRQAL | 410 |
| MNASTRGDRQSL | 411 |
| MNASTRGDRQYL | 412 |
| MNASVRGDRAAL | 413 |
| MNASVRGDRASL | 414 |
| MNASVRGDRAYL | 415 |
| MNASVRGDRQAL | 416 |
| MNASVRGDRQSL | 417 |
| MNASVRGDRQYL | 418 |
| MNGNFRGDRQNL | 419 |
| MNGSARGDRAYL | 420 |
| MNGSARGDRGYL | 421 |
| MNGSARGDRQAL | 422 |
| MNGSARGDRQSL | 423 |
| MNGSARGDRQYL | 424 |
| MNGSGRGDRAAL | 425 |
| MNGSGRGDRAYL | 426 |
| MNGSGRGDRGAL | 427 |
| MNGSGRGDRGSL | 428 |
| MNGSGRGDRGYL | 429 |
| MNGSGRGDRQAL | 430 |
| MNGSGRGDRQSL | 431 |
| MNGSGRGDRQYL | 432 |
| MNGSMRGDRQAL | 433 |
| MNGSMRGDRQSL | 434 |
| MNGSMRGDRQYL | 435 |
| MNGSSRGDRQYL | 436 |
| MNGSTRGDRAYL | 437 |
| MNGSTRGDRGAL | 438 |
| MNGSTRGDRGYL | 439 |
| MNGSTRGDRQAL | 440 |
| MNGSTRGDRQSL | 441 |
| MNGSTRGDRQYL | 442 |
| MNGSVRGDRGYL | 443 |
| MNGSVRGDRQAL | 444 |
| MNGSVRGDRQYL | 445 |
| MNSGMRGDRGAL | 446 |
| MNSSARGDRAAL | 447 |
| MNSSARGDRAYL | 448 |
| MNSSARGDRGSL | 449 |
| MNSSARGDRGYL | 450 |
| MNSSARGDRQAL | 451 |
| MNSSARGDRQSL | 452 |
| MNSSARGDRQYL | 453 |
| MNSSGRGDRAAL | 454 |
| MNSSGRGDRASL | 455 |
| MNSSGRGDRAYL | 456 |
| MNSSGRGDRGAL | 457 |
| MNSSGRGDRGSL | 458 |
| MNSSGRGDRQAL | 459 |
| MNSSGRGDRQSL | 460 |
| MNSSGRGDRQYL | 461 |
| MNSSMRGDRAAL | 462 |
| MNSSMRGDRASL | 463 |
| MNSSMRGDRAYL | 464 |
| MNSSMRGDRGSL | 465 |
| MNSSMRGDRQAL | 466 |
| MNSSMRGDRQSL | 467 |
| MNSSMRGDRQYL | 468 |
| MNSSSRGDRAAL | 469 |
| MNSSTRGDRAAL | 470 |
| MNSSTRGDRASL | 471 |
| MNSSTRGDRQAL | 472 |
| MNSSTRGDRQSL | 473 |
| MNSSTRGDRQYL | 474 |
| MNSSVRGDRAAL | 475 |
| MNSSVRGDRAYL | 476 |
| MNSSVRGDRQAL | 477 |
| MNSSVRGDRQSL | 478 |
| MNSSVRGDRQYL | 479 |
| MNTSARGDRAYL | 480 |
| MNTSGRGDRAAL | 481 |
| MNTSGRGDRQAL | 482 |
| MNTSGRGDRQYL | 483 |
| MNTSMRGDRAYL | 484 |
| MNTSTRGDRQYL | 485 |
| MNYPHRGDRGAL | 486 |
| MNYSARGDRAAL | 487 |
| MNYSARGDRGYL | 488 |
| MNYSARGDRQAL | 489 |
| MNYSARGDRQSL | 490 |
| MNYSGRGDRAAL | 491 |
| MNYSGRGDRASL | 492 |
| MNYSGRGDRGSL | 493 |
| MNYSGRGDRQAL | 494 |
| MNYSGRGDRQSL | 495 |
| MNYSSRGDRQAL | 496 |
| MNYSTRGDRAAL | 497 |
| MNYSTRGDRQAL | 498 |
| MNYSTRGDRQSL | 499 |
| MNYSTRGDRQYL | 500 |
| NNSGMRGDRGAL | 501 |
| QNSGMRGDRGAL | 502 |
| SNASARGDRAAL | 503 |
| SNASARGDRASL | 504 |
| SNASARGDRAYL | 505 |
| SNASARGDRGAL | 506 |
| SNASARGDRGSL | 507 |
| SNASARGDRGYL | 508 |
| SNASARGDRQAL | 509 |
| SNASARGDRQSL | 510 |
| SNASARGDRQYL | 511 |
| SNASGRGDRAAL | 512 |
| SNASGRGDRASL | 513 |
| SNASGRGDRAYL | 514 |
| SNASGRGDRGAL | 515 |
| SNASGRGDRGSL | 516 |
| SNASGRGDRGYL | 517 |
| SNASGRGDRQAL | 518 |
| SNASGRGDRQSL | 519 |
| SNASGRGDRQYL | 520 |
| SNASIRGDRQLL | 521 |
| SNASMRGDRAAL | 522 |
| SNASMRGDRASL | 523 |
| SNASMRGDRGAL | 524 |
| SNASMRGDRGSL | 525 |
| SNASMRGDRQAL | 526 |
| SNASMRGDRQSL | 527 |
| SNASSRGDRAAL | 528 |
| SNASSRGDRASL | 529 |
| SNASSRGDRAYL | 530 |
| SNASSRGDRGAL | 531 |
| SNASSRGDRGSL | 532 |
| SNASSRGDRQAL | 533 |
| SNASSRGDRQSL | 534 |
| SNASSRGDRQYL | 535 |
| SNASTRGDRAAL | 536 |
| SNASTRGDRASL | 537 |
| SNASTRGDRAYL | 538 |
| SNASTRGDRGAL | 539 |
| SNASTRGDRGSL | 540 |
| SNASTRGDRGYL | 541 |
| SNASTRGDRQAL | 542 |
| SNASTRGDRQSL | 543 |
| SNASTRGDRQYL | 544 |
| SNASVRGDRAAL | 545 |
| SNASVRGDRASL | 546 |
| SNASVRGDRAYL | 547 |
| SNASVRGDRGSL | 548 |
| SNASVRGDRGYL | 549 |
| SNASVRGDRQAL | 550 |
| SNASVRGDRQSL | 551 |
| SNASVRGDRQYL | 552 |
| SNGNFRGDRQNL | 553 |
| SNGNVRGDRGML | 554 |
| SNGSARGDRGYL | 555 |
| SNGSARGDRQAL | 556 |
| SNGSARGDRQSL | 557 |
| SNGSARGDRQYL | 558 |
| SNGSGRGDRASL | 559 |
| SNGSGRGDRAYL | 560 |
| SNGSGRGDRGAL | 561 |
| SNGSGRGDRGSL | 562 |
| SNGSGRGDRGYL | 563 |
| SNGSGRGDRQAL | 564 |
| SNGSGRGDRQSL | 565 |
| SNGSGRGDRQYL | 566 |
| SNGSMRGDRAYL | 567 |
| SNGSMRGDRGYL | 568 |
| SNGSMRGDRQAL | 569 |
| SNGSMRGDRQSL | 570 |
| SNGSMRGDRQYL | 571 |
| SNGSSRGDRGYL | 572 |
| SNGSSRGDRQYL | 573 |
| SNGSTRGDRAAL | 574 |
| SNGSTRGDRASL | 575 |
| SNGSTRGDRGAL | 576 |
| SNGSTRGDRGYL | 577 |
| SNGSTRGDRQAL | 578 |
| SNGSTRGDRQSL | 579 |
| SNGSTRGDRQYL | 580 |
| SNGSVRGDRGYL | 581 |
| SNGSVRGDRQAL | 582 |
| SNGSVRGDRQSL | 583 |
| SNGSVRGDRQYL | 584 |
| SNSGMRGDRGAL | 585 |
| SNSSARGDRAYL | 586 |
| SNSSARGDRGAL | 587 |
| SNSSARGDRGIL | 588 |
| SNSSARGDRGSL | 589 |
| SNSSARGDRGYL | 590 |
| SNSSARGDRQAL | 591 |
| SNSSARGDRQSL | 592 |
| SNSSARGDRQYL | 593 |
| SNSSGRGDRAAL | 594 |
| SNSSGRGDRAYL | 595 |
| SNSSGRGDRGAL | 596 |
| SNSSGRGDRGSL | 597 |
| SNSSGRGDRGYL | 598 |
| SNSSGRGDRQAL | 599 |
| SNSSGRGDRQSL | 600 |
| SNSSGRGDRQYL | 601 |
| SNSSMRGDRAAL | 602 |
| SNSSMRGDRASL | 603 |
| SNSSMRGDRAYL | 604 |
| SNSSMRGDRGSL | 605 |
| SNSSMRGDRQAL | 606 |
| SNSSMRGDRQSL | 607 |
| SNSSMRGDRQYL | 608 |
| SNSSSRGDRAAL | 609 |
| SNSSSRGDRGSL | 610 |
| SNSSSRGDRQAL | 611 |
| SNSSSRGDRQSL | 612 |
| SNSSSRGDRQYL | 613 |
| SNSSTRGDRAAL | 614 |
| SNSSTRGDRASL | 615 |
| SNSSTRGDRGSL | 616 |
| SNSSTRGDRGYL | 617 |
| SNSSTRGDRQAL | 618 |
| SNSSTRGDRQSL | 619 |
| SNSSTRGDRQYL | 620 |
| SNSSVRGDRQAL | 621 |
| SNSSVRGDRQSL | 622 |
| SNSSVRGDRQYL | 623 |
| SNTSARGDRAAL | 624 |
| SNTSARGDRGAL | 625 |
| SNTSARGDRGSL | 626 |
| SNTSARGDRGYL | 627 |
| SNTSARGDRQAL | 628 |
| SNTSARGDRQSL | 629 |
| SNTSARGDRQYL | 630 |
| SNTSGRGDRAAL | 631 |
| SNTSGRGDRASL | 632 |
| SNTSGRGDRGAL | 633 |
| SNTSGRGDRGSL | 634 |
| SNTSGRGDRGYL | 635 |
| SNTSGRGDRQAL | 636 |
| SNTSGRGDRQSL | 637 |
| SNTSGRGDRQYL | 638 |
| SNTSMRGDRAYL | 639 |
| SNTSMRGDRGSL | 640 |
| SNTSMRGDRQAL | 641 |
| SNTSMRGDRQSL | 642 |
| SNTSSRGDRQAL | 643 |
| SNTSTRGDRQAL | 644 |
| SNTSTRGDRQSL | 645 |
| SNTSTRGDRQYL | 646 |
| SNTSVRGDRAYL | 647 |
| SNTSVRGDRQAL | 648 |
| SNTSVRGDRQSL | 649 |
| SNTSVRGDRQYL | 650 |
| SNYPHRGDRGAL | 651 |
| SNYSARGDRAAL | 652 |
| SNYSARGDRASL | 653 |
| SNYSARGDRGAL | 654 |
| SNYSARGDRGSL | 655 |
| SNYSARGDRQAL | 656 |
| SNYSARGDRQSL | 657 |
| SNYSARGDRQYL | 658 |
| SNYSGRGDRAAL | 659 |
| SNYSGRGDRGAL | 660 |
| SNYSGRGDRGSL | 661 |
| SNYSGRGDRQAL | 662 |
| SNYSGRGDRQSL | 663 |
| SNYSMRGDRAAL | 664 |
| SNYSMRGDRASL | 665 |
| SNYSMRGDRGSL | 666 |
| SNYSMRGDRQAL | 667 |
| SNYSMRGDRQSL | 668 |
| SNYSSRGDRGAL | 669 |
| SNYSSRGDRGSL | 670 |
| SNYSSRGDRQAL | 671 |
| SNYSSRGDRQSL | 672 |
| SNYSTRGDRAAL | 673 |
| SNYSTRGDRGAL | 674 |
| SNYSTRGDRGSL | 675 |
| SNYSTRGDRQAL | 676 |
| SNYSTRGDRQSL | 677 |
| SNYSTRGDRQYL | 678 |
| SNYSVRGDRQAL | 679 |
| SNYSVRGDRQSL | 680 |
| SNYSVRGDRQYL | 681 |
| TNSGMRGDRGAL | 682 |
| VNASARGDRAAL | 683 |
| VNASARGDRGAL | 684 |
| VNASARGDRGSL | 685 |
| VNASARGDRGYL | 686 |
| VNASARGDRQAL | 687 |
| VNASARGDRQYL | 688 |
| VNASGRGDRAAL | 689 |
| VNASGRGDRAYL | 690 |
| VNASGRGDRGAL | 691 |
| VNASGRGDRGSL | 692 |
| VNASGRGDRGYL | 693 |
| VNASGRGDRQAL | 694 |
| VNASGRGDRQYL | 695 |
| VNASMRGDRAYL | 696 |
| VNASMRGDRQAL | 697 |
| VNASMRGDRQSL | 698 |
| VNASSRGDRGSL | 699 |
| VNASSRGDRQAL | 700 |
| VNASSRGDRQYL | 701 |
| VNASTRGDRAAL | 702 |
| VNASTRGDRGAL | 703 |
| VNASTRGDRGSL | 704 |
| VNASTRGDRGYL | 705 |
| VNASTRGDRQAL | 706 |
| VNASTRGDRQSL | 707 |
| VNASTRGDRQYL | 708 |
| VNASVRGDRGSL | 709 |
| VNASVRGDRGYL | 710 |
| VNASVRGDRQAL | 711 |
| VNASVRGDRQSL | 712 |
| VNASVRGDRQYL | 713 |
| VNGNFRGDRQNL | 714 |
| VNGNVRGDRGML | 715 |
| VNGSARGDRAYL | 716 |
| VNGSARGDRGAL | 717 |
| VNGSARGDRGSL | 718 |
| VNGSARGDRGYL | 719 |
| VNGSARGDRQAL | 720 |
| VNGSARGDRQSL | 721 |
| VNGSARGDRQYL | 722 |
| VNGSGRGDRAAL | 723 |
| VNGSGRGDRASL | 724 |
| VNGSGRGDRAYL | 725 |
| VNGSGRGDRGAL | 726 |
| VNGSGRGDRGSL | 727 |
| VNGSGRGDRGYL | 728 |
| VNGSGRGDRQAL | 729 |
| VNGSGRGDRQSL | 730 |
| VNGSGRGDRQYL | 731 |
| VNGSMRGDRGYL | 732 |
| VNGSMRGDRQAL | 733 |
| VNGSMRGDRQSL | 734 |
| VNGSMRGDRQYL | 735 |
| VNGSSRGDRAYL | 736 |
| VNGSSRGDRGAL | 737 |
| VNGSSRGDRGSL | 738 |
| VNGSSRGDRQAL | 739 |
| VNGSSRGDRQSL | 740 |
| VNGSSRGDRQYL | 741 |
| VNGSTRGDRAAL | 742 |
| VNGSTRGDRAYL | 743 |
| VNGSTRGDRGAL | 744 |
| VNGSTRGDRGYL | 745 |
| VNGSTRGDRQAL | 746 |
| VNGSTRGDRQSL | 747 |
| VNGSTRGDRQYL | 748 |
| VNGSVRGDRAYL | 749 |
| VNGSVRGDRQAL | 750 |
| VNGSVRGDRQSL | 751 |
| VNGSVRGDRQYL | 752 |
| VNSGMRGDRGAL | 753 |
| VNSSARGDRGAL | 754 |
| VNSSARGDRGSL | 755 |
| VNSSARGDRGYL | 756 |
| VNSSARGDRQAL | 757 |
| VNSSARGDRQSL | 758 |
| VNSSARGDRQYL | 759 |
| VNSSGRGDRAAL | 760 |
| VNSSGRGDRAYL | 761 |
| VNSSGRGDRGAL | 762 |
| VNSSGRGDRQAL | 763 |
| VNSSGRGDRQSL | 764 |
| VNSSGRGDRQYL | 765 |
| VNSSMRGDRAYL | 766 |
| VNSSMRGDRQAL | 767 |
| VNSSMRGDRQSL | 768 |
| VNSSMRGDRQYL | 769 |
| VNSSSRGDRGSL | 770 |
| VNSSSRGDRQAL | 771 |
| VNSSSRGDRQSL | 772 |
| VNSSTRGDRGAL | 773 |
| VNSSTRGDRQAL | 774 |
| VNSSTRGDRQSL | 775 |
| VNSSTRGDRQYL | 776 |
| VNSSVRGDRAYL | 777 |
| VNSSVRGDRQAL | 778 |
| VNSSVRGDRQSL | 779 |
| VNTSARGDRAYL | 780 |
| VNTSGRGDRAAL | 781 |
| VNTSGRGDRGAL | 782 |
| VNTSGRGDRGSL | 783 |
| VNTSGRGDRGYL | 784 |
| VNTSGRGDRQAL | 785 |
| VNTSGRGDRQSL | 786 |
| VNTSGRGDRQYL | 787 |
| VNTSTRGDRAYL | 788 |
| VNYPHRGDRGAL | 789 |
| VNYSARGDRGAL | 790 |
| VNYSARGDRGSL | 791 |
| VNYSARGDRQAL | 792 |
| VNYSARGDRQSL | 793 |
| VNYSGRGDRAAL | 794 |
| VNYSGRGDRGAL | 795 |
| VNYSGRGDRQAL | 796 |
| VNYSGRGDRQSL | 797 |
| VNYSGRGDRQYL | 798 |
| VNYSSRGDRGSL | 799 |
| VNYSSRGDRGYL | 800 |

TABLE 2b
Split Amino Acid Sequences from Table 2a

| | SEQ | | SEQ |
|---|---|---|---|
| 5 aa | ID | 7 aa | ID |
| substitution | NO: | insert | NO: |
| ANASA | 801 | RGDRAAL | 1600 |
| ANASA | 802 | RGDRGSL | 1601 |
| ANASA | 803 | RGDRGYL | 1602 |
| ANASA | 804 | RGDRQAL | 1603 |
| ANASA | 805 | RGDRQSL | 1604 |
| ANASA | 806 | RGDRQYL | 1605 |
| ANASG | 807 | RGDRAAL | 1606 |
| ANASG | 808 | RGDRASL | 1607 |
| ANASG | 809 | RGDRAYL | 1608 |
| ANASG | 810 | RGDRGAL | 1609 |
| ANASG | 811 | RGDRGSL | 1610 |
| ANASG | 812 | RGDRGYL | 1611 |
| ANASG | 813 | RGDRQAL | 1612 |
| ANASG | 814 | RGDRQSL | 1613 |
| ANASG | 815 | RGDRQYL | 1614 |
| ANASM | 816 | RGDRAAL | 1615 |
| ANASM | 817 | RGDRASL | 1616 |
| ANASM | 818 | RGDRAYL | 1617 |
| ANASM | 819 | RGDRQAL | 1618 |
| ANASM | 820 | RGDRQSL | 1619 |
| ANASS | 821 | RGDRGSL | 1620 |
| ANASS | 822 | RGDRGYL | 1621 |
| ANASS | 823 | RGDRQAL | 1622 |
| ANASS | 824 | RGDRQSL | 1623 |
| ANASS | 825 | RGDRQYL | 1624 |
| ANAST | 826 | RGDRASL | 1625 |
| ANAST | 827 | RGDRAYL | 1626 |
| ANAST | 828 | RGDRGAL | 1627 |
| ANAST | 829 | RGDRGSL | 1628 |
| ANAST | 830 | RGDRGYL | 1629 |
| ANAST | 831 | RGDRQAL | 1630 |
| ANAST | 832 | RGDRQSL | 1631 |
| ANAST | 833 | RGDRQYL | 1632 |
| ANASV | 834 | RGDRASL | 1633 |
| ANASV | 835 | RGDRGYL | 1634 |
| ANASV | 836 | RGDRQAL | 1635 |
| ANASV | 837 | RGDRQSL | 1636 |
| ANASV | 838 | RGDRQYL | 1637 |
| ANGNF | 839 | RGDRQNL | 1638 |
| ANGNV | 840 | RGDRGML | 1639 |
| ANGSA | 841 | RGDRAYL | 1640 |
| ANGSA | 842 | RGDRGSL | 1641 |
| ANGSA | 843 | RGDRGYL | 1642 |
| ANGSA | 844 | RGDRQAL | 1643 |
| ANGSA | 845 | RGDRQYL | 1644 |
| ANGSG | 846 | RGDRAAL | 1645 |
| ANGSG | 847 | RGDRAYL | 1646 |
| ANGSG | 848 | RGDRGAL | 1647 |
| ANGSG | 849 | RGDRGSL | 1648 |
| ANGSG | 850 | RGDRGYL | 1649 |
| ANGSG | 851 | RGDRQAL | 1650 |
| ANGSG | 852 | RGDRQSL | 1651 |
| ANGSG | 853 | RGDRQYL | 1652 |
| ANGSM | 854 | RGDRGYL | 1653 |
| ANGSM | 855 | RGDRQAL | 1654 |
| ANGSM | 856 | RGDRQSL | 1655 |
| ANGSM | 857 | RGDRQYL | 1656 |
| ANGSS | 858 | RGDRAYL | 1657 |
| ANGSS | 859 | RGDRGSL | 1658 |
| ANGSS | 860 | RGDRGYL | 1659 |
| ANGSS | 861 | RGDRQAL | 1660 |
| ANGSS | 862 | RGDRQSL | 1661 |
| ANGSS | 863 | RGDRQYL | 1662 |
| ANGST | 864 | RGDRAAL | 1663 |
| ANGST | 865 | RGDRAYL | 1664 |
| ANGST | 866 | RGDRGAL | 1665 |
| ANGST | 867 | RGDRGYL | 1666 |
| ANGST | 868 | RGDRQAL | 1667 |
| ANGST | 869 | RGDRQSL | 1668 |
| ANGST | 870 | RGDRQYL | 1669 |
| ANGSV | 871 | RGDRAYL | 1670 |
| ANGSV | 872 | RGDRGYL | 1671 |
| ANGSV | 873 | RGDRQAL | 1672 |
| ANGSV | 874 | RGDRQYL | 1673 |
| ANSGM | 875 | RGDRGAL | 1674 |
| ANSSA | 876 | RGDRGAL | 1675 |
| ANSSA | 877 | RGDRGSL | 1676 |
| ANSSA | 878 | RGDRGYL | 1677 |
| ANSSA | 879 | RGDRQAL | 1678 |
| ANSSA | 880 | RGDRQSL | 1679 |
| ANSSA | 881 | RGDRQYL | 1680 |
| ANSSG | 882 | RGDRAAL | 1681 |
| ANSSG | 883 | RGDRASL | 1682 |
| ANSSG | 884 | RGDRAYL | 1683 |
| ANSSG | 885 | RGDRGAL | 1684 |
| ANSSG | 886 | RGDRGSL | 1685 |
| ANSSG | 887 | RGDRGYL | 1686 |
| ANSSG | 888 | RGDRQAL | 1687 |
| ANSSG | 889 | RGDRQSL | 1688 |
| ANSSG | 890 | RGDRQYL | 1689 |
| ANSSM | 891 | RGDRAYL | 1690 |
| ANSSM | 892 | RGDRQAL | 1691 |
| ANSSM | 893 | RGDRQSL | 1692 |
| ANSSM | 894 | RGDRQYL | 1693 |
| ANSSS | 895 | RGDRGSL | 1694 |
| ANSSS | 896 | RGDRGYL | 1695 |
| ANSSS | 897 | RGDRQAL | 1696 |
| ANSSS | 898 | RGDRQSL | 1697 |
| ANSSS | 899 | RGDRQYL | 1698 |
| ANSST | 900 | RGDRAAL | 1699 |
| ANSST | 901 | RGDRGSL | 1700 |
| ANSST | 902 | RGDRQAL | 1701 |
| ANSST | 903 | RGDRQSL | 1702 |
| ANSST | 904 | RGDRQYL | 1703 |
| ANSSV | 905 | RGDRAYL | 1704 |
| ANSSV | 906 | RGDRGYL | 1705 |
| ANSSV | 907 | RGDRQAL | 1706 |
| ANSSV | 908 | RGDRQSL | 1707 |
| ANSSV | 909 | RGDRQYL | 1708 |
| ANTSA | 910 | RGDRAYL | 1709 |
| ANTSA | 911 | RGDRGAL | 1710 |
| ANTSA | 912 | RGDRGSL | 1711 |
| ANTSA | 913 | RGDRGYL | 1712 |
| ANTSA | 914 | RGDRQAL | 1713 |
| ANTSA | 915 | RGDRQYL | 1714 |
| ANTSG | 916 | RGDRASL | 1715 |
| ANTSG | 917 | RGDRGAL | 1716 |
| ANTSG | 918 | RGDRGSL | 1717 |
| ANTSG | 919 | RGDRGYL | 1718 |
| ANTSG | 920 | RGDRQAL | 1719 |
| ANTSG | 921 | RGDRQSL | 1720 |
| ANTSG | 922 | RGDRQYL | 1721 |
| ANTSM | 923 | RGDRAYL | 1722 |
| ANTSM | 924 | RGDRQAL | 1723 |
| ANTSM | 925 | RGDRQSL | 1724 |
| ANTSM | 926 | RGDRQYL | 1725 |
| ANTSS | 927 | RGDRGSL | 1726 |
| ANTSS | 928 | RGDRQAL | 1727 |
| ANTSS | 929 | RGDRQYL | 1728 |
| ANTST | 930 | RGDRAAL | 1729 |
| ANTST | 931 | RGDRQAL | 1730 |
| ANTST | 932 | RGDRQSL | 1731 |
| ANTST | 933 | RGDRQYL | 1732 |
| ANTSV | 934 | RGDRAYL | 1733 |
| ANTSV | 935 | RGDRGSL | 1734 |
| ANTSV | 936 | RGDRQAL | 1735 |
| ANTSV | 937 | RGDRQSL | 1736 |
| ANTSV | 938 | RGDRQYL | 1737 |
| ANYPH | 939 | RGDRGAL | 1738 |
| ANYSA | 940 | RGDRAAL | 1739 |
| ANYSA | 941 | RGDRASL | 1740 |
| ANYSA | 942 | RGDRGAL | 1741 |
| ANYSA | 943 | RGDRGSL | 1742 |
| ANYSA | 944 | RGDRQAL | 1743 |
| ANYSA | 945 | RGDRQSL | 1744 |
| ANYSG | 946 | RGDRAAL | 1745 |
| ANYSG | 947 | RGDRASL | 1746 |
| ANYSG | 948 | RGDRGAL | 1747 |
| ANYSG | 949 | RGDRGSL | 1748 |
| ANYSG | 950 | RGDRQAL | 1749 |
| ANYSG | 951 | RGDRQSL | 1750 |
| ANYSG | 952 | RGDRQYL | 1751 |
| ANYSM | 953 | RGDRAAL | 1752 |
| ANYSM | 954 | RGDRASL | 1753 |
| ANYSM | 955 | RGDRAYL | 1754 |
| ANYSM | 956 | RGDRQAL | 1755 |
| ANYSM | 957 | RGDRQSL | 1756 |
| ANYSS | 958 | RGDRGAL | 1757 |
| ANYSS | 959 | RGDRGSL | 1758 |
| ANYSS | 960 | RGDRQAL | 1759 |
| ANYSS | 961 | RGDRQSL | 1760 |
| ANYSS | 962 | RGDRQYL | 1761 |
| ANYST | 963 | RGDRQAL | 1762 |
| ANYST | 964 | RGDRQSL | 1763 |
| ANYST | 965 | RGDRQYL | 1764 |
| ANYSV | 966 | RGDRQAL | 1765 |
| ANYSV | 967 | RGDRQSL | 1766 |
| ANYSV | 968 | RGDRQYL | 1767 |
| FNSGM | 969 | RGDRGAL | 1768 |
| GNSGM | 970 | RGDRGAL | 1769 |
| HNSGM | 971 | RGDRGAL | 1770 |
| LNASA | 972 | RGDRAAL | 1771 |
| LNASA | 973 | RGDRASL | 1772 |
| LNASA | 974 | RGDRAYL | 1773 |
| LNASA | 975 | RGDRGAL | 1774 |
| LNASA | 976 | RGDRGSL | 1775 |
| LNASA | 977 | RGDRGYL | 1776 |
| LNASA | 978 | RGDRQAL | 1777 |
| LNASA | 979 | RGDRQSL | 1778 |
| LNASA | 980 | RGDRQYL | 1779 |
| LNASG | 981 | RGDRAAL | 1780 |
| LNASG | 982 | RGDRASL | 1781 |
| LNASG | 983 | RGDRAYL | 1782 |
| LNASG | 984 | RGDRGAL | 1783 |
| LNASG | 985 | RGDRGSL | 1784 |
| LNASG | 986 | RGDRGYL | 1785 |
| LNASG | 987 | RGDRQAL | 1786 |
| LNASG | 988 | RGDRQSL | 1787 |
| LNASG | 989 | RGDRQYL | 1788 |
| LNASI | 990 | RGDRQLL | 1789 |
| LNASM | 991 | RGDRAAL | 1790 |
| LNASM | 992 | RGDRASL | 1791 |
| LNASM | 993 | RGDRAYL | 1792 |
| LNASM | 994 | RGDRGAL | 1793 |
| LNASM | 995 | RGDRGSL | 1794 |
| LNASM | 996 | RGDRGYL | 1795 |
| LNASM | 997 | RGDRQAL | 1796 |
| LNASM | 998 | RGDRQSL | 1797 |
| LNASM | 999 | RGDRQYL | 1798 |
| LNASS | 1000 | RGDRAAL | 1799 |
| LNASS | 1001 | RGDRASL | 1800 |
| LNASS | 1002 | RGDRGAL | 1801 |
| LNASS | 1003 | RGDRGSL | 1802 |
| LNASS | 1004 | RGDRQAL | 1803 |
| LNASS | 1005 | RGDRQSL | 1804 |
| LNASS | 1006 | RGDRQYL | 1805 |
| LNAST | 1007 | RGDRAAL | 1806 |
| LNAST | 1008 | RGDRASL | 1807 |
| LNAST | 1009 | RGDRGAL | 1808 |
| LNAST | 1010 | RGDRGSL | 1809 |
| LNAST | 1011 | RGDRGYL | 1810 |
| LNAST | 1012 | RGDRQAL | 1811 |
| LNAST | 1013 | RGDRQSL | 1812 |
| LNAST | 1014 | RGDRQYL | 1813 |
| LNASV | 1015 | RGDRAAL | 1814 |
| LNASV | 1016 | RGDRASL | 1815 |
| LNASV | 1017 | RGDRGAL | 1816 |
| LNASV | 1018 | RGDRGSL | 1817 |
| LNASV | 1019 | RGDRQAL | 1818 |
| LNASV | 1020 | RGDRQSL | 1819 |
| LNASV | 1021 | RGDRQYL | 1820 |
| LNGNF | 1022 | RGDRQNL | 1821 |
| LNGNV | 1023 | RGDRGML | 1822 |
| LNGSA | 1024 | RGDRAAL | 1823 |
| LNGSA | 1025 | RGDRAYL | 1824 |
| LNGSA | 1026 | RGDRGSL | 1825 |
| LNGSA | 1027 | RGDRGYL | 1826 |
| LNGSA | 1028 | RGDRQAL | 1827 |
| LNGSA | 1029 | RGDRQSL | 1828 |
| LNGSA | 1030 | RGDRQYL | 1829 |
| LNGSG | 1031 | RGDRAAL | 1830 |
| LNGSG | 1032 | RGDRAYL | 1831 |
| LNGSG | 1033 | RGDRGAL | 1832 |
| LNGSG | 1034 | RGDRGSL | 1833 |
| LNGSG | 1035 | RGDRGYL | 1834 |
| LNGSG | 1036 | RGDRQAL | 1835 |
| LNGSG | 1037 | RGDRQYL | 1836 |
| LNGSM | 1038 | RGDRASL | 1837 |
| LNGSM | 1039 | RGDRGYL | 1838 |
| LNGSM | 1040 | RGDRQAL | 1839 |
| LNGSM | 1041 | RGDRQSL | 1840 |
| LNGSM | 1042 | RGDRQYL | 1841 |
| LNGSS | 1043 | RGDRAAL | 1842 |
| LNGSS | 1044 | RGDRAYL | 1843 |
| LNGSS | 1045 | RGDRGAL | 1844 |
| LNGSS | 1046 | RGDRQYL | 1845 |
| LNGST | 1047 | RGDRAAL | 1846 |
| LNGST | 1048 | RGDRASL | 1847 |
| LNGST | 1049 | RGDRAYL | 1848 |
| LNGST | 1050 | RGDRGAL | 1849 |
| LNGST | 1051 | RGDRGSL | 1850 |
| LNGST | 1052 | RGDRGYL | 1851 |
| LNGST | 1053 | RGDRQAL | 1852 |
| LNGST | 1054 | RGDRQSL | 1853 |
| LNGST | 1055 | RGDRQYL | 1854 |
| LNGSV | 1056 | RGDRASL | 1855 |
| LNGSV | 1057 | RGDRAYL | 1856 |
| LNGSV | 1058 | RGDRGYL | 1857 |
| LNGSV | 1059 | RGDRQAL | 1858 |
| LNGSV | 1060 | RGDRQSL | 1859 |
| LNGSV | 1061 | RGDRQYL | 1860 |
| LNSGM | 1062 | RGDRGAL | 1861 |
| LNSSA | 1063 | RGDRASL | 1862 |
| LNSSA | 1064 | RGDRAYL | 1863 |
| LNSSA | 1065 | RGDRGAL | 1864 |
| LNSSA | 1066 | RGDRGIL | 1865 |
| LNSSA | 1067 | RGDRGSL | 1866 |
| LNSSA | 1068 | RGDRGYL | 1867 |
| LNSSA | 1069 | RGDRQAL | 1868 |
| LNSSA | 1070 | RGDRQSL | 1869 |
| LNSSA | 1071 | RGDRQYL | 1870 |
| LNSSG | 1072 | RGDRAAL | 1871 |
| LNSSG | 1073 | RGDRASL | 1872 |
| LNSSG | 1074 | RGDRAYL | 1873 |
| LNSSG | 1075 | RGDRGAL | 1874 |
| LNSSG | 1076 | RGDRGSL | 1875 |
| LNSSG | 1077 | RGDRGYL | 1876 |
| LNSSG | 1078 | RGDRQAL | 1877 |
| LNSSG | 1079 | RGDRQSL | 1878 |
| LNSSG | 1080 | RGDRQYL | 1879 |
| LNSSM | 1081 | RGDRAAL | 1880 |
| LNSSM | 1082 | RGDRASL | 1881 |
| LNSSM | 1083 | RGDRAYL | 1882 |
| LNSSM | 1084 | RGDRGAL | 1883 |
| LNSSM | 1085 | RGDRGSL | 1884 |
| LNSSM | 1086 | RGDRQAL | 1885 |
| LNSSM | 1087 | RGDRQSL | 1886 |
| LNSSM | 1088 | RGDRQYL | 1887 |
| LNSSS | 1089 | RGDRAAL | 1888 |
| LNSSS | 1090 | RGDRGAL | 1889 |
| LNSSS | 1091 | RGDRGSL | 1890 |
| LNSSS | 1092 | RGDRQAL | 1891 |
| LNSSS | 1093 | RGDRQSL | 1892 |
| LNSSS | 1094 | RGDRQYL | 1893 |
| LNSST | 1095 | RGDRAAL | 1894 |
| LNSST | 1096 | RGDRASL | 1895 |
| LNSST | 1097 | RGDRGAL | 1896 |
| LNSST | 1098 | RGDRGSL | 1897 |
| LNSST | 1099 | RGDRQAL | 1898 |
| LNSST | 1100 | RGDRQSL | 1899 |
| LNSST | 1101 | RGDRQYL | 1900 |
| LNSSV | 1102 | RGDRAAL | 1901 |
| LNSSV | 1103 | RGDRASL | 1902 |
| LNSSV | 1104 | RGDRGAL | 1903 |
| LNSSV | 1105 | RGDRGSL | 1904 |
| LNSSV | 1106 | RGDRGYL | 1905 |
| LNSSV | 1107 | RGDRQAL | 1906 |
| LNSSV | 1108 | RGDRQSL | 1907 |
| LNSSV | 1109 | RGDRQYL | 1908 |
| LNTSA | 1110 | RGDRAAL | 1909 |
| LNTSA | 1111 | RGDRASL | 1910 |
| LNTSA | 1112 | RGDRAYL | 1911 |
| LNTSA | 1113 | RGDRGAL | 1912 |
| LNTSA | 1114 | RGDRGSL | 1913 |
| LNTSA | 1115 | RGDRGYL | 1914 |
| LNTSA | 1116 | RGDRQAL | 1915 |
| LNTSA | 1117 | RGDRQSL | 1916 |
| LNTSA | 1118 | RGDRQYL | 1917 |
| LNTSG | 1119 | RGDRAAL | 1918 |
| LNTSG | 1120 | RGDRASL | 1919 |
| LNTSG | 1121 | RGDRAYL | 1920 |
| LNTSG | 1122 | RGDRGAL | 1921 |
| LNTSG | 1123 | RGDRGSL | 1922 |
| LNTSG | 1124 | RGDRGYL | 1923 |
| LNTSG | 1125 | RGDRQAL | 1924 |
| LNTSG | 1126 | RGDRQSL | 1925 |
| LNTSG | 1127 | RGDRQYL | 1926 |
| LNTSM | 1128 | RGDRAAL | 1927 |
| LNTSM | 1129 | RGDRASL | 1928 |
| LNTSM | 1130 | RGDRAYL | 1929 |
| LNTSM | 1131 | RGDRGAL | 1930 |
| LNTSM | 1132 | RGDRGSL | 1931 |
| LNTSM | 1133 | RGDRQAL | 1932 |
| LNTSM | 1134 | RGDRQSL | 1933 |
| LNTSM | 1135 | RGDRQYL | 1934 |
| LNTSS | 1136 | RGDRAAL | 1935 |
| LNTSS | 1137 | RGDRGSL | 1936 |
| LNTSS | 1138 | RGDRQAL | 1937 |
| LNTSS | 1139 | RGDRQSL | 1938 |
| LNTSS | 1140 | RGDRQYL | 1939 |
| LNTST | 1141 | RGDRAAL | 1940 |
| LNTST | 1142 | RGDRASL | 1941 |
| LNTST | 1143 | RGDRGAL | 1942 |
| LNTST | 1144 | RGDRGSL | 1943 |
| LNTST | 1145 | RGDRQAL | 1944 |
| LNTST | 1146 | RGDRQSL | 1945 |
| LNTST | 1147 | RGDRQYL | 1946 |
| LNTSV | 1148 | RGDRAAL | 1947 |
| LNTSV | 1149 | RGDRASL | 1948 |
| LNTSV | 1150 | RGDRAYL | 1949 |
| LNTSV | 1151 | RGDRGAL | 1950 |
| LNTSV | 1152 | RGDRGSL | 1951 |
| LNTSV | 1153 | RGDRQAL | 1952 |
| LNTSV | 1154 | RGDRQSL | 1953 |
| LNYPH | 1155 | RGDRGAL | 1954 |
| LNYSA | 1156 | RGDRGAL | 1955 |
| LNYSA | 1157 | RGDRGSL | 1956 |
| LNYSA | 1158 | RGDRQAL | 1957 |
| LNYSA | 1159 | RGDRQSL | 1958 |
| LNYSA | 1160 | RGDRQYL | 1959 |
| LNYSG | 1161 | RGDRAAL | 1960 |
| LNYSG | 1162 | RGDRASL | 1961 |
| LNYSG | 1163 | RGDRGAL | 1962 |
| LNYSG | 1164 | RGDRGSL | 1963 |
| LNYSG | 1165 | RGDRQAL | 1964 |
| LNYSG | 1166 | RGDRQSL | 1965 |
| LNYSG | 1167 | RGDRQYL | 1966 |
| LNYSM | 1168 | RGDRQAL | 1967 |
| LNYSM | 1169 | RGDRQSL | 1968 |
| LNYSS | 1170 | RGDRGAL | 1969 |
| LNYSS | 1171 | RGDRGSL | 1970 |
| LNYSS | 1172 | RGDRQAL | 1971 |
| LNYSS | 1173 | RGDRQSL | 1972 |
| LNYSS | 1174 | RGDRQYL | 1973 |
| LNYST | 1175 | RGDRASL | 1974 |
| LNYST | 1176 | RGDRGAL | 1975 |
| LNYST | 1177 | RGDRGSL | 1976 |
| LNYST | 1178 | RGDRQAL | 1977 |
| LNYST | 1179 | RGDRQSL | 1978 |
| LNYST | 1180 | RGDRQYL | 1979 |
| LNYSV | 1181 | RGDRQAL | 1980 |
| LNYSV | 1182 | RGDRQSL | 1981 |
| MNASA | 1183 | RGDRAAL | 1982 |
| MNASA | 1184 | RGDRAYL | 1983 |
| MNASA | 1185 | RGDRGSL | 1984 |
| MNASA | 1186 | RGDRGYL | 1985 |
| MNASA | 1187 | RGDRQAL | 1986 |
| MNASA | 1188 | RGDRQSL | 1987 |
| MNASA | 1189 | RGDRQYL | 1988 |
| MNASG | 1190 | RGDRAAL | 1989 |
| MNASG | 1191 | RGDRASL | 1990 |
| MNASG | 1192 | RGDRAYL | 1991 |
| MNASG | 1193 | RGDRGSL | 1992 |
| MNASG | 1194 | RGDRQAL | 1993 |
| MNASG | 1195 | RGDRQSL | 1994 |
| MNASG | 1196 | RGDRQYL | 1995 |
| MNASI | 1197 | RGDRQLL | 1996 |
| MNASM | 1198 | RGDRAAL | 1997 |
| MNASM | 1199 | RGDRAYL | 1998 |
| MNASM | 1200 | RGDRGAL | 1999 |
| MNASM | 1201 | RGDRQAL | 2000 |
| MNASM | 1202 | RGDRQSL | 2001 |
| MNASS | 1203 | RGDRAAL | 2002 |
| MNAST | 1204 | RGDRAAL | 2003 |
| MNAST | 1205 | RGDRASL | 2004 |
| MNAST | 1206 | RGDRAYL | 2005 |
| MNAST | 1207 | RGDRGAL | 2006 |
| MNAST | 1208 | RGDRGSL | 2007 |
| MNAST | 1209 | RGDRQAL | 2008 |
| MNAST | 1210 | RGDRQSL | 2009 |
| MNAST | 1211 | RGDRQYL | 2010 |
| MNASV | 1212 | RGDRAAL | 2011 |
| MNASV | 1213 | RGDRASL | 2012 |
| MNASV | 1214 | RGDRAYL | 2013 |
| MNASV | 1215 | RGDRQAL | 2014 |
| MNASV | 1216 | RGDRQSL | 2015 |
| MNASV | 1217 | RGDRQYL | 2016 |
| MNGNF | 1218 | RGDRQNL | 2017 |
| MNGSA | 1219 | RGDRAYL | 2018 |
| MNGSA | 1220 | RGDRGYL | 2019 |
| MNGSA | 1221 | RGDRQAL | 2020 |
| MNGSA | 1222 | RGDRQSL | 2021 |
| MNGSA | 1223 | RGDRQYL | 2022 |
| MNGSG | 1224 | RGDRAAL | 2023 |
| MNGSG | 1225 | RGDRAYL | 2024 |
| MNGSG | 1226 | RGDRGAL | 2025 |
| MNGSG | 1227 | RGDRGSL | 2026 |
| MNGSG | 1228 | RGDRGYL | 2027 |
| MNGSG | 1229 | RGDRQAL | 2028 |
| MNGSG | 1230 | RGDRQSL | 2029 |
| MNGSG | 1231 | RGDRQYL | 2030 |
| MNGSM | 1232 | RGDRQAL | 2031 |
| MNGSM | 1233 | RGDRQSL | 2032 |
| MNGSM | 1234 | RGDRQYL | 2033 |
| MNGSS | 1235 | RGDRQYL | 2034 |
| MNGST | 1236 | RGDRAYL | 2035 |
| MNGST | 1237 | RGDRGAL | 2036 |
| MNGST | 1238 | RGDRGYL | 2037 |
| MNGST | 1239 | RGDRQAL | 2038 |
| MNGST | 1240 | RGDRQSL | 2039 |
| MNGST | 1241 | RGDRQYL | 2040 |
| MNGSV | 1242 | RGDRGYL | 2041 |
| MNGSV | 1243 | RGDRQAL | 2042 |
| MNGSV | 1244 | RGDRQYL | 2043 |
| MNSGM | 1245 | RGDRGAL | 2044 |
| MNSSA | 1246 | RGDRAAL | 2045 |
| MNSSA | 1247 | RGDRAYL | 2046 |
| MNSSA | 1248 | RGDRGSL | 2047 |
| MNSSA | 1249 | RGDRGYL | 2048 |
| MNSSA | 1250 | RGDRQAL | 2049 |
| MNSSA | 1251 | RGDRQSL | 2050 |
| MNSSA | 1252 | RGDRQYL | 2051 |
| MNSSG | 1253 | RGDRAAL | 2052 |
| MNSSG | 1254 | RGDRASL | 2053 |
| MNSSG | 1255 | RGDRAYL | 2054 |
| MNSSG | 1256 | RGDRGAL | 2055 |
| MNSSG | 1257 | RGDRGSL | 2056 |
| MNSSG | 1258 | RGDRQAL | 2057 |
| MNSSG | 1259 | RGDRQSL | 2058 |
| MNSSG | 1260 | RGDRQYL | 2059 |
| MNSSM | 1261 | RGDRAAL | 2060 |
| MNSSM | 1262 | RGDRASL | 2061 |
| MNSSM | 1263 | RGDRAYL | 2062 |
| MNSSM | 1264 | RGDRGSL | 2063 |
| MNSSM | 1265 | RGDRQAL | 2064 |
| MNSSM | 1266 | RGDRQSL | 2065 |
| MNSSM | 1267 | RGDRQYL | 2066 |
| MNSSS | 1268 | RGDRAAL | 2067 |
| MNSST | 1269 | RGDRAAL | 2068 |
| MNSST | 1270 | RGDRASL | 2069 |
| MNSST | 1271 | RGDRQAL | 2070 |
| MNSST | 1272 | RGDRQSL | 2071 |
| MNSST | 1273 | RGDRQYL | 2072 |
| MNSSV | 1274 | RGDRAAL | 2073 |
| MNSSV | 1275 | RGDRAYL | 2074 |
| MNSSV | 1276 | RGDRQAL | 2075 |
| MNSSV | 1277 | RGDRQSL | 2076 |
| MNSSV | 1278 | RGDRQYL | 2077 |
| MNTSA | 1279 | RGDRAYL | 2078 |
| MNTSG | 1280 | RGDRAAL | 2079 |
| MNTSG | 1281 | RGDRQAL | 2080 |
| MNTSG | 1282 | RGDRQYL | 2081 |
| MNTSM | 1283 | RGDRAYL | 2082 |
| MNTST | 1284 | RGDRQYL | 2083 |
| MNYPH | 1285 | RGDRGAL | 2084 |
| MNYSA | 1286 | RGDRAAL | 2085 |
| MNYSA | 1287 | RGDRGYL | 2086 |
| MNYSA | 1288 | RGDRQAL | 2087 |
| MNYSA | 1289 | RGDRQSL | 2088 |
| MNYSG | 1290 | RGDRAAL | 2089 |
| MNYSG | 1291 | RGDRASL | 2090 |
| MNYSG | 1292 | RGDRGSL | 2091 |
| MNYSG | 1293 | RGDRQAL | 2092 |
| MNYSG | 1294 | RGDRQSL | 2093 |
| MNYSS | 1295 | RGDRQAL | 2094 |
| MNYST | 1296 | RGDRAAL | 2095 |
| MNYST | 1297 | RGDRQAL | 2096 |
| MNYST | 1298 | RGDRQSL | 2097 |
| MNYST | 1299 | RGDRQYL | 2098 |
| NNSGM | 1300 | RGDRGAL | 2099 |
| QNSGM | 1301 | RGDRGAL | 2100 |
| SNASA | 1302 | RGDRAAL | 2101 |
| SNASA | 1303 | RGDRASL | 2102 |
| SNASA | 1304 | RGDRAYL | 2103 |
| SNASA | 1305 | RGDRGAL | 2104 |
| SNASA | 1306 | RGDRGSL | 2105 |
| SNASA | 1307 | RGDRGYL | 2106 |
| SNASA | 1308 | RGDRQAL | 2107 |
| SNASA | 1309 | RGDRQSL | 2108 |
| SNASA | 1310 | RGDRQYL | 2109 |
| SNASG | 1311 | RGDRAAL | 2110 |
| SNASG | 1312 | RGDRASL | 2111 |
| SNASG | 1313 | RGDRAYL | 2112 |
| SNASG | 1314 | RGDRGAL | 2113 |
| SNASG | 1315 | RGDRGSL | 2114 |
| SNASG | 1316 | RGDRGYL | 2115 |
| SNASG | 1317 | RGDRQAL | 2116 |
| SNASG | 1318 | RGDRQSL | 2117 |
| SNASG | 1319 | RGDRQYL | 2118 |
| SNASI | 1320 | RGDRQLL | 2119 |
| SNASM | 1321 | RGDRAAL | 2120 |
| SNASM | 1322 | RGDRASL | 2121 |
| SNASM | 1323 | RGDRGAL | 2122 |
| SNASM | 1324 | RGDRGSL | 2123 |
| SNASM | 1325 | RGDRQAL | 2124 |
| SNASM | 1326 | RGDRQSL | 2125 |
| SNASS | 1327 | RGDRAAL | 2126 |
| SNASS | 1328 | RGDRASL | 2127 |
| SNASS | 1329 | RGDRAYL | 2128 |
| SNASS | 1330 | RGDRGAL | 2129 |
| SNASS | 1331 | RGDRGSL | 2130 |
| SNASS | 1332 | RGDRQAL | 2131 |
| SNASS | 1333 | RGDRQSL | 2132 |
| SNASS | 1334 | RGDRQYL | 2133 |
| SNAST | 1335 | RGDRAAL | 2134 |
| SNAST | 1336 | RGDRASL | 2135 |
| SNAST | 1337 | RGDRAYL | 2136 |
| SNAST | 1338 | RGDRGAL | 2137 |
| SNAST | 1339 | RGDRGSL | 2138 |
| SNAST | 1340 | RGDRGYL | 2139 |
| SNAST | 1341 | RGDRQAL | 2140 |
| SNAST | 1342 | RGDRQSL | 2141 |
| SNAST | 1343 | RGDRQYL | 2142 |
| SNASV | 1344 | RGDRAAL | 2143 |
| SNASV | 1345 | RGDRASL | 2144 |
| SNASV | 1346 | RGDRAYL | 2145 |
| SNASV | 1347 | RGDRGSL | 2146 |
| SNASV | 1348 | RGDRGYL | 2147 |
| SNASV | 1349 | RGDRQAL | 2148 |
| SNASV | 1350 | RGDRQSL | 2149 |
| SNASV | 1351 | RGDRQYL | 2150 |
| SNGNF | 1352 | RGDRQNL | 2151 |
| SNGNV | 1353 | RGDRGML | 2152 |
| SNGSA | 1354 | RGDRGYL | 2153 |
| SNGSA | 1355 | RGDRQAL | 2154 |
| SNGSA | 1356 | RGDRQSL | 2155 |
| SNGSA | 1357 | RGDRQYI | 2156 |
| SNGSG | 1358 | RGDRASL | 2157 |
| SNGSG | 1359 | RGDRAYL | 2158 |
| SNGSG | 1360 | RGDRGAL | 2159 |
| SNGSG | 1361 | RGDRGSL | 2160 |
| SNGSG | 1362 | RGDRGYL | 2161 |
| SNGSG | 1363 | RGDRQAL | 2162 |
| SNGSG | 1364 | RGDRQSL | 2163 |
| SNGSG | 1365 | RGDRQYL | 2164 |
| SNGSM | 1366 | RGDRAYL | 2165 |
| SNGSM | 1367 | RGDRGYL | 2166 |
| SNGSM | 1368 | RGDRQAL | 2167 |
| SNGSM | 1369 | RGDRQSL | 2168 |
| SNGSM | 1370 | RGDRQYL | 2169 |
| SNGSS | 1371 | RGDRGYL | 2170 |
| SNGSS | 1372 | RGDRQYL | 2171 |
| SNGST | 1373 | RGDRAAL | 2172 |
| SNGST | 1374 | RGDRASL | 2173 |
| SNGST | 1375 | RGDRGAL | 2174 |
| SNGST | 1376 | RGDRGYL | 2175 |
| SNGST | 1377 | RGDRQAL | 2176 |
| SNGST | 1378 | RGDRQSL | 2177 |
| SNGST | 1379 | RGDRQYL | 2178 |
| SNGSV | 1380 | RGDRGYL | 2179 |
| SNGSV | 1381 | RGDRQAL | 2180 |
| SNGSV | 1382 | RGDRQSL | 2181 |
| SNGSV | 1383 | RGDRQYL | 2182 |
| SNSGM | 1384 | RGDRGAL | 2183 |
| SNSSA | 1385 | RGDRAYL | 2184 |
| SNSSA | 1386 | RGDRGAL | 2185 |
| SNSSA | 1387 | RGDRGIL | 2186 |
| SNSSA | 1388 | RGDRGSL | 2187 |
| SNSSA | 1389 | RGDRGYL | 2188 |
| SNSSA | 1390 | RGDRQAL | 2189 |
| SNSSA | 1391 | RGDRQSL | 2190 |
| SNSSA | 1392 | RGDRQYL | 2191 |
| SNSSG | 1393 | RGDRAAL | 2192 |
| SNSSG | 1394 | RGDRAYL | 2193 |
| SNSSG | 1395 | RGDRGAL | 2194 |
| SNSSG | 1396 | RGDRGSL | 2195 |
| SNSSG | 1397 | RGDRGYL | 2196 |
| SNSSG | 1398 | RGDRQAL | 2197 |
| SNSSG | 1399 | RGDRQSL | 2198 |
| SNSSG | 1400 | RGDRQYL | 2199 |
| SNSSM | 1401 | RGDRAAL | 2200 |
| SNSSM | 1402 | RGDRASL | 2201 |
| SNSSM | 1403 | RGDRAYL | 2202 |
| SNSSM | 1404 | RGDRGSL | 2203 |
| SNSSM | 1405 | RGDRQAL | 2204 |
| SNSSM | 1406 | RGDRQSL | 2205 |
| SNSSM | 1407 | RGDRQYL | 2206 |
| SNSSS | 1408 | RGDRAAL | 2207 |
| SNSSS | 1409 | RGDRGSL | 2208 |
| SNSSS | 1410 | RGDRQAL | 2209 |
| SNSSS | 1411 | RGDRQSL | 2210 |
| SNSSS | 1412 | RGDRQYL | 2211 |
| SNSST | 1413 | RGDRAAL | 2212 |
| SNSST | 1414 | RGDRASL | 2213 |
| SNSST | 1415 | RGDRGSL | 2214 |
| SNSST | 1416 | RGDRGYL | 2215 |
| SNSST | 1417 | RGDRQAL | 2216 |
| SNSST | 1418 | RGDRQSL | 2217 |
| SNSST | 1419 | RGDRQYL | 2218 |
| SNSSV | 1420 | RGDRQAL | 2219 |
| SNSSV | 1421 | RGDRQSL | 2220 |
| SNSSV | 1422 | RGDRQYL | 2221 |
| SNTSA | 1423 | RGDRAAL | 2222 |
| SNTSA | 1424 | RGDRGAL | 2223 |
| SNTSA | 1425 | RGDRGSL | 2224 |
| SNTSA | 1426 | RGDRGYL | 2225 |
| SNTSA | 1427 | RGDRQAL | 2226 |
| SNTSA | 1428 | RGDRQSL | 2227 |
| SNTSA | 1429 | RGDRQYL | 2228 |
| SNTSG | 1430 | RGDRAAL | 2229 |
| SNTSG | 1431 | RGDRASL | 2230 |
| SNTSG | 1432 | RGDRGAL | 2231 |
| SNTSG | 1433 | RGDRGSL | 2232 |
| SNTSG | 1434 | RGDRGYL | 2233 |
| SNTSG | 1435 | RGDRQAL | 2234 |
| SNTSG | 1436 | RGDRQSL | 2235 |
| SNTSG | 1437 | RGDRQYL | 2236 |
| SNTSM | 1438 | RGDRAYL | 2237 |
| SNTSM | 1439 | RGDRGSL | 2238 |
| SNTSM | 1440 | RGDRQAL | 2239 |
| SNTSM | 1441 | RGDRQSL | 2240 |
| SNTSS | 1442 | RGDRQAL | 2241 |
| SNTST | 1443 | RGDRQAL | 2242 |
| SNTST | 1444 | RGDRQSL | 2243 |
| SNTST | 1445 | RGDRQYL | 2244 |
| SNTSV | 1446 | RGDRAYL | 2245 |
| SNTSV | 1447 | RGDRQAL | 2246 |
| SNTSV | 1448 | RGDRQSL | 2247 |
| SNTSV | 1449 | RGDRQYL | 2248 |
| SNYPH | 1450 | RGDRGAL | 2249 |
| SNYSA | 1451 | RGDRAAL | 2250 |
| SNYSA | 1452 | RGDRASL | 2251 |
| SNYSA | 1453 | RGDRGAL | 2252 |
| SNYSA | 1454 | RGDRGSL | 2253 |
| SNYSA | 1455 | RGDRQAL | 2254 |
| SNYSA | 1456 | RGDRQSL | 2255 |
| SNYSA | 1457 | RGDRQYL | 2256 |
| SNYSG | 1458 | RGDRAAL | 2257 |
| SNYSG | 1459 | RGDRGAL | 2258 |
| SNYSG | 1460 | RGDRGSL | 2259 |
| SNYSG | 1461 | RGDRQAL | 2260 |
| SNYSG | 1462 | RGDRQSL | 2261 |
| SNYSM | 1463 | RGDRAAL | 2262 |
| SNYSM | 1464 | RGDRASL | 2263 |
| SNYSM | 1465 | RGDRGSL | 2264 |
| SNYSM | 1466 | RGDRQAL | 2265 |
| SNYSM | 1467 | RGDRQSL | 2266 |
| SNYSS | 1468 | RGDRGAL | 2267 |
| SNYSS | 1469 | RGDRGSL | 2268 |
| SNYSS | 1470 | RGDRQAL | 2269 |
| SNYSS | 1471 | RGDRQSL | 2270 |
| SNYST | 1472 | RGDRAAL | 2271 |
| SNYST | 1473 | RGDRGAL | 2272 |
| SNYST | 1474 | RGDRGSL | 2273 |
| SNYST | 1475 | RGDRQAL | 2274 |
| SNYST | 1476 | RGDRQSL | 2275 |
| SNYST | 1477 | RGDRQYL | 2276 |
| SNYSV | 1478 | RGDRQAL | 2277 |
| SNYSV | 1479 | RGDRQSL | 2278 |
| SNYSV | 1480 | RGDRQYL | 2279 |
| TNSGM | 1481 | RGDRGAL | 2280 |
| VNASA | 1482 | RGDRAAL | 2281 |
| VNASA | 1483 | RGDRGAL | 2282 |
| VNASA | 1484 | RGDRGSL | 2283 |
| VNASA | 1485 | RGDRGYL | 2284 |
| VNASA | 1486 | RGDRQAL | 2285 |
| VNASA | 1487 | RGDRQYL | 2286 |
| VNASG | 1488 | RGDRAAL | 2287 |
| VNASG | 1489 | RGDRAYL | 2288 |
| VNASG | 1490 | RGDRGAL | 2289 |
| VNASG | 1491 | RGDRGSL | 2290 |
| VNASG | 1492 | RGDRGYL | 2291 |
| VNASG | 1493 | RGDRQAL | 2292 |
| VNASG | 1494 | RGDRQYL | 2293 |
| VNASM | 1495 | RGDRAYL | 2294 |
| VNASM | 1496 | RGDRQAL | 2295 |
| VNASM | 1497 | RGDRQSL | 2296 |
| VNASS | 1498 | RGDRGSL | 2297 |
| VNASS | 1499 | RGDRQAL | 2298 |
| VNASS | 1500 | RGDRQYL | 2299 |
| VNAST | 1501 | RGDRAAL | 2300 |
| VNAST | 1502 | RGDRGAL | 2301 |
| VNAST | 1503 | RGDRGSL | 2302 |
| VNAST | 1504 | RGDRGYL | 2303 |
| VNAST | 1505 | RGDRQAL | 2304 |
| VNAST | 1506 | RGDRQSL | 2305 |
| VNAST | 1507 | RGDRQYL | 2306 |
| VNASV | 1508 | RGDRGSL | 2307 |
| VNASV | 1509 | RGDRGYL | 2308 |
| VNASV | 1510 | RGDRQAL | 2309 |
| VNASV | 1511 | RGDRQSL | 2310 |
| VNASV | 1512 | RGDRQYL | 2311 |
| VNGNF | 1513 | RGDRQNL | 2312 |
| VNGNV | 1514 | RGDRGML | 2313 |
| VNGSA | 1515 | RGDRAYL | 2314 |
| VNGSA | 1516 | RGDRGAL | 2315 |
| VNGSA | 1517 | RGDRGSL | 2316 |
| VNGSA | 1518 | RGDRGYL | 2317 |
| VNGSA | 1519 | RGDRQAL | 2318 |
| VNGSA | 1520 | RGDRQSL | 2319 |
| VNGSA | 1521 | RGDRQYL | 2320 |
| VNGSG | 1522 | RGDRAAL | 2321 |
| VNGSG | 1523 | RGDRASL | 2322 |
| VNGSG | 1524 | RGDRAYL | 2323 |
| VNGSG | 1525 | RGDRGAL | 2324 |
| VNGSG | 1526 | RGDRGSL | 2325 |
| VNGSG | 1527 | RGDRGYL | 2326 |
| VNGSG | 1528 | RGDRQAL | 2327 |
| VNGSG | 1529 | RGDRQSL | 2328 |
| VNGSG | 1530 | RGDRQYL | 2329 |
| VNGSM | 1531 | RGDRGYL | 2330 |
| VNGSM | 1532 | RGDRQAL | 2331 |
| VNGSM | 1533 | RGDRQSL | 2332 |
| VNGSM | 1534 | RGDRQYL | 2333 |
| VNGSS | 1535 | RGDRAYL | 2334 |
| VNGSS | 1536 | RGDRGAL | 2335 |
| VNGSS | 1537 | RGDRGSL | 2336 |
| VNGSS | 1538 | RGDRQAL | 2337 |
| VNGSS | 1539 | RGDRQSL | 2338 |
| VNGSS | 1540 | RGDRQYL | 2339 |
| VNGST | 1541 | RGDRAAL | 2340 |
| VNGST | 1542 | RGDRAYL | 2341 |
| VNGST | 1543 | RGDRGAL | 2342 |
| VNGST | 1544 | RGDRGYL | 2343 |
| VNGST | 1545 | RGDRQAL | 2344 |
| VNGST | 1546 | RGDRQSL | 2345 |
| VNGST | 1547 | RGDRQYL | 2346 |
| VNGSV | 1548 | RGDRAYL | 2347 |
| VNGSV | 1549 | RGDRQAL | 2348 |
| VNGSV | 1550 | RGDRQSL | 2349 |
| VNGSV | 1551 | RGDRQYL | 2350 |
| VNSGM | 1552 | RGDRGAL | 2351 |
| VNSSA | 1553 | RGDRGAL | 2352 |
| VNSSA | 1554 | RGDRGSL | 2353 |
| VNSSA | 1555 | RGDRGYL | 2354 |
| VNSSA | 1556 | RGDRQAL | 2355 |
| VNSSA | 1557 | RGDRQSL | 2356 |
| VNSSA | 1558 | RGDRQYL | 2357 |
| VNSSG | 1559 | RGDRAAL | 2358 |
| VNSSG | 1560 | RGDRAYL | 2359 |
| VNSSG | 1561 | RGDRGAL | 2360 |
| VNSSG | 1562 | RGDRQAL | 2361 |
| VNSSG | 1563 | RGDRQSL | 2362 |
| VNSSG | 1564 | RGDRQYL | 2363 |
| VNSSM | 1565 | RGDRAYL | 2364 |
| VNSSM | 1566 | RGDRQAL | 2365 |
| VNSSM | 1567 | RGDRQSL | 2366 |
| VNSSM | 1568 | RGDRQYL | 2367 |
| VNSSS | 1569 | RGDRGSL | 2368 |
| VNSSS | 1570 | RGDRQAL | 2369 |
| VNSSS | 1571 | RGDRQSL | 2370 |
| VNSST | 1572 | RGDRGAL | 2371 |
| VNSST | 1573 | RGDRQAL | 2372 |
| VNSST | 1574 | RGDRQSL | 2373 |
| VNSST | 1575 | RGDRQYL | 2374 |
| VNSSV | 1576 | RGDRAYL | 2375 |
| VNSSV | 1577 | RGDRQAL | 2376 |
| VNSSV | 1578 | RGDRQSL | 2377 |
| VNTSA | 1579 | RGDRAYL | 2378 |
| VNTSG | 1580 | RGDRAAL | 2379 |
| VNTSG | 1581 | RGDRGAL | 2380 |
| VNTSG | 1582 | RGDRGSL | 2381 |
| VNTSG | 1583 | RGDRGYL | 2382 |
| VNTSG | 1584 | RGDRQAL | 2383 |
| VNTSG | 1585 | RGDRQSL | 2384 |
| VNTSG | 1586 | RGDRQYL | 2385 |
| VNTST | 1587 | RGDRAYL | 2386 |
| VNYPH | 1588 | RGDRGAL | 2387 |
| VNYSA | 1589 | RGDRGAL | 2388 |
| VNYSA | 1590 | RGDRGSL | 2389 |
| VNYSA | 1591 | RGDRQAL | 2390 |
| VNYSA | 1592 | RGDRQSL | 2391 |
| VNYSG | 1593 | RGDRAAL | 2392 |
| VNYSG | 1594 | RGDRGAL | 2393 |
| VNYSG | 1595 | RGDRQAL | 2394 |
| VNYSG | 1596 | RGDRQSL | 2395 |
| VNYSG | 1597 | RGDRQYL | 2396 |
| VNYSS | 1598 | RGDRGSL | 2397 |
| VNYSS | 1599 | RGDRGYL | 2398 |

### BRIEF DESCRIPTION OF THE DRAWINGS

FIGS. 1A-1C show that a cell-based DUX4 knockdown assay identified potent artificial miRNA sequences targeting DUX4. FIG. 1A shows a schematic of lentiviral constructs used in the cell-based DUX4 knockdown assay. FIG. 1B shows flow cytometry workflow and selection of positive and negative cell populations. FIG. 1C shows a graph of top miRNA sequences based on their enrichment in the GFP⁺, mcherry (low) cell population.

FIGS. 2A-2B show that candidate miRNA sequences effectively knockdown DUX4 and reduce expression of hZSCAN4 as a DUX4 target gene in FSHD patient myotubes. FIGS. 2A-2B show graphs of DUX4 (FIG. 2A) and hZSCAN4 (FIG. 2B) expression in FSHD patient myotubes transduced with a candidate miRNA targeting DUX4 (miRNA #1-13). Arrows indicate the top 2 miRNA sequences (miRNA #7 and miRNA #12).

FIGS. 3A-3B show that the top two candidate miRNA sequences do not result in off-target effects based on RNAseq. FIGS. 3A-3B show volcano plots of differentially expressed genes in human myotubes transduced with miRNA #7 (FIG. 3A) or miRNA #12 (FIG. 3B) is candidate miRNA targeting DUX4. Gene expression was analyzed by RNAseq.

FIGS. 4A-4B show that candidate miRNA sequences effectively knockdown DUX4 and reduce expression of DUX4 downstream genes in a mouse model of FSHD. FIGS. 4A-4B show graphs of DUX4 (FIG. 4A) and mKif4 (FIG. 4B) expression in mouse triceps following administration of candidate miRNA sequences.

FIGS. 5A-5C show that candidate miRNA sequences effectively knockdown DUX4 and reduce expression of DUX4 downstream genes in FSHD patient myotubes transduced with candidate miRNA sequences at the time of induction of differentiation. FIG. 5A shows a study design in FSHD patient myotubes in which transduction is performed at the time of differentiation. FIGS. 5B-5C show graphs of DUX4 (FIG. 5B) and DUX4 target gene (FIG. 5C) expression in FSHD patient cells following transduction with miRNA #7 or miRNA #12 vectors.

FIGS. 6A-6C show that candidate miRNA sequences effectively knockdown DUX4 and reduce expression of DUX4 downstream genes in FSHD patient myotubes transduced with candidate miRNA sequences after differentiation. FIG. 6A shows a study design in FSHD patient myotubes in which transduction is performed after differentiation. FIG. 6B-6C show graphs of DUX4 (FIG. 6B) and DUX4 target gene (FIG. 6C) expression in patient cells following transduction with miRNA #7 or miRNA #12 vectors.

FIGS. 7A-7D show that miRNA #7 and miRNA #12 sequences do not result in off-target effects. FIG. 7A shows a volcano plot of differentially expressed genes after transduction of human myotubes with miRNA #7 based on RNAseq. FIG. 7B shows a volcano plot of differentially expressed miRNAs after transduction of human myotubes with miRNA #12 based on RNAseq. FIG. 7C shows a volcano plot of differentially expressed genes after transduction of human myotubes with miRNA #7 based on small RNAseq. FIG. 7D shows a volcano plot of differentially expressed miRNAs after transduction of human myotubes with miRNA #12 based on small RNAseq. No endogenous mRNA or miRNAs met criteria of adjusted p<0.001, 50% decrease or increase.

FIGS. 8A-8F show that administration of a tool compound of composition 1 protects against DUX4 induced damage in a moderate mouse model of FSHD. FIG. 8A shows a study design for testing a tool compound of Composition 1 in a moderate mouse model of FSHD. FIG. 8B shows representative images of skeletal muscle from a mouse model of FSHD treated with a tool compound of Composition 1 or vehicle. FIGS. 8C-8D show graphs of results from treadmill assessments of a mouse model of FSHD administered with a tool compound of Composition 1 or vehicle. FIG. 8E show immunofluorescent images of mouse is skeletal muscle stained for embryonic myosin heavy chain (marker of muscle regeneration) and wheat germ agglutinin (WGA; marker of extracellular matrix and connective tissue in skeletal muscle). FIG. 8F shows a graph of FSHD composite gene expression in skeletal muscle (quadriceps, tibialis anterior (TA), triceps) of mice administered with a tool compound of Composition 1 or vehicle.

FIGS. 9A-9E show that administration of Composition 1 results in dose-dependent protection against DUX4 induced damage in a severe mouse model of FSHD. FIG. 9A shows a study design for testing Composition 1 in a severe mouse model of FSHD. FIGS. 9B-9C show graphs of results from treadmill assessments of mice administered with Composition 1 (3E+12 vg/kg, 1E+13 vg/kg, or 3E+13 vg/kg) or vehicle. FIG. 9D shows a graph of FSHD composite gene expression in skeletal muscle (quadriceps, triceps, tibialis anterior) of mice administered with Composition 1 (3E+12 vg/kg, 1E+13 vg/kg, or 3E+13 vg/kg) or vehicle. FIG. 9E shows immunofluorescent images of mouse skeletal muscle stained for embryonic myosin heavy chain and laminin (marker outlining individual muscle fibers) from a mouse model of FSHD administered with Composition 1 (3E+12 vg/kg, 1E+13 vg/kg, or 3E+13 vg/kg) or vehicle.

FIGS. 10A-10D show that administration of Composition 1 protects against DUX4 induced damage in a mild mouse model of FSHD. FIG. 10A shows a study design for testing Composition 1 in a mild mouse model of FSHD. FIG. 10B shows a graph of FSHD composite gene expression in 14 months old ACTA1-MCM;FLExDUX4 mice following administration of Composition 1 at a dose of 1E+13 vg/kg or 2E+13 vg/kg. FIG. 10C shows representative images of skeletal muscle from mice treated with Composition 1 (2E+13 vg/kg) or vehicle. FIG. 10D shows immunofluorescent images of mouse skeletal muscle stained for embryonic myosin heavy chain (muscle regeneration marker) and laminin (marker outlining individual muscle fibers) from a mild mouse model of FSHD treated with Composition 1 (2E+13 vg/kg) or vehicle.

FIG. 11 shows that therapeutic levels of the DUX4 miRNA #7 can be detected in skeletal muscles of non-human primates (NHPs) injected with Composition 1 at a dose of 2E+13 vg/kg or 4E+13 vg/kg. FIG. 11 shows a graph of average normalized DUX4 targeting miR expression across skeletal muscles (5 muscles in mice and 8 muscles in NHPs).

FIGS. 12A-12E show that administration of Composition 2 rescued biochemical and histological phenotypes in a mouse model of FSHD. FIG. 12A shows a study design for testing Composition 2 in a severe mouse model of FSHD. FIG. 12B shows a graph of time to exhaustion of mice administered Composition 2 or vehicle. FIG. 12C shows a graph of number of stimuli per minute of mice administered Composition 2 or vehicle. FIG. 12D shows a graph of FSHD composite gene expression in skeletal muscle (quadriceps, triceps, tibialis anterior) of mice administered Composition 2 or vehicle. FIG. 12E shows a graph of vector genome detected in the liver of Composition 2-injected mice.

FIG. 13. shows that therapeutic levels of the DUX4 miRNA #12 can be detected in skeletal muscles of non-human primates (NHPs) injected with Composition 2 at a dose of 4E+13 vg/kg. FIG. 13 shows a graph of average normalized DUX4 targeting miR expression across skeletal muscles (5 muscles in mice and 8 muscles in NHPs).

FIGS. 14A-14B show that MyoAAV-LD B used in Composition 1 and Composition 2 effectively transduces muscle fibers after systemic administration in NHPs. FIG. 14A shows a graph of relative transgene mRNA expression. FIG. 14B shows a graph of relative vector genome abundance.

### DETAILED DESCRIPTION

The present disclosure is directed to recombinant nucleic acids, compositions comprising such recombinant nucleic acids (e.g., adeno-associated viral (AAV) compositions), and methods of use thereof for treating facioscapulohumeral muscular dystrophy (FSHD).

Some aspects of the invention provide an AAV particle comprising (e.g., encapsidating) a recombinant nucleic acid comprising a promoter and a pri-miRNA, which comprises a primary microRNA (pri-miRNA) scaffold (e.g., a scaffold derived from an endogenous pri-miRNA such as miR-138 or miR-139), a guide strand that targets a double homeobox protein 4 (DUX4) transcript, and a passenger strand that is at least partially complementary to the guide strand.

### MicroRNA

MicroRNA (miRNA) are small, single-stranded, non-coding RNA molecules. miRNAs base-pair to complementary sequences in mRNA molecules, thereby silencing post-transcriptional regulation of gene expression. Typically, miRNA molecules silence mRNA translation by cleavage of mRNA strand into two pieces or destabilization of the mRNA by shortening its poly(A) tail.

miRNAs resemble small interfering RNAs (siRNAs), however, miRNAs derive from regions of RNA transcripts that fold back on themselves to form short hairpins. miRNA in its final form is a non-coding RNA molecule ~22 nucleotides in length. However, it is initially transcribed as part of one arm of an RNA stem-loop that in turn forms part of a several hundred nucleotide-long miRNA precursor termed a primary miRNA transcript (pri-miRNA). Pri-miRNAs have hairpin structures that are processed by the Drosha enzyme (as part of the microprocessor complex). The microprocessor complex functions by recognizing and cleaving near the junction between hairpin structure and ssRNA. After Drosha processing, the pri-miRNAs are only 60-100 nucleotides long, and are called precursor miRNAs (pre-miRNAs).

At this point, the pre-miRNA is exported to the cytoplasm, where it typically encounters the RNAase enzyme Dicer. Dicer interacts with 5′ and 3′ ends of the hairpin and cuts away the loop joining the 3′ and 5′ arms, resulting in an miRNA:miRNA duplex about 22 nucleotides in length.

After processing, the duplexed miRNA strands are loaded onto an Argonaute (AGO) protein to form a precursor to an RNA-induced silencing complex (RISC). The complex causes the duplex to unwind and the passenger RNA strand is discarded, leaving behind a mature RISC carrying the mature, single stranded miRNA. The miRNA remains part of the RISC as it silences the expression of its target genes. Overall hairpin length and loop size influence the efficiency of Dicer processing. Although either strand of the duplex may potentially act as a functional miRNA, only one strand is generally incorporated into the RNA-induced silencing complex (RISC) where the miRNA and its mRNA target interact.

A variety of other pathways have been discovered, including Drosha-independent pathways (such as the mirtron pathway, snoRNA-derived pathway, and shRNA-derived pathway) and Dicer-independent pathways (such as one that relies on AGO for cleavage, and another which is dependent on tRNaseZ).

Advantageously, artificial miRNA molecules may be engineered to comprise the scaffold of endogenous miRNA and a targeting sequence to a gene of interest. Aspects of the invention comprise miRNA molecules that target gene transcripts using the scaffold of miR-138 or miR-139.

miR-138 is a family of microRNA precursors found in animals, including humans. miR-138 is transcribed as a ~70 nucleotide precursor and subsequently processed by the Dicer enzyme to give a ~22 nucleotide product. The excised region or, mature product, of the miR-138 precursor is the microRNA mir-138.

Endogenously, although miR-138 precursor is expressed ubiquitously, the mature product is found only in certain cell types. In adult mice, endogenously miR-138 is only is expressed in brain tissue. Its expression is not uniform throughout the brain but restricted to distinct neuronal populations. In the zebrafish, miR-138 is expressed in specific domains in the heart and is required to establish appropriate chamber-specific gene expression patterns.

miRNA-based therapies, including miRNA inhibition and miRNA replacement, may be used to treat many diseases such as hepatitis C viral infection, muscular dystrophies, neurodegenerative diseases, peripheral neuropathies, chronic heart failure and post-myocardial infarction remodeling, and cancers. In addition, miRNA directed regulation of gene expression may improve traditional gene therapy approaches in which the vector payload is a protein coding gene.

microRNA sequences are described in U.S. Patent Publication Nos. 2020-0248179, 2019-0300903, 2019-0136235, 2019-0024083, 2017-0029849, and 2014-0322169, the contents of each of which are incorporated by reference herein.

### Myopathies and Muscular Dystrophies

A muscle cell, also known as a myocyte, is a mature contractile cell in the muscle of an animal. In humans and other vertebrates there are three types: skeletal, smooth, and cardiac.

Skeletal muscle cells are large and multinucleated cells containing highly organized contractile proteins that interact with each other to generate force and allow movement of the body. Skeletal muscle contains multiple bundles of muscle fibers, with each muscle fibers composed of myofibrils. The myofibrils are composed of actin and myosin filaments called myofilaments, repeated in units called sarcomeres, which are the basic functional, contractile units of the muscle fiber necessary for muscle contraction. Each muscle cell is innervated from a single synapse of the motor neuron, with the contact point termed the neuromuscular junction, which is the site where muscle contraction is initiated.

Cardiac muscle cells form the cardiac muscle in the walls of the heart chambers and have a single central nucleus. Cardiac muscle cells are joined to neighboring cells by intercalated discs, and when joined in a visible unit they are described as a cardiac muscle fiber.

Skeletal muscle cells and cardiac muscle cells both contain myofibrils and sarcomeres and form a striated muscle tissue. Neuromuscular junction signals are transduced to the contractile apparatus via a process called excitation contraction coupling (ECC), which is mediated through a specialized structure called the triad.

Muscle diseases are collectively referred to as myopathies and/or muscular dystrophies (MD), depending on the underlying genetic cause and the morphological appearance of the abnormal muscle on biopsy. Defects can arise at essentially all points of the contractile process, from the neuromuscular junction, to the triad, to the contractile apparatus itself, and to the specialized matrix-membrane contacts that maintain and preserve membrane integrity.

Muscular dystrophies are characterized by progressive weakness and degeneration of the skeletal muscles that control movement or breathing. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

### Facioscapulohumeral Muscular Dystrophy

Facioscapulohumeral muscular dystrophy (FSHD) is a complex autosomal dominant disorder characterized by progressive and asymmetric weakness of facial, shoulder and limb muscles. Symptoms typically arise in adulthood with most patients showing clinical features before age thirty. About five percent of patients develop symptoms as infants orjuveniles and these are generally more severely affected. Clinical presentation can vary from mild (e.g., some limited muscle weakness) to severe (e.g., wheelchair dependence). Historically, FSHD was classified as the third most common MD, affecting one in 20,000 individuals worldwide. However, recent data indicate FSHD is the most common MD in Europe, suggesting its worldwide incidence could be as high as 1 in 8,333 individuals.

There are two main types of FSHD, which are FSHD type 1 (FSHD1) and FSHD type 2 (FSHD2). Typical FSHD1 cases (also known as FSHD1A) are linked to heterozygous chromosomal deletions that decrease the copy number of 3.3 kilobase (kb) D4Z4 repeats on human chromosome 4q35. Simplistically, normal individuals have 11-100 tandemly repeated D4Z4 copies on both 4q35 alleles, while patients with FSHD have one normal and one contracted allele containing 1-10 repeats. In addition, FSHD-associated D4Z4 contractions must occur on specific disease-permissive chromosome 4q35 backgrounds (called 4qA). Importantly, no genes are completely lost or structurally mutated as a result of FSHD-associated deletions. Instead, genetic changes associated with FSHD give rise to expression of the toxic DUX4 gene, which is damaging to muscle. FSHD2 (also known as FSHD1B) is phenotypically identical to FSHD1, is associated with DUX4 expression, and requires the 4qA chromosomal background. FSHD2 is not associated with D4Z4 repeat contraction, but is is instead caused by mutation in the SMCHD1 gene, which is a chromatin regulator normally involved in repressing the DUX4 locus at 4qA. Mutated SMCHD1 proteins fail to participate in adding heterochromatin to the 4qA DUX4 allele, thereby allowing DUX4 gene expression.

In the leading FSHD pathogenesis model, D4Z4 contractions are proposed to cause epigenetic changes that permit expression of the DUX4 gene. As a result, the aberrant over-expression of otherwise silent or near-silent DUX4 gene, and the genes it regulates, may ultimately cause FSHD. This model is consistent with data showing normal 4q35 D4Z4 repeats have heterochromatin characteristics, while FSHD-linked D4Z4 repeats contain marks more indicative of actively transcribed euchromatin. These transcription-permissive epigenetic changes, coupled with the observation that complete monosomic D4Z4 deletions (i.e., zero repeats) do not cause FSHD, support the hypothesis that D4Z4 repeats harbor potentially myopathic open reading frames (ORFs), which are abnormally expressed in FSHD muscles. This notion was initially considered in 1994, when a D4Z4-localized ORF, called DUX4, was first identified. However, the locus had some characteristics of an unexpressed pseudogene and DUX4 was therefore summarily dismissed as an FSHD candidate. For many years thereafter, the search for FSHD-related genes was mainly focused outside the D4Z4 repeats, and although some intriguing candidates emerged from these studies, no single gene had been conclusively linked to FSHD development. This slow progress led to the re-emergence of DUX4 as an FSHD candidate in 2007.

The role of DUX4 in FSHD pathogenesis can be explained as follows. First, D4Z4 repeats contain identical DUX4 coding regions, and D4Z4 repeats also harbor smaller sense and antisense transcripts, including some resembling microRNAs. Over-expressed DUX4 transcripts and an about 50 kDa full-length DUX4 protein are found in biopsies and cell lines from FSHD patients. These data are consistent with a transcriptional de-repression model of FSHD pathogenesis. In addition, unlike pseudogenes, D4Z4 repeats and DUX4 likely have functional importance, since tandemly arrayed D4Z4 repeats are conserved in at least eleven different placental mammalian species (non-placental animals lack D4Z4 repeats), with the greatest sequence conservation occurring within the DUX4 ORF. Second, over-expressed DUX4 is toxic to tissue culture cells and embryonic progenitors of developing lower organisms in vivo. This toxicity occurs at least partly through a pro-apoptotic mechanism, indicated by caspase-3 activation in DUX4 transfected cells, and presence of TUNEL-positive nuclei in developmentally arrested Xenopus embryos injected with DUX4 mRNA at the two-cell stage. These findings are consistent with studies showing some pro-apoptotic proteins, including caspase-3, are present in FSHD patient muscles. In addition to stimulating apoptosis, DUX4 may negatively regulate myogenesis. Human DUX4 inhibits differentiation of mouse C2C12 myoblasts in vitro, potentially by interfering with PAX3 and/or PAX7, and causes developmental arrest and reduced staining of some muscle markers when delivered to progenitor cells of zebrafish or Xenopus embryos. Finally, aberrant DUX4 function is directly associated with potentially important molecular changes seen in FSHD patient muscles. Specifically, full-length human DUX4 encodes an approximately 50 kDa double homeodomain transcription factor, and DUX4 targets can be found at elevated levels in FSHD patient muscles. These data support that DUX4 catalyzes numerous downstream molecular changes that are incompatible with maintaining normal muscle integrity.

### I. Recombinant Nucleic Acids

Aspects of the present disclosure provide a recombinant nucleic acid comprising a pri-miRNA, which comprises (i) a pri-miRNA scaffold comprising an upstream scaffold, a loop scaffold, and a downstream scaffold, and (ii) a guide strand targeting DUX4, and (iii) a passenger strand, each of which are described herein.

The recombinant nucleic acids described herein can further comprise one or more of the following: at least one DRG de-targeting miRNA binding site, at least one heart de-targeting miRNA binding site, a muscle specific promoter, and a polyadenylation (PolyA) signal, each of which are described herein. Alternatively, or in addition to, the recombinant nucleic acid can further comprise a 5′ inverted terminal repeat (ITR) and a 3′ ITR, each of which are described herein.

(a) Primary MicroRNA (pri-miRNA)

Described herein are pri-miRNAs that include a scaffold sequence (also referred to as a pri-miRNA scaffold or a pri-miRNA scaffold sequence), a guide strand targeting DUX4, and a passenger stand that is at least partially complementary to the guide strand. The guide and the passenger strands are comprised within a pri-miRNA scaffold, which can include an upstream scaffold sequence, a loop scaffold sequence, and a downstream scaffold sequence.

The pri-miRNA scaffold can be derived from any pri-miRNA known in the art or described herein, e.g., any endogenous pri-miRNA or combination of endogenous pri-miRNAs, e.g., a pri-miRNA scaffold derived from pri-miRNA-138, pri-miRNA-139, or a combination thereof. A pri-miRNA scaffold derived from a pri-miRNA refers to a pri-miRNA (e.g., pri-miRNA-138, pri-miRNA-139) in which the stem sequence is replaced with a guide sequence that is at least partially complementary to a target gene (e.g., a sequence targeting DUX4) and a passenger sequence that is at least partially complementary to the guide sequence.

In some embodiments, the pri-miRNA scaffold comprises a scaffold sequence derived from pri-miRNA-138, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In some embodiments, the pri-miRNA scaffold comprises an upstream scaffold sequence comprising or consisting of the nucleic acid sequence of SEQ ID NO: 3929; a loop scaffold sequence comprising or consisting of the nucleic acid sequence of SEQ ID NO: 3930; and a downstream scaffold sequence comprising or consisting of the nucleic acid sequence of SEQ ID NO: 3931.

In some embodiments, the pri-miRNA scaffold comprises a scaffold sequence derived from pri-miRNA-139, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In some embodiments, the pri-miRNA scaffold comprises an upstream scaffold sequence comprising or consisting of the nucleic acid sequence of SEQ ID NO: 3933; a loop scaffold sequence comprising or consisting of the nucleic acid sequence of SEQ ID NO: 3934; and a downstream scaffold sequence comprising or consisting of the nucleic acid sequence of SEQ ID NO: 3935.

In some embodiments, the pri-miRNA scaffold comprises a scaffold sequence derived from pri-miR-138 and pri-miR-139.

For example, the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the pri-miRNA scaffold comprises an upstream scaffold sequence comprising or consisting of the nucleic acid sequence of SEQ ID NO: 3929; a loop scaffold sequence comprising or consisting of the nucleic acid sequence of SEQ ID NO: 3934; and a downstream scaffold sequence comprising or consisting of the nucleic acid sequence of SEQ ID NO: 3935.

In another example, the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the pri-miRNA scaffold comprises an upstream scaffold sequence comprising or consisting of the nucleic acid sequence of SEQ ID NO: 3929; a loop scaffold sequence comprising or consisting of the nucleic acid sequence of SEQ ID NO: 3930; and a downstream scaffold sequence comprising or consisting of the nucleic acid sequence of SEQ ID NO: 3935.

In another example, the pri-miRNA scaffold comprises a scaffold sequence derived from pri-miRNA-138 and pri-miRNA-139, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In another example, the pri-miRNA scaffold comprises an upstream scaffold sequence comprising or consisting of the nucleic acid sequence of SEQ ID NO: 3933; a loop scaffold sequence comprising or consisting of the nucleic acid sequence of SEQ ID NO: 3930; and a downstream scaffold sequence comprising or consisting of the nucleic acid sequence of SEQ ID NO: 3931.

In another example, the pri-miRNA scaffold comprises a scaffold sequence derived from pri-miRNA-138 and pri-miRNA-139, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In another example, the pri-miRNA scaffold comprises an upstream scaffold sequence comprising or consisting of the nucleic acid sequence of SEQ ID NO: 3933; a loop scaffold sequence comprising or consisting of the nucleic acid sequence of SEQ ID NO: 3934; and a downstream scaffold sequence comprising or consisting of the nucleic acid sequence of SEQ ID NO: 3931.

In another example, the pri-miRNA scaffold comprises a scaffold sequence derived from pri-miRNA-138 and pri-miRNA-139, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In another example, the pri-miRNA scaffold comprises an upstream scaffold sequence comprising or consisting of the nucleic acid sequence of SEQ ID NO: 3929; a loop scaffold sequence comprising or consisting of the nucleic acid sequence of SEQ ID NO: 3934; and a downstream scaffold sequence comprising or consisting of the nucleic acid sequence of SEQ ID NO: 3931.

In another example, the pri-miRNA scaffold comprises a scaffold sequence derived from pri-miRNA-138 and pri-miRNA-139, e.g., the pri-miRNA scaffold comprises an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933; a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930; and a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the pri-miRNA scaffold comprises an upstream scaffold sequence comprising or consisting of the nucleic acid sequence of SEQ ID NO: 3933; a loop scaffold sequence comprising or consisting of the nucleic acid sequence of SEQ ID NO: 3930; and a downstream scaffold sequence comprising or consisting of the nucleic acid sequence of SEQ ID NO: 3935.

The guide strand or the passenger strand can be positioned between the upstream scaffold sequence and the loop scaffold sequence in the pri-miRNA described herein. For example, the guide strand is positioned between the upstream scaffold sequence and the loop scaffold sequence, and the passenger strand is positioned between the loop scaffold sequence and the downstream scaffold sequence. Alternatively, the passenger strand is positioned between the upstream scaffold sequence and the loop scaffold sequence, and the guide strand is positioned between the loop scaffold sequence and the downstream scaffold sequence.

Any guide strand targeting DUX4 known in the art or described herein can be included in the pri-miRNA described herein. In some embodiments, the guide strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953. In some embodiments, the guide strand comprises or consists of the nucleic acid sequence of any one of SEQ ID NOs: 2399-3159 and 3952-3953. In some embodiments, the guide strand comprises or consists of the nucleic acid sequence of any one of SEQ ID NO: 2429, SEQ ID NO: 2475, SEQ ID NO: 2869, SEQ ID NO: 3159, or SEQ ID NO: 3047.

Any passenger strand that is at least partially complementary to the guide strand can be included in the pri-miRNA described herein. In some embodiments, the passenger strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920. In some embodiments, the guide strand comprises or consists of the nucleic acid sequence of any one of SEQ ID NOs: 3160-3920. In some embodiments, the guide strand comprises or consists of the nucleic acid sequence of any one of SEQ ID NO: 3190, SEQ ID NO: 3236, SEQ ID NO: 3808, SEQ ID NO: 3630, or SEQ ID NO: 3920.

In some embodiments, the pri-miRNA comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3948. In some embodiments, the pri-miRNA comprises or consists of the nucleic acid sequence of SEQ ID NO: 3948.

In some embodiments, the pri-miRNA comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3949. In some embodiments, the pri-miRNA comprises or consists of the nucleic acid sequence of SEQ ID NO: 3949.

### (b) DRG De-targeting Sites and Heart De-targeting Sites

Recombinant nucleic acids described herein can comprise at least one DRG de-targeting miRNA binding site, e.g., at least one, at least two, at least three, at least four, at least five, or more DRG de-targeting miRNA binding sites.

Any DRG de-targeting miRNA binding site suitable for binding a DRG de-targeting miRNA can be included in the recombinant nucleic acids described herein. In some embodiments, the DRG de-targeting miRNA binding site can comprise or consist of a binding site for miR-338 (e.g., miR-338-3p), miR-138 (e.g., miR-138-5p), or miR-9 (e.g., miR-9-5p).

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923. In some embodiments, the DRG de-targeting miRNA binding site comprises or consists of the nucleic acid sequence of any one of SEQ ID NOs: 3921-3923.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921. In some embodiments, the DRG de-targeting miRNA binding site comprises or consists of the nucleic acid sequence of SEQ ID NOs: 3921.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3922. In some embodiments, the DRG de-targeting miRNA binding site comprises or consists of the nucleic acid sequence of SEQ ID NOs: 3922.

In some embodiments, the DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923. In some embodiments, the DRG de-targeting miRNA binding site comprises or consists of the nucleic acid sequence of SEQ ID NOs: 3923.

In some embodiments, when the recombinant nucleic acid comprises more than one DRG de-targeting miRNA binding site, the more than one DRG de-targeting miRNA binding site can have the same sequence or a different sequence.

For example, when the recombinant nucleic acid comprises a first and a second DRG de-targeting miRNA binding site, each of the DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of any one of SEQ ID NOs: 3921-3923.

Alternatively, the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 3921 and SEQ ID NO: 3922, respectively, or vice versa; the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 3922 and SEQ ID NO: 3923, respectively, or vice versa; or the first and the second DRG de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 3921 and SEQ ID NO: 3923, respectively, or vice versa.

The at least one DRG de-targeting miRNA binding site can be positioned in the recombinant nucleic acids described herein at any location suitable for binding a DRG de-targeting miRNA (e.g., miR-338-3p, miR-138-5p, miR-9-5p).

For example, the DRG de-targeting miRNA binding site can be positioned upstream or downstream of the sequence encoding the engineered pri-miRNA. In another example, when the recombinant nucleic acid comprises more than one DRG de-targeting miRNA binding site, each of the DRG de-targeting miRNA binding sites can be positioned upstream or downstream of the sequence encoding the engineered pri-miRNA. Alternatively, one or more of the DRG de-targeting miRNA binding sites can be positioned upstream of the sequence encoding the engineered pri-miRNA and one or more of the DRG de-targeting miRNA binding sites can be positioned downstream of the sequence encoding the engineered pri-miRNA.

Recombinant nucleic acids described herein can comprise at least one heart de-targeting miRNA binding site, e.g., at least one, at least two, at least three, at least four, at least five, or more heart de-targeting miRNA binding sites.

Any heart de-targeting miRNA binding site suitable for binding a heart de-targeting miRNA can be included in the recombinant nucleic acids described herein. In some embodiments, the heart de-targeting miRNA binding site can comprise or consist of a binding site for miR-221 (e.g., miR-221-3p) or miR-499 (e.g., miR-499a).

In some embodiments, the heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925. In some embodiments, the heart de-targeting miRNA binding site comprises or consists of the nucleic acid sequence of SEQ ID NO: 3924 or SEQ ID NO: 3925.

In some embodiments, the heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924. In some embodiments, the heart de-targeting miRNA binding site comprises or consists of the nucleic acid sequence of SEQ ID NOs: 3924.

In some embodiments, the heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925. In some embodiments, the heart de-targeting miRNA binding site comprises or consists of the nucleic acid sequence of SEQ ID NOs: 3925.

In some embodiments, when the recombinant nucleic acid comprises more than one heart de-targeting miRNA binding site, the more than one heart de-targeting miRNA binding site can have the same sequence or a different sequence.

For example, when the recombinant nucleic acid comprises a first and a second heart de-targeting miRNA binding site, each of the heart de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 3924 or SEQ ID NO: 3925.

Alternatively, the first and the second heart de-targeting miRNA binding sites can comprise or consist of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NO: 3924 and SEQ ID NO: 3925, respectively, or vice versa.

The at least one heart de-targeting miRNA binding site can be positioned in the recombinant nucleic acids described herein at any location suitable for binding a heart de-targeting miRNA (e.g., miR-221-3p, miR-449a).

For example, the at least one heart de-targeting miRNA binding site can be positioned upstream or downstream of the sequence encoding the pri-miRNA. In another example, when the recombinant nucleic acid comprises more than one heart de-targeting miRNA binding site, each of the heart de-targeting miRNA binding sites can be positioned upstream or downstream of the sequence encoding the pri-miRNA. Alternatively, one or more of the heart de-targeting miRNA binding sites can be positioned upstream of the sequence encoding the pri-miRNA and one or more of the heart de-targeting miRNA binding sites can be positioned downstream of the sequence encoding the pri-miRNA.

Recombinant nucleic acids described herein can comprise at least one DRG de-targeting miRNA binding site and at least one heart de-targeting miRNA binding site. The at least one DRG de-targeting miRNA binding site and the at least one heart de-targeting miRNA binding site can be positioned in the recombinant nucleic acids described herein at any location suitable for binding a DRG de-targeting miRNA (e.g., miR-338-3p, miR-138-5p, miR-9-5p) or a heart de-targeting miRNA (e.g., miR-221-3p, miR-449a), respectively.

For example, the at least one DRG de-targeting miRNA binding site and at least one heart de-targeting miRNA binding site can be positioned upstream or downstream of the sequence encoding the pri-miRNA.

In another example, when the recombinant nucleic acid comprises more than one DRG de-targeting miRNA binding site and more than one heart de-targeting miRNA binding site, each of the DRG de-targeting miRNA binding site and each of the heart de-targeting miRNA binding sites can be positioned upstream or downstream of the sequence encoding the pri-miRNA. Alternatively, one or more of the DRG de-targeting miRNA binding sites and one or more of the heart de-targeting miRNA binding sites can be positioned upstream of the sequence encoding the pri-miRNA and one or more of the heart de-targeting miRNA binding sites can be positioned downstream of the sequence encoding the pri-miRNA, or vice versa.

The more than one DRG de-targeting miRNA binding site and more than one heart de-targeting miRNA binding site can be arranged in consecutive or alternating positions.

As used herein, “consecutive positions” refers to an arrangement of DRG de-targeting miRNA binding sites and heart de-targeting miRNA binding sites in which the at least one DRG de-targeting miRNA binding site is followed by at least heart de-targeting miRNA binding site (e.g., a first and a second DRG de-targeting miRNA binding site followed by a first and a second heart de-targeting miRNA binding), or vice versa (e.g., a first and a second heart de-targeting miRNA binding site followed by a first and a second DRG de-targeting miRNA binding).

For example, when the recombinant nucleic acid comprises a first and a second DRG de-targeting miRNA binding site and a first and a second heart de-targeting miRNA binding site arranged in consecutive positions, the first and the second DRG de-targeting miRNA binding site can be followed by the first and the second heart de-targeting miRNA binding, or vice versa.

As used herein, “alternating positions” refers to an arrangement of DRG de-targeting miRNA binding sites and heart de-targeting miRNA binding sites such that there is at least one DRG de-targeting between at least one heart de-targeting site (e.g., heart de-targeting miRNA binding site, DRG de-targeting miRNA binding site, heart de-targeting miRNA binding site) or vice versa (e.g., DRG de-targeting miRNA binding site, heart de-targeting miRNA binding site, DRG de-targeting miRNA binding site).

For example, when the recombinant nucleic acid comprises a first and a second DRG de-targeting miRNA binding site and a first and a second heart de-targeting miRNA binding site arranged in alternating positions, the first DRG de-targeting miRNA binding site can be followed by the first heart de-targeting miRNA binding and the second DRG de-targeting miRNA binding site can be followed by the second heart de-targeting miRNA binding, or vice versa.

### (c) Promoters

Various promoters, including inducible promoters and constitutive promoters, can be used to drive expression from the recombinant nucleic acids described herein. Non-limiting examples of promoters that can be used in the recombinant nucleic acids disclosed herein include a cytomegalovirus (CMV) promoter, a chicken R-actin (CBA) promoter, an alpha-1 antitrypsin (hAAT) promoter, and any promoter derived from an immunoglobulin gene, SV40, or other tissue specific genes.

In some embodiments, the promoter is tissue-specific such that, in a multi-cellular organism, the promoter drives expression only in a subset of specific cells. For example, the promoter is a muscle specific promoter. Non-limiting examples of muscle specific promoters include a CK8 promoter, a desmin promoter, a Mb promoter, a MCK promoter, a MHCK7 promoter, a skeletal muscle alpha actin promoter, or a TTNI2 promoter.

In some embodiments, the promoter comprises a promoter sequence described herein. For example, the promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928. In another example, the promoter comprises or consists of the nucleic acid sequence of any one of SEQ ID NOs: 3926-3928. In some embodiments, the recombinant nucleic acid comprises or consists of the nucleic acid sequence of SEQ ID NO: 3926. In some embodiments, the recombinant nucleic acid comprises or consists of the nucleic acid sequence of SEQ ID NO: 3927. In some embodiments, the recombinant nucleic acid comprises or consists of the nucleic acid sequence of SEQ ID NO: 3928.

### (d) PolyA Signals

Recombinant nucleic acids described herein can further comprise a polyadenylation (PolyA) signal sequence or functional fragment thereof, e.g., a fragment capable of measurably increasing expression as compared to expression in its absence. Non-limiting examples of PolyA signal sequences include a bovine growth hormone polyadenylation (bgh-PolyA) signal, a synthetic PolyA signal, and an SV40 PolyA signal.

In some embodiments, the PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938. In some embodiments, the PolyA signal comprises or consists of the nucleic acid sequence of SEQ ID NO: 3938.

Recombinant nucleic acids described herein can comprise one or more PolyA signal sequences or functional fragments thereof. In some embodiments, the recombinant nucleic acid described herein comprises a polyA sequence between the 3′ end of a sequence encoding an engineered pri-miRNA and the 5′ end of the 3′ ITR.

### (e) ITRs

Recombinant nucleic acids described herein can comprise one or more ITRs, e.g., two ITRs, with one upstream and the other downstream of a sequence encoding a pri-miRNA and/or the other elements discussed above (e.g., a promoter, at least one DRG de-targeting miRNA binding site, at least one heart de-targeting miRNA binding site, a PolyA signal).

An ITR sequence may be wild type, or it may comprise one or more mutations, e.g., by the insertion, deletion, or substitution of nucleotides, as long as the sequences retain one or more function of a wild type ITR, such as providing for functional rescue, replication and packaging. In some embodiments, a recombinant nucleic acid as described herein can comprise two ITR sequences, each of which is wild type, variant, or modified ITR sequences. In some embodiments, a recombinant nucleic acid as described herein can comprise two ITR sequences, which are different (e.g., one wild type ITR sequence and one variant or modified ITR sequence). For example, the “left” or 5′ ITR is a modified ITR sequence that allows for production of self-complementary genomes, and the “right” or 3′ ITR is a wild type ITR sequence. In some embodiments, the “right” or 3′ ITR is a modified ITR sequence that allows for the production of self-complementary genomes, and the “left” or 5′ ITR is a wild type ITR sequence.

In some embodiments, a recombinant nucleic acid can comprise ITR sequences from any AAV serotype suitable for replication and packaging of the virus. Non-limiting examples AAV serotypes include AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, AAV7m8, or any derivative, hybrid or chimeric serotype thereof.

In some embodiments, the recombinant nucleic acid comprises one or more ITRs derived from AAV2. The nucleotides in an ITR sequence may be in a forward or reverse orientation. In some embodiments, the ITR sequences are both wild type, variant, or modified AAV2 ITR sequences. In some embodiments, the ITR sequences are different (e.g., one ITR is a wild type AAV2 sequence and one ITR is variant or modified AAV2 ITR sequence). In some embodiments, the “left” or 5′ ITR is a modified AAV2 ITR sequence that allows for production of self-complementary genomes, and the “right” or 3′ ITR is a wild type AAV2 ITR sequence. In some embodiments, the “right” or 3′ ITR is a modified AAV2 ITR sequence that allows for the production of self-complementary genomes, and the “left” or 5′ ITR is a wild type AAV2 ITR sequence. In some embodiments, the 5′ ITR is an AAV2 5′ ITR as described in McCarty, D. M., et al., Gene Ther (2001), which is incorporated by reference in its entirety. In some embodiments, the 3′ ITR is an AAV2 3′ ITR as described in Rolling, F. & Samulski, R. J. Molecular biotechnology (1995), which is incorporated by reference in its entirety. In some embodiments, the 3′ ITR may comprise a deletion of the terminal resolution site (dTR), which inhibits Rep protein nicking of the single stranded viral genome. The presence of the dTR in the 3′ ITR increases self-complementary binding of the viral genome to itself, which it may do because of its small size that allows for a double-stranded viral genome to be packaged within a viral capsid.

In some embodiments, the recombinant nucleic acid comprises a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937. In some embodiments, the recombinant nucleic acid comprises a 5′ ITR comprising or consisting of the nucleic acid sequence of SEQ ID NO: 3937.

In some embodiments, the recombinant nucleic acid comprises a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939. In some embodiments, the recombinant nucleic acid comprises a 3′ ITR comprising or consisting of the nucleic acid sequence of SEQ ID NO: 3939.

### (f) Example Recombinant Nucleic Acids

Non-limiting examples of recombinant nucleic acids for use in methods described herein are provided below.

For example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In another example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931.

In another example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190; and
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3933;
a guide strand targeting DUX4 comprising or consisting of the nucleic acid sequence of SEQ ID NO: 2475;
a loop scaffold comprising or consisting of the nucleic acid sequence of SEQ ID NO: 3934;
a passenger strand comprising or consisting of the nucleic acid sequence of SEQ ID NO: 3236; and
a downstream scaffold comprising or consisting of the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3933;
a guide strand targeting DUX4 comprising or consisting of the nucleic acid sequence of SEQ ID NO: 3159;
a loop scaffold comprising or consisting of the nucleic acid sequence of SEQ ID NO: 3934;
a passenger strand comprising or consisting of the nucleic acid sequence of SEQ ID NO: 3920; and
a downstream scaffold comprising or consisting of the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3929;
a guide strand targeting DUX4 comprising or consisting of the nucleic acid sequence of SEQ ID NO: 3047;
a loop scaffold comprising or consisting of the nucleic acid sequence of SEQ ID NO: 3930;
a passenger strand comprising or consisting of the nucleic acid sequence of SEQ ID NO: 3808; and
a downstream scaffold comprising or consisting of the nucleic acid sequence of SEQ ID NO: 3931.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3929;
a guide strand targeting DUX4 comprising or consisting of the nucleic acid sequence of SEQ ID NO: 2475;
a loop scaffold comprising or consisting of the nucleic acid sequence of SEQ ID NO: 3930;
a passenger strand comprising or consisting of the nucleic acid sequence of SEQ ID NO: 3236; and
a downstream scaffold comprising or consisting of the nucleic acid sequence of SEQ ID NO: 3931.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3933;
a guide strand targeting DUX4 comprising or consisting of the nucleic acid sequence of SEQ ID NO: 2869;
a loop scaffold comprising or consisting of the nucleic acid sequence of SEQ ID NO: 3934;
a passenger strand comprising or consisting of the nucleic acid sequence of SEQ ID NO: 3630; and
a downstream scaffold comprising or consisting of the nucleic acid sequence of SEQ ID NO: 3935.

In another example, the recombinant nucleic acid comprises a sequence encoding a pri-miRNA comprising or consisting of, optionally from 5′ to 3′:
an upstream scaffold comprising or consisting of the nucleic acid sequence of SEQ ID NO: 3933;
a guide strand targeting DUX4 comprising or consisting of the nucleic acid sequence of SEQ ID NO: 2429;
a loop scaffold comprising or consisting of the nucleic acid sequence of SEQ ID NO: 3934;
a passenger strand comprising or consisting of the nucleic acid sequence of SEQ ID NO: 3190; and
a downstream scaffold comprising or consisting of the nucleic acid sequence of SEQ ID NO: 3935.

In another example, the recombinant nucleic acid comprises, optionally from 5′ to 3′: a 5′ ITR;
a muscle specific promoter;
an upstream scaffold;
a guide strand targeting DUX4;
a loop scaffold;
a passenger strand;
a downstream scaffold;
a first DRG de-targeting miRNA binding site;
a first heart de-targeting miRNA binding site;
a second DRG de-targeting miRNA binding site;
a second heart de-targeting miRNA binding site;
a PolyA signal; and
a 3′ ITR.

In another example, the recombinant nucleic acid comprises, optionally from 5′ to 3′: a 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928;
an upstream scaffold comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933;
a guide strand targeting DUX4 comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953;
a loop scaffold comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and
a passenger strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920;
a downstream scaffold comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935;
a first DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
a first heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
a second DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
a second heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
a PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3928;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises, optionally from 5′ to 3′: a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3940-3947.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3940.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3941.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3942.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3943.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3944.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3945.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3946.

In another example, the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3947.

In another example, the recombinant nucleic acid comprises or consists of the nucleic acid sequence of any one of SEQ ID NOs: 3940-3947.

In another example, the recombinant nucleic acid comprises or consists of the nucleic acid sequence of SEQ ID NO: 3940.

In another example, the recombinant nucleic acid comprises or consists of the nucleic acid sequence of SEQ ID NO: 3941.

In another example, the recombinant nucleic acid comprises or consists of the nucleic acid sequence of SEQ ID NO: 3942.

In another example, the recombinant nucleic acid comprises or consists of the nucleic acid sequence of SEQ ID NO: 3943.

In another example, the recombinant nucleic acid comprises or consists of the nucleic acid sequence of SEQ ID NO: 3944.

In another example, the recombinant nucleic acid comprises or consists of the nucleic acid sequence of SEQ ID NO: 3945.

In another example, the recombinant nucleic acid comprises or consists of the nucleic acid sequence of SEQ ID NO: 3946.

In another example, the recombinant nucleic acid comprises or consists of the nucleic acid sequence of SEQ ID NO: 3947.

### II. Recombinant Expression Vectors

Any of the recombinant nucleic acids comprising any sequence encoding a pri-miRNA described herein can be delivered to a cell (e.g., a muscle cell), a tissue (e.g., muscle tissue), or subject (e.g., human subject) without or with the use of a delivery system (e.g., recombinant expression vector (e.g., viral vector (e.g., AAV (e.g., rAAV))).

### (a) Recombinant Expression Vectors

Any of the recombinant nucleic acids described herein can be included in a recombinant expression vector (e.g., a viral vector (e.g., an AAV vector (e.g., a rAAV vector))). Recombinant expression vectors can be delivered into a cell, a tissue, or a subject using any method known in the art or described herein. A recombinant expression vector can be an organic or inorganic molecule. A recombinant expression vector can be a small molecule (i.e., <5 kD) or a macromolecule (i.e., >5 kD). The recombinant expression vector can comprise DNA, RNA, or both DNA and RNA. The recombinant expression vector can be a DNA vector, a circular vector, or a plasmid. The recombinant expression vector can be double stranded or single stranded.

In some embodiments, a recombinant expression vector (e.g., a viral vector (e.g., an AAV (e.g., a rAAV))) disclosed herein can exhibit higher expression of the recombinant nucleic acid (e.g., expression of pri-miRNA) in a specific tissue type (e.g., muscle) as compared to the expression of the same vector in a different tissue type (e.g., liver). For example, the recombinant expression vector can exhibit higher expression of the engineered pri-miRNA in a skeletal muscle tissue or cell as compared to the expression of the same vector in a non-skeletal muscle tissue (e.g., heart tissue, liver tissue) or cell (e.g., heart cell, liver cell). In some embodiments, the vector exhibits at least about 1.2 fold, at least about 1.5 fold, at least about 1.75 fold, at least about 2 fold, at least about 2.5 fold, at least about 3 fold, at least about 4 fold higher expression of the recombinant nucleic acid (e.g., expression of an engineered pri-miRNA) in a skeletal muscle tissue or cell as compared to the expression of the same vector in a non-skeletal muscle tissue (e.g., heart tissue, liver tissue) or cell (e.g., heart cell, liver cell).

In some embodiments, the recombinant expression vector is a viral vector. Non-limiting examples of viral vectors for use in compositions and methods described herein include retroviral vectors, adenoviral vectors, lentiviral vectors, adeno-associated viral (AAVs) vectors, herpes simplex viral (HSV) vectors, alphaviral vectors, pox viral vectors, murine leukemia viral vectors, and hybrid viral vectors.

### (b) Adeno Associated Virus Vectors

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV 12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

Previous approaches to identify AAV sequences correlated with tropism have relied upon the comparison of highly related extant serotypes with distinct characteristics, random domain swaps between unrelated serotypes, or consideration of higher-order structure, to identify motifs that define liver tropism. For example, mapping determinants of AAV tropism have been carried out by comparing highly related serotypes. One such example is the single-amino acid change (E531K) between AAV1 and AAV6 that improves murine liver transduction in AAV1. See, e.g., Wu et al. (2006) J. Virol., 80(22):11393-7, incorporated by reference herein. Another example is a reciprocal domain swap between AAV2 and AAV8 that alters tropism, but fails to define any robust specific tissue-targeting motifs. See, e.g., Raupp et al. (201) J. Virol., 86(17):9396-408, incorporated by reference herein. Further, global consideration of structure has only highlighted gross differences between better- or worse-liver-transducers that are more observational than useful in practice. Nam et al (2007) J. Virol., 81(22):12260-71.

AAVs exhibiting modified tissue tropism that may be used with the present invention are described in U.S. Pat. Nos. 9,695,220, 9,719,070; 10,119,125; 10,526,584; U.S. Patent Application Publication No. 2018-0369414; U.S. Patent Application Publication No. 2020-0123504; U.S. Patent Application Publication No. 2020-0318082; PCT International Patent Application Publication No. WO 2015/054653; PCT International Patent Application Publication No. WO 2016/179496; PCT International Patent Application Publication No. WO 2017/100791; and PCT International Patent Application Publication No. WO 2019/217911, the entirety of the contents of each of which are incorporated by reference herein.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV1, AAV2, AAV3, AAV4, AAV5, AAV6, AAV8, AAV9 or any combinations thereof. In some embodiments, the AAV can be AAV1, AAV2, AAV5, AAV9 or any combination thereof. One can select the AAV of the AAV with regard to the cells to be targeted, e.g., one can select AAV serotypes 1, 2, 5, 9 or a hybrid capsid AAV1, AAV2, AAV5, AAV9 or any combination thereof for targeting brain and/or neuronal cells; and one can select AAV4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV1, AAV2, AAV5 or any combination thereof. In some embodiments, an AAV vector or system thereof capable of producing AAV particles capable of targeting cardiac tissue can be configured to generate an AAV particle having an AAV4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see, e.g., Srivastava. 2017. Curr. Opin. Virol. 21:75-80.) By including an engineered AAV capsid and/or capsid protein variant of wild-type AAV-9 as described herein, the tropism for nervous cells might be reduced or eliminated and/or the muscle specificity increased such that the nervous specificity appears reduced in comparison, thus enhancing the specificity for muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV9 serotype that is used to package a genome that contains components (e.g., rep elements) from an AAV2 serotype. As with wild-type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as International Patent Application Publications WO 2021/050974, WO 2021/077000 and WO 2022/020616, the contents of which are incorporated by reference herein.

Additional AAV vectors are described in International Patent Application Publication WO 2019/2071632, the contents of which are incorporated by reference herein.

Further AAV vectors are described in International Patent Application Publications WO 2020/086881 and WO 2020/235543, the contents of each of which are incorporated by reference herein.

Further AAV vectors are described in International Patent Application Publications WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### III. Viral Particles and Capsid Proteins

Any of the recombinant nucleic acids or recombinant expression vectors (e.g., recombinant expression vector (e.g., viral vector (e.g., AAV (e.g., rAAV))) described herein can be packaged or encapsulated in a viral particle (e.g., AAV particle). Viral particles for use in compositions and methods described herein can include any viral capsid protein (e.g., AAV capsid protein or variant thereof) known in the art or described herein.

In some embodiments, a viral particle (e.g., an AAV particle (e.g., a rAAV particle)) disclosed herein can exhibit higher expression of the recombinant nucleic acid (e.g., expression of a pri-miRNA) in a specific tissue type as compared to the expression of the same viral particle in a different tissue type. For example, the viral particle can exhibit higher expression of a pri-miRNA in a skeletal muscle tissue or cell as compared to the expression of the same viral particle in a non-skeletal muscle tissue (e.g., heart tissue, liver tissue) or cell (e.g., heart cell, liver cell). In some embodiments, the viral particle exhibits at least about 1.2 fold, at least about 1.5 fold, at least about 1.75 fold, at least about 2 fold, at least about 2.5 fold, at least about 3 fold, at least about 4 fold higher expression of the recombinant nucleic acid (e.g., expression of a pri-miRNA) in a skeletal muscle tissue or cell as compared to the expression of the same viral vector in a non-skeletal muscle tissue (e.g., heart tissue, liver tissue) or cell (e.g., heart cell, liver cell).

Viral particles described herein can include any virus suitable for delivery of a transgene, e.g., retroviruses, adenovirus, lentivirus, AAV, and murine leukemia viruses. In some embodiments, the viral particle is a recombinant adenovirus comprising a recombinant nucleic acid or a recombinant expression vector described herein. In some embodiments, the viral particle is a recombinant AAV comprising a recombinant nucleic acid or a recombinant AAV vector described herein. The AAV particle can be a scAAV or a ssAVV.

AAV particles described herein can include one or more AAV capsid proteins. In some embodiments, the one or more AAV capsid proteins can be from one or more AAV serotypes. Non-limiting examples of AAV serotypes include AAV1, AAV2, AAV3, AAV4, AAV5, AAV6, AAV7, AAV8, AAV9, AAV10, AAV11, AAV12, AAVrh8, AAVfh10, AAV-DJ, AAV-DJ/8, AAV-PHP.B, AAV-PHP.B2, AAV-PHP.B3, AAV-PHP.A, AAV-PHP.eB, AAV-PHP.S, and functional variants of any AAV serotypes.

In some embodiments, the AAV particle comprises a capsid protein comprising an amino acid sequence of formula (I) X₁NX₂X₃X₄RGDRX₅X₆L (SEQ ID NO: 1), wherein each of X₁-X₆ is any amino acid. In some embodiments, X₁ is A, I, F, G, H, L, M, Q, S, T, or V. In some embodiments, X₂ is A, G, S, T, or Y. In some embodiments, X₃ is S, N, G, or P. In some embodiments, X₄ is A, G, H, I, M, S, T, or V. In some embodiments, X₅ is A, G, or Q. In some embodiments, X₆ is A, I, L, M, N, S, or Y.

The amino acid sequence of formula (I) can be in a hypervariable region IV (HVR IV) relative to a wild type AAV9 capsid. For example, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L is inserted after amino acid 455.

In some embodiments, the AAV particle comprises a capsid protein comprising an amino acid sequence of any one of SEQ ID NOs: 801-1599. In some embodiments, the AAV particle comprises a capsid protein comprising an amino acid sequence of any one of SEQ ID NOs: 1600-2398. In some embodiments, the AAV particle comprises or consists of a capsid protein comprising an amino acid sequence of any one of SEQ ID NOs: 2-800. In some embodiments, the AAV particle comprises a capsid protein comprising an amino acid sequence comprising or consisting of SEQ ID NOs: 191, 198, 199, 215, 256, 379, 544, 550, or 748. In some embodiments, the AAV particle comprises a capsid protein comprising an amino acid sequence comprising or consisting of SEQ ID NO: 191. In some embodiments, the AAV particle comprises a capsid protein comprising an amino acid sequence comprising or consisting of SEQ ID NO: 198. In some embodiments, the AAV particle comprises a capsid protein comprising an amino acid sequence comprising or consisting of SEQ ID NO: 199. In some embodiments, the AAV particle comprises a capsid protein comprising an amino acid sequence comprising or consisting of SEQ ID NO: 215. In some embodiments, the AAV particle comprises a capsid protein comprising an amino acid sequence comprising or consisting of SEQ ID NO: 256. In some embodiments, the AAV particle comprises a capsid protein comprising an amino acid sequence comprising or consisting of SEQ ID NO: 379. In some embodiments, the AAV particle comprises a capsid protein comprising an amino acid sequence comprising or consisting of SEQ ID NO: 544. In some embodiments, the AAV particle comprises a capsid protein comprising an amino acid sequence comprising or consisting of SEQ ID NO: 550. In some embodiments, the AAV particle comprises a capsid protein comprising an amino acid sequence comprising or consisting of SEQ ID NO: 748.

Any of the capsid proteins described herein can further include a deletion. For example, the capsid protein can further comprise a deletion of amino acid G267 relative to a wild type AAV9 vector.

The capsid protein is the shell or coating of the virus that enables its delivery into the host. Without the protein, the nucleic acids would be destroyed by the host without entering into the host cells and beginning transcription and translation. The capsid protein may be in the natural conformation of a naturally occurring AAV, or it may be modified.

In certain example embodiments, the AAV capsid protein is an engineered AAV capsid protein having reduced or eliminated uptake in a non-muscle cell as compared to a corresponding wild-type AAV capsid polypeptide.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter.

Suitable tissue specific promoters and constitutive promoters are discussed elsewhere herein and are generally known in the art and can be commercially available. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter.

Described herein are various embodiments of engineered viral capsids, such as adeno-associated virus (AAV) capsids, that can be engineered to confer cell-specific tropism, such as muscle specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-specific tropism, reduced immunogenicity, or both to the engineered viral particle. The engineered viral capsids described herein can include one or more engineered viral capsid proteins described herein. The engineered viral capsids described herein can include one or more engineered viral capsid proteins described herein that can contain a muscle-specific targeting moiety containing or composed of an n-mer motif described elsewhere herein.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV1, AAV2, AAV3, AAV4, AAV5, AAV6, AAV8, AAV9 or any combination thereof. In some embodiments, the serotype of the wild-type AAV capsid can be AAV9. The engineered AAV capsids can have a different tropism than that of the reference wild-type AAV capsid.

The engineered viral capsid can contain 1-60 engineered capsid proteins. In some embodiments, the engineered viral capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered viral capsid can contain 0-59 wild-type viral capsid proteins. In some embodiments, the engineered viral capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type viral capsid proteins.

In some embodiments, the engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV is capsid can contain 0-59 wild type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins.

In some embodiments, the engineered viral capsid protein can have an n-mer amino acid motif, where n can be at least 3 amino acids. In some embodiments, n can be 3, 4, 5, 6, 7, 8, 9, 10, 11, 12, 13, 14, or 15 amino acids. In some embodiments, an engineered AAV capsid can have a 6-mer or 7-mer amino acid motif. In some embodiments, the n-mer amino acid motif can be inserted between two amino acids in the wild-type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild type AAV viral protein contains an eight-stranded beta-barrel motif (betaB to betaI) and an alpha-helix (alphaA) that are conserved in autonomous parvovirus capsids (see, e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see, e.g., Weitzman and Linden. 2011. “Adeno-Associated Virus Biology.” In Snyder, R. O., Moullier, P. (eds.) Totowa, NJ: Humana Press). In some embodiments, one or more n-mer motifs can be inserted between two amino acids in one or more of the 12 variable regions in the wild-type AVV capsid proteins. In some embodiments, the one or more n-mer motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid protein. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer inserted between amino acids 588 and 589 of an AAV9 viral protein. In some embodiments, the engineered capsid can have a 7-mer motif inserted between amino acids 588 and 589 of an AAV9 viral protein. In other embodiments, the motif inserted is a 10-mer motif, with replacement of amino acids 586-88 and an insertion before 589. It will be appreciated that n-mers can be inserted in analogous positions in AAV viral proteins of other serotypes. In some embodiments as previously discussed, the n-mer(s) can be inserted between any two contiguous amino acids within the AAV viral protein and in some embodiments the insertion is made in a variable region.

In some embodiments, the first 1, 2, 3, or 4 amino acids of an n-mer motif can replace 1, 2, 3, or 4 amino acids of a polypeptide into which it is inserted and preceding the insertion site. In some embodiments, the amino acids of the n-mer motif that replace 1 or more amino acids of the polypeptide into which the n-mer motif is inserted come before or immediately before an “RGDR” (SEQ ID NO: 3950) in an n-mer motif. The first three amino acids can replace 1-3 amino acids into a polypeptide to which they may be inserted. Using an AAV as another non-limiting example, one or more of the n-mer motifs can be inserted into, e.g., an AAV9 capsid prolylpeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the n-mer motif is inserted are replaced by the n-mer motif.

In some embodiments, the AAV capsids or other viral capsids or compositions can be muscle specific. In some embodiments, muscle-specificity of the engineered AAV or other viral capsid or other composition is conferred by a muscle specific n-mer motif incorporated in the engineered AAV or other viral capsid or other composition described herein. While not intending to be bound by theory, it is believed that the n-mer motif confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a muscle cell. In some embodiments, the cell surface receptor is AAV receptor (AAVR). In some embodiments, the cell surface receptor is a muscle cell specific AAV receptor. In some embodiments, the cell surface receptor or other is molecule is a cell surface receptor or other molecule selectively expressed on the surface of a muscle cell. In some embodiments, the cell surface receptor or molecule is an integrin or dimer thereof. In some embodiments, the cell surface receptor or molecule is an Vb6 integrin heterodimer.

In some embodiments, a muscle specific engineered viral particle or other composition described herein containing the muscle-specific capsid, n-mer motif, or muscle-specific targeting moiety described herein can have an increased uptake, delivery rate, transduction rate, efficiency, amount, or a combination thereof in a muscle cell as compared to other cells types and/or other virus particles (including but not limited to AAVs) and other compositions that do not contain the muscle-specific n-mer motif of the present invention.

First- and second-generation muscle specific AAV capsids were developed using a muscle specific promoter and the resulting capsid libraries were screened in mice and non-human primates as described elsewhere herein and/or in, e. g, U.S. Provisional Application Ser. Nos. 62/899,453, 62/916,207, 63/018,454, and 63/242,008. First and second generation myoAAV capsids were further optimized in mice and non-human primates as previously described to generate enhanced myoAAV capsids.

### IV. Pharmaceutical Compositions

Any of the recombinant nucleic acids, the expression vectors (e.g., AAV vectors), or the AAV particles described herein can be included in a pharmaceutical composition comprising a pharmaceutically acceptable carrier.

Some embodiments of the invention may include any acceptable form of providing the AAV vector to a subject. For example, the AAV vector may be provided to the subject in the form of a composition or formulation comprising the AAV vector. The expression vector (e.g., AAV vector) of this invention can be formulated and administered to treat a variety of disease states by any means that produces contact of the active ingredient with the agent's site of action in the body of the subject. The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more muscle-specific targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10, 1×10⁹, 1×10¹⁰ or more cells per nL, μL, mL, or L.

In some embodiments, where engineered AAV capsid particles are included in the formulation, the formulation can contain 1 to 1×10², 1×10³, 1×10⁴, 1×10⁵, 1×10⁶, 1 ×10⁷, 1× 10⁸, 1×10⁹, 1× 10¹⁰, 1× 10¹¹, 1× 10¹², 1× 10¹³, 1× 10¹⁴, 1× 10¹⁵, 1× 10¹⁶, 1× 10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10², 1×10³, 1×10⁴, 1×10⁵, 1×10⁶, 1×10⁷, 1×10⁸, 1×10⁹, 1×10¹⁰, 1 × 10¹¹, 1× 10¹², 1× 10¹³, 1× 10¹⁴, 1× 10¹⁵, 1× 10¹⁶, 1× 10¹⁷, 1× 10¹⁸, 1× 10¹⁹, or 1×10²⁰ is transducing units (TU)/mL of the engineered AAV capsid particles.

### (a) Pharmaceutically Acceptable Carriers and Auxiliary Ingredients and Agents

Any recombinant nucleic acids, expression vectors (e.g., AAV vectors), or AAV particles described herein can be included in a pharmaceutical composition comprising a pharmaceutically acceptable carrier.

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intravenous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle and compositions, formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and is thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain antioxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### V. Methods of Treatment and Use

Aspects of the present disclosure provide methods for treating FSHD (e.g., FSHD type 1 (FSHD1) and/or FSHD type 2 (FSHD2)) in a subject in need thereof using any of the recombinant nucleic acids, the expression vectors (e.g., the AAV vectors), the AAV particles, or pharmaceutical compositions thereof described herein.

### (a) Subjects

A subject can be any subject for whom diagnosis, treatment, or therapy is desired. A subject can be a human or a non-human primate. A subject can be any age. In some embodiments, the subject is a human subject that is any age (e.g., a human subject that is between the ages of 5 and 25).

A subject (e.g., human subject) to be treated by the methods described herein can be a subject having, suspected of having, or at risk for having FSHD (e.g., FSHD1, FSHD2, or FSHD1+2). A subject (e.g., human subject) suspected of having FSHD might show one or more symptoms of FSHD, e.g., facial weakness, shoulder and upper arm weakness, lower leg is weakness, abdominal weakness, hip weakness, scoliosis, muscle atrophy, hearing loss, vision loss, and muscle pain. A subject (e.g., human subject) at risk for FSHD can be a subject having one or more risk factors for FSHD, e.g., one or more deletions of a D4Z4 repeat in chromosome 4, one or more mutations in the structural maintenance of chromosomes flexible hinge domain containing 1 (SMCHD1) gene, one or more mutations in the DNA (cytosine-5)-methyltransferase 3B (DNMT3B) gene, one or more mutations in the LRIF1 gene, or a combination of any of these. A subject (e.g., human subject) who needs the treatment described herein can be identified by routine medical examination, e.g., laboratory tests (e.g., blood tests), genetic testing (e.g., testing for one or more deletions of a D4Z4 repeat in chromosome 4), or muscle biopsy.

Any of the methods described herein can further comprise a step of identifying a subject (e.g., a human subject) for treatment based on identifying one or more mutations (e.g., one or more mutations of a D4Z4 repeat in chromosome 4). In some embodiments, presence of one or more mutations (e.g., one or more mutations of a D4Z4 repeat in chromosome 4) is determined in a biological sample (e.g., a blood sample) obtained from a subject (e.g., a human subject).

### (b) Administration

Methods described herein encompass administering an effective amount of a recombinant nucleic acid, an expression vector (e.g., an AAV vector), an AAV particle, or a pharmaceutical composition thereof to a subject, e.g., a subject in need thereof, for example, a human subject, by a variety of methods. The route of administration can be intramuscular administration, subcutaneous injection, intrathecal injection, or intravenous (IV) administration.

An effective amount refers to the amount of a recombinant nucleic acid, an expression vector (e.g., an AAV vector), an AAV particle, or a pharmaceutical composition thereof needed to prevent or alleviate at least one or more signs or symptoms of FSHD, and relates to a sufficient amount of a recombinant nucleic acid, an expression vector (e.g., an AAV vector), an AAV particle, or pharmaceutical compositions thereof that provides the desired effect, e.g., to treat a subject (e.g., human subject) having FSHD. An effective amount also can include an amount sufficient to prevent or delay the development of a symptom of FSHD, alter the course of a symptom of FSHD (e.g., slow the progression of a symptom of FSHD), or reverse a symptom of FSHD.

Methods described herein comprise administering (e.g., intramuscularly or intravenously) to a subject (e.g., a human subject) a recombinant nucleic acid, an expression vector (e.g., an AAV vector), an AAV particle, or a pharmaceutical composition described herein one or more times. In some embodiments, an effective amount of a recombinant nucleic acid, an expression vector (e.g., an AAV vector), an AAV particle, or a pharmaceutical composition thereof is administered to a subject (e.g., a human subject) no more than once or more than once (e.g., administered twice, administered three times, or administered four times).

Any of the methods for treating FSHD described herein can be used in combination therapies. For example, any of the methods for treating FSHD described herein can be co-used with other therapeutic agents for treating FSHD, or for enhancing efficacy of the compositions described herein (e.g., the recombinant nucleic acids, the expression vectors (e.g., the AAV vectors), or pharmaceutical compositions) and/or reducing side effects of the compositions described herein (e.g., the recombinant nucleic acids, the expression vectors (e.g., the AAV vectors), or pharmaceutical compositions).

Any of the recombinant nucleic acids, the expression vectors (e.g., the AAV vectors), or pharmaceutical compositions described herein can be used as a medicament or in the manufacture of a medicament for the treatment of FSHD (e.g., FSHD1, FSHD2, or FSHD1+2), or ameliorating one or more symptoms thereof in a subject.

### VI. Kits for Therapeutic Use

The present disclosure provides kits for treating FSHD (e.g., FSHD1, FSHD2, or FSHD1+2). Such kits can include any of the recombinant nucleic acids, AAV vectors, AAV particles, or pharmaceutical compositions thereof described herein. In some embodiments, the kit can additionally comprise instructions for practicing any of the methods described herein. The instructions may include information as to dosage, dosing schedule, and route of administration. Instructions supplied in the kits of the disclosure can be written instructions on a label or a package insert.

### OTHER EMBODIMENTS

Without further elaboration, it is believed that one skilled in the art can, based on the above description, utilize the present disclosure to its fullest extent. The specific embodiments herein are to be construed as merely illustrative, and not limitative of the remainder of the disclosure in any way whatsoever. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

In any of the aspects and embodiments described herein, the nucleic acid sequences contemplated can be DNA, RNA, or modified versions thereof. Modified nucleic acids can be distinguished from naturally occurring nucleic acids by modifications to the backbone of the polynucleotide chain, for example, peptide nucleic acids (PNA), morpholinos, locked nucleic acids (LNA), glycol nucleic acids (GNA) and threose nucleic acid (TNA). Modified nucleic acids can also include analogs with modifications to the four nucleobases. In some embodiments, the nucleic acids are PNAs. In some embodiments, the nucleic acids are LNAs. In some embodiments, the nucleic acids are morpholinos. In some embodiments, the nucleic acids are in a single-stranded form. In some embodiments, the nucleic acids are in double-stranded form. In some embodiments, the nucleic acids are linear. In some embodiments, the nucleic acids are circular. In some embodiments, the nucleic acids are plasmids.

Non-limiting embodiments of the present disclosure include:

Embodiment 1 is a composition comprising an adeno-associated virus (AAV) particle comprising an engineered capsid protein comprising the amino acid sequence RGDR (SEQ ID NO: 3950); and a nucleic acid molecule comprising a promoter; a primary microRNA (pri-miRNA) scaffold having the scaffold sequence of miR-138 or miR-139 and a guide sequence within the scaffold that targets a double homeobox 4 (DUX4) transcript; a dorsal root ganglia (DRG) de-targeting miRNA binding site; and a heart de-targeting miRNA binding site.

Embodiment 2 is the composition of Embodiment 1, wherein the guide sequence comprises a sequence selected from among SEQ ID NOs: 2399-3159.

Embodiment 3 is the composition of Embodiment 2, wherein the DRG de-targeting miRNA binding site comprises a sequence selected from among SEQ ID NOs: 3921-3923.

Embodiment 4 is the composition of Embodiment 3, wherein the heart de-targeting miRNA binding site comprises a sequence selected from among SEQ ID NO: 3924 and SEQ ID NO: 3925.

Embodiment 5 is the composition of Embodiment 4, wherein the promoter comprises the sequence having at least 95% sequence identity with a sequence selected from among of SEQ ID NOs: 3926-3928.

Embodiment 6 is the composition of Embodiment 5, wherein the nucleic acid molecule comprises, in order, a first inverted terminal repeat (ITR) region; the promoter; the primary microRNA (pri-miRNA) comprising in order: an upstream scaffold; the guide sequence; a short hairpin loop; a passenger sequence; and a downstream scaffold, a first DRG de-targeting miRNA binding site; a first heart de-targeting miRNA binding site; a second DRG de-targeting miRNA binding site; a second heart de-targeting miRNA binding site; a poly adenylation signal; and a second ITR region.

Embodiment 7 is the composition of Embodiment 6, wherein the nucleic acid molecule comprises the sequence selected from among SEQ ID NOs: 3940-3947.

Embodiment 8 is the composition of Embodiment 1, wherein the capsid protein comprises an amino acid sequence selected from Table 2a in hypervariable region IV (HVR IV) relative to wild-type AAV9, wherein the capsid protein comprises substitutions at amino acids 451-455 relative to a wild-type AAV9 vector capsid selected from column 1 of Table 2b; and a 7-mer insert selected from column 2 of Table 2b inserted after amino acid 455 relative to a wild-type AAV9 vector.

Embodiment 9 is the composition of Embodiment 8, wherein the capsid protein further comprises a deletion of G267 relative to wild-type AAV9 capsid or equivalent position in another AAV capsid.

Embodiment 10 is the composition of Embodiment 9, wherein the AAV vector exhibits reduced liver tropism and increased muscle tropism as compared to a wild type AAV vector.

Embodiment 11 is a method of treating a subject suffering from facioscapulohumeral muscular dystrophy (FSHD), the method comprising providing to the subject a composition comprising an adeno −associated virus (AAV) particle comprising an engineered capsid protein comprising the amino acid sequence RGDR; and a nucleic acid molecule comprising a promoter; a primary microRNA (pri-miRNA) scaffold having the scaffold sequence of miR-138 or miR-139 and a guide sequence within the scaffold that targets a double homeobox 4 (DUX4) transcript; a dorsal root ganglia (DRG) de-targeting miRNA binding site; and a heart de-targeting miRNA binding site.

Embodiment 12 is the method of Embodiment 11, wherein the guide sequence comprises a sequence selected from among SEQ ID NOs: 2399-3159.

Embodiment 13 is the method of Embodiment 12, wherein the DRG de-targeting miRNA binding site comprises a sequence selected from among SEQ ID Nos: 3921-3923.

Embodiment 14 is the method of Embodiment 13, wherein the heart de-targeting miRNA binding site comprises a sequence selected from among SEQ ID NO: 3924 and SEQ ID NO: 3925.

Embodiment 15 is the method of Embodiment 14, wherein the promoter comprises the sequence having at least 95% sequence identity with a sequence selected from among of SEQ ID NOs: 3926-3928.

Embodiment 16 is the method of Embodiment 15, wherein the nucleic acid molecule comprises, in order, a first inverted terminal repeat (ITR) region; the promoter; the primary microRNA (pri-miRNA) comprising in order: an upstream scaffold; the guide sequence; a short hairpin loop; a passenger sequence; and a downstream scaffold, a first DRG de-targeting miRNA binding site; a first heart de-targeting miRNA binding site; a second DRG de-targeting miRNA binding site; a second heart de-targeting miRNA binding site; a poly adenylation signal; and a second ITR region.

Embodiment 17 is the method of Embodiment 16, wherein the nucleic acid molecule comprises the sequence selected from among SEQ ID NOs: 3940-3947.

Embodiment 18 is the method of Embodiment 11, wherein the capsid protein comprises an amino acid sequence selected from Tables 2a in hypervariable region IV (HVR IV) relative to wild-type AAV9, wherein the capsid protein comprises substitutions at amino acids 451-455 relative to a wild-type AAV9 vector capsid selected from column 1 of Table 2b; and a 7-mer insert selected from column 2 of Table 2b inserted after amino acid 455 relative to a wild-type AAV9 vector.

Embodiment 19 is the method of Embodiment 18, wherein the capsid protein further comprises a deletion of G267 relative to wild-type AAV9 capsid or equivalent position in another AAV capsid.

Embodiment 20 is the method of Embodiment 19, wherein the AAV vector exhibits reduced liver tropism and increased muscle tropism as compared to a wild type AAV vector.

### EXPERIMENTAL EXAMPLES

In order that the invention described may be more fully understood, the following examples are set forth. The examples described in this application are offered to illustrate the methods and compositions provided herein and are not to be construed in any way as limiting their scope.

### Example 1: Exemplary Recombinant Nucleic Acids and AAV Compositions

Exemplary AAV compositions of the invention were engineered comprising the capsid protein MyoAAV-LD B comprising an RGDR motif and a recombinant nucleic acid (also referred to as cargo) described herein.

The capsid protein MyoAAV-LD B can be described relative to a wild type AAV9, specifically the capsid protein MyoAAV-LD B includes the amino acid sequence of LNAST (SEQ ID NO: 1014) at positions 451-455, the amino acid sequence of RGDRQYL (SEQ ID NO: 1813) inserted after amino acid 455, and a deletion of the amino acid G267 relative to the wild type AAV9 capsid protein, and the remainder of the capsid is otherwise identical to wild type AAV9.

The capsid encapsidated a recombinant nucleic acid comprising a promoter comprising a sequence selected from any one of SEQ ID NOs: 3926-3928, a microRNA (miRNA) scaffold comprising the scaffold miR-138 (SEQ ID NO: 3932) or miR-139 (SEQ ID NO: 3936) together with a guide that targets a double homeobox protein 4 (DUX4) transcript comprising a sequence selected from any one of SEQ ID NOs: 2399-3159 and 3952-3953, a first and second DRG de-targeting miR binding site comprising a sequence selected from any one of SEQ ID NOs: 3921-3923, and a first and second heart de-targeting miRNA binding site comprising a sequence selected from SEQ ID NO: 2924 or SEQ ID NO: 2925.

Exemplary recombinant nucleic acids are described below:

Nucleic acid 1
(SEQ ID NO: 3940)
TCTCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAG
AGGGAGTCATATAACATGAAGATCATCTGAATTCGTAGCTTTAGCCGGCT
TGCTTGCTTTTGGAGTCTGCCCCTCGTATGTACAGATCGGGGTCAGCCTG
AGACGTGGGGGGTTCCTTCCCAGGCGTTGCAGCTCTCTTTCCTCCCTCAC
TCTCCAGTTCCTCTGGCTCAGTGAGGGATTTGGGATTTCTGTGTGTGGGG
CATGGACAGATACTACTCTTAGTGAAAAGCTGGACAAGGTGGAGTAGAGC
ATCAAGGGTGAGTGCCACTACGGGTCTAGGCTGCCCATGTAAGGAGGCAA
GGCCTGGGGACACCCGAGATGCCTGGTTATAATTAACCCAGACATGTGGC
TGCCCCCCCCCCCCCAACACCTGCTGCCTGCTAAAAATAACCCTGTCCCT
GGTGGATATCAAGGCTGTGGGGGACTGAGGGCAGGCTGTAACAGGCTTGG
GGGCCAGGGCTTATACGTGCCTGGGACTCCCAAAGTATTACTGTTCCATG
TTCCCGGCGAAGGGCCAGCTGTCCCCCGCCAGCTAGACTCAGCACTTAGT
TTAGGAACCAGTGAGCAAGTCAGCCCTTGGGGCAGCCCATACAAGGCCAT
GGGGCTGGGCAAGCTGCACGCCTGGGTCCGGGGTGGGCACGGTGCCCGGG
CAACGAGCTGAAAGCTCATCTACTCTCAGGGGCCCCTCCCTGGGGACAGC
CCCTCCTGGCTAGTCACACCCTGTAGGCTCCTCTATATAACCCAGGGGCA
CAGGGGCTGCCCTCATTCTACCACCACCTCCACAGCACAGACAGACACTC
AGGAGCCAGCCAGCCATCTACTGGGGACCAGCTATGGTAAGGGATGAGAG
AAGCCAACAGTTGTCCCAAGGGTCCCCCTCAGGGAGTGGAGGTTCCCCTG
GGAGGCGCCAGGACAGGCGCAGGTGTATCCGGTGTGGGCGAAGGCGACCT
GTGGCTCGGAGGCGGTCGCCTaaGgCCACACCGGTAACAACTGAAGCCAG
AGTCTTCGAAGGGTGGTGGGCTAAGGGGGAAGAGGGCATGGTGGGCAAAA
GAAAGGGGGTGGGGGAGAGAGAAAAGGAGGACAAGGGGGACTACTCTCAT
ACAGCTAGATAACCAAAGAAATATTCAAACATCACTGCAAGTCTTAATGC
TTATCAACAAAATCACTGATGCTGGAACTCTACGAAACCCAGCAGACAAT
GTAGCTAGAATATCTAGAGCTCGCTGATCAGCCTCGACTGTGCCTTCTAG
TTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGA
CAGCAAGGGGGAGGATTGGGAAGAGAATAGCAGGCATGCTGGGGAAGGAA
CCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGG
CCTCAGTGAGCGAGCGAGCGCGC

| Nucleic acid | |
|---|---|
| cargo 1 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | GTAGCTTTAGCCGGCTTGCTTGCTTTTGGAGTCTGCCCCTCGTATGTACAGATCG |
| | GGGTCAGCCTGAGACGTGGGCGGGTTCCTTCCCAGGCGTTGCAGCTCTCTTTCCT |
| | CCCTCACTCTCCAGTTCCTCTGGCTCAGTGAGGGATTTGGGATTTCTGTGTGTGG |
| | GGCATGGACAGATACTACTCTTAGTGAAAAGCTGGACAAGGTGGAGTAGAGCATC |
| | AAGGGTGAGTGCCACTACGGGTCTAGGCTGCCCATGTAAGGAGGCAAGGCCTGGG |
| | GACACCCGAGATGCCTGGTTATAATTAACCCAGACATGTGGCTGCCCCCCCCCCC |
| | CCAACACCTGCTGCCTGCTAAAAATAACCCTGTCCCTGGTGGATATCAAGGCTGT |
| | GGGGGACTGAGGGCAGGCTGTAACAGGCTTGGGGGCCAGGGCTTATACGTGCCTG |
| | GGACTCCCAAAGTATTACTGTTCCATGTTCCCGGCGAAGGGCCAGCTGTCCCCCG |
| | CCAGCTAGACTCAGCACTTAGTTTAGGAACCAGTGAGCAAGTCAGCCCTTGGGGC |
| | AGCCCATACAAGGCCATGGGGCTGGGCAAGCTGCACGCCTGGGTCCGGGGTGGGC |
| | ACGGTGCCCGGGCAACGAGCTGAAAGCTCATCTACTCTCAGGGGCCCCTCCCTGG |
| | GGACAGCCCCTCCTGGCTAGTCACACCCTGTAGGCTCCTCTATATAACCCAGGGG |
| | CACAGGGGCTGCCCTCATTCTACCACCACCTCCACAGCACAGACAGACACTCAGG |
| | AGCCAGCCAGC (SEQ ID NO: 3927) |
| upstream miR | GGGGACCAGCTATGGTAAGGGATGAGAGAAGCCAACAGTTGTCCCAAGGGTCCCC |
| scaffold | CTCAGGGAGTGGAGGTTCCCCTGGGAGGCGCCAGGACAGGCGCAGGTGTAT |
| | (SEQ ID NO: 3933) |
| guide sequence | CCGGTGTGGGCGAAGGCGACC (SEQ ID NO: 2475) |
| hairpin loop | TGTGGCTCGGAGGC (SEQ ID NO: 3934) |
| passenger | GGTCGCCTaaGgCCACACCGG (SEQ ID NO: 3236) |
| sequence | |
| downstream | TAACAACTGAAGCCAGAGTCTTCGAAGGGTGGTGGGCTAAGGGGGAAGAGGGCAT |
| scaffold | GGTGGGCAAAAGAAAGGGGGTGGGGGAGAGAGAAAAGGAGGACAAGGGGG |
| | (SEQ ID NO: 3935) |
| first DRG de- | TCATACAGCTAGATAACCAAAGA (SEQ ID NO: 3923) |
| targeting miRNA | |
| binding site | |
| First heart de- | AAACATCACTGCAAGTCTTAA (SEQ ID NO: 3925) |
| targeting miRNA | |
| binding site | |
| second DRG de- | CAACAAAATCACTGATGCTGGA (SEQ ID NO: 3921) |
| targeting miRNA | |
| binding site | |
| Second heart | GAAACCCAGCAGACAATGTAGCT (SEQ ID NO: 3924) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

Nucleic acid 2
(SEQ ID NO: 3941)
TCTCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAG
AGGGAGTCATATAACATGAAGATCATCTGAATTCGGAAGAGAAGGTGACC
CTTACCCAGTTGTTCAACTCACCCTTCAGATTAAAAATAACTAAGGTAAG
GGCCTGGGTAGGGGAGGTGGTGTGAGAGGGTCCTGTCTCTCCTCTATCTG
CCCATCGGCCCTTTGGGGAGGAGGAATGTGCCCAAGGACTAAAAAAAGGC
CCTGGAGCCAGAGGGGCGAGGGCAGCAGACCTTTCATGGGCAAACCTCAG
GGCTGCTGGCCACTACGGGTCTAGGCTGCCCATGTAAGGAGGCAAGGCCT
GGGGACACCCGAGATGCCTGGTTATAATTAACCCAGACATGTGGCTGCCC
CCCCCCCCCCAACACCTGCTGCCTGCTAAAAATAACCCTGTCCCTGGTGG
ATATCAAGGCTGTGGGGGACTGAGGGCAGGCTGTAACAGGCTTGGGGGCC
AGGGCTTATACGTGCCTGGGACTCCCAAAGTATTACTGTTCCATGTTCCC
GGCGAAGGGCCAGCTGTCCCCCGCCAGCTAGACTCAGCACTTAGTTTAGG
AACCAGTGAGCAAGTCAGCCCTTGGGGCAGCCCATACAAGGCCATGGGGC
TGGGCAAGCTGCACGCCTGGGTCCGGGGTGGGCACGGTGCCCGGGCAACG
AGCTGAAAGCTCATCTACTCTCAGGGGCCCCTCCCTGGGGACAGCCCCTC
CTGGCTAGTCACACCCTGTAGGCTCCTCTATATAACCCAGGGGCACAGGG
GCTGCCCTCATTCTACCACCACCTCCACAGCACAGACAGACACTCACGAG
CCAGCCATCTACTGGGGACCAGCTATGGTAAGGGATGAGAGAAGCCAACA
GTTGTCCCAAGGGTCCCCCTCAGGGAGTGGAGGTTCCCCTGGGAGGCGCC
AGGACAGGCGCAGGTGTATTTCGATTCTGAAACCAGATCTTGTGGCTCGG
AGGCAGATCTGGAGTAAGAATCGAATAACAACTGAAGCCAGAGTCTTCGA
AGGGTGGTGGGCTAAGGGGGAAGAGGGCATGGTGGGCAAAAGAAAGGGGG
TGGGGGAGAGAGAAAAGGAGGACAAGGGGGACTACTCCAACAAAATCACT
GATGCTGGAAATATTCGAAACCCAGCAGACAATGTAGCTTGCTTATCAAC
AAAATCACTGATGCTGGAACTCTACGAAACCCAGCAGACAATGTAGCTAG
AATATCTAGAGCTCGCTGATCAGCCTCGACTGTGCCTTCTAGTTGCCAGC
CATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGG
GGGAGGATTGGGAAGAGAATAGCAGGCATGCTGGGGAAGGAACCCCTAGT
GATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTG
AGCGAGCGAGCGCGC

| Nucleic acid | |
|---|---|
| cargo 2 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | GGAAGAGAAGGTGACCCTTACCCAGTTGTTCAACTCACCCTTCAGATTAAAAATA |
| | ACTAAGGTAAGGGCCTGGGTAGGGGAGGTGGTGTGAGAGGGTCCTGTCTCTCCTC |
| | TATCTGCCCATCGGCCCTTTGGGGAGGAGGAATGTGCCCAAGGACTAAAAAAAGG |
| | CCCTGGAGCCAGAGGGGCGAGGGCAGCAGACCTTTCATGGGCAAACCTCAGGGCT |
| | GCTGGCCACTACGGGTCTAGGCTGCCCATGTAAGGAGGCAAGGCCTGGGGACACC |
| | CGAGATGCCTGGTTATAATTAACCCAGACATGTGGCTGCCCCCCCCCCCCCAACA |
| | CCTGCTGCCTGCTAAAAATAACCCTGTCCCTGGTGGATATCAAGGCTGTGGGGGA |
| | CTGAGGGCAGGCTGTAACAGGCTTGGGGGCCAGGGCTTATACGTGCCTGGGACTC |
| | CCAAAGTATTACTGTTCCATGTTCCCGGCGAAGGGCCAGCTGTCCCCCGCCAGCT |
| | AGACTCAGCACTTAGTTTAGGAACCAGTGAGCAAGTCAGCCCTTGGGGCAGCCCA |
| | TACAAGGCCATGGGGCTGGGCAAGCTGCACGCCTGGGTCCGGGGTGGGCACGGTG |
| | CCCGGGCAACGAGCTGAAAGCTCATCTACTCTCAGGGGCCCCTCCCTGGGGACAG |
| | CCCCTCCTGGCTAGTCACACCCTGTAGGCTCCTCTATATAACCCAGGGGCACAGG |
| | GGCTGCCCTCATTCTACCACCACCTCCACAGCACAGACAGACACTCACGAGCCAG |
| | C (SEQ ID NO: 3926) |
| upstream miR | GGGGACCAGCTATGGTAAGGGATGAGAGAAGCCAACAGTTGTCCCAAGGGTCCCC |
| scaffold | CTCAGGGAGTGGAGGTTCCCCTGGGAGGCGCCAGGACAGGCGCAGGTGTAT |
| | (SEQ ID NO: 3933) |
| guide sequence | TTCGATTCTGAAACCAGATCT (SEQ ID NO: 3159) |
| hairpin loop | TGTGGCTCGGAGGC (SEQ ID NO: 3934) |
| passenger | AGATCTGGAGTAAGAATCGAA (SEQ ID NO: 3920) |
| sequence | |
| downstream | TAACAACTGAAGCCAGAGTCTTCGAAGGGTGGTGGGCTAAGGGGGAAGAGGGCAT |
| scaffold | GGTGGGCAAAAGAAAGGGGGTGGGGGAGAGAGAAAAGGAGGACAAGGGGG (SEQ |
| | ID NO: 3935) |
| first DRG de- | CAACAAAATCACTGATGCTGGA (SEQ ID NO: 3921) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CAACAAAATCACTGATGCTGGA (SEQ ID NO: 3921) |
| targeting miRNA | |
| binding site | |
| Second heart | GAAACCCAGCAGACAATGTAGCT (SEQ ID NO: 3924) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

Nucleic acid 3
(SEQ ID NO: 3942)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCCCCCTGACCTTTGCGG
TGGAAATCCTGAGCCCAGGCTGAGATGAGCTCAGCTGATCCTTCTCTCAGAGACAGCTGGAGTACAGTGCTGCTC
AGCCCCCAGCTCCTGAGTGTCTGGAGCAAACAGCTGGGAGAGAGGTAGAATTCACTCCTGGATTAGAGCCAAAAA
CTGCCTGGCAGAAACCCCCAGGATCCTGTTTCTCCTGAGGGAGGAGCGGGGGAGCCCGGGGGCGAGAAACCCACA
GCACAGGGAAGGGAAGGGCAGCCTTGAAACTGCAAGTTGGGAGATCACGGTTTCCTCACGTGGCCACCATGAGGG
CTGAGGCTGTAGGCTGGCCGCTCCTGCCCCTCCCGACCACCCCCTCAGGCTTCCAGCCTCCTTGCTCAGCCACCT
AGGCTGGGCTTTCAGGTTCACAGGCAGCCGGCCCTACAACATGGAGGCCCAGGCTGGGCTTTCTTCAGGAGTGGC
ATCTAAACTTAGGAGAGGGGAACACGTTTCACCCGAATCTGGGCTAGCAGTGTGTTGGTTAAATGGCCCCTGAGT
AGGTGTGGGGCATGGCAGAGGTGCAGGAGTGGGTGTCCGAGGATGCAGCCAACTCATCTCCAAACAGCTCCTTTT
CTTTTCCCCAGTGGGCTGCATTCCCAGACTGGTATTTTAGTTGGTGTGACAGAGTCCCAGAGTCATCTGGCCCAG
GGGCTTGCACCTGCGCCAGTCCTTCCCTGTCCACCCCTGCCCGTCTCCAGGCGCTCCTATAAAGGGCTGAGCAGT
TCAGCTCTTCTCACTGGGGGGTGTGAGGAACAGGCATCTACTGGACCACCCAGCAGTGATGCACGTAGAGCAGAG
GTGCAGTGATGCACGTACAGCAGAGGCGACCCCTTCTCCATACTTCAGAGACCTCTAGCATGGTGTTGTGGGACT
GTCCCGGGTGCCTGGCCCTTTTGCCAATCAGAGAAAAGGGCCAaaCtCCCGGGACACCACACTGCACTGCAAGCA
GCGAGCAGCAAGCAACAAGCATCCAGACACCTGAGCACCAGGATGCCAGCAAGAGCACCTGCCTGTGAGGAAACC
TCCCAGCCACTACTCCAACAAAATCACTGATGCTGGAAATATTCGAAACCCAGCAGACAATGTAGCTTGCTTATC
GGCCTGATTCACAACACCAGCTACTCTACAAACATCACTGCAAGTCTTAAAGAATATCTAGAGCTCGCTGATCAG
CCTCGACTGTGCCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGAGAATAGCAGGCATGCTGGGGAAGGAACCCCTA
GTGATGGAGTTGGCCACTCCCTCTCTGCGCGCTCGCTCGCTCACTGAGGCCGGGCGACCAAAGGTCGCCCGACGC
CCGGGCTTTGCCCGGGCGGCCTCAGTGAGCGAGCGAGCGCGC

| Nucleic acid | |
|---|---|
| cargo 3 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | CCCCTGACCTTTGCGGTGGAAATCCTGAGCCCAGGCTGAGATGAGCTCAGCTGAT |
| | CCTTCTCTCAGAGACAGCTGGAGTACAGTGCTGCTCAGCCCCCAGCTCCTGAGTG |
| | TCTGGAGCAAACAGCTGGGAGAGAGGTAGAATTCACTCCTGGATTAGAGCCAAAA |
| | ACTGCCTGGCAGAAACCCCCAGGATCCTGTTTCTCCTGAGGGAGGAGCGGGGGAG |
| | CCCGGGGGCGAGAAACCCACAGCACAGGGAAGGGAAGGGCAGCCTTGAAACTGCA |
| | AGTTGGGAGATCACGGTTTCCTCACGTGGCCACCATGAGGGCTGAGGCTGTAGGC |
| | TGGCCGCTCCTGCCCCTCCCGACCACCCCCTCAGGCTTCCAGCCTCCTTGCTCAG |
| | CCACCTAGGCTGGGCTTTCAGGTTCACAGGCAGCCGGCCCTACAACATGGAGGCC |
| | CAGGCTGGGCTTTCTTCAGGAGTGGCATCTAAACTTAGGAGAGGGGAACACGTTT |
| | CACCCGAATCTGGGCTAGCAGTGTGTTGGTTAAATGGCCCCTGAGTAGGTGTGGG |
| | GCATGGCAGAGGTGCAGGAGTGGGTGTCCGAGGATGCAGCCAACTCATCTCCAAA |
| | CAGCTCCTTTTCTTTTCCCCAGTGGGCTGCATTCCCAGACTGGTATTTTAGTTGG |
| | TGTGACAGAGTCCCAGAGTCATCTGGCCCAGGGGCTTGCACCTGCGCCAGTCCTT |
| | CCCTGTCCACCCCTGCCCGTCTCCAGGCGCTCCTATAAAGGGCTGAGCAGTTCAG |
| | CTCTTCTCACTGGGGGGTGTGAGGAACAGG (SEQ ID NO: 3928) |
| upstream miR | GGACCACCCAGCAGTGATGCACGTAGAGCAGAGGTGCAGTGATGCACGTACAGCA |
| scaffold | GAGGCGACCCCTTCTCCATACTTCAGAGACCTCTAGCATGGTGTTGTGGGAC |
| | (SEQ ID NO: 3929) |
| guide sequence | TGTCCCGGGTGCCTGGCCCTT (SEQ ID NO: 3047) |
| hairpin loop | TTGCCAATCAGAGAA (SEQ ID NO: 3930) |
| passenger | AAGGGCCAaaCtCCCGGGACA (SEQ ID NO: 3808) |
| sequence | |
| downstream | CCACACTGCACTGCAAGCAGCGAGCAGCAAGCAACAAGCATCCAGACACCTGAGC |
| scaffold | ACCAGGATGCCAGCAAGAGCACCTGCCTGTGAGGAAACCTCCCAGCC (SEQ ID |
| | NO: 3931) |
| first DRG de- | CAACAAAATCACTGATGCTGGA (SEQ ID NO: 3921) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CGGCCTGATTCACAACACCAGCT (SEQ ID NO: 3922) |
| targeting miRNA | |
| binding site | |
| Second heart | AAACATCACTGCAAGTCTTAA (SEQ ID NO: 3925) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

Nucleic acid 4
(SEQ ID NO: 3943)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCGGAAGAGAAGGTGACC
CTTACCCAGTTGTTCAACTCACCCTTCAGATTAAAAATAACTAAGGTAAGGGCCTGGGTAGGGGAGGTGGTGTGA
GAGGGTCCTGTCTCTCCTCTATCTGCCCATCGGCCCTTTGGGGAGGAGGAATGTGCCCAAGGACTAAAAAAAGGC
CCTGGAGCCAGAGGGGCGAGGGCAGCAGACCTTTCATGGGCAAACCTCAGGGCTGCTGGCCACTACGGGTCTAGG
CTGCCCATGTAAGGAGGCAAGGCCTGGGGACACCCGAGATGCCTGGTTATAATTAACCCAGACATGTGGCTGCCC
CCCCCCCCCCAACACCTGCTGCCTGCTAAAAATAACCCTGTCCCTGGTGGATATCAAGGCTGTGGGGGACTGAGG
GCAGGCTGTAACAGGCTTGGGGGCCAGGGCTTATACGTGCCTGGGACTCCCAAAGTATTACTGTTCCATGTTCCC
GGCGAAGGGCCAGCTGTCCCCCGCCAGCTAGACTCAGCACTTAGTTTAGGAACCAGTGAGCAAGTCAGCCCTTGG
GGCAGCCCATACAAGGCCATGGGGCTGGGCAAGCTGCACGCCTGGGTCCGGGGTGGGCACGGTGCCCGGGCAACG
AGCTGAAAGCTCATCTACTCTCAGGGGCCCCTCCCTGGGGACAGCCCCTCCTGGCTAGTCACACCCTGTAGGCTC
CTCTATATAACCCAGGGGCACAGGGGCTGCCCTCATTCTACCACCACCTCCACAGCACAGACAGACACTCACGAG
CCAGCCATCTACTGGACCACCCAGCAGTGATGCACGTAGAGCAGAGGTGCAGTGATGCACGTACAGCAGAGGCGA
CCCCTTCTCCATACTTCAGAGACCTCTAGCATGGTGTTGTGGGACTGTCCCGGGTGCCTGGCCCTTTTGCCAATC
AGAGAAAAGGGCCAaaCtCCCGGGACACCACACTGCACTGCAAGCAGCGAGCAGCAAGCAACAAGCATCCAGACA
CCTGAGCACCAGGATGCCAGCAAGAGCACCTGCCTGTGAGGAAACCTCCCAGCCACTACTCCAACAAAATCACTG
ATGCTGGAAATATTCAAACATCACTGCAAGTCTTAATGCTTATCGGCCTGATTCACAACACCAGCTACTCTACAA
ACATCACTGCAAGTCTTAAAGAATATCTAGAGCTCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGGGGCAGGACAGCAAGGGGGA
GGATTGGGAAGAGAATAGCAGGCATGCTGGGGAAGGAACCCCTAGTGATGGAGTTGGCCACTCCCTCTCTGCGCG
CTCGCTCGCTCACTGAGGCCGGGCGACCAAAGGTCGCCCGACGCCCGGGCTTTGCCCGGGCGGCCTCAGTGAGCG
AGCGAGCGCGC

| Nucleic acid | |
|---|---|
| cargo 4 | |
| annotated | Sequence |
| Self- | TCTCTGCGCGCTCGCTCGCTCACTGAGGCCGCCCGGGCAAAGCCCGGGCGTCGGGC |
| complimentary | GACCTTTGGTCGCCCGGCCTCAGTGAGCGAGCGAGCGCGCAGAGAGGGAGT (SEQ |
| AAV2 ITR | ID NO: 3937) |
| promoter | GGAAGAGAAGGTGACCCTTACCCAGTTGTTCAACTCACCCTTCAGATTAAAAATAA |
| | CTAAGGTAAGGGCCTGGGTAGGGGAGGTGGTGTGAGAGGGTCCTGTCTCTCCTCTA |
| | TCTGCCCATCGGCCCTTTGGGGAGGAGGAATGTGCCCAAGGACTAAAAAAAGGCCC |
| | TGGAGCCAGAGGGGCGAGGGCAGCAGACCTTTCATGGGCAAACCTCAGGGCTGCTG |
| | GCCACTACGGGTCTAGGCTGCCCATGTAAGGAGGCAAGGCCTGGGGACACCCGAGA |
| | TGCCTGGTTATAATTAACCCAGACATGTGGCTGCCCCCCCCCCCCCAACACCTGCT |
| | GCCTGCTAAAAATAACCCTGTCCCTGGTGGATATCAAGGCTGTGGGGGACTGAGGG |
| | CAGGCTGTAACAGGCTTGGGGGCCAGGGCTTATACGTGCCTGGGACTCCCAAAGTA |
| | TTACTGTTCCATGTTCCCGGCGAAGGGCCAGCTGTCCCCCGCCAGCTAGACTCAGC |
| | ACTTAGTTTAGGAACCAGTGAGCAAGTCAGCCCTTGGGGCAGCCCATACAAGGCCA |
| | TGGGGCTGGGCAAGCTGCACGCCTGGGTCCGGGGTGGGCACGGTGCCCGGGCAACG |
| | AGCTGAAAGCTCATCTACTCTCAGGGGCCCCTCCCTGGGGACAGCCCCTCCTGGCT |
| | AGTCACACCCTGTAGGCTCCTCTATATAACCCAGGGGCACAGGGGCTGCCCTCATT |
| | CTACCACCACCTCCACAGCACAGACAGACACTCACGAGCCAGC (SEQ ID NO: |
| | 3926) |
| upstream miR | GGACCACCCAGCAGTGATGCACGTAGAGCAGAGGTGCAGTGATGCACGTACAGCAG |
| scaffold | AGGCGACCCCTTCTCCATACTTCAGAGACCTCTAGCATGGTGTTGTGGGAC (SEQ |
| | ID NO: 3929) |
| guide sequence | TGTCCCGGGTGCCTGGCCCTT (SEQ ID NO: 3047) |
| hairpin loop | TTGCCAATCAGAGAA (SEQ ID NO: 3930) |
| passenger | AAGGGCCAaaCtCCCGGGACA (SEQ ID NO: 3808) |
| sequence | |
| downstream | CCACACTGCACTGCAAGCAGCGAGCAGCAAGCAACAAGCATCCAGACACCTGAGCA |
| scaffold | CCAGGATGCCAGCAAGAGCACCTGCCTGTGAGGAAACCTCCCAGCC (SEQ ID |
| | NO: 3931) |
| first DRG de- | CAACAAAATCACTGATGCTGGA (SEQ ID NO: 3921) |
| targeting miRNA | |
| binding site | |
| First heart de- | AAACATCACTGCAAGTCTTAA (SEQ ID NO: 3925) |
| targeting miRNA | |
| binding site | |
| second DRG de- | CGGCCTGATTCACAACACCAGCT (SEQ ID NO: 3922) |
| targeting miRNA | |
| binding site | |
| Second heart | AAACATCACTGCAAGTCTTAA (SEQ ID NO: 3925) |
| de-targeting | |
| miRNA binding | |
| site | |
| polyA signal | CTAGAGCTCGCTGATCAGCCTCGACTGTGCCTTCTAGTTGCCAGCCATCTGTTGTT |
| | TGCCCCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTCTGG |
| | GGGGTGGGGTGGGGCAGGACAGCAAGGGGGAGGATTGGGAAGAGAATAGCAGGCAT |
| | GCTGGGGA (SEQ ID NO: 3938) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 3939) |

Nucleic acid 5
(SEQ ID NO: 3944)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCGTAGCTTTAGCCGGCT
TGCTTGCTTTTGGAGTCTGCCCCTCGTATGTACAGATCGGGGTCAGCCTGAGACGTGGGCGGGTTCCTTCCCAGG
CGTTGCAGCTCTCTTTCCTCCCTCACTCTCCAGTTCCTCTGGCTCAGTGAGGGATTTGGGATTTCTGTGTGTGGG
GCATGGACAGATACTACTCTTAGTGAAAAGCTGGACAAGGTGGAGTAGAGCATCAAGGGTGAGTGCCACTACGGG
TCTAGGCTGCCCATGTAAGGAGGCAAGGCCTGGGGACACCCGAGATGCCTGGTTATAATTAACCCAGACATGTGG
CTGCCCCCCCCCCCCCAACACCTGCTGCCTGCTAAAAATAACCCTGTCCCTGGTGGATATCAAGGCTGTGGGGGA
CTGAGGGCAGGCTGTAACAGGCTTGGGGGCCAGGGCTTATACGTGCCTGGGACTCCCAAAGTATTACTGTTCCAT
GTTCCCGGCGAAGGGCCAGCTGTCCCCCGCCAGCTAGACTCAGCACTTAGTTTAGGAACCAGTGAGCAAGTCAGC
CCTTGGGGCAGCCCATACAAGGCCATGGGGCTGGGCAAGCTGCACGCCTGGGTCCGGGGTGGGCACGGTGCCCGG
GCAACGAGCTGAAAGCTCATCTACTCTCAGGGGCCCCTCCCTGGGGACAGCCCCTCCTGGCTAGTCACACCCTGT
AGGCTCCTCTATATAACCCAGGGGCACAGGGGCTGCCCTCATTCTACCACCACCTCCACAGCACAGACAGACACT
CAGGAGCCAGCCAGCCATCTACTGGACCACCCAGCAGTGATGCACGTAGAGCAGAGGTGCAGTGATGCACGTACA
GCAGAGGCGACCCCTTCTCCATACTTCAGAGACCTCTAGCATGGTGTTGTGGGACCCGGTGTGGGCGAAGGCGAC
CTTGCCAATCAGAGAAGGTCGCCTaaGgCCACACCGGCCACACTGCACTGCAAGCAGCGAGCAGCAAGCAACAAG
CATCCAGACACCTGAGCACCAGGATGCCAGCAAGAGCACCTGCCTGTGAGGAAACCTCCCAGCCACTACTCCAAC
AAAATCACTGATGCTGGAAATATTCGAAACCCAGCAGACAATGTAGCTTGCTTATCGGCCTGATTCACAACACCA
GCTACTCTACAAACATCACTGCAAGTCTTAAAGAATATCTAGAGCTCGCTGATCAGCCTCGACTGTGCCTTCTAG
TTGCCAGCCATCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGGTGGGGCAGGA
CAGCAAGGGGGAGGATTGGGAAGAGAATAGCAGGCATGCTGGGGAAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGC

| Nucleic acid | |
|---|---|
| cargo 5 | |
| annotated | Sequence |
| Self- | TCTCTGCGCGCTCGCTCGCTCACTGAGGCCGCCCGGGCAAAGCCCGGGCGTCGGGC |
| complimentary | GACCTTTGGTCGCCCGGCCTCAGTGAGCGAGCGAGCGCGCAGAGAGGGAGT (SEQ |
| AAV2 ITR | ID NO: 3937) |
| promoter | GTAGCTTTAGCCGGCTTGCTTGCTTTTGGAGTCTGCCCCTCGTATGTACAGATCGG |
| | GGTCAGCCTGAGACGTGGGCGGGTTCCTTCCCAGGCGTTGCAGCTCTCTTTCCTCC |
| | CTCACTCTCCAGTTCCTCTGGCTCAGTGAGGGATTTGGGATTTCTGTGTGTGGGGC |
| | ATGGACAGATACTACTCTTAGTGAAAAGCTGGACAAGGTGGAGTAGAGCATCAAGG |
| | GTGAGTGCCACTACGGGTCTAGGCTGCCCATGTAAGGAGGCAAGGCCTGGGGACAC |
| | CCGAGATGCCTGGTTATAATTAACCCAGACATGTGGCTGCCCCCCCCCCCCCAACA |
| | CCTGCTGCCTGCTAAAAATAACCCTGTCCCTGGTGGATATCAAGGCTGTGGGGGAC |
| | TGAGGGCAGGCTGTAACAGGCTTGGGGGCCAGGGCTTATACGTGCCTGGGACTCCC |
| | AAAGTATTACTGTTCCATGTTCCCGGCGAAGGGCCAGCTGTCCCCCGCCAGCTAGA |
| | CTCAGCACTTAGTTTAGGAACCAGTGAGCAAGTCAGCCCTTGGGGCAGCCCATACA |
| | AGGCCATGGGGCTGGGCAAGCTGCACGCCTGGGTCCGGGGTGGGCACGGTGCCCGG |
| | GCAACGAGCTGAAAGCTCATCTACTCTCAGGGGCCCCTCCCTGGGGACAGCCCCTC |
| | CTGGCTAGTCACACCCTGTAGGCTCCTCTATATAACCCAGGGGCACAGGGGCTGCC |
| | CTCATTCTACCACCACCTCCACAGCACAGACAGACACTCAGGAGCCAGCCAGC |
| | (SEQ ID NO: 3927) |
| upstream miR | GGACCACCCAGCAGTGATGCACGTAGAGCAGAGGTGCAGTGATGCACGTACAGCAG |
| scaffold | AGGCGACCCCTTCTCCATACTTCAGAGACCTCTAGCATGGTGTTGTGGGAC (SEQ |
| | ID NO: 3929) |
| guide sequence | CCGGTGTGGGCGAAGGCGACC (SEQ ID NO: 2475) |
| hairpin loop | TTGCCAATCAGAGAA (SEQ ID NO: 3930) |
| passenger | GGTCGCCTaaGgCCACACCGG (SEQ ID NO: 3236) |
| sequence | |
| downstream | CCACACTGCACTGCAAGCAGCGAGCAGCAAGCAACAAGCATCCAGACACCTGAGCA |
| scaffold | CCAGGATGCCAGCAAGAGCACCTGCCTGTGAGGAAACCTCCCAGCC (SEQ ID |
| | NO: 3931) |
| first DRG de- | CAACAAAATCACTGATGCTGGA (SEQ ID NO: 3921) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CGGCCTGATTCACAACACCAGCT (SEQ ID NO: 3922) |
| targeting miRNA | |
| binding site | |
| Second heart | AAACATCACTGCAAGTCTTAA (SEQ ID NO: 3925) |
| de-targeting | |
| miRNA binding | |
| site | |
| polyA signal | CTAGAGCTCGCTGATCAGCCTCGACTGTGCCTTCTAGTTGCCAGCCATCTGTTGTT |
| | TGCCCCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTCTGG |
| | GGGGTGGGGTGGGGCAGGACAGCAAGGGGGAGGATTGGGAAGAGAATAGCAGGCAT |
| | GCTGGGGA (SEQ ID NO: 3938) |
| AAV2 ITR | AGGAACCCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGCCTCAGT |
| | GAGCGAGCGAGCGCGC (SEQ ID NO: 3939) |

Nucleic acid 6
(SEQ ID NO: 3945)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCGGAAGAGAAGGTGACC
CTTACCCAGTTGTTCAACTCACCCTTCAGATTAAAAATAACTAAGGTAAGGGCCTGGGTAGGGGAGGTGGTGTGA
GAGGGTCCTGTCTCTCCTCTATCTGCCCATCGGCCCTTTGGGGAGGAGGAATGTGCCCAAGGACTAAAAAAAGGC
CCTGGAGCCAGAGGGGCGAGGGCAGCAGACCTTTCATGGGCAAACCTCAGGGCTGCTGGCCACTACGGGTCTAGG
CTGCCCATGTAAGGAGGCAAGGCCTGGGGACACCCGAGATGCCTGGTTATAATTAACCCAGACATGTGGCTGCCC
CCCCCCCCCCAACACCTGCTGCCTGCTAAAAATAACCCTGTCCCTGGTGGATATCAAGGCTGTGGGGGACTGAGG
GCAGGCTGTAACAGGCTTGGGGGCCAGGGCTTATACGTGCCTGGGACTCCCAAAGTATTACTGTTCCATGTTCCC
GGCGAAGGGCCAGCTGTCCCCCGCCAGCTAGACTCAGCACTTAGTTTAGGAACCAGTGAGCAAGTCAGCCCTTGG
GGCAGCCCATACAAGGCCATGGGGCTGGGCAAGCTGCACGCCTGGGTCCGGGGTGGGCACGGTGCCCGGGCAACG
AGCTGAAAGCTCATCTACTCTCAGGGGCCCCTCCCTGGGGACAGCCCCTCCTGGCTAGTCACACCCTGTAGGCTC
CTCTATATAACCCAGGGGCACAGGGGCTGCCCTCATTCTACCACCACCTCCACAGCACAGACAGACACTCACGAG
CCAGCCATCTACTGGGGACCAGCTATGGTAAGGGATGAGAGAAGCCAACAGTTGTCCCAAGGGTCCCCCTCAGGG
AGTGGAGGTTCCCCTGGGAGGCGCCAGGACAGGCGCAGGTGTATGAAACCAGATCTGAATCCTGGTGTGGCTCGG
AGGCCCAGGATTACGCTCTGGTTTCTAACAACTGAAGCCAGAGTCTTCGAAGGGTGGTGGGCTAAGGGGGAAGAG
GGCATGGTGGGCAAAAGAAAGGGGGTGGGGGAGAGAGAAAAGGAGGACAAGGGGGACTACTCCAACAAAATCACT
GATGCTGGAAATATTCGAAACCCAGCAGACAATGTAGCTTGCTTATCAACAAAATCACTGATGCTGGAACTCTAC
GAAACCCAGCAGACAATGTAGCTAGAATATCTAGAGCTCGCTGATCAGCCTCGACTGTGCCTTCTAGTTGCCAGC
CATCTGTTGTTTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAGGACAGCAAGG
GGGAGGATTGGGAAGAGAATAGCAGGCATGCTGGGGAAGGAACCCCTAGTGATGGAGTTGGCCACTCCCTCTCTG
CGCGCTCGCTCGCTCACTGAGGCCGGGCGACCAAAGGTCGCCCGACGCCCGGGCTTTGCCCGGGCGGCCTCAGTG
AGCGAGCGAGCGCGC

| Nucleic acid | |
|---|---|
| cargo 6 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | GGAAGAGAAGGTGACCCTTACCCAGTTGTTCAACTCACCCTTCAGATTAAAAATA |
| | ACTAAGGTAAGGGCCTGGGTAGGGGAGGTGGTGTGAGAGGGTCCTGTCTCTCCTC |
| | TATCTGCCCATCGGCCCTTTGGGGAGGAGGAATGTGCCCAAGGACTAAAAAAAGG |
| | CCCTGGAGCCAGAGGGGCGAGGGCAGCAGACCTTTCATGGGCAAACCTCAGGGCT |
| | GCTGGCCACTACGGGTCTAGGCTGCCCATGTAAGGAGGCAAGGCCTGGGGACACC |
| | CGAGATGCCTGGTTATAATTAACCCAGACATGTGGCTGCCCCCCCCCCCCCAACA |
| | CCTGCTGCCTGCTAAAAATAACCCTGTCCCTGGTGGATATCAAGGCTGTGGGGGA |
| | CTGAGGGCAGGCTGTAACAGGCTTGGGGGCCAGGGCTTATACGTGCCTGGGACTC |
| | CCAAAGTATTACTGTTCCATGTTCCCGGCGAAGGGCCAGCTGTCCCCCGCCAGCT |
| | AGACTCAGCACTTAGTTTAGGAACCAGTGAGCAAGTCAGCCCTTGGGGCAGCCCA |
| | TACAAGGCCATGGGGCTGGGCAAGCTGCACGCCTGGGTCCGGGGTGGGCACGGTG |
| | CCCGGGCAACGAGCTGAAAGCTCATCTACTCTCAGGGGCCCCTCCCTGGGGACAG |
| | CCCCTCCTGGCTAGTCACACCCTGTAGGCTCCTCTATATAACCCAGGGGCACAGG |
| | GGCTGCCCTCATTCTACCACCACCTCCACAGCACAGACAGACACTCACGAGCCAG |
| | C (SEQ ID NO: 3926) |
| upstream miR | GGGGACCAGCTATGGTAAGGGATGAGAGAAGCCAACAGTTGTCCCAAGGGTCCCC |
| scaffold | CTCAGGGAGTGGAGGTTCCCCTGGGAGGCGCCAGGACAGGCGCAGGTGTAT |
| | (SEQ ID NO: 3933) |
| guide sequence | GAAACCAGATCTGAATCCTGG (SEQ ID NO: 2869) |
| hairpin loop | TGTGGCTCGGAGGC (SEQ ID NO: 3934) |
| passenger | CCAGGATTACGCTCTGGTTTC (SEQ ID NO: 3630) |
| sequence | |
| downstream | TAACAACTGAAGCCAGAGTCTTCGAAGGGTGGTGGGCTAAGGGGGAAGAGGGCAT |
| scaffold | GGTGGGCAAAAGAAAGGGGGTGGGGGAGAGAGAAAAGGAGGACAAGGGGG (SEQ |
| | ID NO: 3935) |
| first DRG de- | CAACAAAATCACTGATGCTGGA (SEQ ID NO: 3921) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CAACAAAATCACTGATGCTGGA (SEQ ID NO: 3921) |
| targeting miRNA | |
| binding site | |
| Second heart | GAAACCCAGCAGACAATGTAGCT (SEQ ID NO: 3924) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

Nucleic acid 7
(SEQ ID NO: 3946)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCGTAGCTTTAGCCGGCT
TGCTTGCTTTTGGAGTCTGCCCCTCGTATGTACAGATCGGGGTCAGCCTGAGACGTGGGCGGGTTCCTTCCCAGG
CGTTGCAGCTCTCTTTCCTCCCTCACTCTCCAGTTCCTCTGGCTCAGTGAGGGATTTGGGATTTCTGTGTGTGGG
GCATGGACAGATACTACTCTTAGTGAAAAGCTGGACAAGGTGGAGTAGAGCATCAAGGGTGAGTGCCACTACGGG
TCTAGGCTGCCCATGTAAGGAGGCAAGGCCTGGGGACACCCGAGATGCCTGGTTATAATTAACCCAGACATGTGG
CTGCCCCCCCCCCCCCAACACCTGCTGCCTGCTAAAAATAACCCTGTCCCTGGTGGATATCAAGGCTGTGGGGGA
CTGAGGGCAGGCTGTAACAGGCTTGGGGGCCAGGGCTTATACGTGCCTGGGACTCCCAAAGTATTACTGTTCCAT
GTTCCCGGCGAAGGGCCAGCTGTCCCCCGCCAGCTAGACTCAGCACTTAGTTTAGGAACCAGTGAGCAAGTCAGC
CCTTGGGGCAGCCCATACAAGGCCATGGGGCTGGGCAAGCTGCACGCCTGGGTCCGGGGTGGGCACGGTGCCCGG
GCAACGAGCTGAAAGCTCATCTACTCTCAGGGGCCCCTCCCTGGGGACAGCCCCTCCTGGCTAGTCACACCCTGT
AGGCTCCTCTATATAACCCAGGGGCACAGGGGCTGCCCTCATTCTACCACCACCTCCACAGCACAGACAGACACT
CAGGAGCCAGCCAGCCATCTACTGGGGACCAGCTATGGTAAGGGATGAGAGAAGCCAACAGTTGTCCCAAGGGTC
CCCCTCAGGGAGTGGAGGTTCCCCTGGGAGGCGCCAGGACAGGCGCAGGTGTATGGCCTTCTGGCGGGCCGCGTC
TGTGGCTCGGAGGCGACGCGGCagGgCAGAAGGCCTAACAACTGAAGCCAGAGTCTTCGAAGGGTGGTGGGCTAA
GGGGGAAGAGGGCATGGTGGGCAAAAGAAAGGGGGTGGGGGAGAGAGAAAAGGAGGACAAGGGGGACTACTCTCA
TACAGCTAGATAACCAAAGAAATATTCGAAACCCAGCAGACAATGTAGCTTGCTTATCAACAAAATCACTGATGC
TGGAACTCTACAAACATCACTGCAAGTCTTAAAGAATATCTAGAGCTCGCTGATCAGCCTCGACTGTGCCTTCTA
GTTGCCAGCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTGGGGCAGG
ACAGCAAGGGGGAGGATTGGGAAGAGAATAGCAGGCATGCTGGGGAAGGAACCCCTAGTGATGGAGTTGGCCACT
CCCTCTCTGCGCGCTCGCTCGCTCACTGAGGCCGGGCGACCAAAGGTCGCCCGACGCCCGGGCTTTGCCCGGGCG
GCCTCAGTGAGCGAGCGAGCGCGC

| Nucleic acid | |
|---|---|
| cargo 7 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | GTAGCTTTAGCCGGCTTGCTTGCTTTTGGAGTCTGCCCCTCGTATGTACAGATCG |
| | GGGTCAGCCTGAGACGTGGGCGGGTTCCTTCCCAGGCGTTGCAGCTCTCTTTCCT |
| | CCCTCACTCTCCAGTTCCTCTGGCTCAGTGAGGGATTTGGGATTTCTGTGTGTGG |
| | GGCATGGACAGATACTACTCTTAGTGAAAAGCTGGACAAGGTGGAGTAGAGCATC |
| | AAGGGTGAGTGCCACTACGGGTCTAGGCTGCCCATGTAAGGAGGCAAGGCCTGGG |
| | GACACCCGAGATGCCTGGTTATAATTAACCCAGACATGTGGCTGCCCCCCCCCCC |
| | CCAACACCTGCTGCCTGCTAAAAATAACCCTGTCCCTGGTGGATATCAAGGCTGT |
| | GGGGGACTGAGGGCAGGCTGTAACAGGCTTGGGGGCCAGGGCTTATACGTGCCTG |
| | GGACTCCCAAAGTATTACTGTTCCATGTTCCCGGCGAAGGGCCAGCTGTCCCCCG |
| | CCAGCTAGACTCAGCACTTAGTTTAGGAACCAGTGAGCAAGTCAGCCCTTGGGGC |
| | AGCCCATACAAGGCCATGGGGCTGGGCAAGCTGCACGCCTGGGTCCGGGGTGGGC |
| | ACGGTGCCCGGGCAACGAGCTGAAAGCTCATCTACTCTCAGGGGCCCCTCCCTGG |
| | GGACAGCCCCTCCTGGCTAGTCACACCCTGTAGGCTCCTCTATATAACCCAGGGG |
| | CACAGGGGCTGCCCTCATTCTACCACCACCTCCACAGCACAGACAGACACTCAGG |
| | AGCCAGCCAGC (SEQ ID NO: 3927) |
| upstream miR | GGGGACCAGCTATGGTAAGGGATGAGAGAAGCCAACAGTTGTCCCAAGGGTCCCC |
| scaffold | CTCAGGGAGTGGAGGTTCCCCTGGGAGGCGCCAGGACAGGCGCAGGTGTAT |
| | (SEQ ID NO: 3933) |
| guide sequence | GGCCTTCTGGCGGGCCGCGTC (SEQ ID NO: 2429) |
| hairpin loop | TGTGGCTCGGAGGC (SEQ ID NO: 3934) |
| passenger | GACGCGGCagGgCAGAAGGCC (SEQ ID NO: 3190) |
| sequence | |
| downstream | TAACAACTGAAGCCAGAGTCTTCGAAGGGTGGTGGGCTAAGGGGGAAGAGGGCAT |
| scaffold | GGTGGGCAAAAGAAAGGGGGTGGGGGAGAGAGAAAAGGAGGACAAGGGGG (SEQ |
| | ID NO: 3935) |
| first DRG de- | TCATACAGCTAGATAACCAAAGA (SEQ ID NO: 3923) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CAACAAAATCACTGATGCTGGA (SEQ ID NO: 3921) |
| targeting miRNA | |
| binding site | |
| Second heart | AAACATCACTGCAAGTCTTAA (SEQ ID NO: 3925) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

Nucleic acid 8
(SEQ ID NO: 3947)
TCTCTGCGCGCTCGCTCGCTCACTGAGGCCGCCCGGGCAAAGCCCGGGCGTCGGGCGACCTTTGGTCGCCCGGCC
TCAGTGAGCGAGCGAGCGCGCAGAGAGGGAGTCATATAACATGAAGATCATCTGAATTCGTAGCTTTAGCCGGCT
TGCTTGCTTTTGGAGTCTGCCCCTCGTATGTACAGATCGGGGTCAGCCTGAGACGTGGGCGGGTTCCTTCCCAGG
CGTTGCAGCTCTCTTTCCTCCCTCACTCTCCAGTTCCTCTGGCTCAGTGAGGGATTTGGGATTTCTGTGTGTGGG
GCATGGACAGATACTACTCTTAGTGAAAAGCTGGACAAGGTGGAGTAGAGCATCAAGGGTGAGTGCCACTACGGG
TCTAGGCTGCCCATGTAAGGAGGCAAGGCCTGGGGACACCCGAGATGCCTGGTTATAATTAACCCAGACATGTGG
CTGCCCCCCCCCCCCCAACACCTGCTGCCTGCTAAAAATAACCCTGTCCCTGGTGGATATCAAGGCTGTGGGGGA
CTGAGGGCAGGCTGTAACAGGCTTGGGGGCCAGGGCTTATACGTGCCTGGGACTCCCAAAGTATTACTGTTCCAT
GTTCCCGGCGAAGGGCCAGCTGTCCCCCGCCAGCTAGACTCAGCACTTAGTTTAGGAACCAGTGAGCAAGTCAGC
CCTTGGGGCAGCCCATACAAGGCCATGGGGCTGGGCAAGCTGCACGCCTGGGTCCGGGGTGGGCACGGTGCCCGG
GCAACGAGCTGAAAGCTCATCTACTCTCAGGGGCCCCTCCCTGGGGACAGCCCCTCCTGGCTAGTCACACCCTGT
AGGCTCCTCTATATAACCCAGGGGCACAGGGGCTGCCCTCATTCTACCACCACCTCCACAGCACAGACAGACACT
CAGGAGCCAGCCAGCCATCTACTGGACCACCCAGCAGTGATGCACGTAGAGCAGAGGTGCAGTGATGCACGTACA
GCAGAGGCGACCCCTTCTCCATACTTCAGAGACCTCTAGCATGGTGTTGTGGGACCCGGTGTGGGCGAAGGCGAC
CTTGCCAATCAGAGAAGGTCGCCTaaGqCCACACCGGCCACACTGCACTGCAAGCAGCGAGCAGCAAGCAACAAG
CATCCAGACACCTGAGCACCAGGATGCCAGCAAGAGCACCTGCCTGTGAGGAAACCTCCCAGCCACTACTCTCAT
ACAGCTAGATAACCAAAGAAATATTCGAAACCCAGCAGACAATGTAGCTTGCTTATCAACAAAATCACTGATGCT
GGAACTCTACAAACATCACTGCAAGTCTTAAAGAATATCTAGAGCTCGCTGATCAGCCTCGACTGTGCCTTCTAG
TTGCCAGCCATCTGTTGTTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGGGGGTGGGGTGGGGCAGGA
CAGCAAGGGGGAGGATTGGGAAGAGAATAGCAGGCATGCTGGGGAAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGC

| Nucleic acid | |
|---|---|
| cargo 8 | |
| annotated | Sequence |
| Self- | TCTCTGCGCGCTCGCTCGCTCACTGAGGCCGCCCGGGCAAAGCCCGGGCGTCGGG |
| complimentary | CGACCTTTGGTCGCCCGGCCTCAGTGAGCGAGCGAGCGCGCAGAGAGGGAGT |
| AAV2 ITR | (SEQ ID NO: 3937) |
| promoter | GTAGCTTTAGCCGGCTTGCTTGCTTTTGGAGTCTGCCCCTCGTATGTACAGATCG |
| | GGGTCAGCCTGAGACGTGGGCGGGTTCCTTCCCAGGCGTTGCAGCTCTCTTTCCT |
| | CCCTCACTCTCCAGTTCCTCTGGCTCAGTGAGGGATTTGGGATTTCTGTGTGTGG |
| | GGCATGGACAGATACTACTCTTAGTGAAAAGCTGGACAAGGTGGAGTAGAGCATC |
| | AAGGGTGAGTGCCACTACGGGTCTAGGCTGCCCATGTAAGGAGGCAAGGCCTGGG |
| | GACACCCGAGATGCCTGGTTATAATTAACCCAGACATGTGGCTGCCCCCCCCCCC |
| | CCAACACCTGCTGCCTGCTAAAAATAACCCTGTCCCTGGTGGATATCAAGGCTGT |
| | GGGGGACTGAGGGCAGGCTGTAACAGGCTTGGGGGCCAGGGCTTATACGTGCCTG |
| | GGACTCCCAAAGTATTACTGTTCCATGTTCCCGGCGAAGGGCCAGCTGTCCCCCG |
| | CCAGCTAGACTCAGCACTTAGTTTAGGAACCAGTGAGCAAGTCAGCCCTTGGGGC |
| | AGCCCATACAAGGCCATGGGGCTGGGCAAGCTGCACGCCTGGGTCCGGGGTGGGC |
| | ACGGTGCCCGGGCAACGAGCTGAAAGCTCATCTACTCTCAGGGGCCCCTCCCTGG |
| | GGACAGCCCCTCCTGGCTAGTCACACCCTGTAGGCTCCTCTATATAACCCAGGGG |
| | CACAGGGGCTGCCCTCATTCTACCACCACCTCCACAGCACAGACAGACACTCAGG |
| | AGCCAGCCAGC (SEQ ID NO: 3927) |
| upstream miR | GGACCACCCAGCAGTGATGCACGTAGAGCAGAGGTGCAGTGATGCACGTACAGCA |
| scaffold | GAGGCGACCCCTTCTCCATACTTCAGAGACCTCTAGCATGGTGTTGTGGGAC |
| | (SEQ ID NO: 3929) |
| guide sequence | CCGGTGTGGGCGAAGGCGACC (SEQ ID NO: 2475) |
| hairpin loop | TTGCCAATCAGAGAA (SEQ ID NO: 3930) |
| passenger | GGTCGCCTaaGgCCACACCGG (SEQ ID NO: 3236) |
| sequence | |
| downstream | CCACACTGCACTGCAAGCAGCGAGCAGCAAGCAACAAGCATCCAGACACCTGAGC |
| scaffold | ACCAGGATGCCAGCAAGAGCACCTGCCTGTGAGGAAACCTCCCAGCC (SEQ ID |
| | NO: 3931) |
| first DRG de- | TCATACAGCTAGATAACCAAAGA (SEQ ID NO: 3923) |
| targeting miRNA | |
| binding site | |
| First heart de- | GAAACCCAGCAGACAATGTAGCT (SEQ ID NO: 3924) |
| targeting miRNA | |
| binding site | |
| second DRG de- | CAACAAAATCACTGATGCTGGA (SEQ ID NO: 3921) |
| targeting miRNA | |
| binding site | |
| Second heart | AAACATCACTGCAAGTCTTAA (SEQ ID NO: 3925) |
| de-targeting | |
| miRNA binding | |
| site | |
| polyA signal | CTAGAGCTCGCTGATCAGCCTCGACTGTGCCTTCTAGTTGCCAGCCATCTGTTGT |
| | TTGCCCCTCCCCCGTGCCTTCCTTGACCCTGGAAGGTGCCACTCCCACTGTCCTT |
| | TCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGAGAATAGCAG |
| | GCATGCTGGGGA (SEQ ID NO: 3938) |
| AAV2 ITR | AGGAACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCGGCCTCA |
| | GTGAGCGAGCGAGCGCGC (SEQ ID NO: 3939) |

### Example 2: Materials and Methods

The following Materials and Methods were used in experiments described herein.

Cell lines: HEK293 was purchased from ATCC (ATCC, CRL-1573). FSHD patient myoblasts were obtained from the Leiden Institute.

Cynomolgus Macaques: Juvenile male non-human primates were injected intravenously with Composition 1 or Composition 2 at the indicated dose.

Gene Expression: DUX4 or DUX4 target gene (ZSCAN4, CCNA1, MBD3L2, TRIM43, KHDC1L) expression was quantified using a standard tagman assay. Expression was normalized to gene expression in cells treated with a scrambled control sequence or mice treated with vehicle.

Mice: Bitransgenic ACTA1-MCM and FLExDUX4 animals were purchased from JAX and bred internally to generate the ACTA-MCM;FLExDUX4 animals. Mice were injected retro-orbitally with Composition 1 or Composition 2 at the indicated dose.

Small RNASeq and Off-Target Analysis: RNA was extracted using an Trizol (Invitrogen, 15596026). For small RNAseq, sequencing libraries were prepared using TruSeq Small RNA Library Prep Kits (Illumina, RS-200-0012), gel extracted using the QIAquick Gel Extraction Kit (Qiagen, 28704), then pooled and sequenced on an Illumina Nextseq 2000 platform using Nextseq 2000 P2 reagents (Illumina, 20046811). Manufacturer protocols were used for all kits.

Tissue Staining: Muscles harvested from mice were frozen in liquid nitrogen-cold isopentane immediately after dissection. Tissues were sectioned using CM1860 cryostat (Leica Biosciences) at the thickness of 12 mm. Tissues were stained with hematoxylin and eosin (H&E) or primary antibodies (primary antibodies specific for, embryonic myosin heavy chain, wheat germ agglutinin (WGA), or laminin) and fluorescently labeled secondary antibodies.

Treadmill Assessments: For treadmill runs, multiple days of acclimation were performed prior to data collection. During the testing phase the treadmill was set to a 10° incline with the initial speed set to 5 m/min during the first minute. Speed was increased by 0.5 m/min each minute for up to 20 additional minutes. The maximum speed reached was 15 m/min with a maximum distance of approximately 200 m. Signs of exhaustion were considered as either three consecutive visits of two seconds or more on the shock grid without returning to the treadmill or greater than five seconds continuously spent on the shock grid.

Vector Genome Quantification: For the vector genome quantification experiments, total DNA was extracted from tissue samples and DNA concentration was determined by Qubit dsDNA high-sensitivity assay kit (Thermo Fisher Scientific) using the QUBIT™ 4.0 Fluorometer (ThemoFisher Scientific). DNA was diluted to 0.5 ng/μL.

The VCN was quantified by digital droplet polymerase chain reaction (ddPCR) using ddPCR Supermix for Probes (no dUTP) (Bio-Rad Laboratories, Inc.) and an assay targeting a region in the viral genome. The VCN was normalized by the number of diploid genomes using the endogenous mouse telomerase gene (mTERT). All primers and probes were synthesized by Thermo Fisher Scientific. The ddPCR was performed using the QX200 ddPCR system (Bio-Rad Laboratories, Inc).

### Example 3: Identification of Candidate miRNA Sequences Targeting DUX4

Stringent in vitro and in vivo screening was utilized to identify the most potent and selective miRNA knockdown sequences to target DUX4, resulting in the identification of the top miRNA knockdown sequence.

Greater than 14,000 candidate miRNA sequences were screened in a cell-based DUX4 knockdown assay. In brief, a HEK293 cell line stably expressing mCherry-tagged DUX4 (Hox1 mutant) was generated. The DUX4 (Hox1 mutant), in which the first homeobox DNA-binding domain is mutated, showed remarkably reduced toxicity compared to the full-length DUX4, and was therefore used in the cell-based DUX4 knockdown assay. HEK293 cells stably expressing mCherry-tagged DUX4 (Hox1 mutant) were transduced with GFP-tagged DUX4 miRNA library sequences. Cells were analyzed by fluorescence-activated cell sorting (FACS) to identify GFP⁺, mCherry (low) and GFP⁺, mCherry (high) cell populations. Next generation sequencing (NGS) was used to identify the top miRNA sequences. An in silico selection of candidate sequences was conducted to exclude sequences that had a perfect 21-mer match to an off-target RNA in human cells. The top 13 lead sequences based on potency (DUX4 knockdown) were identified. The 13 top miRNA sequences were identified based on their enrichment in the GFP⁺ and mCherry (low) cell population. miRNA #6 sequence was included as a positive control.

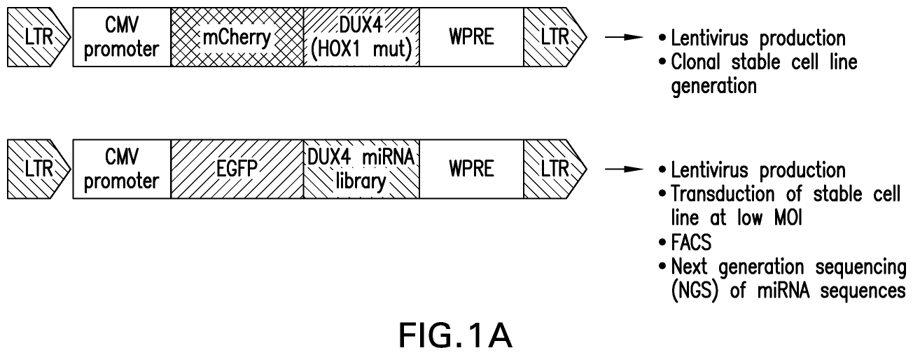

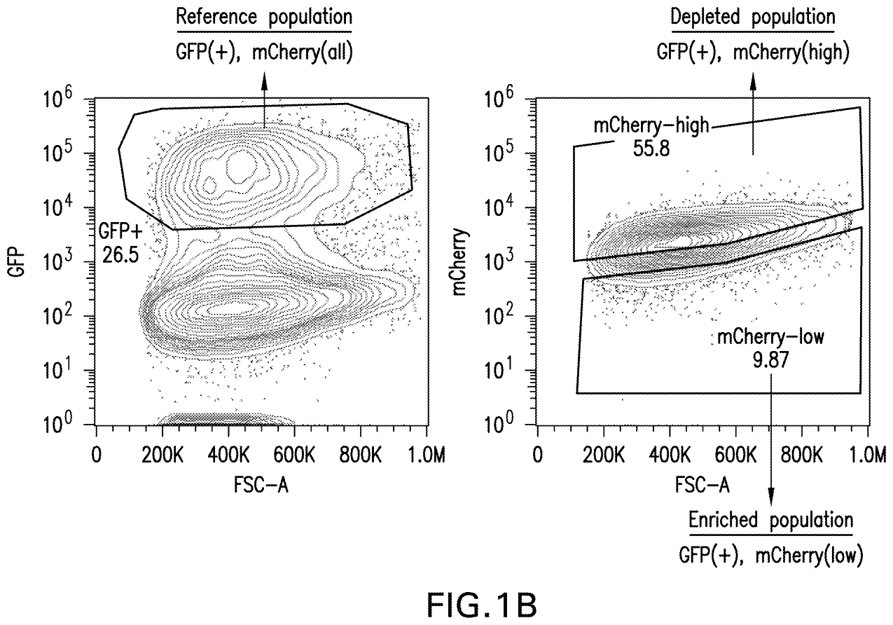

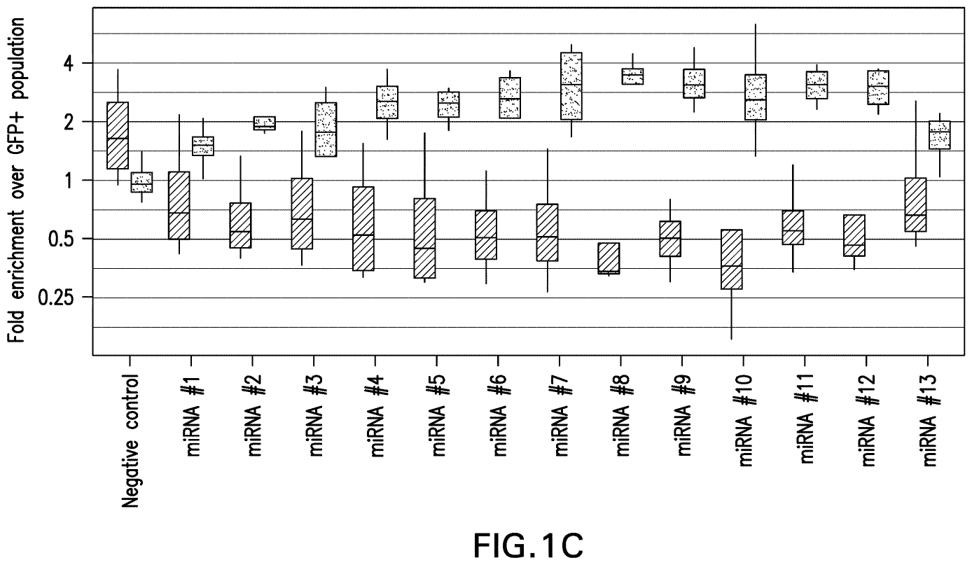

FIG. 1A shows a schematic of lentiviral constructs used for screening miRNA sequences targeting DUX4. FIG. 1B shows flow cytometry workflow and selection of positive and negative cell populations. FIG. 1C shows enrichment over the GFP⁺ population of the top 13 miRNA sequences targeting DUX4.

### Example 4: Candidate miRNA Sequences Effectively Knockdown DUX4 and Reduce Expression of hZSCAN4 in FSHD1 Patient Myotubes

Knockdown of DUX4 by the top 13 candidate miRNA sequences was individually tested in FSHD patient myotubes. Expression of DUX4 and hZSCAN4 (a DUX4 downstream gene) was measured in FSHD patient myotubes transduced with one of the 13 candidate miRNA sequences, which were tested at 3 different doses (1E+4 vg/cell, 3E+4 vg/cell, 1E+5 vg/cell). Data was normalized to expression data from FSHD patient myotubes transduced with a scrambled control sequence.

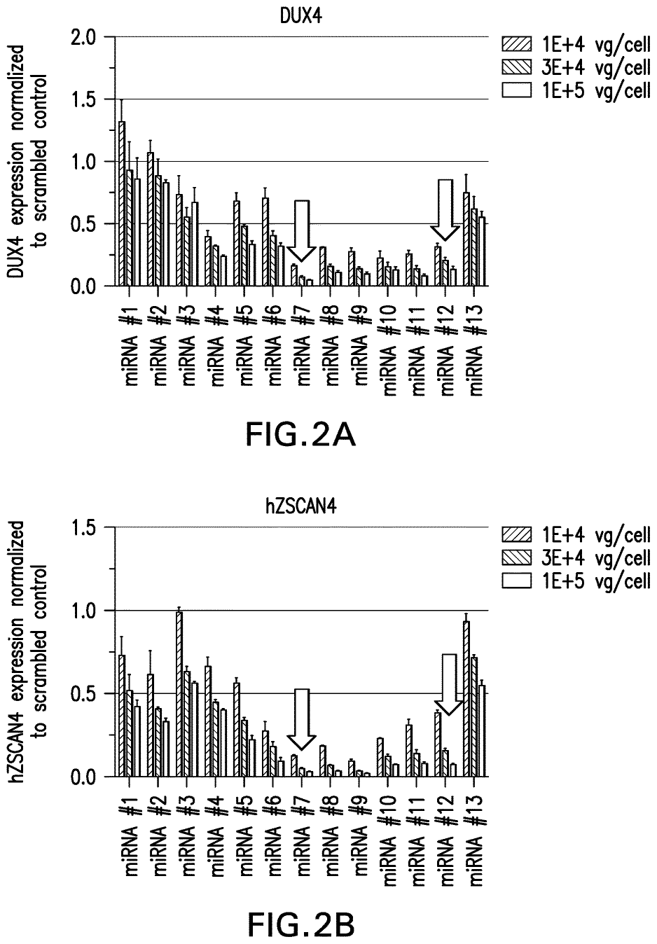

FIGS. 2A-2B show graphs of DUX4 expression (FIG. 2A) and hZSCAN4 expression (FIG. 2B) in FSHD patient myotubes transduced with candidate miRNA knockdown sequences, including the miRNA #7 (SEQ ID NO: 2869) and miRNA #12 (SEQ ID NO: 3159).

These results demonstrate that effective knockdown of DUX4 and the downstream gene hZSCAN4 can be achieved using the candidate miRNA sequences. In these assays, the most significant knockdown of DUX4 was achieved using miRNA #7, miRNA #8, miRNA #9, miRNA #10, miRNA #11, or miRNA #12. These results also demonstrate that significantly greater knockdown of DUX4 was achieved using the candidate miRNA sequences (miRNA #7, miRNA #8, miRNA #9, miRNA #10, miRNA #11, or miRNA #12) compared to the positive control sequence (miRNA #6).

TABLE 3
Top miRNA Sequences Targeting DUX4 tested in vitro

| miRNA | |
|---|---|
| Sequences | |
| Targeting | |
| DUX4 | Sequence |
| miRNA #1 | TCCGTCGCCGTCCTCGTCCCC (SEQ ID NO: 2743) |
| miRNA #2 | TCCAAACGAGTCTCCGTCGCC (SEQ ID NO: 2783) |
| miRNA #3 | GTCCAAACGAGTCTCCGTCGC (SEQ ID NO: 2791) |
| miRNA #4 | AGATCTGAATCCTGGACTCCG (SEQ ID NO: 2831) |
| miRNA #5 | CAGATCTGAATCCTGGACTCC (SEQ ID NO: 2839) |
| miRNA #6 | AACCAGATCTGAATCCTGGAC (SEQ ID NO: 3954) |
| miRNA #7 | GAAACCAGATCTGAATCCTGG (SEQ ID NO: 2869) |
| miRNA #8 | TGAAACCAGATCTGAATCCTG (SEQ ID NO: 2871) |
| miRNA #10 | TTCTGAAACCAGATCTGAATC (SEQ ID NO: 3952) |
| miRNA #11 | TCGATTCTGAAACCAGATCTG (SEQ ID NO: 3953) |
| miRNA #12 | TTCGATTCTGAAACCAGATCT (SEQ ID NO: 3159) |
| miRNA #13 | CCACCGAGGAGCCTGAGGGTG (SEQ ID NO: 2935) |

### Example 5: Candidate miRNA Sequences do not Result in Off-Target Effects Based on RNAseq

RNAseq was used to measure off-target expression changes in human myotubes transduced with miRNA #7 (SEQ ID NO: 2869) or miRNA #12 (SEQ ID NO: 3159). Myotubes transduced with a scrambled sequence were used as a control to identify genes differentially expressed in the presence of miRNA #7 or miRNA #12.

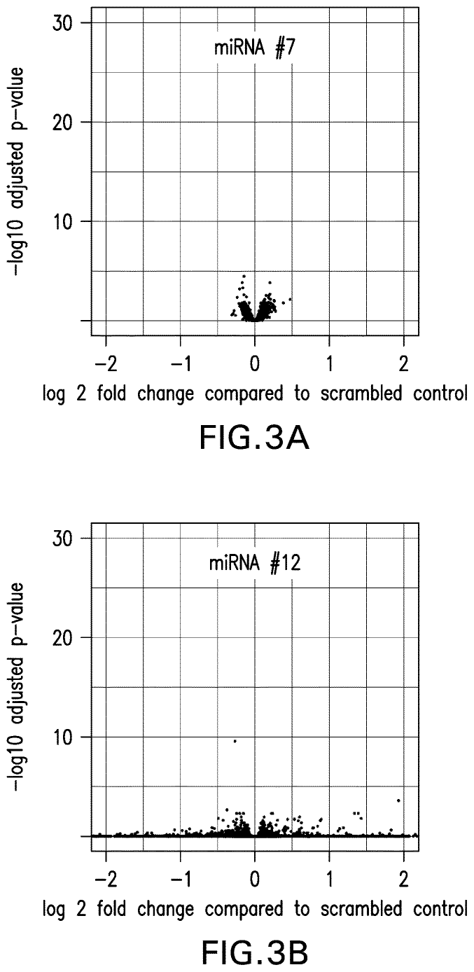

FIGS. 3A-3B show volcano plots of differentially expressed genes in human primary myotubes transduced with miRNA #7 (FIG. 3A) or miRNA #12 (FIG. 3B). These results demonstrate that the miRNA #7 and miRNA #12 sequences showed no detectable off-target effects in human primary myotubes.

### Example 6: Candidate miRNA Sequences Effectively Knockdown DUX4 and Reduce Expression of DUX4 Downstream Genes in a FSHD Mouse Model

The top 5 candidate miRNA sequences (miRNA #7, miRNA #8, miRNA #10, miRNA #11, and miRNA #12, Table 3) were tested in ACTA1-MCM;FLExDUX4 mice, which is a mouse model of FSHD. MCM;FLExDUX4 mice express DUX4 and DUX4 downstream genes, such as Kif4, following tamoxifen induction. Mice were treated with a candidate miRNA sequence or vehicle as a control.

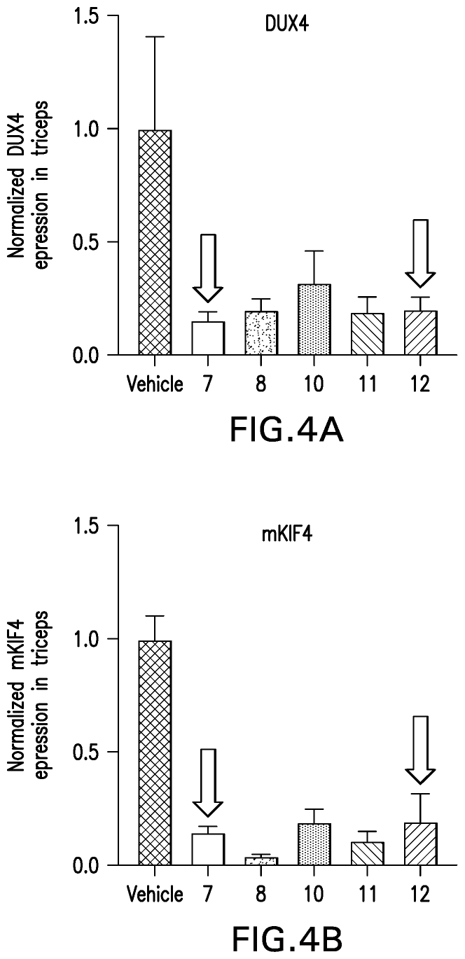

FIGS. 4A-4B show graphs of DUX4 expression (FIG. 4A) and mKif4 expression (FIG. 4B) in mouse triceps following administration of a candidate miRNA sequence. DUX4 and mKif4 expression was reduced in mice administered with a candidate miRNA sequence compared to mice administered with vehicle (FIGS. 4A-4B). Thus, in vivo efficacy and potency was confirmed for the candidate miRNA sequences including the miRNA #7 and miRNA #12 sequences.

TABLE 4
Top miRNA Sequences Targeting DUX4 tested in vivo

| miRNA | |
|---|---|
| Sequences | |
| Targeting | |
| DUX4 | Sequence |
| miRNA #7 | GAAACCAGATCTGAATCCTGG (SEQ ID NO: 2869) |
| miRNA #8 | TGAAACCAGATCTGAATCCTG (SEQ ID NO: 2871) |
| miRNA #10 | TTCTGAAACCAGATCTGAATC (SEQ ID NO: 3952) |
| miRNA #11 | TCGATTCTGAAACCAGATCTG (SEQ ID NO: 3953) |
| miRNA #12 | TTCGATTCTGAAACCAGATCT (SEQ ID NO: 3159) |

### Example 7: Composition 1 and Composition 2 Effectively Knockdown DUX4 and Reduce Expression of DUX4 Downstream Genes in FSHD1 Patient Myotubes Regardless of Timing of Transduction

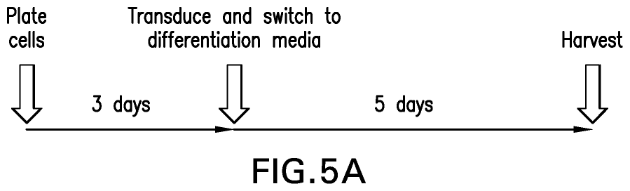

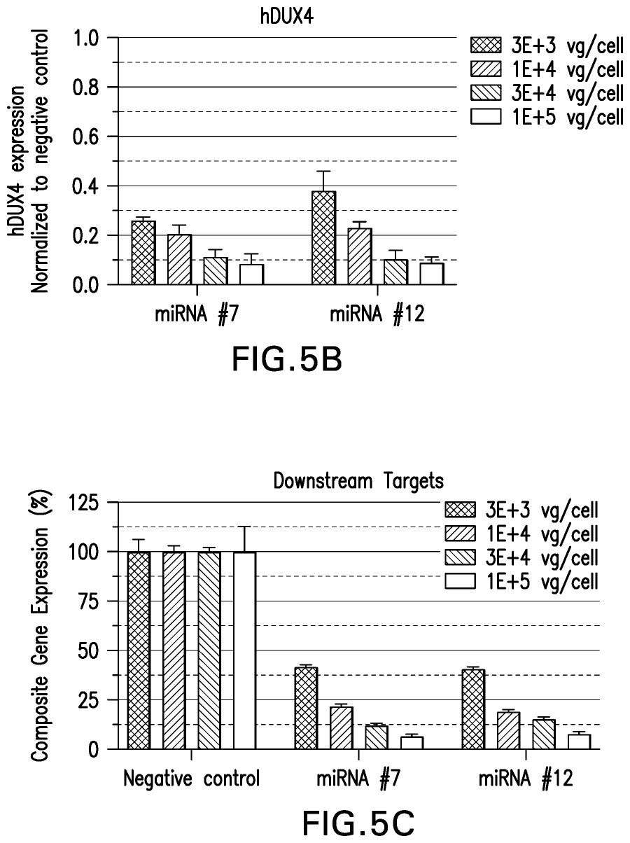

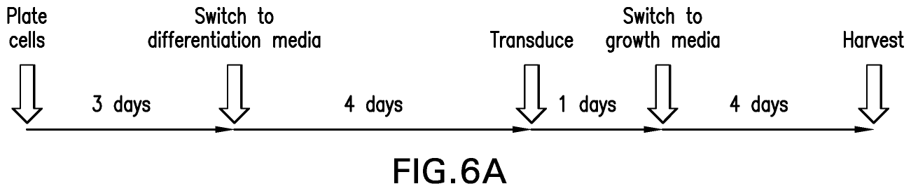

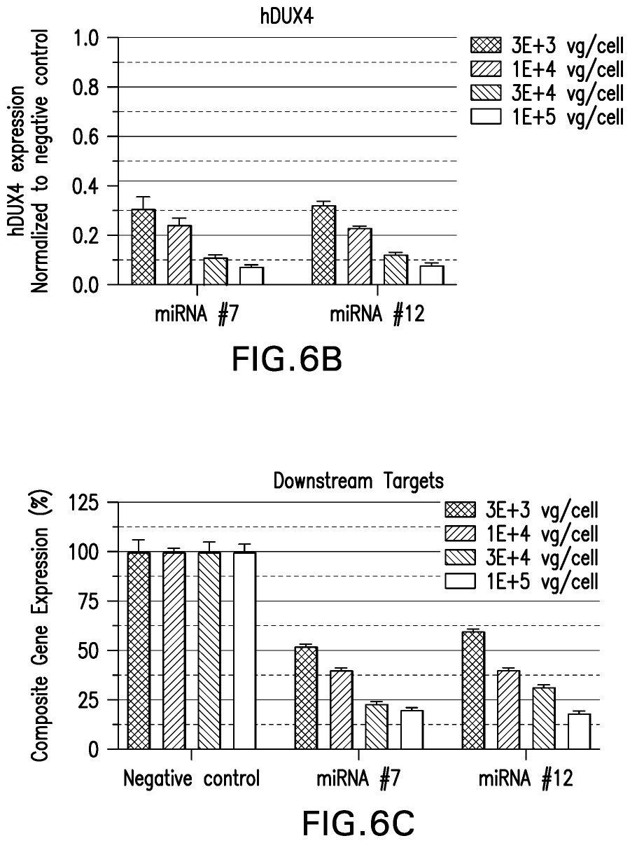

Expression of DUX4 and DUX4 downstream genes (also referred to as FSHD composite gene expression) was measured in FSHD patient myotubes transduced with Composition 1 (miRNA #7; SEQ ID NO: 2869) or Composition 2 (miRNA #12; SEQ ID NO: 3159) encapsidated in MyoAAV-LD B. FSHD composite gene expression was calculated by averaging normalized expression of several DUX4 downstream genes including CCNA1, MBD3L2, ZSCAN4, TRIM43, and KHDC1L. Expression data was normalized to expression data from cells transduced with a scrambled sequence. Transduction was performed at the time of inducing differentiation (FIGS. 5A-5C) or 4 days after differentiation (FIGS. 6A-6C).

FIG. 5A shows a study design in FSHD patient myotubes in which transduction is performed at the time of differentiation. Cells from muscles of FSHD patients were plated and, after 3 days, cells were transduced with different doses (3E+3 vg/cell, 1E+4 vg/cell, 3E+4 vg/cell, 1E+5 vg/cell) of the lead and backup candidate miRNAs and switched to differentiation media at the same time. After 5 days, cells were harvested and human DUX4 (hDUX4) expression as well as expression of DUX4 target genes was quantified and normalized to control cells transduced with a scrambled sequence. FIGS. 5B-5C show graphs of DUX4 expression (FIG. 5B) and DUX4 composite gene expression (FIG. 5C) in FSHD patient myotubes transduced with the lead or backup miRNA sequences at the time of differentiation.

These results demonstrate that treatment of FSHD patient myotubes with the lead or the backup miRNA sequence at the time of differentiation can reduce DUX4 and DUX4 downstream target gene expression in a dose dependent manner.

FIG. 6A shows a study design in FSHD patient myotubes in which transduction is performed after differentiation. Cells from muscles of FSHD patients were plated and switched to differentiation media after 3 days. After 4 days in differentiation media, cells were transduced with different doses (3E+3 vg/cell, 1E+4 vg/cell, 3E+4 vg/cell, 1E+5 vg/cell) of Composition 1 or Composition 2. 1 day after transduction, media was switched to a growth media. After 4 days in growth media, cells were harvested and human DUX4 (hDUX4) as well as DUX4 target gene expression was quantified and normalized to control cells transduced with a scrambled sequence. FIGS. 6B-6C show graphs of DUX4 expression (FIG. 6B) and DUX4 composite gene expression (FIG. 6C) in FSHD patient myotubes transduced with Composition 1 or Composition 2 after differentiation.

These results demonstrate that treatment of FSHD patient myotubes with Composition 1 or Composition 2 after differentiation can reduce DUX4 and DUX4 downstream target gene expression in a dose dependent manner.

Taken together, these results demonstrate that Composition 1 and Composition 2 can effectively decrease expression of DUX4 and DUX4 downstream target genes, regardless of the timing of transduction of the candidate miRNA. Thus, Composition 1 and Composition 2 can protect primary and differentiated myotubes from DUX4-mediated toxicity.

### Example 8: Lead miRNA Sequences does not Result in Off-Target Effects Based on RNAseq and Small RNAseq

RNAseq and small RNAseq were performed to assess differential gene and endogenous miRNA expression between myotubes transduced with the lead candidates or a scrambled control.

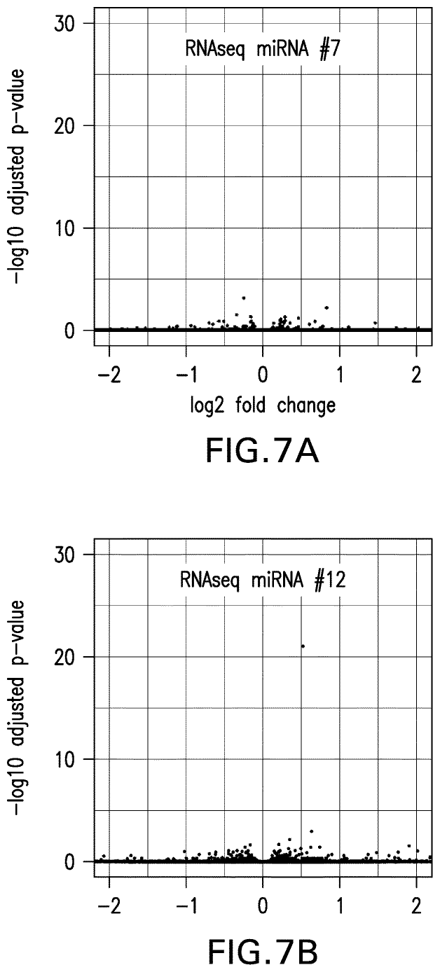

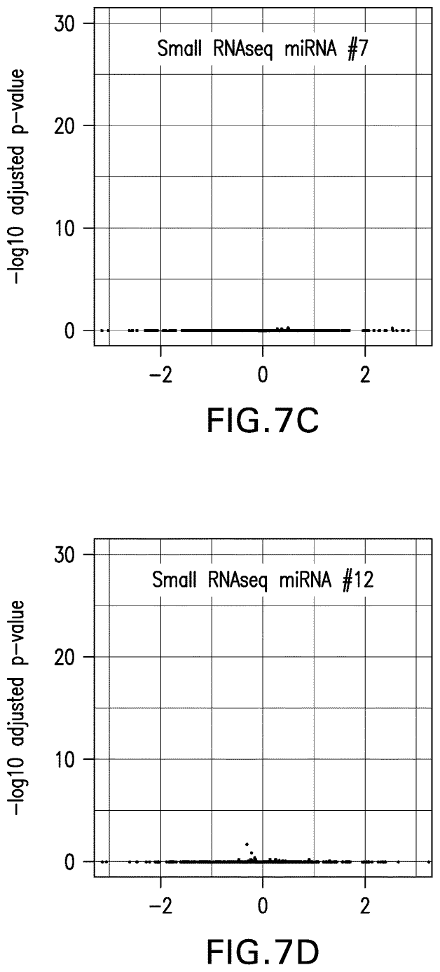

FIGS. 7A-7B show volcano plots of differentially expressed genes and differentially expressed miRNAs in human myotubes transduced with Composition 1, Composition 2 or a scrambled control based on RNAseq (FIG. 7A-7B) and small RNAseq (FIG. 7C-7D).

No endogenous mRNA or miRNAs met criteria of adjusted p<0.001, 50% increase or decrease in expression. These results demonstrate that the miRNAs encoded by Composition 1 and Compostions 2 do not result in off-target effects, such as altering off-target gene expression or altering miRNA expression, in human myotubes.

### Example 9: a Tool Compound of Composition 1 Protects Against DUX4 Induced Damage in a Moderate Mouse Model of FSHD

A tool compound of Composition 1 was tested in a mouse model of FSHD, specifically ACTA1-MCM;FLExDUX4 mice in which tunable levels of DUX4 and DUX4 downstream gene expression can be induced with tamoxifen (higher doses of tamoxifen induce high levels of DUX4 and DUX4 downstream gene expression corresponding to a more severe model of FSHD). The tool compound of Composition 1 differs from Composition 1 in that the tool compound of Composition 1 includes restriction sites that were removed from Composition 1.

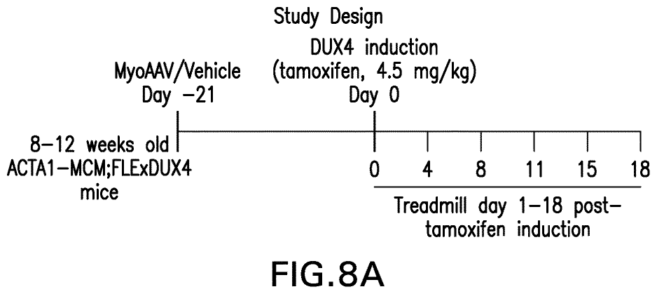

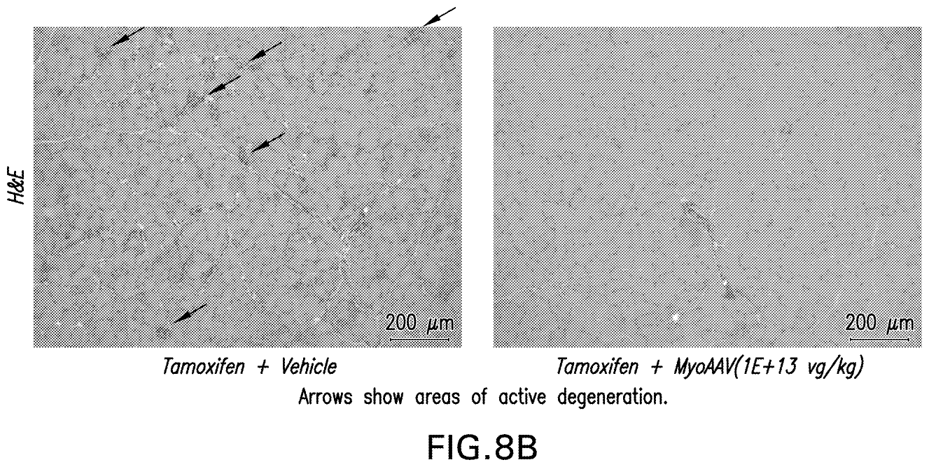

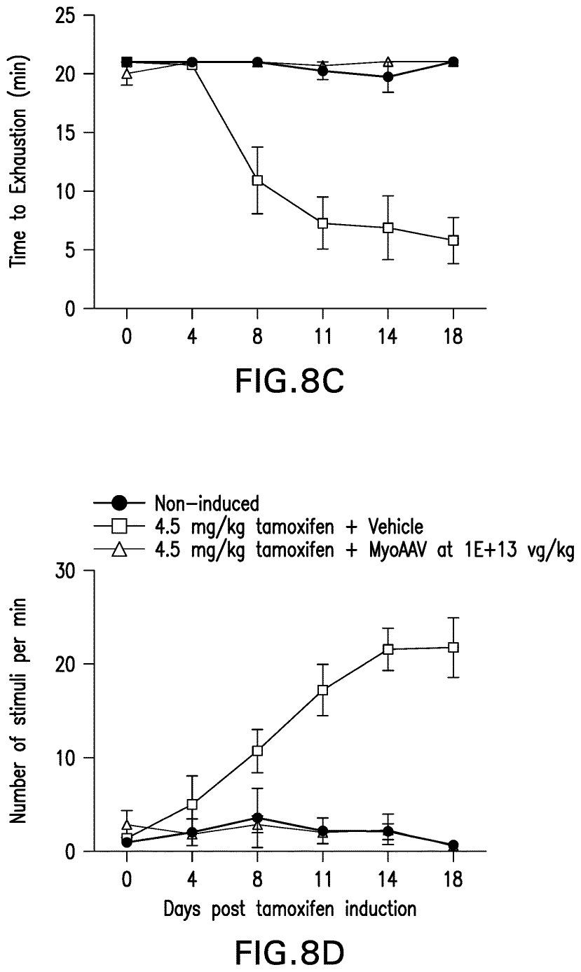

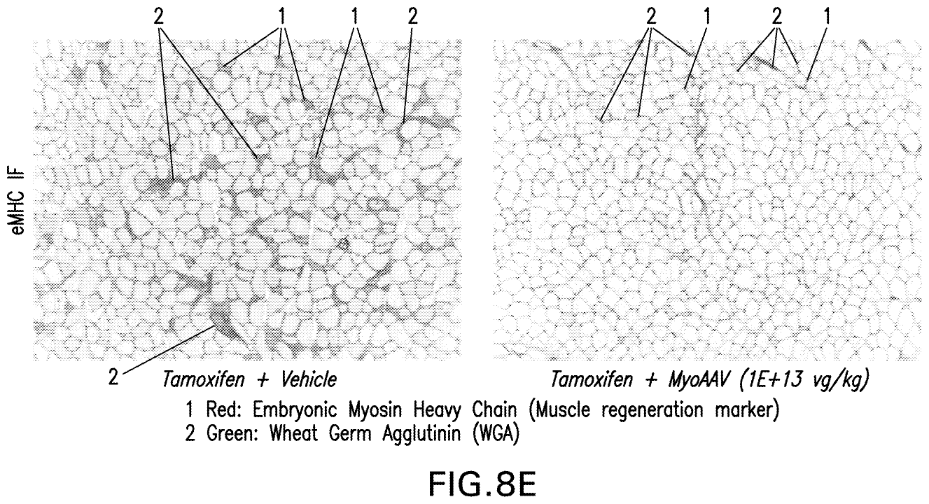

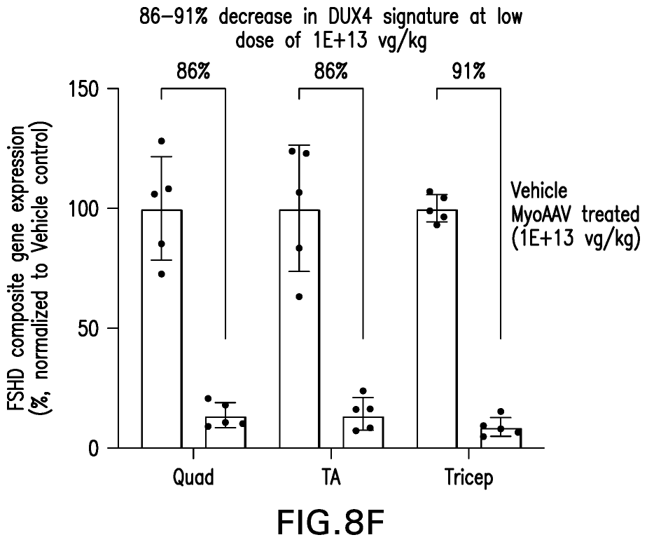

A tool compound of Composition 1 (dosed at 1E+13 vg/kg) or vehicle was administered to 8-12 week old ACTA1-MCM;FLExDUX4 mice. After 21 days, mice were treated with tamoxifen (4.5 mg/kg) to induce DUX4 expression. Mice were assessed on a treadmill on days 1-18 post-tamoxifen induction. Mice were necropsied on day 19 post-taxmoxifen induction and skeletal muscles were assessed for DUX4 downstream gene expression and muscle regeneration. FIG. 8A shows a study design for testing a tool compound of Composition 1 in a moderate mouse model of FSHD.

FIG. 8B shows representative images of skeletal muscle from mice treated with a tool compound of Composition 1 or vehicle. Muscle tissues were stained with hematoxylin and eosin (H&E). Active muscle degeneration following tamoxifen induced DUX4 expression was observed in mice administered vehicle (FIG. 8B, left panel), but not mice administered with a tool compound of Composition 1 (FIG. 8B, right panel). Arrows show an area of active degeneration. These results demonstrate that the administration of the lead candidate prevented visible muscle degeneration in a moderate mouse model of FSHD.

FIGS. 8C-8D show graphs of results from treadmill assessments of mice administered with a tool compound of Composition 1 or vehicle prior to tamoxifen induced DUX4 expression. As shown in FIGS. 8C-8D, administration of a tool compound of Composition 1 prevented a decrease in time to exhaustion (FIG. 8C) and a decrease in number of stimuli per minute (FIG. 8D) in a moderate mouse model of FSHD. Mice treated with a tool compound of Composition 1 showed similar time to exhaustion and number of stimuli per minute as non-induced mice (FIGS. 8C-8D).

FIG. 8E shows immunofluorescent images of mouse skeletal muscle stained for embryonic myosin heavy chain (marker of muscle regeneration) and wheat germ agglutinin (WGA; marker of extracellular matrix and connective tissue in skeletal muscle). As shown in FIG. 8E, Mice injected with a tool compound of composition 1 had significantly less embroyonic myosin heavy chain in analyzed tissues indicating that administration of the lead candidate prevented the need for muscle regeneration in mouse tissue.

FIG. 8F shows a graph of FSHD composite gene expression in skeletal muscle (quadriceps, tibialis anterior (TA), triceps) of mice administered a tool compound of Composition 1 or vehicle prior to tamoxifen induced DUX4 expression. As shown in FIG. 8F, administration of a tool compound of Composition 1 resulted in an 86-91% decrease in FSHD composite gene expression in mouse skeletal muscles including quadriceps, TA, and triceps.

Taken together, these results demonstrate that mice injected with a tool compound of Composition 1 showed improved muscle histology and function (e.g., less active degeneration, longer time to exhaustion, lower number of stimuli received per minute, lower muscle regeneration marker, and lower FSHD composite gene expression) compared to vehicle-injected mice. Thus, these results demonstrate that a tool compound of Composition 1 can protect against DUX4 induced muscle damage in a moderate mouse model of FSHD.

### Example 10: Administration of Composition 1 Results in Dose-Dependent Protection Against DUX4 Induced Damage in a Severe Mouse Model of FSHD

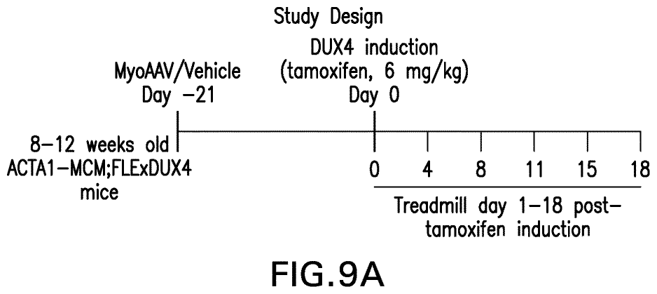

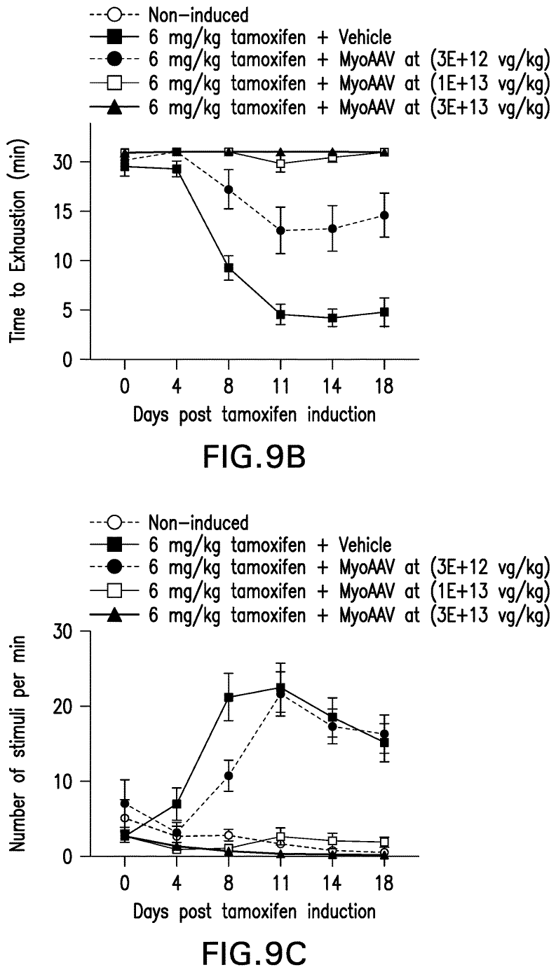

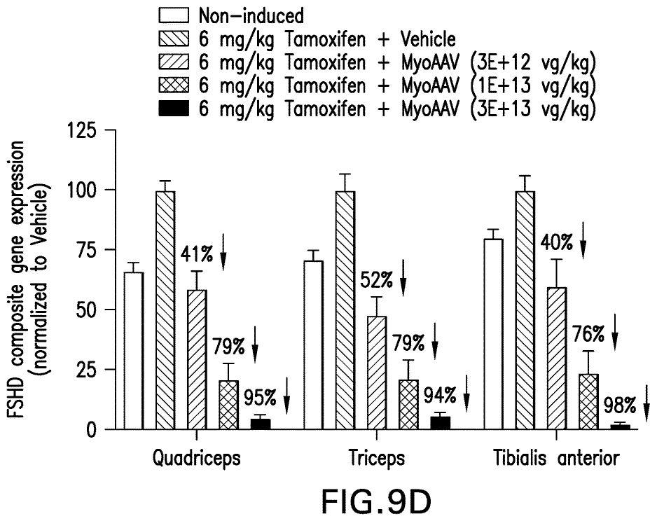

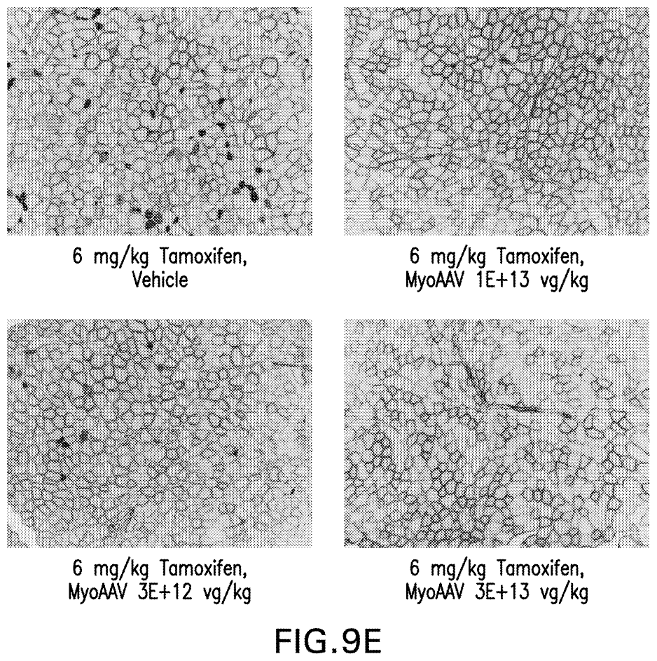

Composition 1 (dosed at 3E+12 vg/kg, 1E+13 vg/kg, or 3E+13 vg/kg) or vehicle was administered to 8-12 week old ACTA1-MCM;FLExDUX4 mice. After 21 days, mice were treated with tamoxifen (6 mg/kg) to induce DUX4 expression. Mice were assessed on a treadmill on days 1-18 post-tamoxifen induction. Mice were necropsied on day 19 post-taxmoxifen induction and skeletal muscles were assessed for DUX4 downstream gene expression and muscle regeneration. FIG. 9A shows a study design for testing Composition 1 in a severe mouse model of FSHD.

FIGS. 9B-9C show graphs of results from treadmill assessments of mice administered Composition 1 (dosed at 3E+12 vg/kg, 1E+13 vg/kg, or 3E+13 vg/kg) or vehicle prior to tamoxifen induced DUX4 expression. As shown in FIGS. 9B-9C, administration of Composition 1 showed a dose escalating response in preventing a decrease in time to exhaustion (FIG. 9B) and number of stimuli per minute (FIG. 9C) in a severe mouse model of FSHD.

FIG. 9D shows a graph of FSHD composite gene expression in skeletal muscle (quadriceps, triceps, tibialis anterior) of mice administered Composition 1 or vehicle prior to tamoxifen induced DUX4 expression. As shown in FIG. 9D, administration of Composition 1 showed a dose escalating response in decreasing expression of DUX4 target genes in muscles of ACTA1-MCM;FLExDUX4 mice.

FIG. 9E shows immunofluorescent images of mouse skeletal muscle stained for embryonic myosin heavy chain (marker of muscle regeneration) and laminin (marker outlining individual muscle fibers). As shown in FIG. 9E, administration of Composition 1 showed a dose escalating response in preventing the need for muscle regeneration in mouse tissue.

Taken together, these results demonstrate that Composition 1-injected mice showed a dose-dependent improvement in muscle histology and function (e.g., less active degeneration, longer time to exhaustion, lower number of stimuli received per minute, lower muscle regeneration marker, and lower FSHD composite gene expression) compared to vehicle-injected mice. Thus, these results demonstrate that Composition 1 can provide dose-dependent protection against DUX4 induced muscle damage in a severe mouse model of FSHD.

### Example 11: Composition 1 Protects Against DUX4 Induced Damage in a Mild Mouse Model of FSHD

Composition 1 was tested in a mild mouse model of FSHD, specifically old, non-induced ACTA1-MCM;FLExDUX4 mice in which DUX4 and DUX4 downstream genes are expressed at low, leaky expression levels.

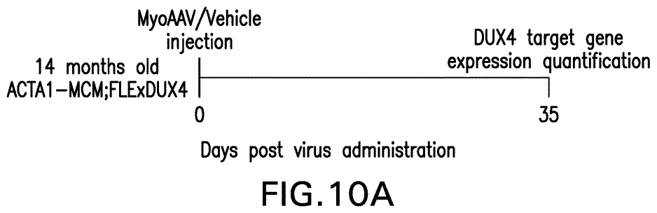

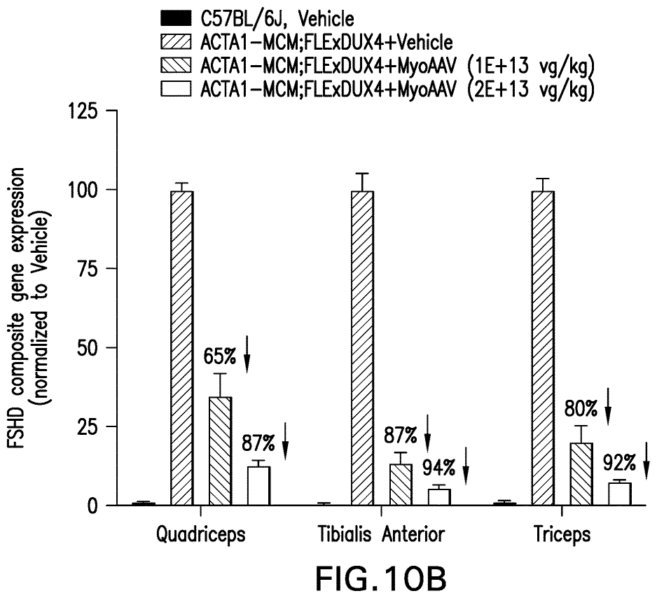

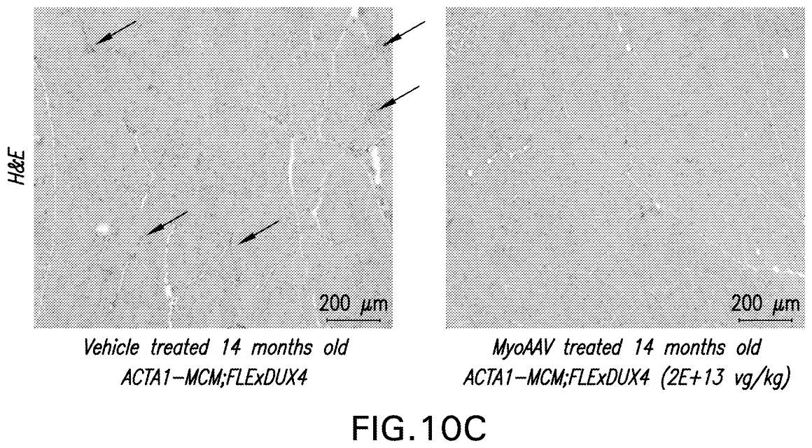

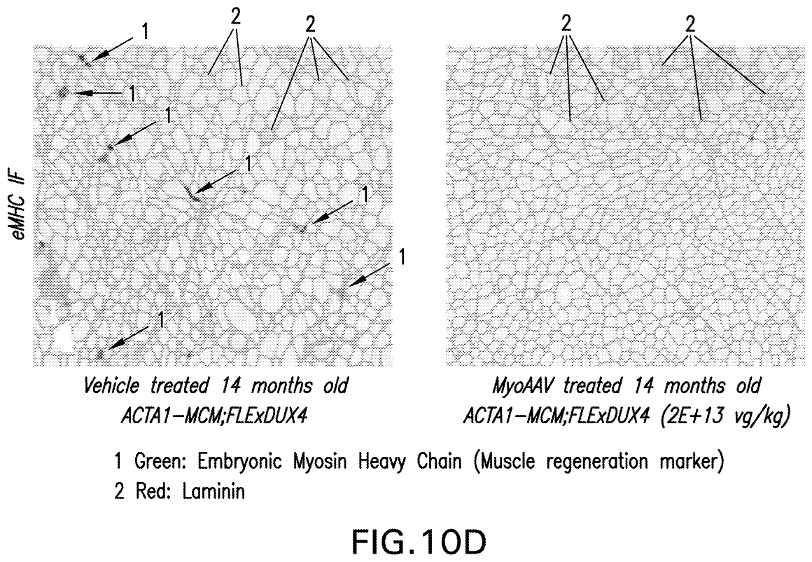

Composition 1 (dosed at 1E+13 vg/kg or 2E+13 vg/kg) or vehicle was administered to 14 months old ACTA1-MCM;FLExDUX4 mice. After 35 days, mice were necropsied, and skeletal muscles were assessed for DUX4 downstream gene expression and muscle regeneration. FIG. 10A shows a study design for testing Composition 1 in a mild mouse model of FSHD.

FIG. 10B shows a graph of FSHD composite gene expression in 14 months old ACTA1-MCM;FLExDUX4 mice following administration of Composition 1 at a dose of 1E+13 vg/kg or 2E+13 vg/kg. FIG. 10C shows representative images of skeletal muscle from mice treated with Composition 1 (2E+13 vg/kg) or vehicle. Muscle tissues were stained with hematoxylin and eosin (H&E). Active muscle degeneration following tamoxifen induced DUX4 expression was observed in mice administered vehicle (FIG. 10C, left panel), but not mice administered Composition 1 (FIG. 10C, right panel). Arrows show an area of active degeneration. These results demonstrate that administration of the lead candidate prevented visible muscle degeneration in a mild mouse model of FSHD.

FIG. 10D shows immunofluorescent images of mouse skeletal muscle stained for embryonic myosin heavy chain (muscle regeneration marker) and laminin (marker outlining individual muscle fibers) from mice administered vehicle or Composition 1 (2E+13 vg/kg). As shown in FIG. 10D, administration of Composition 1 showed prevented the need for muscle regeneration in mouse tissue in a mild mouse model of FSHD.

Taken together, these results demonstrate that Composition 1-injected mice showed improved muscle histology and function (e.g., lower FSHD composite gene expression, less active degeneration, and lower muscle regeneration marker) compared to vehicle-injected mice. Thus, these results demonstrate that Composition 1 can protect against DUX4 induced muscle damage in a mild mouse model of FSHD.

### Example 12: Composition 1 Expresses DUX4 Targeting miRNA at Therapeutic Levels in Skeletal Muscles of Mice and Non-Human Primates (NHPs)

Biodistribution and safety of Composition 1 was assessed in ACTA1-MCM;FLExDUX4 mice (dosed at 3E+12 vg/kg, 1E+13 vg/kg, or 3E+13 vg/kg) and NHPs (dosed at 2E+13 vg/kg or 4E+13 vg/kg). Mice and NHPs were administered the indicated dose and then necropsied 20 days later for mice and 90 days later for NHPs. DUX4 miRNA expression was quantified in 5 different muscle tissues from mice and 8 different muscle tissues from NHPs.

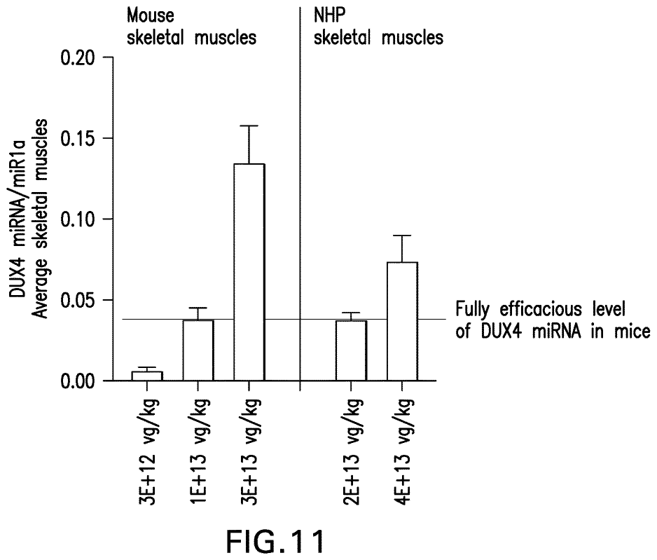

FIG. 11 shows a graph of average normalized DUX4 targeting miR expression across skeletal muscles (5 muscles in mice and 8 muscles in NHPs) at different doses of Composition 1. Mice administered 1E+13 vg/kg of Composition 1 showed full functional recovery, e.g., as determined by rescue of defects in treadmill assessments (see FIGS. 9B-9C). These results demonstrate that Composition 1 can express therapeutic levels of the lead DUX4 miRNA in skeletal muscles of mice and NHPs.

### Example 13: Administration of Composition 2 Rescued Biochemical and Histological Phenotypes in a Severe Mouse Model of FSHD

The ability of Composition 2 to protect muscle against DUX4 mediated damage and to reduce expression of DUX4 biomarkers was tested in the ACTA1-MCM:FLExDUX4 mouse model of FSHD. Composition 2 includes the cargo nucleic acid sequence (SEQ ID NO: 3941) and the AAV capsid protein MyoAAV-LD B, both of which are described herein.

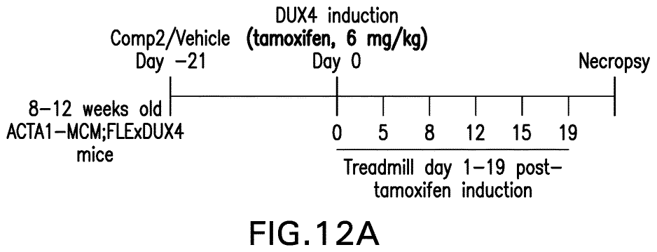

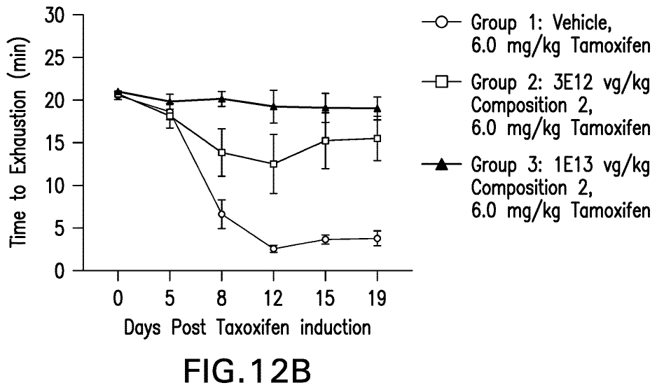

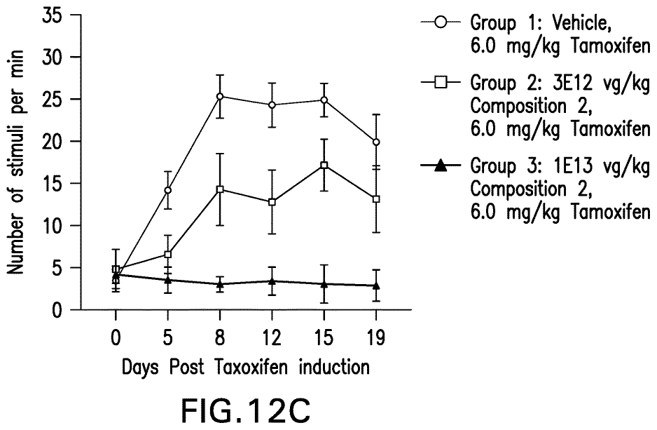

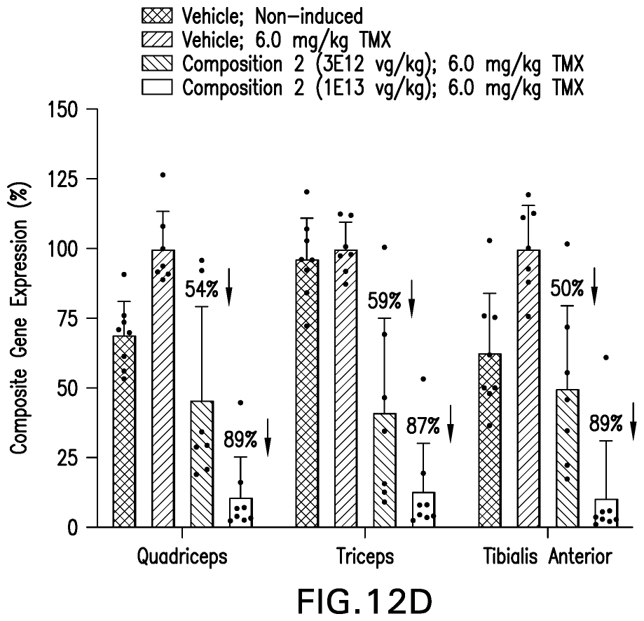

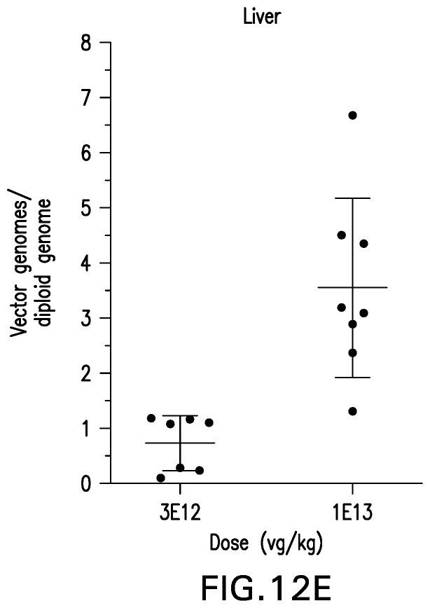

Composition 2 (dosed at 3E12 vg/kg or 1E+13 vg/kg) or vehicle was administered to 8-12 week old ACTA1-MCM;FLExDUX4 mice. After 21 days, mice were treated with tamoxifen (6.0 mg/kg) to induce DUX4 expression. Mice were assessed on a treadmill on days 1-19 post-tamoxifen induction. Mice were necropsied on day 20 post-taxmoxifen induction. Skeletal muscles were assessed for DUX4 downstream gene expression. Transduction of liver tissue was measured as the abundance of liver genome per diploid genome. FIG. 12A shows a study design for testing Composition 2 in a severe mouse model of FSHD.

FIGS. 12B-12C show graphs of results from treadmill assessments of mice administered Composition 2 prior to tamoxifen induced DUX4 expression. As shown in FIGS. 12B-12C, administration of Composition 2 prevented a decrease in time to exhaustion (FIG. 12B) and a decrease in number of stimuli per minute (FIG. 12C) in a severe mouse model of FSHD.

FIG. 12D shows a graph of FSHD composite gene expression in skeletal muscle (quadriceps, triceps, tibialis anterior) of mice administered Composition 2 or vehicle. As shown in FIG. 12D, administration of Composition 2 at a dose of 3E+12 vg/kg or 1E+13 vg/kg resulted in 50-59% or 87-89% decrease in FSHD composite gene expression, respectively.

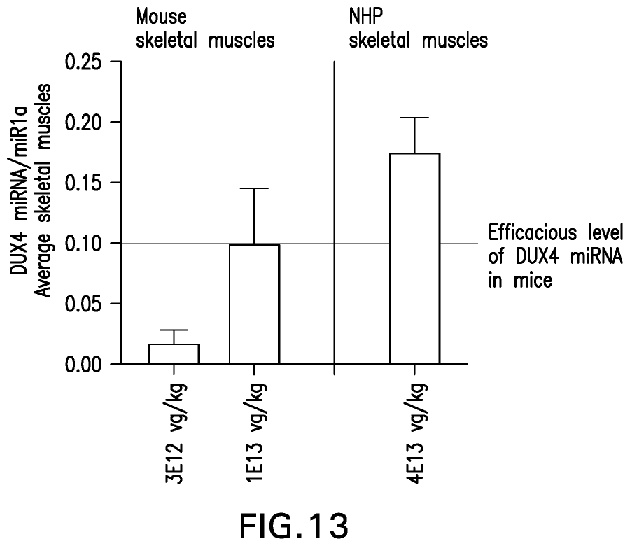

FIG. 12E shows a graph of vector genome detected in the liver of Composition 2-injected mice. The number of vector genome per diploid genome was about 1 for mice injected with Composition 2 at a dose of 3E+12 vg/kg and about 3.5 for Composition 2 at a dose of 3E+12 vg/kg (FIG. 13E).

Taken together, these results demonstrate that treatment with Composition 2 can protect against DUX4 mediated damage to skeletal muscle with minimal transduction of the liver in a severe mouse model of FSHD.

### Example 14: MyoAAV-LD B Used in Composition 1 and Composition 2 Transduces Muscle Fibers After Systemic Administration in NHPs

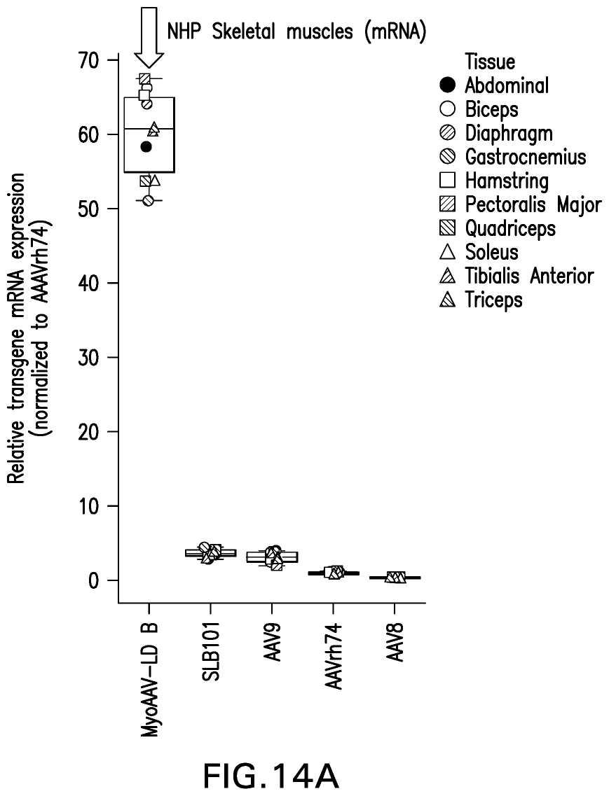

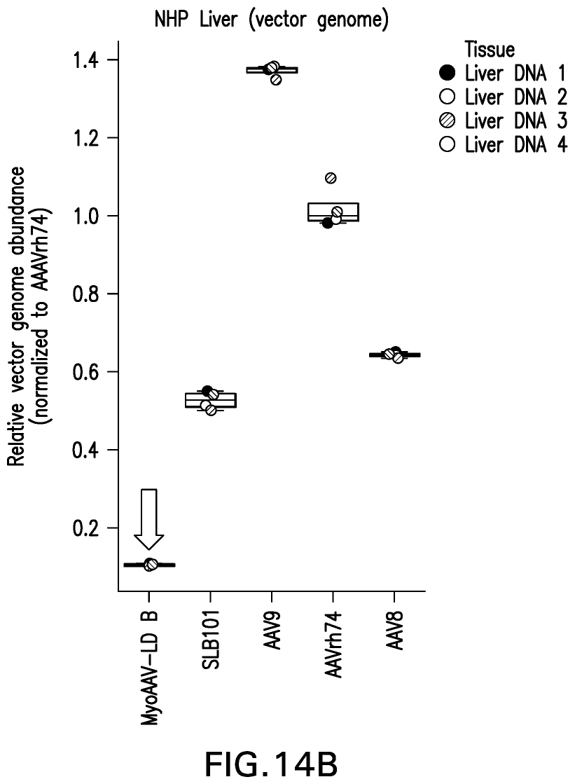

MyoAAV-LD B was benchmarked against naturally occurring (AAV8, AAV9) and engineered (SLB101, AAVrh74) AAV capsids in NHPs. A uniquely barcoded transgene was packaged in each capsid and the pool of capsids was injected into NHPs. Abundance of transgene mRNA was measured by next-generation sequencing (NGS). MyoAAV-LD B showed ~60 times higher transgene expression (FIG. 14A) and ~10 times lower vector genome levels in the liver compared to AAVrh74 (FIG. 14B).

Taken together, these results demonstrate that MyoAAV-LD B, which is used in Composition 1 and Composition 2, significantly outperforms competitor capsids in transducing NHP muscle and in de-targeting from the liver after systemic administration.

### Example 15: Safe Gene Therapy Candidate for Facioscapulohumeral Muscular Dystrophy (FSHD) Through High Throughput AAV Capsid and Cargo Engineering

Facioscapulohumeral muscular dystrophy (FSHD) is an autosomal-dominant genetic disorder affecting approximately 1 in 8,000 individuals. FSHD patients typically exhibit progressive muscle wasting in the face, shoulders, upper arms, legs, and abdomen. Approximately 20% of patients will eventually require the use of a wheelchair. De-repression of the transcription factor DUX4 has been identified as the cause of muscle degeneration in FSHD. Insufficient epigenetic silencing of the DUX4 gene in patient muscles results in aberrant expression of DUX4, which is toxic to mature myofibers. Therefore, knockdown of the DUX4 transcript has been evaluated as a therapeutic strategy for FSHD and has shown efficacy in preclinical models.

Efficacy of a novel MyoAAV-LD-mediated RNAi therapy for FSHD described here was demonstrated, both in vitro in FSHD patient-derived myotubes and in vivo in the ACTA1-MCM;FLExDUX4 mouse model. Biodistribution and safety was evaluated in non-human primates (NHPs). First, a high throughput cell-based tiling screen to identify the most potent DUX4-targeting artificial miRNA sequences was conducted. Top hits from this screen were validated in FSHD patient-derived myotubes. These cells contain a disease-causing contraction in the D4Z4 region of chromosome 4q that is responsible for epigenetic silencing of DUX4, and upon differentiation, express DUX4 and its transcriptionally regulated target genes. Candidate miRNA sequences were ranked based on the magnitude of DUX4 and DUX4 target gene knockdown achieved in these cells, and the top miRNAs were evaluated for off-target effects in control human myotubes by RNAseq and small RNAseq. Lead DUX4 targeting miRNA of the invention were selected based on high potency in FSHD myotubes and lack of significant off-target effects in control human myotubes.

In separate experiments, a potent liver de-targeted, muscle-tropic MyoAAV-LD capsid variant (MyoAAV-LD B) was evolved in non-human primates. The transgene encoding the lead miRNA was packaged into MyoAAV-LD B and efficacy of this development candidate tested in vitro and in vivo. Dose-dependent knockdown of DUX4 and DUX4 target genes in FSHD patient-derived myotubes and minimal off-target effects in control human myotubes after transduction was demonstrated. To assess in vivo efficacy, ACTA1-MCM;FLExDUX4 mice were used, a bi-transgenic line with inducible expression of DUX4 in skeletal muscles, which reproduces disease-relevant biochemical, histological, and functional phenotypes of FSHD. These mice were injected with increasing doses of the lead candidate or vehicle, and DUX4 expression was induced in the mice by injecting tamoxifen. Treatment with the lead candidate dose-dependently protected mice from severe impairment in treadmill time to exhaustion, protected their muscles from DUX4-mediated degeneration and regeneration, and blocked elevations in DUX4 and DUX4 target genes, all of which were observed in the vehicle-treated mice. Similar treatment also rescued biochemical and histological phenotypes in aged, non-induced ACTA1-MCM;FLExDUX4 mice. Finally, biodistribution and safety of the lead candidate was assessed in NHPs at 2E+13 vg/kg and 4E+13 vg/kg. miRNA expression levels were achieved in NHP skeletal muscles comparable to those shown to be efficacious in the ACTA1-MCM;FLExDUX4 mice. The lead candidate was safe and well tolerated in the NHP study.

### CONCLUSION

AAV compositions described herein (combining an engineered liver de-targeted MyoAAV capsid comprising the amino acid sequence RGDR, a promoter, and a miR candidate described herein comprising the scaffold miR-138 or miR-139 together with a guide that targets a DUX4 transcript) results in decreased toxic DUX4 expression.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1.-10. (canceled)

11. A recombinant nucleic acid comprising a primary microRNA (pri-miRNA), wherein the pri-miRNA comprises:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920;
wherein the guide strand is positioned between the upstream scaffold sequence and the loop scaffold sequence, and the passenger strand is positioned between the loop scaffold sequence and the downstream scaffold sequence.

12. The recombinant nucleic acid claim 11, wherein the recombinant nucleic acid is selected from:
(a) a recombinant nucleic acid comprising:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
(b) a recombinant nucleic acid comprising:
a upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920;
(c) a recombinant nucleic acid comprising:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
(d) a recombinant nucleic acid comprising:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
(e) a recombinant nucleic acid comprising:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630; and
(f) a recombinant nucleic acid comprising:
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a guide strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429; and
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190.

13. The recombinant nucleic acid of claim 12, wherein the recombinant nucleic acid is selected from:
(a) a recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2475; and
a passenger strand comprising or consisting of the nucleic acid sequence of SEQ ID NO: 3236;
(b) a recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 3159; and
a passenger strand comprising or consisting of the nucleic acid sequence of SEQ ID NO: 3920;
(c) a recombinant nucleic acid comprising:
an upstream scaffold sequence comprising or consisting of the nucleic acid sequence of SEQ ID NO: 3929;
a loop scaffold sequence comprising or consisting of the nucleic acid sequence of SEQ ID NO: 3930;
a downstream scaffold sequence comprising or consisting of the nucleic acid sequence of SEQ ID NO: 3931;
a guide strand comprising or consisting of the nucleic acid sequence of SEQ ID NO: 3047; and
a passenger strand comprising or consisting of the nucleic acid sequence of SEQ ID NO: 3808;
(d) a recombinant nucleic acid comprising:
an upstream scaffold sequence comprising or consisting of the nucleic acid sequence of SEQ ID NO: 3929;
a loop scaffold sequence comprising or consisting of the nucleic acid sequence of SEQ ID NO: 3930;
a downstream scaffold sequence comprising or consisting of the nucleic acid sequence of SEQ ID NO: 3931;
a guide strand comprising or consisting of the nucleic acid sequence of SEQ ID NO: 2475; and
a passenger strand comprising or consisting of the nucleic acid sequence of SEQ ID NO: 3236;
(e) a recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2869; and
a passenger strand comprising or consisting of the nucleic acid sequence of SEQ ID NO: 3630; and
(f) a recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2429; and
a passenger strand comprising or consisting of the nucleic acid sequence of SEQ ID NO: 3190.

14. The recombinant nucleic acid of claim 11, further comprising at least one dorsal root ganglia (DRG) de-targeting miRNA binding site.

15. The recombinant nucleic acid of claim 14, wherein the at least one DRG de-targeting miRNA binding site comprises a binding site for miR-338, miR-138, or miR-9.

16. The recombinant nucleic acid of claim 14, wherein the at least one DRG de-targeting miRNA binding site comprises a binding site for miR-338-3p, miR-138-5p, or miR-9-5p.

17. The recombinant nucleic acid of claim 14, wherein the at least one DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923.

18. The recombinant nucleic acid of claim 14, wherein the at least one DRG de-targeting miRNA binding site comprises or consists of the nucleic acid sequence of any one of SEQ ID NOs: 3921-3923.

19. The recombinant nucleic acid of claim 14, wherein the at least one DRG de-targeting miRNA binding site comprises a first and a second DRG de-targeting miRNA binding site.

20. The recombinant nucleic acid of claim 14, wherein each of the first and the second DRG de-targeting miRNA binding sites comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923.

21. The recombinant nucleic acid of claim 14, wherein each of the first and the second DRG de-targeting miRNA binding sites comprises or consists of the nucleic acid sequence of any one of SEQ ID NOs: 3921-3923.

22. The recombinant nucleic acid of claim 14, wherein each of the first and the second DRG de-targeting miRNA binding sites comprises or consists of the nucleic acid sequence of SEQ ID NO: 3921.

23. The recombinant nucleic acid of claim 11, further comprising at least one heart de-targeting miRNA binding site.

24. The recombinant nucleic acid of claim 23, wherein the at least one heart de-targeting miRNA binding site comprises a binding site for miR-221 or miR-499.

25. The recombinant nucleic acid of claim 23, wherein the at least one heart de-targeting miRNA binding site comprises a binding site for miR-221-3p or miR-499a.

26. The recombinant nucleic acid of claim 23, wherein the at least one heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925.

27. The recombinant nucleic acid of claim 23, wherein the at least one heart de-targeting miRNA binding site comprises or consists of the nucleic acid sequence of SEQ ID NO: 3924 or SEQ ID NO: 3925.

28. The recombinant nucleic acid of claim 23, wherein the at least one heart de-targeting miRNA binding site comprises a first and a second heart de-targeting miRNA binding site.

29. The recombinant nucleic acid of claim 23, wherein each of the first and the second heart de-targeting miRNA binding sites comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925.

30. The recombinant nucleic acid of claim 23, wherein each of the first and the second heart de-targeting miRNA binding sites comprises or consists of the nucleic acid sequence of SEQ ID NO: 3924 or SEQ ID NO: 3925.

31. The recombinant nucleic acid of claim 23, wherein each of the first and the second heart de-targeting miRNA binding sites comprises or consists of the nucleic acid sequence of SEQ ID NO: 3924.

32. The recombinant nucleic acid of claim 23, wherein the recombinant nucleic acid comprises at least two DRG de-targeting miRNA binding sites and at least two heart de-targeting miRNA binding sites, which are in alternating positions.

33. The recombinant nucleic acid of claim 11, wherein the recombinant nucleic acid comprises, from 5′ to 3′:
a first DRG de-targeting miRNA binding site,
a first heart de-targeting miRNA binding site,
a second DRG de-targeting miRNA binding site, and
a second heart de-targeting miRNA binding site.

34. The recombinant nucleic acid of claim 11, further comprising a muscle specific promoter.

35. The recombinant nucleic acid of claim 34, wherein the muscle specific promoter comprises a CK8 promoter, a desmin promoter, a Mb promoter, a MCK promoter, a MHCK7 promoter, a skeletal muscle alpha actin promoter, or a TTNI2 promoter.

36. The recombinant nucleic acid of claim 34, wherein the muscle specific promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928.

37. The recombinant nucleic acid of claim 34, wherein the muscle specific promoter comprises or consists of the nucleic acid sequence of any one of SEQ ID NOs: 3926-3928.

38. The recombinant nucleic acid of claim 34, wherein the muscle specific promoter comprises or consists of the nucleic acid sequence of SEQ ID NO: 3926.

39. The recombinant nucleic acid of claim 11, further comprising a polyadenylation (PolyA) signal.

40. The recombinant nucleic acid of claim 39, wherein the PolyA signal comprises a bovine growth hormone polyadenylation (bgh-PolyA) signal, a synthetic PolyA signal, or an SV40 PolyA signal.

41. The recombinant nucleic acid of claim 39, wherein the PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938.

42. The recombinant nucleic acid of claim 39, wherein the PolyA signal comprises or consists of the nucleic acid sequence of SEQ ID NO: 3938.

43. The recombinant nucleic acid of claim 11, further comprising a 5′ inverted terminal repeat (ITR) and a 3′ ITR.

44. The recombinant nucleic acid of claim 43, wherein the 5′ ITR and/or the 3′ ITR is an adeno-associated virus 2 (AAV2) ITR, or a variant or fragment thereof.

45. The recombinant nucleic acid of claim 43, wherein the 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937.

46. The recombinant nucleic acid of claim 43, wherein the 5′ ITR comprises or consists of the nucleic acid sequence of SEQ ID NO: 3937.

47. The recombinant nucleic acid of claim 43, wherein the 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

48. The recombinant nucleic acid of claim 43, wherein the 3′ ITR comprises or consists of the nucleic acid sequence of SEQ ID NO: 3939.

49. The recombinant nucleic acid of claim 11, wherein the recombinant nucleic acid comprises, optionally from 5′ to 3′:
a 5′ ITR;
a muscle specific promoter;
an upstream scaffold sequence;
a guide strand targeting a DUX4 sequence;
a loop scaffold sequence;
a passenger strand;
a downstream scaffold sequence;
a first DRG de-targeting miRNA binding site;
a first heart de-targeting miRNA binding site;
a second DRG de-targeting miRNA binding site;
a second heart de-targeting miRNA binding site;
a PolyA signal; and
a 3′ ITR.

50. The recombinant nucleic acid of claim 49, wherein:
the 5′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
the muscle specific promoter comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3926-3928;
the upstream scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929 or SEQ ID NO: 3933;
the guide strand targeting DUX4 comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 2399-3159 and 3952-3953;
a loop scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930 or SEQ ID NO: 3934; and
the passenger strand comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3160-3920;
the downstream scaffold sequence comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931 or SEQ ID NO: 3935;
the first DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
the first heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
the second DRG de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3921-3923;
the second heart de-targeting miRNA binding site comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924 or SEQ ID NO: 3925;
the PolyA signal comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
the 3′ ITR comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

51. The recombinant nucleic acid of claim 49, wherein the recombinant nucleic acid is selected from:
(a) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(b) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3159;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3920;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(c) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3928;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(d) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3047;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3808;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(e) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3922;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(f) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3926;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2869;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3630;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3921;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939;
(g) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3933;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2429;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3934;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3190;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3935;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939; and
(h) a recombinant nucleic acid comprising:
a 5′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3937;
a promoter comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3927;
an upstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3929;
a guide strand targeting DUX4 comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 2475;
a loop scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3930;
a passenger strand comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3236;
a downstream scaffold sequence comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3931;
a first DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3923;
a first heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3924;
a second DRG de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NOs: 3921;
a second heart de-targeting miRNA binding site comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3925;
a PolyA signal comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3938; and
a 3′ ITR comprising or consisting of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3939.

52. The recombinant nucleic acid of claim 49, wherein the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of any one of SEQ ID NOs: 3940-3947.

53. The recombinant nucleic acid of claim 49, wherein the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3941.

54. The recombinant nucleic acid of claim 49, wherein the recombinant nucleic acid comprises or consists of a nucleic acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 3945.

55. The recombinant nucleic acid of claim 49, wherein the recombinant nucleic acid comprises or consists of the nucleic acid sequence of any one of SEQ ID NOs: 3940-3947.

56. The recombinant nucleic acid of claim 49, wherein the recombinant nucleic acid comprises or consists of the nucleic acid sequence of SEQ ID NO: 3941.

57. The recombinant nucleic acid of claim 49, wherein the recombinant nucleic acid comprises or consists of the nucleic acid sequence of SEQ ID NO: 3945.

58. An adeno-associated virus (AAV) particle comprising the recombinant nucleic acid of claim 11 and a capsid protein.

59. The AAV particle of claim 58, wherein the capsid protein is derived from a serotype selected from AAV1, AAV2, AAV3, AAV4, AAV5, AAV6, AAV6.2, AAV7, AAV8, AAV9, AAV10, AAV11, AAV12, AAV13, AAVrh, AAVrh10, AAVrh74, AAV-DJ, AAV-DJ/8, Anc80, and AAV7m8, or a derivative, hybrid or chimeric serotype thereof.

60. The AAV particle of claim 58, wherein the AAV particle has increased muscle tropism compared to a wild type AAV9 particle.

61. The AAV particle of claim 58, wherein the AAV particle has reduced liver tropism compared to a wild type AAV9 particle.

62. The AAV particle of claim 58, wherein the capsid protein comprises an amino acid sequence of formula (I):
X₁NX₂X₃X₄RGDRX₅X₆L, (I)
wherein each of X₁-X₆ is any amino acid.

63. The AAV particle of claim 62, wherein the amino acid sequence of formula (I) is in a hypervariable region IV (HVR IV) relative to a wild type AAV9 capsid.

64. The AAV particle of claim 62, wherein, relative to a wild type AAV9 capsid, X₁ is a substitution at amino acid 451, X₂ is a substitution at amino acid 453, X₃ is a substitution at amino acid 454, X₄ is a substitution at amino acid 455, and RGDRX₅X₆L is inserted after amino acid 455.

65. The AAV particle of claim 62, wherein:
X₁ is A, I, F, G, H, L, M, Q, S, T, or V;
X₂ is A, G, S, T, or Y;
X₃ is S, N, G, or P;
X₄ is A, G, H, I, M, S, T, or V;
X₅ is A, G, or Q; and
X₆ is A, I, L, M, N, S, or Y.

66. The AAV particle of claim 62, wherein the amino acid sequence of formula (I) comprises of any one of SEQ ID NOs: 801-1599.

67. The AAV particle of claim 62, wherein the amino acid sequence of formula (I) comprises of any one of SEQ ID NOs: 1600-2398.

68. The AAV particle of claim 62, wherein the amino acid sequence of formula (I) comprises or consists of any one of SEQ ID NOs: 2-800.

69. The AAV particle of claim 62, wherein the amino acid sequence of formula (I) comprises or consists of SEQ ID NOs: 191, 198, 199, 215, 256, 379, 544, 550, or 748.

70. The AAV particle of claim 62, wherein the capsid protein further comprises a deletion of amino acid G267 relative to a wild type AAV9 vector.

71. The AAV particle of claim 62, wherein the amino acid sequence of formula (I) comprises or consists of SEQ ID NO: 215, the capsid protein further comprises a deletion of amino acid G267 relative to a wild type AAV9 vector, and the remainder of the capsid is otherwise identical to the wild type AAV9.

72. A pharmaceutical composition comprising the recombinant nucleic acid of claim 11, and a pharmaceutically acceptable carrier.

73. A method of treating facioscapulohumeral muscular dystrophy (FSHD) in a subject in need thereof, the method comprising administering to the subject an effective amount of the recombinant nucleic acid of claim 11.

74. The method of claim 73, wherein the subject is a human subject.

75. The method of claim 73, wherein the subject has one or more deletions of a D4Z4 repeat in chromosome 4.

76. The method of claim 73, wherein the subject has one or more deletions in a SMCHD1 gene.

77. The method of claim 73, wherein the subject has one or more deletions in a DNMT3B gene.

78. The method of claim 73, wherein the subject has FSHD type 1 (FSHD1), FSHD type 2 (FSHD2), or a combination thereof (FSHD1+2).

79. The method of claim 73, wherein the administering comprises intramuscular administration, subcutaneous injection, intravenous injection, or intravenous (IV) administration.

80.-81. (canceled)

82. A kit comprising the recombinant nucleic acid of claim 11.

83. The kit of claim 81, further comprising instructions for use in treating facioscapulohumeral muscular dystrophy (FSHD) in a subject in need thereof.

84. A recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2475; and
a passenger strand comprising or consisting of the nucleic acid sequence of SEQ ID NO: 3236.

85. A recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 3159; and
a passenger strand comprising or consisting of the nucleic acid sequence of SEQ ID NO: 3920.

86. A recombinant nucleic acid comprising:
an upstream scaffold sequence comprising or consisting of the nucleic acid sequence of SEQ ID NO: 3929;
a loop scaffold sequence comprising or consisting of the nucleic acid sequence of SEQ ID NO: 3930;
a downstream scaffold sequence comprising or consisting of the nucleic acid sequence of SEQ ID NO: 3931;
a guide strand comprising or consisting of the nucleic acid sequence of SEQ ID NO: 3047; and
a passenger strand comprising or consisting of the nucleic acid sequence of SEQ ID NO: 3808.

87. A recombinant nucleic acid comprising:
an upstream scaffold sequence comprising or consisting of the nucleic acid sequence of SEQ ID NO: 3929;
a loop scaffold sequence comprising or consisting of the nucleic acid sequence of SEQ ID NO: 3930;
a downstream scaffold sequence comprising or consisting of the nucleic acid sequence of SEQ ID NO: 3931;
a guide strand comprising or consisting of the nucleic acid sequence of SEQ ID NO: 2475; and
a passenger strand comprising or consisting of the nucleic acid sequence of SEQ ID NO: 3236.

88. A recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2869; and
a passenger strand comprising or consisting of the nucleic acid sequence of SEQ ID NO: 3630.

89. A recombinant nucleic acid comprising:
an upstream scaffold sequence comprising or consisting of the nucleic acid sequence of SEQ ID NO: 3933;
a loop scaffold sequence comprising or consisting of the nucleic acid sequence of SEQ ID NO: 3934;
a downstream scaffold sequence comprising or consisting of the nucleic acid sequence of SEQ ID NO: 3935;
a guide strand comprising or consisting of the nucleic acid sequence of SEQ ID NO: 2429; and
a passenger strand comprising or consisting of the nucleic acid sequence of SEQ ID NO: 3190.

90. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3927;
an upstream scaffold sequence comprising or consisting of the nucleic acid sequence of SEQ ID NO: 3933;
a guide strand targeting DUX4 comprising or consisting of the nucleic acid sequence of SEQ ID NO: 2475;
a loop scaffold sequence comprising or consisting of the nucleic acid sequence of SEQ ID NO: 3934;
a passenger strand comprising or consisting of the nucleic acid sequence of SEQ ID NO: 3236;
a downstream scaffold sequence comprising or consisting of the nucleic acid sequence of SEQ ID NO: 3935;
a first DRG de-targeting miRNA binding site comprising or consisting of the nucleic acid sequence of SEQ ID NO: 3923;
a first heart de-targeting miRNA binding site comprising or consisting of the nucleic acid sequence of SEQ ID NO: 3925;
a second DRG de-targeting miRNA binding site comprising or consisting of the nucleic acid sequence of SEQ ID NOs: 3921;
a second heart de-targeting miRNA binding site comprising or consisting of the nucleic acid sequence of SEQ ID NO: 3924;
a PolyA signal comprising or consisting of the nucleic acid sequence of SEQ ID NO: 3938; and
a 3′ ITR comprising or consisting of the nucleic acid sequence of SEQ ID NO: 3939.

91. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3926;
an upstream scaffold sequence comprising or consisting of the nucleic acid sequence of SEQ ID NO: 3933;
a guide strand targeting DUX4 comprising or consisting of the nucleic acid sequence of SEQ ID NO: 3159;
a loop scaffold sequence comprising or consisting of the nucleic acid sequence of SEQ ID NO: 3934;
a passenger strand comprising or consisting of the nucleic acid sequence of SEQ ID NO: 3920;
a downstream scaffold sequence comprising or consisting of the nucleic acid sequence of SEQ ID NO: 3935;
a first DRG de-targeting miRNA binding site comprising or consisting of the nucleic acid sequence of SEQ ID NO: 3921;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3921;
a second heart de-targeting miRNA binding site comprising or consisting of the nucleic acid sequence of SEQ ID NO: 3924;
a PolyA signal comprising or consisting of the nucleic acid sequence of SEQ ID NO: 3938; and
a 3′ ITR comprising or consisting of the nucleic acid sequence of SEQ ID NO: 3939.

92. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3928;
an upstream scaffold sequence comprising or consisting of the nucleic acid sequence of SEQ ID NO: 3929;
a guide strand targeting DUX4 comprising or consisting of the nucleic acid sequence of SEQ ID NO: 3047;
a loop scaffold sequence comprising or consisting of the nucleic acid sequence of SEQ ID NO: 3930;
a passenger strand comprising or consisting of the nucleic acid sequence of SEQ ID NO: 3808;
a downstream scaffold sequence comprising or consisting of the nucleic acid sequence of SEQ ID NO: 3931;
a first DRG de-targeting miRNA binding site comprising or consisting of the nucleic acid sequence of SEQ ID NO: 3921;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3922;
a second heart de-targeting miRNA binding site comprising or consisting of the nucleic acid sequence of SEQ ID NO: 3925;
a PolyA signal comprising or consisting of the nucleic acid sequence of SEQ ID NO: 3938; and
a 3′ ITR comprising or consisting of the nucleic acid sequence of SEQ ID NO: 3939.

93. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3926;
an upstream scaffold sequence comprising or consisting of the nucleic acid sequence of SEQ ID NO: 3929;
a guide strand targeting DUX4 comprising or consisting of the nucleic acid sequence of SEQ ID NO: 3047;
a loop scaffold sequence comprising or consisting of the nucleic acid sequence of SEQ ID NO: 3930;
a passenger strand comprising or consisting of the nucleic acid sequence of SEQ ID NO: 3808;
a downstream scaffold sequence comprising or consisting of the nucleic acid sequence of SEQ ID NO: 3931;
a first DRG de-targeting miRNA binding site comprising or consisting of the nucleic acid sequence of SEQ ID NO: 3921;
a first heart de-targeting miRNA binding site comprising or consisting of the nucleic acid sequence of SEQ ID NO: 3925;
a second DRG de-targeting miRNA binding site comprising or consisting of the nucleic acid sequence of SEQ ID NOs: 3922;
a second heart de-targeting miRNA binding site comprising or consisting of the nucleic acid sequence of SEQ ID NO: 3925;
a PolyA signal comprising or consisting of the nucleic acid sequence of SEQ ID NO: 3938; and
a 3′ ITR comprising or consisting of the nucleic acid sequence of SEQ ID NO: 3939.

94. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3927;
an upstream scaffold sequence comprising or consisting of the nucleic acid sequence of SEQ ID NO: 3929;
a guide strand targeting DUX4 comprising or consisting of the nucleic acid sequence of SEQ ID NO: 2475;
a loop scaffold sequence comprising or consisting of the nucleic acid sequence of SEQ ID NO: 3930;
a passenger strand comprising or consisting of the nucleic acid sequence of SEQ ID NO: 3236;
a downstream scaffold sequence comprising or consisting of the nucleic acid sequence of SEQ ID NO: 3931;
a first DRG de-targeting miRNA binding site comprising or consisting of the nucleic acid sequence of SEQ ID NO: 3921;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3922;
a second heart de-targeting miRNA binding site comprising or consisting of the nucleic acid sequence of SEQ ID NO: 3925;
a PolyA signal comprising or consisting of the nucleic acid sequence of SEQ ID NO: 3938; and
a 3′ ITR comprising or consisting of the nucleic acid sequence of SEQ ID NO: 3939.

95. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3926;
an upstream scaffold sequence comprising or consisting of the nucleic acid sequence of SEQ ID NO: 3933;
a guide strand targeting DUX4 comprising or consisting of the nucleic acid sequence of SEQ ID NO: 2869;
a loop scaffold sequence comprising or consisting of the nucleic acid sequence of SEQ ID NO: 3934;
a passenger strand comprising or consisting of the nucleic acid sequence of SEQ ID NO: 3630;
a downstream scaffold sequence comprising or consisting of the nucleic acid sequence of SEQ ID NO: 3935;
a first DRG de-targeting miRNA binding site comprising or consisting of the nucleic acid sequence of SEQ ID NO: 3921;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3921;
a second heart de-targeting miRNA binding site comprising or consisting of the nucleic acid sequence of SEQ ID NO: 3924;
a PolyA signal comprising or consisting of the nucleic acid sequence of SEQ ID NO: 3938; and
a 3′ ITR comprising or consisting of the nucleic acid sequence of SEQ ID NO: 3939.

96. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3927;
an upstream scaffold sequence comprising or consisting of the nucleic acid sequence of SEQ ID NO: 3933;
a guide strand targeting DUX4 comprising or consisting of the nucleic acid sequence of SEQ ID NO: 2429;
a loop scaffold sequence comprising or consisting of the nucleic acid sequence of SEQ ID NO: 3934;
a passenger strand comprising or consisting of the nucleic acid sequence of SEQ ID NO: 3190;
a downstream scaffold sequence comprising or consisting of the nucleic acid sequence of SEQ ID NO: 3935;
a first DRG de-targeting miRNA binding site comprising or consisting of the nucleic acid sequence of SEQ ID NO: 3923;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3921;
a second heart de-targeting miRNA binding site comprising or consisting of the nucleic acid sequence of SEQ ID NO: 3925;
a PolyA signal comprising or consisting of the nucleic acid sequence of SEQ ID NO: 3938; and
a 3′ ITR comprising or consisting of the nucleic acid sequence of SEQ ID NO: 3939.

97. A recombinant nucleic acid comprising:
a 5′ ITR comprising or consisting of the nucleic acid sequence of SEQ ID NO: 3937;
a promoter comprising or consisting of the nucleic acid sequence of SEQ ID NO: 3927;
an upstream scaffold sequence comprising or consisting of the nucleic acid sequence of SEQ ID NO: 3929;
a guide strand targeting DUX4 comprising or consisting of the nucleic acid sequence of SEQ ID NO: 2475;
a loop scaffold sequence comprising or consisting of the nucleic acid sequence of SEQ ID NO: 3930;
a passenger strand comprising or consisting of the nucleic acid sequence of SEQ ID NO: 3236;
a downstream scaffold sequence comprising or consisting of the nucleic acid sequence of SEQ ID NO: 3931;
a first DRG de-targeting miRNA binding site comprising or consisting of the nucleic acid sequence of SEQ ID NO: 3923;
a first heart de-targeting miRNA binding site comprising or consisting of the nucleic acid sequence of SEQ ID NO: 3924;
a second DRG de-targeting miRNA binding site comprising or consisting of the nucleic acid sequence of SEQ ID NOs: 3921;
a second heart de-targeting miRNA binding site comprising or consisting of the nucleic acid sequence of SEQ ID NO: 3925;
a PolyA signal comprising or consisting of the nucleic acid sequence of SEQ ID NO: 3938; and
a 3′ ITR comprising or consisting of the nucleic acid sequence of SEQ ID NO: 3939.

98. A recombinant nucleic acid comprising or consisting of the nucleic acid sequence of SEQ ID NO: 3940.

99. A recombinant nucleic acid comprising or consisting of the nucleic acid sequence of SEQ ID NO: 3941.

100. A recombinant nucleic acid comprising or consisting of the nucleic acid sequence of SEQ ID NO: 3942.

101. A recombinant nucleic acid comprising or consisting of the nucleic acid sequence of SEQ ID NO: 3943.

102. A recombinant nucleic acid comprising or consisting of the nucleic acid sequence of SEQ ID NO: 3944.

103. A recombinant nucleic acid comprising or consisting of the nucleic acid sequence of SEQ ID NO: 3945.

104. A recombinant nucleic acid comprising or consisting of the nucleic acid sequence of SEQ ID NO: 3946.

105. A recombinant nucleic acid comprising or consisting of the nucleic acid sequence of SEQ ID NO: 3947.

106. An adeno-associated virus (AAV) particle, wherein the AAV particle comprises:
a recombinant nucleic acid comprising the nucleic acid sequence of SEQ ID NO: 3940; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

107. An adeno-associated virus (AAV) particle, wherein the AAV particle comprises:
a recombinant nucleic acid comprising the nucleic acid sequence of SEQ ID NO: 3941; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

108. An adeno-associated virus (AAV) particle, wherein the AAV particle comprises:
a recombinant nucleic acid comprising the nucleic acid sequence of SEQ ID NO: 3942; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

109. An adeno-associated virus (AAV) particle, wherein the AAV particle comprises:
a recombinant nucleic acid comprising the nucleic acid sequence of SEQ ID NO: 3943; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

110. An adeno-associated virus (AAV) particle, wherein the AAV particle comprises:
a recombinant nucleic acid comprising the nucleic acid sequence of SEQ ID NO: 3944; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

111. An adeno-associated virus (AAV) particle, wherein the AAV particle comprises:
a recombinant nucleic acid comprising the nucleic acid sequence of SEQ ID NO: 3945; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

112. An adeno-associated virus (AAV) particle, wherein the AAV particle comprises:
a recombinant nucleic acid comprising the nucleic acid sequence of SEQ ID NO: 3946; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

113. An adeno-associated virus (AAV) particle, wherein the AAV particle comprises:
a recombinant nucleic acid comprising the nucleic acid sequence of SEQ ID NO: 3947; and
a capsid protein comprising the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

114. An adeno-associated virus (AAV) particle, wherein the AAV particle comprises:
a recombinant nucleic acid comprising the nucleic acid sequence of SEQ ID NO: 3941; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

115. An adeno-associated virus (AAV) particle, wherein the AAV particle comprises:
a recombinant nucleic acid comprising the nucleic acid sequence of SEQ ID NO: 3942; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

116. An adeno-associated virus (AAV) particle, wherein the AAV particle comprises:
a recombinant nucleic acid comprising the nucleic acid sequence of SEQ ID NO: 3943; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

117. An adeno-associated virus (AAV) particle, wherein the AAV particle comprises:
a recombinant nucleic acid comprising the nucleic acid sequence of SEQ ID NO: 3944; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

118. An adeno-associated virus (AAV) particle, wherein the AAV particle comprises:
a recombinant nucleic acid comprising the nucleic acid sequence of SEQ ID NO: 3945; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

119. An adeno-associated virus (AAV) particle, wherein the AAV particle comprises:
a recombinant nucleic acid comprising the nucleic acid sequence of SEQ ID NO: 3946; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

120. An adeno-associated virus (AAV) particle, wherein the AAV particle comprises:
a recombinant nucleic acid comprising the nucleic acid sequence of SEQ ID NO: 3947; and
a capsid protein comprising the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

121. A pharmaceutical composition comprising a plurality of the AAV particles of claim 58 and a pharmaceutically acceptable carrier.

122. A pharmaceutical composition comprising a plurality of the AAV particles of claim 106 and a pharmaceutically acceptable carrier.

123. A pharmaceutical composition comprising a plurality of the AAV particles of claim 107 and a pharmaceutically acceptable carrier.

124. A pharmaceutical composition comprising a plurality of the AAV particles of claim 108 and a pharmaceutically acceptable carrier.

125. A pharmaceutical composition comprising a plurality of the AAV particles of claim 109 and a pharmaceutically acceptable carrier.

126. A pharmaceutical composition comprising a plurality of the AAV particles of claim 110 and a pharmaceutically acceptable carrier.

127. A pharmaceutical composition comprising a plurality of the AAV particles of claim 111 and a pharmaceutically acceptable carrier.

128. A pharmaceutical composition comprising a plurality of the AAV particles of claim 112 and a pharmaceutically acceptable carrier.

129. A pharmaceutical composition comprising a plurality of the AAV particles of claim 113 and a pharmaceutically acceptable carrier.

130. A pharmaceutical composition comprising a plurality of the AAV particles of claim 114 and a pharmaceutically acceptable carrier.

131. A pharmaceutical composition comprising a plurality of the AAV particles of claim 115 and a pharmaceutically acceptable carrier.

132. A pharmaceutical composition comprising a plurality of the AAV particles of claim 116 and a pharmaceutically acceptable carrier.

133. A pharmaceutical composition comprising a plurality of the AAV particles of claim 117 and a pharmaceutically acceptable carrier.

134. A pharmaceutical composition comprising a plurality of the AAV particles of claim 118 and a pharmaceutically acceptable carrier.

135. A pharmaceutical composition comprising a plurality of the AAV particles of claim 119 and a pharmaceutically acceptable carrier.

136. A pharmaceutical composition comprising a plurality of the AAV particles of claim 120 and a pharmaceutically acceptable carrier.

137. A method of treating facioscapulohumeral muscular dystrophy (FSHD) in a subject in need thereof, the method comprising administering to the subject an effective amount of the pharmaceutical composition of claim 121.

138. A method of treating facioscapulohumeral muscular dystrophy (FSHD) in a subject in need thereof, the method comprising administering to the subject an effective amount of the pharmaceutical composition of claim 122.

139. A method of treating facioscapulohumeral muscular dystrophy (FSHD) in a subject in need thereof, the method comprising administering to the subject an effective amount of the pharmaceutical composition of claim 123.

140. A method of treating facioscapulohumeral muscular dystrophy (FSHD) in a subject in need thereof, the method comprising administering to the subject an effective amount of the pharmaceutical composition of claim 124.

141. A method of treating facioscapulohumeral muscular dystrophy (FSHD) in a subject in need thereof, the method comprising administering to the subject an effective amount of the pharmaceutical composition of claim 125.

142. A method of treating facioscapulohumeral muscular dystrophy (FSHD) in a subject in need thereof, the method comprising administering to the subject an effective amount of the pharmaceutical composition of claim 126.

143. A method of treating facioscapulohumeral muscular dystrophy (FSHD) in a subject in need thereof, the method comprising administering to the subject an effective amount of the pharmaceutical composition of claim 127.

144. A method of treating facioscapulohumeral muscular dystrophy (FSHD) in a subject in need thereof, the method comprising administering to the subject an effective amount of the pharmaceutical composition of claim 128.

145. A method of treating facioscapulohumeral muscular dystrophy (FSHD) in a subject in need thereof, the method comprising administering to the subject an effective amount of the pharmaceutical composition of claim 129.

146. A method of treating facioscapulohumeral muscular dystrophy (FSHD) in a subject in need thereof, the method comprising administering to the subject an effective amount of the pharmaceutical composition of claim 130.

147. A method of treating facioscapulohumeral muscular dystrophy (FSHD) in a subject in need thereof, the method comprising administering to the subject an effective amount of the pharmaceutical composition of claim 131.

148. A method of treating facioscapulohumeral muscular dystrophy (FSHD) in a subject in need thereof, the method comprising administering to the subject an effective amount of the pharmaceutical composition of claim 132.

149. A method of treating facioscapulohumeral muscular dystrophy (FSHD) in a subject in need thereof, the method comprising administering to the subject an effective amount of the pharmaceutical composition of claim 133.

150. A method of treating facioscapulohumeral muscular dystrophy (FSHD) in a subject in need thereof, the method comprising administering to the subject an effective amount of the pharmaceutical composition of claim 134.

151. A method of treating facioscapulohumeral muscular dystrophy (FSHD) in a subject in need thereof, the method comprising administering to the subject an effective amount of the pharmaceutical composition of claim 135.

152. A method of treating facioscapulohumeral muscular dystrophy (FSHD) in a subject in need thereof, the method comprising administering to the subject an effective amount of the pharmaceutical composition of claim 136.

153. A kit comprising the pharmaceutical composition of claim 121.

154. A kit comprising the pharmaceutical composition of claim 122.

155. A kit comprising the pharmaceutical composition of claim 123.

156. A kit comprising the pharmaceutical composition of claim 124.

157. A kit comprising the pharmaceutical composition of claim 125.

158. A kit comprising the pharmaceutical composition of claim 126.

159. A kit comprising the pharmaceutical composition of claim 127.

160. A kit comprising the pharmaceutical composition of claim 128.

161. A kit comprising the pharmaceutical composition of claim 129.

162. A kit comprising the pharmaceutical composition of claim 130.

163. A kit comprising the pharmaceutical composition of claim 131.

164. A kit comprising the pharmaceutical composition of claim 132.

165. A kit comprising the pharmaceutical composition of claim 133.

166. A kit comprising the pharmaceutical composition of claim 134.

167. A kit comprising the pharmaceutical composition of claim 135.

168. A kit comprising the pharmaceutical composition of claim 136.

169. The AAV particle of claim 58, wherein the amino acid sequence of formula (I) comprises or consists of the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, RGDRQYL (SEQ ID NO: 1813) is inserted after amino acid 455, and G at amino acid 267 is deleted.

170. The AAV particle of claim 58, wherein the amino acid sequence of formula (I) comprises or consists of the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, RGDRQSL (SEQ ID NO: 1797) is inserted after amino acid 455, and G at amino acid 267 is deleted.

171. The AAV particle of claim 58, wherein the amino acid sequence of formula (I) comprises or consists of the amino acid sequence LNGSTRGDRQYL (SEQ ID NO: 256), wherein, relative to a wild-type AAV9 capsid protein, L is substituted for I at amino acid 451, T is substituted for G at amino acid 455, RGDRQYL (SEQ ID NO: 1813) is inserted after amino acid 455, and G at amino acid 267 is deleted.

172. The AAV particle of claim 58, wherein the amino acid sequence of formula (I) comprises or consists of the amino acid sequence SNASTRGDRQYL (SEQ ID NO: 544), wherein, relative to a wild-type AAV9 capsid protein, S is substituted for I at amino acid 451, A is substituted for G at amino acid 453, T is substituted for G at amino acid 455, and RGDRQYL (SEQ ID NO: 1813) is inserted after amino acid 455, and G at amino acid 267 is deleted.

173. The AAV particle of claim 58, wherein the amino acid sequence of formula (I) comprises or consists of the amino acid sequence VNGSTRGDRQYL (SEQ ID NO: 748), wherein, relative to a wild-type AAV9 capsid protein, V is substituted for I at amino acid 451, T is substituted for G at amino acid 455, and RGDRQYL (SEQ ID NO: 1813) is inserted after amino acid 455, and G at amino acid 267 is deleted.
